(19) United States
(12) Patent Application Publication
(10) Pub. No.: US 2008/0103123 A1
(43) Pub. Date: May 1, 2008

(54) New compounds

(75) Inventors: Peter Brandt, Uppsala (SE); Rikard Emond, Saltsjobaden (SE); Gary Johansson, Solna (SE); Lars Johansson, Bromma (SE); Tobias Koolmeister, Uppsala (SE); Bjorn Nilsson, Stockholm (SE); Teresa Sandvall, Stockholm (SE); Michael Weber, Kista (SE)

Correspondence Address: EDWARDS ANGELL PALMER & DODGE LLP, P.O. BOX 55874, BOSTON, MA, 02205, US

(73) Assignee: Biovitrum, Stockholm (SE)

(21) Appl. No.: 11/897,436

(22) Filed: Aug. 29, 2007

Related U.S. Application Data
(60) Provisional application No. 60/860,737, filed on Nov. 21, 2006.

(30) Foreign Application Priority Data
Aug. 30, 2006 (SE) 0601775-0

(51) Int. Cl. A61K 31/4545; A61K 31/397; A61K 31/5355; A61K 31/5377; A61P 3/00; C07D 211/22; C07D 413/14

(52) U.S. Cl. 514/210.2; 514/228.8; 514/235.5; 514/318; 544/130; 544/96; 546/194

(57) ABSTRACT

The present invention relates to compounds of Formula (Ia) 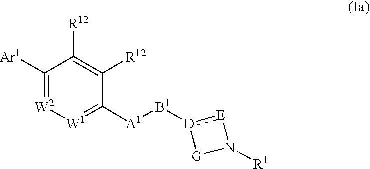
and pharmaceutically acceptable salts, hydrates, geometrical isomers, racemates, tautomers, optical isomers and N-oxides thereof, wherein one of W¹ and W² is N and the other is CR¹². The invention also relates to pharmaceutical compositions comprising these compounds, and to the use of these compounds for the prophylaxis and treatment of medical conditions relating to disorders of the G-protein-coupled receptor GPR119, such as diabetes and obesity.

### CROSS-REFERENCE TO RELATED APPLICATIONS

This application claims benefit of U.S. provisional application 60/860,737 filed Nov. 21, 2006, and Swedish application 0601775-0 filed Aug. 30, 2006, the entire contents of each which is herein incorporated by reference.

### FIELD OF INVENTION

The present invention relates to certain novel compounds, to pharmaceutical compositions comprising these novel compounds, and to the use of these compounds for the prophylaxis and treatment of medical conditions relating to disorders of the G-protein-coupled receptor GPR119 such as diabetes and obesity.

### BACKGROUND ART

Diabetes mellitus is a group of disorders characterized by abnormal glucose homeostasis resulting in high levels of blood glucose. The most common cases of diabetes mellitus are Type 1 (also referred to as insulin-dependent diabetes mellitus or IDDM) and Type 2 diabetes (also referred to as non-insulin-dependent diabetes mellitus or NIDDM). Type 2 diabetes accounts for approximately 90% of all diabetic cases. Type 2 diabetes is a serious progressive disease that results in the development of microvascular complications (e.g. retinopathy, neuropathy, nephropathy) as well as macrovascular complications (e.g. accelerated atherosclerosis, coronary heart disease, stroke). More than 75% of people with Type 2 diabetes die of cardiovascular diseases.

The increasing prevalence of obesity together with an ageing population is contributing to the predicted explosion in diabetes across the globe. Current projections suggest that 300 million people worldwide have diabetes by 2025.

The pathogenesis of Type 2 diabetes involves insulin resistance, insulin secretory dysfunction (i.e. pancreatic beta cell dysfunction) and hepatic glucose overproduction. Insulin resistance is highly correlated with obesity. Accumulating reports suggest insulin resistance to be central to a cluster of metabolic abnormalities—including dyslipidemia, hypertension, endothelial dysfunction, reduced fibrinolysis, and chronic systemic inflammation—that together are responsible for the increased cardiovascular risk.

Current antidiabetic therapy is targeting the defects mentioned above. For instance, sulphonylureas increase production of endogenous insulin. However, this enhanced insulin production is not glucose dependent and there is risk for developing hypoglycaemia. Metformin lowers hepatic glucose output. Thiazolidindiones (TZDs) reduce insulin resistance in muscle and liver and suppress inflammatory responses. A major side effect of TZDs is weight gain due to fluid retention and increase in total body fat. An earlier drug in this class, troglitazone, was withdrawn due to rare but serious cases of hepatotoxicity. Current therapies have limited durability and/or significant side effects.

The widespread availability and increased consumption of Western diet combined with the adoption of a sedentary life-style has increased the number of obese people. Obesity is linked to a wide range of medical complications, such as diabetes, cardiovascular disease and cancer. In addition, being overweight can exacerbate the development of osteoporosis and asthma. Obesity is also proven to double the risk of hypertension. Obesity has only recently been regarded as a disease in the sense of being a specific target for medical therapy. Current therapies for obesity are based on diet and exercise and stomach surgery for extremely obese patients. Two weight loss medications are today available for long-term use. Sibutramine, a serotonin- and noradrenaline-reuptake inhibitor, controls appetite by producing a feeling of satiety. However, a prominent side effect is hypertension. Orlistat inhibits the lipase-mediated breakdown of fat in the gastrointestinal tract, thereby limiting caloric intake resulting in weight loss. However, approximately 20% of the patients using Orlistat develop faecal incontinence and urgency. Thus, there is an unmet medical need for new and novel antidiabetic and antiobesity therapies.

GPR119 (GenBank No. NM 178471) is a G-protein coupled receptor identified as SNORF25 in WO 00/50562. In humans, GPR119 is selectively expressed in pancreas and gastrointestinal tract. Activation of GPR119 by lysophosphatidylcholine (LPC) induces glucose-dependent insulin secretion from pancreatic beta-cells (Soga et al., Biochem. Biophys. Res. Commun. 326, 744-751, 2005). GPR119 agonists stimulate insulin secretion in rat islets and reduce blood glucose in diabetic Lepr^{db/db} mice (WO 2004/065380).

Another endogenous ligand for GPR119, oleoylethanolamide (OEA), and a small molecule GPR119 agonist, PSN632408, both suppress food intake and reduce body weight gain in rat (Overton et al., Cell Metabolism 3, 167-175, 2006). Taken together, these data suggest that GPR119 is an interesting target for treating diabetes and/or obesity.

WO 2004/065380, WO 2004/076413, WO 2005/007647, WO 2005/007658 and WO 2005/121121 discloses compounds that are modulators of the Rup3 receptor, also referred to as SNORF25 (WO 00/50562) or as GPR119 (Fredriksson et al., FEBS Lett, 554, 381-388, 2003), and which inter alia may be used for the treatment of metabolic disorders and complications thereof, such as, diabetes and obesity.

WO 2005/061489, WO 2006/067531, WO 2006/067532 and WO 2006/070208 disclose compounds that are agonists of GPR116, also referred to as SNORF25 or as GPR119 (see Overton et al, Cell Metabolism 3, 167-175, 2006), and which inter alia may be used for the treatment of metabolic disorders and complications thereof, such as diabetes and obesity.

WO 2006/076231 discloses a synergistic effect of a GPR119 agonist in combination with a DPP-IV inhibitor, in lowering elevated glucose levels in mice. Further, a synergistic effect with the said combination is shown in increasing blood GLP-1 levels after glucose challenge in mice.

### DISCLOSURE OF THE INVENTION

It has surprisingly been found that compounds of the general Formula (Ia) to (Ie) are active as agonists of GPR119 and are potentially useful in the treatment or prophylaxis of disorders relating to GPR119. Examples of such disorders include Type 1 diabetes, Type 2 diabetes, inadequate glucose tolerance, insulin resistance, hyperglycemia, hyperlipidemia, hypercholesterolemia, dyslipidemia, syndrome X, metabolic syndrome, obesity, hypertension, chronic systemic inflammation, retinopathy, neuropathy, nephropathy, atherosclerosis, reduced fibrinolysis, and endothelial dysfunction.

### DEFINITIONS

The following definitions shall apply throughout the specification and the appended claims.

Unless otherwise stated or indicated, the term “C₁₋₆-alkyl” denotes a straight or branched alkyl group having from 1 to 6 carbon atoms. For parts of the range “C₁₋₆-alkyl”, all subgroups thereof are contemplated, such as C₁₋₅-alkyl, C₁₋₄-alkyl, C₁₋₃-alkyl, C₁₋₂-alkyl, C₂₋₆-alkyl, C₂₋₅-alkyl, C₂₋₄-alkyl, C₂₋₃-alkyl, C₃₋₆-alkyl, C₄₋₅-alkyl, etc. Examples of said C₁₋₆-alkyl include methyl, ethyl, n-propyl, isopropyl, n-butyl, isobutyl, sec-butyl, t-butyl and straight- and branched-chain pentyl and hexyl.

Unless otherwise stated or indicated, the term “cyano-C₁₋₆-alkyl” denotes a C₁₋₆-alkyl group, as defined above, substituted with a cyano group. Exemplary cyano-C₁₋₆-alkyl groups include 2-cyanoethyl and 3-cyanopropyl.

Unless otherwise stated or indicated, the term “amino-C₁₋₆-alkyl” denotes a C₁₋₆-alkyl group, as defined above, substituted with an amino group. Exemplary amino-C₁₋₆-alkyl groups include 2-aminoethyl and 3-aminopropyl.

Unless otherwise stated or indicated, the term “hydroxy-C₁₋₆-alkyl” denotes a straight or branched alkyl group that has a hydrogen atom thereof replaced with OH. Examples of said hydroxy-C₁₋₆-alkyl include hydroxymethyl, 2-hydroxyethyl, 2-hydroxypropyl, 3-hydroxy-3-methylbutyl, 2-hydroxybutyl and 2-hydroxy-2-methylpropyl.

Derived expressions such as “C₁₋₆-alkoxy”, “C₁₋₆-alkylthio” and “C₁₋₆-alkylamino” are to be construed accordingly where an C₁₋₆-alkyl group is attached to the remainder of the molecule through an oxygen, sulfur or nitrogen atom, respectively. For parts of the range “C₁₋₆-alkoxy” all subgroups thereof are contemplated such as C₁₋₅-alkoxy, C₁₋₄-alkoxy, C₁₋₃-alkoxy, C₁₋₂-alkoxy, C₂₋₆-alkoxy, C₂₋₅-alkoxy, C₂₋₄-alkoxy, C₂₋₃-alkoxy, C₃₋₆-alkoxy, C₄₋₅-alkoxy, etc. Examples of said “C₁₋₆-alkoxy” include methoxy, ethoxy, n-propoxy, isopropoxy, n-butoxy, isobutoxy, sec-butoxy, t-butoxy and straight- and branched-chain pentoxy and hexoxy etc. Subgroups of “C₁₋₆-alkylthio” and “C₁₋₆-alkylamino” are to be construed accordingly.

Unless otherwise stated or indicated, the term “C₁₋₄-alkylsulfinyl” denotes a group C₁₋₄-alkyl-S(O)—. Exemplary C₁₋₄-alkylsulfinyl groups include methylsulfinyl and ethylsulfinyl.

Unless otherwise stated or indicated, the term “dihydroxy-C₂₋₆-alkyl” denotes a C₂₋₆-alkyl group which is disubstituted with hydroxy and wherein said hydroxy groups are attached to different carbon atoms. Exemplary dihydroxy-C₂₋₆-alkyl groups include 2,3-dihydroxypropyl and 2,4-dihydroxybutyl.

Unless otherwise stated or indicated, the term “di(C₁₋₄-alkyl)amino” denotes a group (C₁₋₄-alkyl)₂N—, wherein the two alkyl portions may be the same or different. Exemplary di(C₁₋₄-alkyl)amino groups include N,N-dimethylamino, N-ethyl-N-methylamino and N,N-diethylamino.

Unless otherwise stated or indicated, the term “di(C₁₋₄-alkyl)amino-C₂₋₄-alkyl” denotes a group di(C₁₋₄-alkyl)amino, as defined above, attached to a C₂₋₄-alkyl group. Exemplary di(C₁₋₄-alkyl)amino-C₂₋₄-alkyl groups include 2-(dimethylamino)ethyl and 3-(diethylamino)propyl.

Unless otherwise stated or indicated, the term “fluoro-C₁₋₆-alkyl” denotes a C₁₋₆-alkyl group substituted by one or more fluorine atoms. Examples of said fluoro-C₁₋₆-alkyl include 2-fluoroethyl, fluoromethyl, 2-fluoro-1-(fluoromethyl)ethyl, trifluoromethyl, 3,3,3-trifluoropropyl and 2,2,2-trifluoroethyl. Likewise, “aryl-C₁₋₆-alkyl” means a C₁₋₆-alkyl group substituted by an aryl group. Examples include benzyl, 2-phenylethyl, 1-phenylethyl and 2-methyl-2-phenylpropyl.

Unless otherwise stated or indicated, the term “arylcarbonyl-C₁₋₄-alkyl” denotes an arylcarbonyl group (e.g., benzoyl) that is attached through a C₁₋₄-alkyl group. Examples of said arylcarbonyl-C₁₋₄-alkyl include 3-oxo-3-phenylpropyl, 2-oxo-2-phenylethyl and 1-methyl-3-oxo-3-phenylpropyl.

Unless otherwise stated or indicated, the term “heteroarylcarbonyl-C₁₋₄-alkyl” denotes a heteroarylcarbonyl group (e.g., 3-pyridinylcarbonyl) that is attached through a C₁₋₄-alkyl group. Examples of said heteroarylcarbonyl-C₁₋₄-alkyl include 3-oxo-3-(3-pyridinyl)-propyl, 2-oxo-2-(3-pyridinyl)ethyl and 1-methyl-3-oxo-3-(3-pyridinyl)propyl.

Unless otherwise stated or indicated, the term “C₁₋₆-alkoxy-C₂₋₆-alkyl” denotes a straight or branched alkoxy group having from 1 to 6 carbon atoms connected to an alkyl group having from 2 to 6 carbon atoms. Examples of said C₁₋₆-alkoxy-C₂₋₆-alkyl include methoxyethyl, ethoxyethyl, isopropoxyethyl, n-butoxyethyl, t-butoxyethyl and straight- and branched-chain pentoxyethyl. For parts of the range “C₁₋₆-alkoxy-C₂₋₆-alkyl” all subgroups thereof are contemplated such as C₁₋₅-alkoxy-C₂₋₆-alkyl, C₁₋₄-alkoxy-C₂₋₆-alkyl, C₁₋₃-alkoxy-C₂₋₆-alkyl, C₁₋₂-alkoxy-C₂₋₆-alkyl, C₂₋₆-alkoxy-C₂₋₆-alkyl, C₂₋₅-alkoxy-C₂₋₆-alkyl, C₂₋₄-alkoxy-C₂₋₆-alkyl, C₂₋₃-alkoxy-C₂₋₆-alkyl, C₃₋₆-alkoxy-C₂₋₆-alkyl, C₄₋₅-alkoxy-C₂₋₆-alkyl, C₁₋₆-alkoxy-C₂₋₅-alkyl, C₁₋₆-alkoxy-C₂₋₄-alkyl, etc.

Unless otherwise stated or indicated, the term “C₂₋₆-alkenyl” denotes a straight or branched hydrocarbon chain radical containing one carbon-carbon double bond and having from 2 to 6 carbon atoms. Examples of said C₂₋₆-alkenyl include vinyl, allyl, 2,3-dimethylallyl, 1-butenyl, 1-pentenyl, and 1-hexenyl. For parts of the range “C₂₋₆-alkenyl”, all subgroups thereof are contemplated such as C₂₋₅-alkenyl, C₂₋₄-alkenyl, C₂₋₃-alkenyl, C₃₋₆-alkenyl, C₄₋₅-alkenyl, etc. Likewise, “aryl-C₂₋₆-alkenyl” means a C₂₋₆-alkenyl group substituted by an aryl group. Examples of said aryl-C₂₋₆-alkenyl include styryl and cinnamyl.

Unless otherwise stated or indicated, the term “C₂₋₆-alkynyl” denotes a straight or branched hydrocarbon chain radical containing one carbon-carbon triple bond and having from 2 to 6 carbon atoms. Examples of said C₂₋₆-alkynyl include ethynyl, 1-propynyl, 2-propynyl, 1-butynyl, 2-butynyl, and 1-methylprop-2-yn-1-yl.

Likewise, aryl-C₂₋₆-alkynyl means a C₂₋₆-alkynyl group substituted by an aryl group. Examples of said aryl-C₂₋₆-alkynyl include phenylethynyl, 3-phenyl-1-propyn-1-yl, 3-phenyl-2-propyn-1-yl and 4-phenyl-2-butyn-1-yl.

The term “oxo” denotes

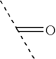

Unless otherwise stated or indicated, the term “C₃₋₇-cycloalkyl” denotes a cyclic alkyl group having a ring size from 3 to 7 carbon atoms and includes cyclopropyl, cyclobutyl, cyclopentyl, cyclohexyl, and cycloheptyl. For parts of the range “C₃₋₇-cycloalkyl” all subgroups thereof are contemplated such as C₃₋₆-cycloalkyl, C₃₋₅-cycloalkyl, C₃₋₄-cycloalkyl, C₄₋₇-cycloalkyl, C₄₋₆-cycloalkyl, C₄₋₅-cycloalkyl, C₅₋₇-cycloalkyl, C₆₋₇-cycloalkyl.

Unless otherwise stated or indicated, the term “C₃₋₇-cycloalkyl-C₁₄-alkyl” denotes a C₃₋₇-cycloalkyl group attached to a C₁₋₄-alkyl group. Exemplary C₃₋₇-cycloalkyl-C₁₋₄-alkyl groups include cyclopropylmethyl, 1-cyclopropylethyl, cyclohexylmethyl and 2-cyclohexylethyl. When the cycloalkyl portion as part of the group C₃₋₇-cycloalkyl-C₁₋₄-alkyl is substituted with methyl, examples of such groups include (1-methylcyclopropyl)methyl and 2-(4-methylcyclohexyl)ethyl.

Unless otherwise stated or indicated, the term “C₇₋₈-bicyclyl” denotes a carbobicyclic saturated aliphatic ring system in which two non-adjacent carbon atoms of a monocyclic ring are linked by an alkylene bridge of between one and three additional carbon atoms. Examples of said C₇₋₈-bicyclyl include radicals obtainable from bicyclo[3.1.1]heptane, bicyclo[2.2.1]heptane (norbornane) and bicyclo[2.2.2]octane.

Unless otherwise stated or indicated, the term C₇₋₈-bicyclylalkyl means a C₁₋₆-alkyl group substituted by a C₇₋₈-bicyclyl group as defined above. An exemplary C₇₋₈-bicyclylalkyl group is bicyclo[2.2.1]hept-2-ylmethyl (2-norbonylmethyl).

Unless otherwise stated or indicated, the term “C₅₋₈-cycloalkenyl” denotes a monocyclic or bicyclic alkenyl group of 5 to 8 carbon atoms having one carbon-carbon double bond. Examples of monocyclic cycloalkenyl groups are cyclopent-3-en-1-yl and cyclohexen-1-yl. An exemplary bicyclic cycloalkenyl group is bicyclo[2.2.1]hept-5-en-2-yl(norbornen-2-yl).

Unless otherwise stated or indicated, the term “oxo-C₄₋₆-cycloalkyl” refers to a C₄₋₆-cycloalkyl wherein one of the ring carbons is a carbonyl. Examples of “oxo-C₄₋₆-cycloalkyl” include 2-oxocyclobutyl, 3-oxocyclobutyl, 2-oxocyclopentyl and 4-oxocyclohexyl.

Unless otherwise stated or indicated, the term “fluoro-C₃₋₆-cycloalkyl” denotes a C₃₋₆-cycloalkyl group substituted by one or two fluorine atoms. Examples of said “fluoro-C₃₋₆-cycloalkyl” include 2,2-difluorocyclopropyl and 4-fluorocyclohexyl.

Unless otherwise stated or indicated, the term “C₁₋₃-alkoxy-C₄₋₆-cycloalkyl” denotes a C₄₋₆-cycloalkyl group substituted by a C₁₋₃-alkoxy group. Examples of said “C₁₋₃-alkoxy-C₄₋₆-cycloalkyl” include 4-methoxycyclohexyl and 2-ethoxycyclopentyl.

Unless otherwise stated or indicated, the term “methyl-C₃₋₆-cycloalkyl” denotes a C₃₋₆-cycloalkyl group substituted by one or two methyl groups. Examples of said “methyl-C₃₋₆-cycloalkyl” include 4-methylcyclohexyl and 3,3-dimethylcyclopentyl.

Unless otherwise stated or indicated, the term “acyl”, which may be straight or branched, denotes a carbonyl group that is attached through its carbon atom to a hydrogen atom to form a C₁-acyl group (i.e., a formyl group) or to an alkyl group, where alkyl is defined as above. For parts of the range “C₁₋₆-acyl” all subgroups thereof are contemplated such as C₁₋₅-acyl, C₁₋₄-acyl, C₁₋₃-acyl, C₁₋₂-acyl, C₂₋₆-acyl, C₂₋₅-acyl, C₂₋₄-acyl, C₂₋₃-acyl, C₃₋₆-acyl, C₄₋₅-acyl, etc. Exemplary acyl groups include formyl, acetyl (i.e., C₂-acyl), propanoyl, butanoyl, pentanoyl, hexanoyl.

Unless otherwise stated or indicated, the term “C₂₋₆-acyl-C₁₋₆-alkyl” refers to a group C₁₋₅-alkyl-(C═O)—C₁₋₆-alkyl. Exemplary C₂₋₆-acyl-C₁₋₆-alkyl groups include 2-acetylethyl and 3-acetylpropyl.

Unless otherwise stated or indicated, the term “C₁₋₆-alkylsulfonyl”, which may be straight or branched, denotes a hydrocarbon having from 1 to 6 carbon atoms with a sulfonyl group. For parts of the range “C₁₋₆-alkylsulfonyl” all subgroups thereof are contemplated such as C₁₋₅-alkylsulfonyl, C₁₋₄-alkylsulfonyl, C₁₋₃-alkylsulfonyl, C₁₋₂-alkylsulfonyl, C₂₋₆-alkylsulfonyl, C₂₋₅-alkylsulfonyl, C₂₋₄-alkylsulfonyl, C₂₋₃-alkylsulfonyl, C₃₋₆-alkylsulfonyl, C₄₋₅-alkylsulfonyl, etc. Exemplary C₁₋₆-alkylsulfonyl groups include methylsulfonyl, ethylsulfonyl, propylsulfonyl, n-butylsulfonyl, sec-butylsulfonyl, tert-butylsulfonyl, pentylsulfonyl and hexylsulfonyl.

Unless otherwise stated or indicated, the term “hydroxy-C₂₋₄-alkylsulfonyl” denotes a C₂₋₄-alkylsulfonyl group as defined above substituted with a hydroxy group. Examples of said hydroxy-C₂₋₄-alkylsulfonyl include hydroxymethylsulfonyl and 2-hydroxyethylsulfonyl.

Unless otherwise stated or indicated, the term “C₁₋₄-alkylsulfonamido” denotes a group C₁₋₄-alkyl-SO—NH—. Exemplary C₁₋₄-alkylsulfonamido groups include methylsulfonylamino and ethylsulfonylamino.

Unless otherwise stated or indicated, the term “C₁₋₃-alkylene” refers to the diradicals methylene (—CH₂—), ethylene (—CH₂—CH₂—) and propylene (—CH₂—CH₂—CH₂—). In case the group denoted by E in Formula (Ia) forms a double bond with D, then E is a trivalent radical selected from (═CH₂—CH₂—) and (═CH₂—CH₂—CH₂—).

Unless otherwise stated or indicated, the term “halogen” shall mean fluorine, chlorine, bromine or iodine.

Unless otherwise stated or indicated, the term “aryl” refers to a hydrocarbon ring system having at least one aromatic ring, preferably mono- or bicyclic. Examples of aryls are phenyl, indenyl, 2,3-dihydroindenyl(indanyl), 1-naphthyl, 2-naphthyl or 1,2,3,4-tetrahydronaphthyl.

Unless otherwise stated or indicated, the term “heteroaryl” refers to a mono- or bicyclic heteroaromatic ring system having 5 to 10 ring atoms in which one or more of the ring atoms are other than carbon, such as nitrogen, sulphur or oxygen. Only one ring need be aromatic and said heteroaryl moiety can be linked to the remainder of the molecule via a carbon or nitrogen atom in any ring. Examples of heteroaryl groups include furyl, pyrrolyl, thienyl, oxazolyl, isoxazolyl, imidazolyl, thiazolyl, isothiazolyl, pyridyl, pyrimidinyl, quinazolinyl, indolyl, isoindolyl, 1,3-dihydro-isoindolyl, pyrazolyl, pyridazinyl, quinolinyl, quinoxalinyl, thiadiazolyl, benzofuranyl, 2,3-dihydrobenzofuranyl, 1,3-benzodioxolyl, 1,4-benzodioxinyl, 2,3-dihydro-1,4-benzodioxinyl, benzothiazolyl, benzimidazolyl, benzothiadiazolyl, benzotriazolyl, indolinyl, isoindolinyl, and chromanyl groups.

Unless otherwise stated or indicated, the term “heterocyclyl” or “heterocyclic ring” refers to a non-aromatic fully saturated or partially unsaturated monocyclic ring system having 4 to 7 ring atoms with at least one heteroatom such as O, N, or S, and the remaining ring atoms are carbon. Examples of heterocyclic groups include piperidinyl, tetrahydropyranyl, tetrahydrofuranyl, oxetanyl, azepinyl, azetidinyl, pyrrolidinyl, morpholinyl, imidazolinyl, imidazolidinyl, thiomorpholinyl, pyranyl, dioxanyl, piperazinyl and 5,6-dihydro-4H-1,3-oxazin-2-yl. When present, the sulfur atom may be in an oxidized form (i.e., S═O or O═S═O). Exemplary heterocyclic groups containing sulfur in oxidized form are 1,1-dioxido-thiomorpholinyl and 1,1-dioxido-isothiazolidinyl.

When two groups R⁵, two groups R^{5A}, two groups R⁹ or two groups R^{9A} described herein form a heterocyclic ring and said heterocyclic ring is substituted with one or two oxo groups, examples of such groups include 2-pyrrolidon-1-yl, 2-piperidon-1-yl, 2-azetidinon-1-yl, 2,5-dioxopyrrolidin-1-yl and hydantoin-1-yl (i.e., 2,5-dioxoimidazolidin-1-yl).

When two groups R⁵, two groups R^{5A}, two groups R⁹ or two groups R^{9A} described herein form a heterocyclic ring and said heterocyclic ring is substituted with one or two fluoro atoms, examples of such groups include 4-fluoropiperidin-1-yl, 4,4-difluoropiperidin-1-yl, 3-fluoropyrrolidin-1-yl and 3,3-difluoropyrrolin-1-yl.

When two groups R⁵, two groups R^{5A}, two groups R⁹ or two groups R^{9A} described herein form a heterocyclic ring and said heterocyclic ring is substituted with hydroxy, examples of such groups include 4-hydroxypiperidin-1-yl, 3-hydroxypiperidin-1-yl, 3-hydroxypyrrolidin-1-yl and 3-hydroxyazetidin-1-yl.

When two groups R⁵, two groups R^{5A}, two groups R⁹ or two groups R^{9A} described herein form a heterocyclic ring and said heterocyclic ring is substituted with amino, examples of such groups include 4-aminopiperidin-1-yl, 3-aminopiperidin-1-yl, and 3-aminopyrrolidin-1-yl.

When two groups R⁵, two groups R^{5A}, two groups R⁹ or two groups R^{9A} described herein form a heterocyclic ring and said heterocyclic ring is substituted with hydroxymethyl, examples of such groups include 2-(hydroxymethyl)pyrrolidin-1-yl, 2-(hydroxymethyl)-morpholin-4-yl and 4-(hydroxymethyl)piperidin-1-yl.

When two groups R⁵, two groups R^{5A}, two groups R⁹ or two groups R^{9A} described herein form a heterocyclic ring and said heterocyclic ring is substituted with methylamino or dimethylamino, examples of such groups include 3-dimethylaminopyrrolidin-1-yl and 3-methylaminopyrrolidin-1-yl.

Unless otherwise stated or indicated, the term “heteroaryl-C₁₋₄-alkyl” denotes a heteroaryl group that is attached through a C₁₋₄-alkyl group. Examples of said heteroaryl-C₁₋₄-alkyl include 2-(pyridin-2-yl)ethyl and 1,3 benzodioxol-5-ylmethyl.

“C-heterocyclyl” indicates bonding via a carbon atom of said heterocyclyl, for example piperidin-4-yl, tetrahydrofuran-2-yl, oxetan-3-yl, tetrahydrofuran-3-yl and 5,6-dihydro-4H-1,3-oxazin-2-yl, while “N-heterocyclyl” indicates bonding through nitrogen in a nitrogen-containing heterocyclyl group, for example piperidin-1-yl and piperazin-1-yl. When C-heterocyclyl is substituted by C₁₋₄-alkyl, said C₁₋₄-alkyl is attached to a ring nitrogen atom or a ring carbon atom thereof. Exemplary C-heterocyclyl groups substituted by C₁₋₄-alkyl include 1-methylpiperidin-4-yl and 3-methyloxetan-3-yl.

Unless otherwise stated or indicated, the term “N-heterocyclyl-C₂₋₄-alkyl” refers to a nitrogen-containing heterocyclyl group that is directly linked to a C₂₋₄-alkyl group via a nitrogen atom of said heterocyclyl. Exemplary N-heterocyclyl-C₂₋₄-alkyl groups include 2-(pyrrolidin-1-yl)ethyl, 3-(4-morpholinyl)propyl, 2-(piperazin-1-yl)ethyl and 2-(4-morpholinyl)ethyl.

When heterocyclyl as part of the group N-heterocyclyl-C₂₋₄-alkyl is substituted by methyl, said heterocyclyl is selected from 1-piperazinyl or 1-homopiperazinyl and said methyl is attached to the 4-position of the piperazine or homopiperazine ring. Exemplary N-heterocyclyl-C₂₋₄-alkyl groups wherein heterocyclyl is substituted with methyl are 2-(4-methylpiperazin-1-yl)ethyl, 2-(4-methylhomopiperazin-1-yl)ethyl.

Unless otherwise stated or indicated, the term “C-heterocyclyl-C₁₋₄-alkyl” refers to a heterocyclyl group that is directly linked to a C₁₋₄-alkyl group via a carbon atom of said heterocyclyl. Exemplary C-heterocyclyl-C₁₋₄-alkyl groups include tetrahydropyran-4-ylmethyl, piperidin-4-ylmethyl, tetrahydrofuran-2-ylmethyl, oxetan-3-ylmethyl and 2-(piperidinyl-4-yl)ethyl.

When heterocyclyl as part of the group C-heterocyclyl-C₁₋₄-alkyl is substituted by methyl, said methyl is attached to a ring nitrogen atom or ring carbon atom thereof. Exemplary C-heterocyclyl-C₁₋₄-alkyl groups wherein heterocyclyl is substituted with methyl are 2-(1-methylpiperidin-4-yl)ethyl and 3-methyloxetan-3-ylmethyl.

Unless otherwise stated or indicated, the term “oxo-N-heterocyclyl” denotes a nitrogen-containing heterocyclyl group that is substituted with one or two oxo groups.

Unless otherwise stated or indicated, the term “oxo-N-heterocyclyl-C₂₋₄-alkyl” refers to an oxo-N-heterocyclyl group that is directly linked to a C₂₋₄-alkyl group through a nitrogen atom of its heterocyclyl portion and where oxo-N-heterocyclyl is as defined above. Exemplary oxo-N-heterocyclyl-C₂₋₄-alkyl groups include 2-(2-pyrrolidon-1-yl)ethyl, 3-(2-pyrrolidon-1-yl)propyl and 2-(2,5-dioxoimidazolidin-1-yl)ethyl.

Unless otherwise stated or indicated, the term “fluoro-N-heterocyclyl” denotes a nitrogen-containing heterocyclyl group that is substituted at a position other than alpha to a ring heteroatom with one or two fluorine atoms.

Unless otherwise stated or indicated, the term “fluoro-N-heterocyclyl-C₂₋₄-alkyl” refers to a fluoro-N-heterocyclyl group that is directly linked to a C₂₋₄-alkyl group through a nitrogen atom of its heterocyclyl portion and where fluoro-N-heterocyclyl is as defined above. Exemplary fluoro-N-heterocyclyl-C₂₋₄-alkyl groups include 2-(3-fluoropyrrolidin-1-yl)-ethyl and 3-(3-fluoropyrrolidin-1-yl)propyl.

Unless otherwise stated or indicated, the term “hydroxy-N-heterocyclyl” denotes a nitrogen-containing heterocyclyl group that is substituted at a position other than alpha to a ring heteroatom with a hydroxy group.

Unless otherwise stated or indicated, the term “hydroxy-N-heterocyclyl-C₂₋₄-alkyl” refers to a hydroxy-N-heterocyclyl group that is directly linked to a C₂₋₄-alkyl group through a nitrogen atom of its heterocyclyl portion and where hydroxy-N-heterocyclyl is as defined above. Exemplary hydroxy-N-heterocyclyl-C₂₋₄-alkyl groups include 2-(4-hydroxypiperidin-1-yl)ethyl and 3-(3-hydroxypiperidin-1-yl)propyl.

Unless otherwise stated or indicated, the term “amino-N-heterocyclyl” denotes a nitrogen-containing heterocyclyl group that is substituted at a position other than alpha to a ring heteroatom with an amino group.

Unless otherwise stated or indicated, the term “amino-N-heterocyclyl-C₂₋₄-alkyl” refers to a amino-N-heterocyclyl group that is directly linked to a C₂₋₄-alkyl group through a nitrogen atom of its heterocyclyl portion and where amino-N-heterocyclyl is as defined above. Exemplary amino-N-heterocyclyl-C₂₋₄-alkyl groups include 2-(4-aminopiperidin-1-yl)ethyl and 3-(3-aminopiperidin-1-yl)propyl.

Unless otherwise stated or indicated, the term “azabicyclyl” denotes a bicyclic heterocyclyl group with seven or eight atoms (including bridgehead atoms), wherein at least one ring member is a nitrogen atom and the remainder ring atoms being carbon. The said azabicyclyl may optionally contain a carbon-carbon double bond. Examples of azabicyclyl groups include carbon radicals obtainable from 1-azabicyclo[2.2.2]octane, 1-azabicyclo[2.2.1]heptane and azabicyclo[2.2.2]oct-2-ene.

“C-heterocyclylsulfonyl” refers to a heterocyclyl group that is directly bonded to SO₂ via a carbon atom. Exemplary C-heterocyclylsulfonyl groups include 4-piperidinylsulfonyl and tetrahydropyran-4-ylsulfonyl.

When C-heterocyclylsulfonyl is substituted by C₁₋₄-alkyl, said heterocyclyl is selected from a nitrogen-containing heterocyclyl, and said C₁₋₄-alkyl is attached to a ring nitrogen atom thereof. An exemplary C-heterocyclylsulfonyl group substituted by C₁₋₄-alkyl includes 1-methylpiperidin-4-ylsulfonyl.

Unless otherwise stated or indicated, the term “C₂₋₄-acylamino” denotes a group R^{b}(C═O)NH— wherein R^{b} is selected from C₁₋₃-alkyl. Exemplary C₂₋₄-acylamino groups include acetylamino and propionylamino.

Unless otherwise stated or indicated, the term “C₂₋₄-acylamino-C₁₋₄-alkyl” denotes a C₂₋₄ acylamino group, as defined above, attached to a C₁₋₄-alkyl group. Exemplary C₂₋₄-acylamino-C₁₋₄-alkyl groups include (acetylamino)methyl and 2-(acetylamino)ethyl.

Unless otherwise stated or indicated, the term “aminocarbonyl” refers to the radical NH₂(C═O)—.

Unless otherwise stated or indicated, the term “aminocarbonyl-C₁₋₄-alkyl” denotes a C₁₋₄-alkyl group, as defined above, substituted with an aminocarbonyl group. Exemplary aminocarbonyl-C₁₋₄-alkyl groups include 2-(aminocarbonyl)ethyl and 3-(aminocarbonyl)-propyl.

Unless otherwise stated or indicated, the term “carboxy” denotes a group —C(O)OH.

Unless otherwise stated or indicated, the term “carboxy-C₁₋₃-alkyl” refers to a carboxy group, as defined above, attached to a C₁₋₃-alkyl group. Exemplary carboxy-C₁₋₃-alkyl groups include 2-carboxyethyl and 3-carboxypropyl.

Unless otherwise stated or indicated, the term “carboxy-C₁₋₃-alkylcarbonylamino” refers to a carboxy-C₁₋₃-alkyl groups, as defined above, attached to the carbonyl carbon of carbonylamino (i.e., —C(O)NH—). Exemplary carboxy-C₁₋₃-alkylcarbonylamino groups include (2-carboxyethyl)carbonylamino and (3-carboxypropyl)carbonylamino. “C-heterocyclylcarbonyl” refers to a heterocyclyl group that is directly bonded to a carbonyl group via a carbon atom while “N-heterocyclylcarbonyl” refers to a nitrogen-containing heterocyclyl group that is directly bonded to a carbonyl group via a nitrogen atom. Examples of N-heterocyclylcarbonyl groups include 1-piperidinylcarbonyl, 1-piperazinylcarbonyl and 1-pyrrolidincarbonyl. Exemplary C-heterocyclylcarbonyl groups include 3-piperidinylcarbonyl, 4-piperidinylcarbonyl and tetrahydropyranyl-4-ylcarbonyl.

When C-heterocyclylcarbonyl is substituted by C₁₋₄-alkyl, said heterocyclyl is selected from a nitrogen-containing heterocyclyl, and said C₁₋₄-alkyl is attached to a ring nitrogen atom thereof. An exemplary C-heterocyclylcarbonyl group substituted by C₁₋₄-alkyl includes 1-methylpiperidin-4-ylcarbonyl.

The term “N-heterocyclylcarbonyl-C₂₋₄-alkyl” refers to a N-heterocyclylcarbonyl group that is directly linked to a C₂₋₄-alkyl group through its carbonyl carbon atom and where N-heterocyclylcarbonyl is as defined above. Exemplary N-heterocyclylcarbonyl-C₂₋₄-alkyl groups include 2-(pyrrolidin-1-ylcarbonyl)ethyl, 2-(piperazin-1-ylcarbonyl)ethyl and 2-(piperidin-1-ylcarbonyl)ethyl.

When heterocyclyl as part of the group N-heterocyclylcarbonyl-C₂₋₄-alkyl is substituted by methyl, said heterocyclyl is selected from 1-piperazinyl or 1-homopiperazinyl and said methyl is attached to the 4-position of the piperazine or homopiperazine ring. Exemplary N-heterocyclylcarbonyl-C₂₋₄-alkyl groups wherein heterocyclyl is substituted with methyl are 2-(4-methylpiperazin-1-ylcarbonyl)ethyl, 2-(4-methylhomopiperazin-1-ylcarbonyl)-ethyl.

The term “C-heterocyclylcarbonyl-C₂₋₄-alkyl” refers to a C-heterocyclylcarbonyl group that is directly linked to a C₂₋₄-alkyl group through its carbonyl carbon atom and where C-heterocyclylcarbonyl is as defined above. Exemplary C-heterocyclylcarbonyl-C₂₋₄-alkyl groups include 2-(tetrahydropyran-4-ylcarbonyl)ethyl, 2-(piperidin-3-ylcarbonyl)ethyl and 2-(piperidin-4-ylcarbonyl)ethyl.

When heterocyclyl as part of the group C-heterocyclylcarbonyl-C₂₋₄-alkyl is substituted by methyl, said heterocyclyl is selected from a nitrogen-containing heterocyclyl and said methyl is attached to a ring nitrogen atom thereof. An exemplary C-heterocyclylcarbonyl-C₂₋₄-alkyl group wherein heterocyclyl is substituted with methyl is 2-(1-methylpiperidin-4-ylcarbonyl)ethyl.

The term “C-heterocyclyloxy” refers to a heterocyclic group that is directly bonded to an oxygen atom via a carbon atom. Examples of C-heterocyclyloxy groups include 3-piperidinyloxy, 4-piperidinyloxy, 3-tetrahydrofuranyloxy, and 4-tetrahydropyranyloxy.

When C-heterocyclyloxy is substituted by C₁₋₄-alkyl, said heterocyclyl is selected from a nitrogen-containing heterocyclyl, and said C₁₋₄-alkyl is attached to a ring nitrogen atom thereof. An exemplary C-heterocyclyloxy group substituted by C₁₋₄-alkyl includes 1-methylpiperidin-4-yloxy.

The term “hydroxy-C₂₋₄-alkoxy-C₁₋₄-alkyl” refers to a hydroxy-C₂₋₄-alkoxy group that is directly attached to a C₁₋₄-alkyl group. Representative examples of such groups include:

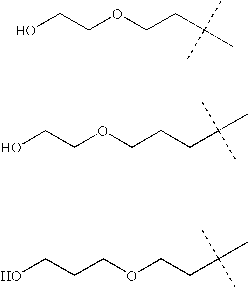

The term “phosphonooxy” refers to a group with the following chemical structure:

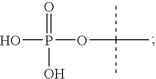

The term “amidino” refers to a group with the following chemical structure:

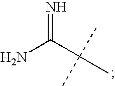

The term “guanidino” refers to a group with the following chemical structure:

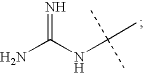

The chemical formula —C(OH)CH₃CF₃ refers to a group with the following chemical structure:

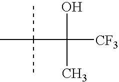

The term [C(OH)CH₃CF₃]—C₁₋₆-alkyl refers to a —C(OH)CH₃CF₃ group that is directly attached to a C₁₋₆-alkyl group. Representative examples of such groups include:

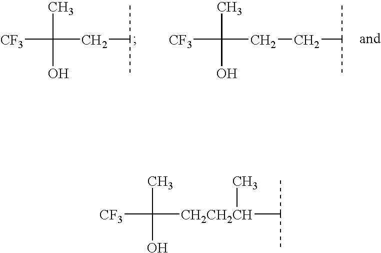

The chemical formula CF₃SO₃ refers to a group with the following chemical structure:

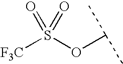

The carbon-carbon double or triple bonds present in the groups C₃₋₆-alkenyl, C₃₋₆-alkynyl, aryl-C₃₋₆-alkenyl and aryl-C₃₋₆-alkynyl as values for R² are meant to be located at positions other than conjugated with a carbonyl group or adjacent to a nitrogen, oxygen or sulfur atom.

“Optional” or “optionally” means that the subsequently described event or circumstance may but need not occur, and that the description includes instances where the event or circumstance occurs and instances in which it does not.

“Pharmaceutically acceptable” means being useful in preparing a pharmaceutical composition that is generally safe, non-toxic and neither biologically nor otherwise undesirable and includes being useful for veterinary use as well as human pharmaceutical use.

“Treatment” as used herein includes prophylaxis of the named disorder or condition, or amelioration or elimination of the disorder once it has been established.

“An effective amount” refers to an amount of a compound that confers a therapeutic effect on the treated subject. The therapeutic effect may be objective (i.e., measurable by some test or marker) or subjective (i.e., subject gives an indication of or feels an effect).

The term “Syndrome X” refers to a syndrome comprising of some or all of the following diseases: 1) dyslipoproteinemia (combined hypercholesterolemia-hypertriglyceridemia, low HDL-cholesterol), 2) obesity (in particular upper body obesity), 3) impaired glucose tolerance (IGT) leading to noninsulin-dependent diabetes mellitus (NIDDM), 4) essential hypertension and (5) thrombogenic/fibrinolytic defects.

The term “prodrug forms” means a pharmacologically acceptable derivative, such as an ester or an amide, which derivative is biotransformed in the body to form the active drug. Reference is made to Goodman and Gilman's, The Pharmacological basis of Therapeutics, 8th ed., Mc-Graw-Hill, Int. Ed. 1992, “Biotransformation of Drugs”, p. 13.

The following abbreviations have been used:
BH₃.SMe₂ means borane-methyl sulphide complex (2.0M sol. in THF)
BOC means tert-butyloxycarbonyl,
Brine means water saturated or nearly saturated with sodium chloride,
DCM means dichloromethane,
DME means 1,2-dimethoxyethane,
DMF means dimethylformamide,
DMSO means dimethyl sulphoxide,
EDC means N-(3-dimethylaminopropyl)-N′-ethylcarbodiimide or 1-ethyl-3-(3-dimethylaminopropyl)carbodiimide hydrochloride
EDTA means ethylenediamine tetraacetic acid,
ESI means electrospray ionization,
EtOH means ethanol,
EtOAc means ethyl acetate,
HDL means High-Density Lipoprotein,
HOBT means 1-hydroxybenzotriazole hydrate,
HPLC means High Performance Liquid Chromatography,
HRESIMS means High-Resolution Electrospray Ionization Mass Spectra,
LCMS means Liquid Chromatography Mass Spectrometry,
LRESIMS means Low-Resolution Electrospray Ionization Mass Spectra,
MeCN means acetonitrile,
MeOH means methanol,
PdCl₂(dppf).DCM means [1,1′-bis(diphenylphosphino)-ferrocene]dichloropalladium(II) complex with DCM (1:1),
r.t. means room temperature,
R_{T} means retention time,
R_{TA} means retention time system A,
R_{TB} means retention time system B,
TBTU means N,N,N′,N′-tetramethyl-O-(benzotriazol-1-yl)uronium tetrafluoroborate,
t-BuOK means potassium tert-butoxide,
TEA means triethylamine,
TFA means trifluoroacetic acid,
THF means tetrahydrofuran.

The term “leaving group” refers to a group to be displaced from a molecule during a nucleophilic displacement reaction. Examples of leaving groups are iodide, bromide, chloride, methanesulfonyloxy, hydroxy, methoxy, thiomethoxy, toluenesulfonyloxy(tosyl) and trifluoromethanesulfonyloxy(triflate), or suitable protonated forms thereof (e.g., H₂O, MeOH).

The term “coupling agent” refers to a substance capable of catalyzing a coupling reaction, such as amidation, or esterification. Examples of coupling agents include, but are not limited to, carbonyldiimidazole, dicyclohexylcarbodiimide, pyridine, 4-dimethylaminopyridine, and triphenylphosphine. Another example of a coupling agent is 1-ethyl-3-(3-dimethylaminopropyl)carbodiimide hydrochloride, which is used in the presence of 1-hydroxybenzotriazole and a base such as triethylamine.

The terms “exo” and “endo” are stereochemical prefixes that describe the relative configuration of a substituent on a bridge (not a bridgehead) of a bicyclic system such as 1-azabicyclo[2.2.1]heptane and bicyclo[2.2.1]heptane. If a substituent is oriented toward the larger of the other bridges, it is endo. If a substituent is oriented toward the smaller bridge it is exo. Both exo and endo forms and their mixtures are part of the present invention.

In a first aspect, the present invention provides a compound of Formula (Ia), 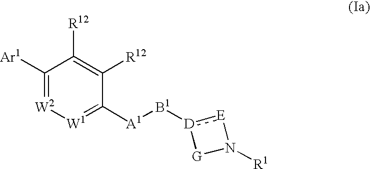
and pharmaceutically acceptable salt, hydrates, geometrical isomers, racemates, tautomers, optical isomers and N-oxides thereof; wherein:
one of W¹ and W² is N and the other is CR¹²;
A¹ is CH₂, O, NR¹⁰, S, S(O) or S(O)₂;
B¹ is CH₂, O, NR¹⁰, S, S(O), S(O)₂, C(O) or CONR¹⁰, provided that when B¹ is O, NR¹⁰, S, S(O), S(O)₂, C(O) or CONR¹⁰, then A¹ is CH₂;
D is N, C or CR¹¹, provided that D must be CR¹¹ and said R¹¹ must be hydrogen or methyl when B¹ is selected from O, NR¹⁰, S, S(O), S(O)₂, and CONR¹⁰; [symbol]
is a single bond when D is N or CR¹¹ or a double bond when D is C;
E and G are independently C₁₋₃-alkylene, each optionally substituted with a substituent independently selected from the group consisting of C₁₋₃-alkyl, C₁₋₄-alkoxy, carboxy, fluoro-C₁₋₃-alkyl, hydroxy, hydroxymethyl, and fluoro, provided that the ring formed by D, E, N and G has not more than 7 ring atoms, and further provided that the said ring has 6 or 7 ring atoms when D is N, and yet further provided that the total number of substituents on E and G are not more than 2;
R¹ is C(O)OR², C(O)R², S(O)₂R², C(O)NR²R³ or —CH₂—C(O)NR²R³; or a 5- or 6-membered heteroaryl group linked via a ring carbon atom, wherein the said heteroaryl group is optionally substituted with C₁₋₄-alkyl;
Ar¹ is phenyl which is optionally substituted in one or more positions with a substituent independently selected from:
(a) CF₃SO₃,
(b) halogen selected from chlorine, bromine and fluorine,
(c) C₁₋₄-alkylsulfinyl,
(d) —S(O)₂R⁴,
(e) —S(O)₂NR⁵R⁵,
(f) —NR⁶S(O)₂R⁴,
(g) —CH₂—NR⁶C(O)R⁴,
(h) —NR⁶C(O)R⁴,
(i) —C(O)NR⁵R⁵,
(j) —CH₂—C(O)NR⁵R⁵,
(k) —C(O)R⁴,
(l) H₂N—C(O)O—,
(m) CH₃—NH—C(O)O—,
(n) (CH₃)₂NC(O)O—,
(o) CH₃OC(O)NH—,
(p) C-heterocyclyl, optionally substituted with C₁₋₄-alkyl,
(q) —CN,
(r) —OR⁸,
(s) —SCF₃,
(t) —NO₂,
(u) phosphonooxy,
(v) C-heterocyclylsulfonyl, optionally substituted with C₁₋₄-alkyl,
(w) —NR⁵R⁵,
(x) —C(OH)CH₃CF₃,
(y) [C(OH)CH₃CF₃]—C₁₋₆-alkyl,
(z) cyano-C₁₋₆-alkyl,
(aa) guanidino,
(bb) amidino,
(cc) C₁₋₆-alkyl,
(dd) C₁₋₄-alkoxy-C₁₋₄-alkyl,
(ee) fluoro-C₁₋₄-alkyl,
(ff) C₂₋₆-alkenyl,
(gg) fluoro-C₂₋₄-alkenyl,
(hh) hydroxy-C₁₋₆-alkyl,
(ii) C₁₋₄-alkylsulfonyl-C₁₋₄-alkyl,
(jj) hydroxy-C₂₋₄-alkoxy-C₁₋₄-alkyl,
(kk) C₂₋₃-acyl-C₁₋₃-alkyl,
(ll) C₂₋₆-alkynyl,
(mm) hydroxy-C₃₋₆-cycloalkyl,
(nn) fluoro-C₃₋₆-cycloalkyl,
(oo) methyl-C₃₋₆-cycloalkyl,
(pp) C-heterocyclylcarbonyl, optionally substituted with C₁₋₄-alkyl,
(qq) C₃₋₆-cycloalkyl,
(rr) C₃₋₆-cycloalkyl-C₁₋₄-alkyl,
(ss) R⁵R⁵N—C₁₋₂-alkyl,
(tt) —C(O)OR⁷,
(uu) —CH₂C(O)OR⁷,
(vv) aryl,
(ww) aryl-C₁₋₄-alkyl,
(xx) aryl-C₂₋₄-alkenyl,
(yy) aryl-C₂₋₄-alkynyl,
(zz) heteroaryl,
(aaa) heteroaryl-C₁₋₄-alkyl,
(bbb) heteroaryl-C₂₋₄-alkenyl, and
(ccc) heteroaryl-C₂₋₄-alkynyl,
   wherein any aryl or heteroaryl residue, alone or as part of another group, as substituent on Ar¹ is optionally substituted in one or more positions with a substituent independently selected from the group Z¹ consisting of:
(a) halogen selected from chlorine and fluorine,
(b) C₁₋₄-alkyl,
(c) hydroxy,
(d) C₁₋₄-alkoxy,
(e) —OCF₃,
(f) —SCF₃,
(g) —CN,
(h) —C(OH)CH₃CF₃,
(i) hydroxy-C₁₋₄-alkyl,
(j) —CF₃,
(k) —S(O)₂CH₃,
(l) —S(O)₂NH₂,
(m) —S(O)₂NHCH₃,
(n) —S(O)₂N(CH₃)₂,
(o) —N(CH₃)S(O)₂CH₃,
(p) —N(CH₃)C(O)CH₃,
(q) —C(O)NH₂,
(r) —C(O)NHCH₃,
(s) —C(O)N(CH₃)₂,
(t) —C(O)CH₃,
(u) —NH₂,
(v) —NHCH₃,
(w) —N(CH₃)₂,
(x) —NO₂, and
(y) methoxycarbonyl;
   R² is selected from:
(a) C₁₋₆-alkyl,
(b) C₁₋₆-alkoxy-C₂₋₆-alkyl,
(c) hydroxy-C₂₋₆-alkyl,
(d) fluoro-C₂₋₆-alkyl,
(e) C₃₋₆-alkynyl,
(f) C₃₋₆-alkenyl,
(g) C₃₋₇-cycloalkyl,
(h) C₅₋₈-cycloalkenyl,
(i) NR⁹R⁹, provided that R¹ is not selected from C(O)OR², C(O)NR²R³ and —CH₂—C(O)NR²R³,
(j) C-heterocyclyl, optionally substituted with C₁₋₄-alkyl,
(k) C₇₋₈-bicyclyl, optionally substituted with hydroxy,
(l) C₇₋₈-bicyclylmethyl,
(m) azabicyclyl, optionally substituted with hydroxy,
(n) C₃₋₇-cycloalkyl-C₁₋₄-alkyl, wherein cycloalkyl is optionally substituted with methyl,
(o) C₁₋₆-alkylsulfonyl-C₂₋₆-alkyl,
(p) C₂₋₃-acyl-C₁₋₄-alkyl,
(q) arylcarbonyl-C₁₋₄-alkyl,
(r) heteroarylcarbonyl-C₁₋₄-alkyl,
(s) [C(OH)CH₃CF₃]—C₁₋₆-alkyl,
(t) N-heterocyclylcarbonyl-C₂₋₄-alkyl, wherein heterocyclyl is optionally substituted with methyl,
(u) C-heterocyclylcarbonyl-C₂₋₄-alkyl, wherein heterocyclyl is optionally substituted with methyl,
(v) aminocarbonyl-C₂₋₆-alkyl,
(w) C₁₋₃-alkylaminocarbonyl-C₂₋₆-alkyl,
(x) di(C₁₋₃-alkyl)aminocarbonyl-C₂₋₆-alkyl,
(y) hydroxy-C₂₋₄-alkoxy-C₂₋₄-alkyl,
(z) hydroxy-C₄₋₆-cycloalkyl,
(aa) oxo-C₄₋₆-cycloalkyl,
(bb) fluoro-C₄₋₆-cycloalkyl,
(cc) C₁₋₃-alkoxy-C₄₋₆-cycloalkyl,
(dd) methyl-C₃₋₆-cycloalkyl,
(ee) oxo-N-heterocyclyl-C₂₋₄-alkyl,
(ff) fluoro-N-heterocyclyl-C₂₋₄-alkyl,
(gg) amino-N-heterocyclyl-C₂₋₄-alkyl,
(hh) hydroxy-N-heterocyclyl-C₂₋₄-alkyl,
(ii) N-heterocyclyl-C₂₋₄-alkyl, wherein heterocyclyl is optionally substituted with methyl,
(jj) C-heterocyclyl-C₁₋₄-alkyl, wherein heterocyclyl is optionally substituted with methyl,
(kk) aryl,
(ll) aryl-C₁₋₄-alkyl,
(mm) aryl-C₃₋₆-alkenyl,
(nn) aryl-C₃₋₆-alkynyl,
(oo) heteroaryl,
(pp) heteroaryl-C₁₋₄-alkyl,
(qq) heteroaryl-C₃₋₆-alkenyl, and
(rr) heteroaryl-C₃₋₆-alkynyl,
   wherein any aryl or heteroaryl residue, alone or as part of another group, is optionally independently substituted in one or more position with a substituent selected from the group Z¹ as defined above;
   R³ is selected from:
(a) hydrogen,
(b) C₁₋₆-alkyl,
(c) fluoro-C₂₋₆-alkyl,
(d) hydroxy-C₂₋₆-alkyl,
(e) C₁₋₆-alkoxy-C₂₋₆-alkyl,
(f) amino-C₂₋₆-alkyl,
(g) C₁₋₃-alkylamino-C₂₋₆-alkyl,
(h) di(C₁₋₃-alkyl)amino-C₂₋₆-alkyl,
(i) cyano-C₁₋₆-alkyl, and
(j) C₁₋₆-alkylsulfonyl-C₂₋₆-alkyl;
   R⁴ is independently selected from:
(a) C₁₋₆-alkyl,
(b) fluoro-C₁₋₆-alkyl,
(c) hydroxy-C₂₋₆-alkyl,
(d) C₁₋₄-alkoxy-C₂₋₄-alkyl,
(e) C₂₋₄-acyl-C₁₋₄-alkyl,
(f) carboxy-C₁₋₃-alkyl,
(g) C₃₋₆-cycloalkyl,
(h) oxo-C₄₋₆-cycloalkyl,
(i) hydroxy-C₄₋₆-cycloalkyl,
(j) fluoro-C₄₋₆-cycloalkyl,
(k) methyl-C₃₋₆-cycloalkyl,
(l) N-heterocyclylcarbonyl-C₂₋₄-alkyl, wherein heterocyclyl is optionally substituted with methyl,
(m) oxo-N-heterocyclyl-C₂₋₄-alkyl,
(n) fluoro-N-heterocyclyl-C₂₋₄-alkyl,
(o) hydroxy-N-heterocyclyl-C₂₋₄-alkyl,
(p) amino-N-heterocyclyl-C₂₋₄-alkyl,
(q) aminocarbonyl-C₂₋₄-alkyl,
(r) C₁₋₃-alkylaminocarbonyl-C₂₋₄-alkyl,
(s) di(C₁₋₃-alkyl)aminocarbonyl-C₂₋₄-alkyl,
(t) C₂₋₃-acylamino-C₂₋₄-alkyl,
(u) hydroxy-C₂₋₄-alkoxy-C₂₋₄-alkyl,
(v) C-heterocyclylcarbonyl-C₂₋₄-alkyl, wherein heterocyclyl is optionally substituted with methyl,
(w) C₃₋₆-cycloalkyl-C₁₋₂-alkyl,
(x) aryl,
(y) aryl-C₁₋₂-alkyl,
(z) heteroaryl, and
(aa) heteroaryl-C₁₋₂-alkyl,
   wherein any aryl or heteroaryl residue, alone or as part of another group, is optionally substituted in one or more positions with a substituent independently selected from the group Z² consisting of:
(a) halogen selected from chlorine and fluorine,
(b) C₁₋₄-alkoxy,
(c) hydroxymethyl,
(d) —CN,
(e) —CF₃,
(f) C₁₋₄-alkyl,
(g) —OCF₃, and
(h) —C(O)CH₃;
   R⁵ is each independently selected from:
(a) hydrogen,
(b) C₁₋₆-alkyl,
(c) C₃₋₄-cycloalkyl,
(d) fluoro-C₂₋₄-alkyl,
(e) amino-C₂₋₆-alkyl,
(f) cyano-C₁₋₆-alkyl,
(g) hydroxy-C₂₋₆-alkyl,
(h) dihydroxy-C₂₋₆-alkyl,
(i) C₁₋₄-alkoxy-C₂₋₄-alkyl,
(j) C₁₋₄-alkylamino-C₂₋₄-alkyl,
(k) di(C₁₋₄-alkyl)amino-C₂₋₄-alkyl,
(l) aminocarbonyl-C₁₋₄-alkyl,
(m) C₂₋₃-acylamino-C₂₋₄-alkyl,
(n) C₁₋₄-alkylthio-C₂₋₄-alkyl,
(o) C₂₋₄-acyl-C₁₋₄-alkyl, and
(p) C₁₋₄-alkylsulfonyl-C₁₋₄-alkyl,
   or two R⁵ groups together with the nitrogen to which they are attached form a heterocyclic ring, wherein said heterocyclic ring may be optionally substituted with:
   i) a substituent selected from:
(aa) hydroxy,
(bb) amino,
(cc) methylamino,
(dd) dimethylamino,
(ee) hydroxymethyl, and
(ff) aminomethyl;
   ii) one or two oxo groups; or
   iii) one or two fluorine atoms, provided that when the substituent is selected from fluorine, hydroxy, amino, methylamino and dimethylamino, said substituent is attached to the heterocyclic ring at a position other than alpha to a heteroatom; and when the two R⁵ groups form a piperazine ring, the nitrogen of the piperazine ring that allows the substitution is optionally substituted with C₁₋₄-alkyl;
   R⁶ is independently selected from:
(a) hydrogen,
(b) C₁₋₄-alkyl, and
(c) hydroxy-C₂₋₄-alkyl;
   R⁷ is independently selected from:
(a) hydrogen, and
(b) C₁₋₄-alkyl;
   R⁸ is independently selected from:
(a) hydrogen,
(b) C₁₋₆-alkyl,
(c) fluoro-C₁₋₆-alkyl,
(d) hydroxy-C₂₋₆-alkyl,
(e) amino-C₂₋₆-alkyl,
(f) C₁₋₃-alkylamino-C₂₋₄-alkyl,
(g) di(C₁₋₃-dialkyl)amino-C₂₋₄-alkyl,
(h) C₁₋₄-alkylsulfonyl-C₂₋₄-alkyl,
(i) N-heterocyclyl-C₂₋₄-alkyl, wherein heterocyclyl is optionally substituted with methyl,
(j) C-heterocyclyl, optionally substituted with methyl,
(k) C₂₋₃-acylamino-C₂₋₄-alkyl,
(l) [C(OH)CH₃CF₃]—C₁₋₆-alkyl,
(m) C₃₋₆-cycloalkyl,
(n) methyl-C₃₋₆-cycloalkyl,
(o) C₃₋₆-cycloalkyl-C₁₋₂-alkyl,
(p) aryl, and
(q) heteroaryl,
   wherein any aryl or heteroaryl residue is optionally independently substituted in one or two positions with a substituent selected from the group Z² as defined above;
   R⁹ is each independently selected from:
(a) C₁₋₄-alkoxy-C₂₋₄-alkyl,
(b) amino-C₂₋₄-alkyl,
(c) C₁₋₄-alkylamino-C₂₋₄-alkyl,
(d) di(C₁₋₄-alkyl)amino-C₂₋₄-alkyl,
(e) C₂₋₃-acylamino-C₂₋₄-alkyl,
(f) C₁₋₄-alkylthio-C₂₋₄-alkyl, and
(g) C₂₋₄-acyl-C₁₋₄-alkyl,
   or two R⁹ groups together with the nitrogen to which they are attached form a heterocyclic ring, wherein said heterocyclic ring may be optionally substituted with:
   i) a substituent selected from:
(aa) hydroxy,
(bb) amino,
(cc) methylamino,
(dd) dimethylamino,
(ee) hydroxymethyl, and
(ff) aminomethyl;
   ii) one or two oxo groups; or
   iii) one or two fluorine atoms, provided that when the substituent is selected from fluorine, hydroxy, amino, methylamino and dimethylamino, said substituent is attached to the heterocyclic ring at a position other than alpha to a heteroatom; and when the two R⁹ groups form a piperazine ring, the nitrogen of the piperazine ring that allows the substitution is optionally substituted with C₁₋₄-alkyl;
   R¹⁰ is independently selected from:
(a) hydrogen,
(b) C₁₋₆-alkyl,
(c) cyclopropyl,
(d) cyclobutyl,
(e) cyclopropylmethyl,
(f) fluoro-C₂₋₆-alkyl,
(g) hydroxy-C₂₋₆-alkyl,
(h) C₁₋₂-alkoxy-C₂₋₆-alkyl,
(i) amino-C₂₋₆-alkyl,
(j) di(C₁₋₃-alkyl)amino-C₂₋₆-alkyl,
(k) C₁₋₃-alkylamino-C₂₋₆-alkyl,
(l) cyano-C₁₋₄-alkyl,
(m) C₂₋₆-acyl,
(n) C₂₋₆-acyl-C₁₋₆-alkyl,
(o) C₁₋₆-alkylsulfonyl-C₁₋₆-alkyl, and
(p) tetrahydrofuran-2-ylmethyl;
   R¹¹ is selected from:
(a) hydrogen,
(b) hydroxy,
(c) fluorine,
(d) C₁₋₄-alkoxy, and
(e) methyl;
   R¹² is each independently selected from:
(a) hydrogen,
(b) halogen selected from chlorine and fluorine,
(c) —S(O)₂CH₃,
(d) —S(O)₂CF₃,
(e) —OS(O)₂CF₃,
(f) —S(O)NH₂,
(g) —S(O)₂NHCH₃,
(h) —S(O)₂N(CH₃)₂,
(i) —NHS(O)₂CH₃,
(j) —N(CH₃)S(O)₂CH₃,
(k) —NHC(O)CH₃,
(l) —N(CH₃)C(O)CH₃,
(m) —C(O)NH₂,
(n) —C(O)NHCH₃,
(o) —C(O)N(CH₃)₂,
(p) —CN,
(q) —CF₃,
(r) guanidino,
(s) amidino,
(t) —OH,
(u) C₁₋₄-alkoxy,
(v) —OCF₃,
(w) C₃₋₅-cycloalkyloxy,
(x) —SCF₃,
(y) —NO₂,
(z) —NR⁵R⁵, wherein each R⁵ is independently selected from the group consisting of hydrogen and C₁₋₄-alkyl; or two R⁵ groups together with the nitrogen to which they are attached form a pyrrolidine or an azetidine ring,
(aa) —C(OH)CH₃CF₃,
(bb) C₁₋₃-alkyl,
(cc) C₁₋₃-alkoxy-C₁₋₂-alkyl,
(dd) C₂₋₃-acyl,
(ee) C₂₋₃-alkenyl,
(ff) hydroxy-C₁₋₄-alkyl,
(gg) fluoro-C₂₋₃-alkyl,
(hh) C₂₋₃-alkynyl, and
(ii) C₃₋₅-cycloalkyl.

A preferred group of compounds of the invention are compounds of Formula (Ib): 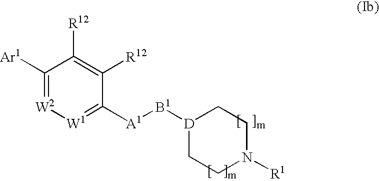
and pharmaceutically acceptable salts, hydrates, geometrical isomers, racemates, tautomers, optical isomers and N-oxides thereof; wherein:
one of W¹ and W² is N and the other is CR¹²;
A¹ is CH₂, O, NR¹⁰, S, S(O) or S(O)₂;
B¹ is CH₂, O, NR¹⁰, S, S(O), S(O)₂, C(O) or CONR¹⁰, provided that when B¹ is O, NR¹⁰, S, S(O), S(O)₂, C(O) or CONR¹⁰, then A¹ is CH₂;
m is each independently 0 or 1;
D is N or CR¹¹, provided that D must be CR¹¹ and said R¹¹ must be hydrogen or methyl when B¹ is selected from O, NR¹⁰, S, S(O), S(O)₂, and CONR¹⁰, and further provided that each m is 1 when D is N;
Ar¹, Z¹, Z², R¹ to R⁹ and R¹² are as defined in Formula (Ia);
R¹⁰ is independently selected from:
(a) hydrogen,
(b) C₁₋₄-alkyl,
(c) cyclopropyl,
(d) cyclobutyl,
(e) cyclopropylmethyl,
(f) fluoro-C₂₋₄-alkyl,
(g) C₁₋₂-alkoxy-C₂₋₃-alkyl,
(h) hydroxy-C₂₋₄-alkyl,
(i) C₂₋₃-acyl,
(j) amino-C₂₋₄-alkyl,
(k) methylamino-C₂₋₄-alkyl,
(l) dimethylamino-C₂₋₄-alkyl,
(m) cyano-C₁₋₄-alkyl, and
(n) tetrahydrofuran-2-ylmethyl;
   R¹¹ is selected from:
(a) hydrogen,
(b) hydroxy,
(c) fluorine, and
(d) methyl.

A further preferred group of compounds of the invention are compounds of Formula (Ic): 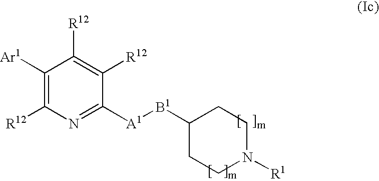
and pharmaceutically acceptable salts, hydrates, geometrical isomers, racemates, tautomers, optical isomers and N-oxides thereof, wherein:
A¹ is CH₂, O or NR¹⁰;
B¹ is CH₂, O or NR¹⁰, provided that when B¹ is O or NR¹⁰, then A¹ is CH₂;
m is each independently 0 or 1;
Z¹, Z², R¹ to R⁷, R⁹ and R¹² are as defined in Formula (Ia), provided that at least two of R¹² are hydrogen;
R¹⁰ is as defined in Formula (Ib);
Ar¹ is phenyl, which is optionally substituted in one or two positions with a substituent independently selected from the group Z³ consisting of:
(a) CF₃SO₃,
(b) halogen selected from bromine, chlorine and fluorine,
(c) C₁₋₄-alkylsulfinyl,
(d) —S(O)₂R⁴,
(e) —S(O)₂NR⁵R⁵,
(f) —NR⁶S(O)₂R⁴,
(g) —NR⁶C(O)R⁴,
(h) —CH₂—NR⁶C(O)R⁴,
(i) —C(O)NR⁵R⁵,
(j) —CH₂—C(O)NR⁵R⁵,
(k) —C(O)R⁴,
(l) H₂N—C(O)O—,
(m) CH₃—NH—C(O)O—,
(n) (CH₃)₂NC(O)O—,
(o) —NHC(O)OCH₃,
(p) C-heterocyclyl, optionally substituted with methyl,
(q) —CN,
(r) —OR⁸,
(s) —SCF₃,
(t) —NO₂,
(u) phosphonooxy,
(v) C-heterocyclylsulfonyl, optionally substituted with methyl,
(w) —NR⁵R⁵,
(x) —C(OH)CH₃CF₃,
(y) cyano-C₁₋₆-alkyl,
(z) guanidino,
(aa) amidino,
(bb) C₁₋₆-alkyl,
(cc) C₁₋₄-alkoxy-C₁₋₄-alkyl,
(dd) fluoro-C₁₋₄-alkyl,
(ee) C₂₋₆-alkenyl,
(ff) fluoro-C₂₋₄-alkenyl,
(gg) hydroxy-C₁₋₆-alkyl,
(hh) C₁₋₄-alkylsulfonyl-C₁₋₄-alkyl,
(ii) hydroxy-C₂₋₄-alkoxy-C₁₋₄-alkyl,
(jj) C₂₋₃-acyl-C₁₋₃-alkyl,
(kk) C₂₋₆-alkynyl,
(ll) C₃₋₆-cycloalkyl,
(mm) hydroxy-C₃₋₆-cycloalkyl,
(nn) fluoro-C₃₋₆-cycloalkyl,
(oo) methyl-C₃₋₆-cycloalkyl,
(pp) C-heterocyclylcarbonyl, optionally substituted with methyl,
(qq) C₃₋₆-cycloalkyl-C₁₋₄-alkyl,
(rr) R⁵R⁵N—C₁₋₂-alkyl,
(ss) —C(O)OR⁷,
(tt)-CH₂C(O)OR⁷,
(uu) aryl, and
(vv) heteroaryl,
   wherein any aryl or heteroaryl residue as substituent on Ar¹ is optionally substituted in one or more positions with a substituent independently selected from the group Z¹ as defined herein for Formula (Ia);
   R⁸ is independently selected from:
(a) hydrogen,
(b) C₁₋₄-alkyl,
(c) CF₃,
(d) C₃₋₅-cycloalkyl,
(e) methyl-C₃₋₅-cycloalkyl, and
(f) C-heterocyclyl, optionally substituted with methyl.

A preferred subgroup of compounds of Formula (Ic) consists of compounds wherein:

A¹ is CH₂ and B¹ is O or NR¹⁰, or

A¹ is O or NR¹⁰ and B¹ is CH₂;

m is each 1;

Ar¹ is phenyl, which is optionally substituted in one or two positions with a substituent independently selected from the group Z⁴ consisting of:

(a) halogen selected from chlorine and fluorine,
(b) C₁₋₄-alkylsulfonyl,
(c) C₁₋₄-alkylsulfinyl,
(d) hydroxy-C₂₋₄-alkylsulfonyl,
(e) C₃₋₅-cycloalkylsulfonyl,
(f) methyl-C₃₋₅-cycloalkylsulfonyl,
(g) trifluoromethylsulfonyl,
(h) —S(O)₂NR^{5A}R^{5A},
(i) C₁₋₄-alkylsulfonamido,
(j) C₂₋₄-acylamino,
(k) C₂₋₄-acylaminomethyl,
(l) carboxy-C₁₋₃-alkylcarbonylamino,
(m) —C(O)NR^{5A}R^{5A},
(n) —CH₂—C(O)NR^{5A}R^{5A},
(o) —NHC(O)OCH₃,
(p) C₂₋₄-acyl,
(q) C₃₋₅-cycloalkylcarbonyl,
(r) C₁₋₄-alkoxy,
(s) C₃₋₅-cycloalkyloxy,
(t) C-heterocyclyl,
(u) —CN,
(v) —OH,
(w) —OCF₃,
(x) —CF₃,
(y) —NO₂,
(z) —NR^{5A}R^{5A},
(aa) —C(OH)CH₃CF₃,
(bb) cyano-C₁₋₂-alkyl,
(cc) C₁₋₄-alkyl,
(dd) C₃₋₅-cycloalkyl,
(ee) C₁₋₂-alkoxy-C₁₋₂-alkyl,
(ff) vinyl,
(gg) ethynyl,
(hh) hydroxy-C₁₋₂-alkyl,
(ii) C-heterocyclyloxy, optionally substituted with methyl,
(jj) R^{5A}R^{5A}N—C₁₋₂-alkyl,
(kk) —C(O)OR^{7A}, and
(ll) —CH₂C(O)OR^{7A}.
   R¹ is a group R^{1A} selected from C(O)OR^{2A}, C(O)R^{2A}, S(O)₂R^{2A}, C(O)NR^{2A}R^{3A}, —CH₂—C(O)NR^{2A}R^{3A}, or a 5- or 6-membered heteroaryl group linked via a ring carbon atom, wherein the said heteroaryl group is optionally substituted with C₁₋₄-alkyl;
   R^{2A} is selected from:
(a) C₁₋₆-alkyl,
(b) C₁₋₆-alkoxy-C₂₋₆-alkyl,
(c) hydroxy-C₂₋₆-alkyl,
(d) hydroxy-C₂₋₄-alkoxy-C₂₋₄-alkyl,
(e) fluoro-C₂₋₆-alkyl,
(f) C₃₋₆-alkynyl,
(g) C₃₋₇-cycloalkyl,
(h) C₅₋₈-cycloalkenyl,
(i) NR^{9A}R^{9A} provided that R^{1A} is not selected from C(O)OR^{2A}, C(O)NR^{2A}R^{3A} and —CH₂—C(O)NR^{2A}R^{3A},
(j) C-heterocyclyl, optionally substituted with methyl,
(k) C₇₋₈-bicyclyl,
(l) 2-norbornylmethyl,
(m) azabicyclyl,
(n) C₃₋₆-cycloalkyl-C₁₋₄-alkyl, wherein cycloalkyl is optionally substituted with methyl,
(o) C₂₋₃-acyl-C₁₋₄-alkyl,
(p) arylcarbonyl-C₁₋₄-alkyl,
(q) heteroarylcarbonyl-C₁₋₄-alkyl,
(r) [C(OH)CH₃CF₃]—C₁₋₆-alkyl,
(s) N-heterocyclylcarbonyl-C₂₋₄-alkyl, wherein heterocyclyl is optionally substituted with methyl,
(t) hydroxy-C₄₋₆-cycloalkyl,
(u) oxo-C₄₋₆-cycloalkyl,
(v) fluoro-C₄₋₆-cycloalkyl,
(w) methoxy-C₄₋₆-cycloalkyl,
(x) methyl-C₃₋₆-cycloalkyl,
(y) oxo-N-heterocyclyl-C₂₋₄-alkyl,
(z) hydroxy-N-heterocyclyl-C₂₋₄-alkyl,
(aa) fluoro-N-heterocyclyl-C₂₋₄-alkyl,
(bb) amino-N-heterocyclyl-C₂₋₄-alkyl,
(cc) N-heterocyclyl-C₂₋₄-alkyl, wherein heterocyclyl is optionally substituted with methyl,
(dd) C-heterocyclyl-C₁₋₄-alkyl, wherein heterocyclyl is optionally substituted with methyl,
(ee) aryl,
(ff) aryl-C₁₋₄-alkyl,
(gg) heteroaryl, and
(hh) heteroaryl-C₁₋₄-alkyl,
   wherein any aryl or heteroaryl residue, alone or as a part of another group, is optionally substituted in one or more positions with a substituent independently selected from the group Z⁵ consisting of:
(a) halogen selected from chlorine and fluorine,
(b) methyl,
(c) ethyl,
(d) methoxy,
(e) ethoxy,
(f) isopropoxy,
(g) hydroxy,
(h) —OCF₃,
(i) —CF₃,
(j) —CN,
(k) —C(OH)CH₃CF₃,
(l) dimethylamino,
(m) hydroxymethyl,
(n) —S(O)₂CH₃,
(o) —C(O)CH₃, and
(p) —C(O)NH₂;
   R^{3A} is selected from:
(a) hydrogen,
(b) C₁₋₄-alkyl,
(c) hydroxy-C₂₋₄-alkyl, and
(d) methoxy-C₂₋₄-alkyl;
   R^{5A} is each independently selected from:
(a) hydrogen,
(b) C₁₋₃-alkyl,
(c) C₁₋₂-alkoxy-C₂₋₄-alkyl,
(d) C₃₋₄-cycloalkyl,
(e) hydroxy-C₂₋₄-alkyl,
(f) cyano-C₁₋₃-alkyl,
(g) dihydroxy-C₂₋₄-alkyl,
(h) aminocarbonyl-C₁₋₂-alkyl, and
(i) di(C₁₋₂-alkyl)amino-C₂₋₃-alkyl;
   or two R^{5A} groups together with the nitrogen to which they are attached form a heterocyclic ring, wherein said heterocyclic ring may be optionally substituted with:
   i) a substituent selected from:
(aa) hydroxy,
(bb) amino,
(cc) methylamino,
(dd) dimethylamino,
(ee) hydroxymethyl, and
(ff) aminomethyl;
   ii) one or two oxo groups; or
   iii) one or two fluorine atoms, provided that when the substituent is selected from fluorine, hydroxy, amino, methylamino and dimethylamino, said substituent is attached to the heterocyclic ring at a position other than alpha to a heteroatom; and when the two R^{5A} groups form a piperazine ring, the nitrogen of the piperazine ring that allows the substitution is optionally substituted with methyl;
   R^{7A} is independently from:
(a) hydrogen, and
(b) C₁₋₄-alkyl;

Two groups R^{9A} together with the nitrogen to which they are attached form a heterocyclic ring, wherein said heterocyclic ring may be optionally substituted with: i) one hydroxy or amino group, ii) one or two fluorine atoms, or iii) one or two oxo groups, provided that when the substituent is selected from fluorine, hydroxy and amino, said substituent is attached to the heterocyclic ring at a position other than alpha to a heteroatom; and when the two R^{9A} groups form a piperazine ring, the nitrogen of the piperazine ring that allows the substitution is optionally substituted with methyl;

R¹⁰ is independently selected from:

(a) hydrogen, and
(b) C₁₋₃-alkyl;
   R¹² is independently selected from:
(a) hydrogen, and
(b) —NO₂.

In a more preferred subgroup of compounds of Formula (Ic), Ar¹ is selected from methylsulfonylphenyl, (morpholin-4-ylsulfonyl)phenyl and cyanophenyl. More preferably, Ar¹ is selected from 4-methylsulfonylphenyl, 4-(morpholin-4-ylsulfonyl)phenyl and 4-cyanophenyl;

In another more preferred subgroup of compounds of Formula (Ic), R^{1A} is selected from C(O)OR^{2A} and C(O)R^{2A}.

In one embodiment, R^{1A} is C(O)OR^{2A}, wherein R^{2A} is selected from tert-butyl, benzyl, isobutyl, ethyl, 4-methoxyphenyl, 2-propynyl, isopropyl, cyclobutyl, 1-cyclopropylethyl, (1S,2R,4R)-bicyclo[2.2.1]hept-2-yl, (3-methyloxetan-3-yl)methyl, (1-methylcyclopropyl)methyl and 3-hydroxy-3-methylbutyl.

In another embodiment, R^{1A} is C(O)R^{2A}, wherein R^{2A} is selected from 2-(3-chloro-4-methoxyphenyl)ethyl, bicyclo[2.2.1]hept-2-yl, cyclohexylmethyl, 5-isopropoxy-pyridin-2-yl, cyclohexyl, 4-methoxycyclohexyl, 3-cyanophenyl, 2-hydroxy-2-methyl-propyl, 3,3,3-trifluoro-2-hydroxy-2-methyl-propyl, 3-acetylphenyl, phenyl, 3-dimethylaminophenyl, 3-oxo-3-phenylpropyl, 2-pyridinyl, 3-hydroxy-2-pyridinyl, 4-isopropoxyphenyl, 2-cyclopentylethyl, (2,3,6-trifluorophenyl)methyl and n-butyl;

In yet another more preferred subgroup of compounds of Formula (Ic), R¹⁰ is selected from hydrogen and methyl.

Particularly preferred compounds of Formula (Ic) are the compounds selected from the group consisting of:
tert-Butyl 4-[({5-[4-(methylsulfonyl)phenyl]pyridin-2-yl}oxy)methyl]piperidine-1-carboxylate;
Benzyl 4-[({5-[4-(methylsulfonyl)phenyl]pyridin-2-yl}oxy)methyl]piperidine-1-carboxylate;
2-({1-[3-(3-Chloro-4-methoxyphenyl)propanoyl]piperidin-4-yl}methoxy)-5-[4-(methylsulfonyl)phenyl]pyridine;
2-{[1-(Bicyclo[2.2.1]hept-2-ylcarbonyl)piperidin-4-yl]methoxy}-5-[4-(methylsulfonyl)phenyl]pyridine;
2-{[1-(Cyclohexylacetyl)piperidin-4-yl]methoxy}-5-[4-(methylsulfonyl)phenyl]-pyridine;
5-Isopropoxy-2-({4-[({5-[4-(methylsulfonyl)phenyl]pyridin-2-yl}oxy)methyl]-piperidin-1-yl}carbonyl)pyridine;
2-{[1-(Cyclohexylcarbonyl)piperidin-4-yl]methoxy}-5-[4-(methylsulfonyl)phenyl]-pyridine;
2-({1-[(4-Methoxycyclohexyl)carbonyl]piperidin-4-yl}methoxy)-5-[4-(methyl sulfonyl)phenyl]pyridine;
3-({4-[({5-[4-(Methylsulfonyl)phenyl]pyridin-2-yl}oxy)methyl]piperidin-1-yl}-carbonyl)benzonitrile;
2-Methyl-4-{4-[({5-[4-(methylsulfonyl)phenyl]pyridin-2-yl}oxy)methyl]piperidin-1-yl}-4-oxobutan-2-ol;
1,1,1-Trifluoro-2-methyl-4-{4-[({5-[4-(methylsulfonyl)phenyl]pyridin-2-yl}oxy)-methyl]piperidin-1-yl}-4-oxobutan-2-ol;
1-[3-({4-[({5-[4-(Methylsulfonyl)phenyl]pyridin-2-yl}oxy)methyl]piperidin-1-yl}-carbonyl)phenyl]ethanone;
tert-Butyl 4-({[5-(4-cyanophenyl)pyridin-2-yl]oxy}methyl)piperidine-1-carboxylate;
tert-Butyl 4-[({5-[4-(morpholin-4-ylsulfonyl)phenyl]pyridin-2-yl}oxy)methyl]-piperidine-1-carboxylate;
2-[(1-Benzoylpiperidin-4-yl)methoxy]-5-[4-(methylsulfonyl)phenyl]pyridine;
N,N-Dimethyl-3-({4-[({5-[4-(methylsulfonyl)phenyl]pyridin-2-yl}oxy)methyl]-piperidin-1-yl}carbonyl)aniline trifluoroacetate;
4-{4-[({5-[4-(Methylsulfonyl)phenyl]pyridin-2-yl}oxy)methyl]piperidin-1-yl}-4-oxo-1-phenylbutan-1-one;
5-[4-(Methylsulfonyl)phenyl]-2-{[1-(pyridin-2-ylcarbonyl)piperidin-4-yl]methoxy}-pyridine;
2-({4-[({5-[4-(Methylsulfonyl)phenyl]pyridin-2-yl}oxy)methyl]piperidin-1-yl}-carbonyl)pyridin-3-ol;
2-{[1-(4-Isopropoxybenzoyl)piperidin-4-yl]methoxy}-5-[4-(methylsulfonyl)phenyl]-pyridine;
tert-Butyl 4-[({5-[4-(methylsulfonyl)phenyl]-3-nitropyridin-2-yl}oxy)methyl]-piperidine-1-carboxylate;
2-{[1-(Cyclohexylacetyl)piperidin-4-yl]methoxy}-5-[4-(methylsulfonyl)phenyl]-3-nitropyridine;
2-{[1-(Bicyclo[2.2.1]hept-2-ylcarbonyl)piperidin-4-yl]methoxy}-5-[4-(methylsulfonyl)phenyl]-3-nitropyridine;
tert-Butyl 4-[({5-[4-(methylsulfonyl)phenyl]pyridin-2-yl}amino)methyl]piperidine-1-carboxylate;
Isobutyl 4-[({5-[4-(methylsulfonyl)phenyl]pyridin-2-yl}amino)methyl]piperidine-1-carboxylate;
Benzyl 4-[({5-[4-(methylsulfonyl)phenyl]pyridin-2-yl}amino)methyl]piperidine-1-carboxylate;
Ethyl 4-[({5-[4-(methylsulfonyl)phenyl]pyridin-2-yl}amino)methyl]piperidine-1-carboxylate;
N-{[1-(Cyclohexylcarbonyl)piperidin-4-yl]methyl}-5-[4-(methylsulfonyl)phenyl]-pyridin-2-amine;
N-{[1-(Cyclohexylacetyl)piperidin-4-yl]methyl}-5-[4-(methylsulfonyl)phenyl]-pyridin-2-amine;
N-{[1-(3-Cyclopentylpropanoyl)piperidin-4-yl]methyl}-5-[4-(methylsulfonyl)-phenyl]pyridin-2-amine;
5-[4-(Methylsulfonyl)phenyl]-N-({1-[(2,3,6-trifluorophenyl)acetyl]piperidin-4-yl}-methyl)pyridin-2-amine;
5-[4-(Methylsulfonyl)phenyl]-N-[(1-pentanoylpiperidin-4-yl)methyl]pyridin-2-amine;
tert-Butyl 4-[(methyl {5-[4-(methylsulfonyl)phenyl]pyridin-2-yl}amino)methyl]-piperidine-1-carboxylate;
tert-Butyl 4-({5-[4-(methyl sulfonyl)phenyl]pyridin-2-yl}methoxy)piperidine-1-carboxylate;
4-Methoxyphenyl 4-({5-[4-(methylsulfonyl)phenyl]pyridin-2-yl}methoxy)-piperidine-1-carboxylate;
Prop-2-yn-1-yl 4-({5-[4-(methylsulfonyl)phenyl]pyridin-2-yl}methoxy)piperidine-1-carboxylate;
2-({[1-(Bicyclo[2.2.1]hept-2-yl carbonyl)piperidin-4-yl]oxy}methyl)-5-[4-(methylsulfonyl)phenyl]pyridine;
Isopropyl 4-({5-[4-(methylsulfonyl)phenyl]pyridin-2-yl}methoxy)piperidine-1-carboxylate;
tert-Butyl 4-[methyl({5-[4-(methylsulfonyl)phenyl]pyridin-2-yl}methyl)amino]-piperidine-1-carboxylate;
(1S,2R,4R)-Bicyclo[2.2.1]hept-2-yl 4-[methyl({5-[4-(methylsulfonyl)phenyl]pyridin-2-yl}methyl)amino]piperidine-1-carboxylate;
(3-Methyloxetan-3-yl)methyl 4-[methyl({5-[4-(methylsulfonyl)phenyl]pyridin-2-yl}-methyl)amino]piperidine-1-carboxylate;
(1-Methylcyclopropyl)methyl 4-[methyl({5-[4-(methylsulfonyl)phenyl]pyridin-2-yl}methyl)amino]piperidine-1-carboxylate;
tert-Butyl 4-[{[5-(4-cyanophenyl)pyridin-2-yl]methyl}(methyl)amino]piperidine-1-carboxylate;
Isobutyl 4-[methyl({5-[4-(methylsulfonyl)phenyl]pyridin-2-yl}methyl)amino]-piperidine-1-carboxylate;
Cyclobutyl 4-[methyl({5-[4-(methylsulfonyl)phenyl]pyridin-2-yl}methyl)amino]-piperidine-1-carboxylate;
1-Cyclopropylethyl 4-[methyl({5-[4-(methylsulfonyl)phenyl]pyridin-2-yl}methyl)-amino]piperidine-1-carboxylate;
Isopropyl 4-[methyl({5-[4-(methylsulfonyl)phenyl]pyridin-2-yl}methyl)amino]-piperidine-1-carboxylate; and
3-Hydroxy-3-methylbutyl 4-[methyl({5-[4-(methylsulfonyl)phenyl]pyridin-2-yl}-methyl)amino]piperidine-1-carboxylate.

A further preferred group of compounds of the invention are compounds of Formula (Id): 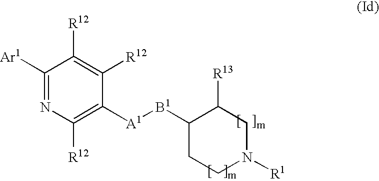
and pharmaceutically acceptable salts, hydrates, geometrical isomers, racemates, tautomers, optical isomers and N-oxides thereof; wherein:
A¹ is CH₂, O or NR¹⁰;
B¹ is CH₂, O or NR¹⁰, provided that when B¹ is O or NR¹⁰, then A¹ is CH₂;
m is each independently 0 or 1;
Z¹, Z², R¹ to R⁷, R⁹ and R¹² are as defined in Formula (Ia), provided that at least two of R² are hydrogen;
R⁸ is as defined in Formula (Ic);
R¹⁰ is as defined in Formula (Ib);
R¹³ is hydrogen or methyl;
Ar¹ is phenyl, which is optionally substituted in one or two positions with a substituent independently selected from the group Z³ as defined in Formula (Ic).

A preferred subgroup of compounds of Formula (Id) consists of compounds wherein:

A¹ is CH₂ and B¹ is O or NR¹⁰, or

A¹ is O or NR¹⁰ and B¹ is CH₂;

m is each 1;

Ar¹ is phenyl, which is optionally substituted in one or two positions with a substituent independently selected from the group Z⁴ as defined in Formula (Ic);

Z⁵ is as defined in Formula (Ic);

R¹ is a group R^{1A}, wherein R^{1A} is as defined in Formula (Ic);

R^{2A}, R^{3A}, R^{4A}, R^{7A} and R^{9A} are as defined in Formula (Ic);

R¹⁰ is independently selected from:

(a) hydrogen,
(b) C₁₋₄-alkyl,
(c) cyclopropyl,
(d) cyclobutyl,
(e) cyclopropylmethyl,
(f) fluoro-C₂₋₄-alkyl,
(g) hydroxy-C₂₋₄-alkyl,
(h) cyano-C₁₋₄-alkyl, and
(i) tetrahydrofuran-2-ylmethyl;
   R¹² is each hydrogen;

In a more preferred subgroup of compounds of Formula (Id), Ar¹ is selected from methylsulfonylphenyl, cyanophenyl, [(dimethylamino)carbonyl]phenyl, (morpholin-4-ylcarbonyl)phenyl, aminocarbonyl)phenyl, [(2-hydroxyethyl)aminocarbonyl]phenyl, [(methoxycarbonyl)amino]phenyl, [(2-hydroxyethyl)sulfonyl]phenyl, carboxyphenyl, fluoro[(propylamino)carbonyl]phenyl, [(cyclopropylamino)carbonyl]phenyl, [(ethylamino)carbonyl]phenyl, [(methylamino)carbonyl]phenyl, [(2-cyanoethyl)aminocarbonyl]-phenyl, (5,6-dihydro-4H-1,3-oxazin-2-yl)phenyl, (acetylamino)phenyl, [(2-methoxyethyl)-aminocarbonyl]phenyl, [(2-hydroxyethyl)aminocarbonyl]phenyl, [(2-hydroxybutyl)aminocarbonyl]phenyl, [(acetylamino)methyl]phenyl, [(4-methylpiperazin-1-yl)carbonyl]phenyl, [2-(hydroxymethyl)morpholin-4-ylcarbonyl]phenyl, [(2-amino-2-oxoethyl)aminocarbonyl]phenyl, [(2-carboxyethyl)carbonylamino]phenyl, (cyanomethyl)phenyl, (methylsulfinyl)phenyl, fluoro(methylsulfonyl)phenyl, (aminocarbonyl)fluorophenyl, (azetidin-1-ylsulfonyl)phenyl, (carboxylmethyl)phenyl, [2-(4-hydroxypiperidin-1-yl)-2-oxoethyl]-phenyl, {2-[2-(hydroxymethyl)morpholin-4-yl]-2-oxoethyl}phenyl and [2-(3-hydroxyazetidin-1-yl)-2-oxoethyl]phenyl.

More preferably, Ar¹ is selected from 4-methylsulfonylphenyl, 4-cyanophenyl, 4-[(dimethylamino)carbonyl]phenyl, 4-(morpholin-4-ylcarbonyl)phenyl, 4-(aminocarbonyl)-phenyl, 3-{[(2-hydroxyethyl)amino]carbonyl}phenyl, 3-(aminocarbonyl)phenyl, 4-[(methoxycarbonyl)amino]phenyl, 4-[(2-hydroxyethyl)sulfonyl]phenyl, 4-carboxyphenyl, 3-fluoro-4-[(propylamino)carbonyl]phenyl, 4-[(cyclopropylamino)carbonyl]phenyl, 4-[(ethylamino)carbonyl]phenyl, 4-[(methylamino)carbonyl]phenyl, 4-{[(2-cyanoethyl)-amino]carbonyl}phenyl, 4-(5,6-dihydro-4H-1,3-oxazin-2-yl)phenyl, 4-(acetylamino)-phenyl, 4-{[(2-methoxyethyl)amino]carbonyl}phenyl, 4-{[(2-hydroxyethyl)amino]-carbonyl}phenyl, 4-{[(2-hydroxybutyl)amino]carbonyl}phenyl, 4-[(acetylamino)methyl]-phenyl, 4-[(4-methylpiperazin-1-yl)carbonyl]phenyl, 4-{[2-(hydroxymethyl)morpholin-4-yl]carbonyl}phenyl, 4-{[(2-amino-2-oxoethyl)amino]carbonyl}phenyl, 4-[(2-carboxyethyl)carbonylamino]phenyl, 4-(cyanomethyl)phenyl, 4-(methylsulfinyl)phenyl, 3-[(acetylamino)methyl]phenyl, 3-(cyanomethyl)phenyl, 2-fluoro-4-(methylsulfonyl)phenyl, 4-(aminocarbonyl)-3-fluorophenyl, 4-(azetidin-1-ylsulfonyl)phenyl, 4-(carboxymethyl)-phenyl, 4-[2-(4-hydroxypiperidin-1-yl)-2-oxoethyl]phenyl, 4-{2-[2-(hydroxymethyl)-morpholin-4-yl]-2-oxoethyl}phenyl and 4-[2-(3-hydroxyazetidin-1-yl)-2-oxoethyl]phenyl.

In another more preferred subgroup of compounds of Formula (Id), R^{1A} is selected from C(O)OR^{2A} and C(O)R^{2A}.

In one embodiment, R^{1A} is C(O)OR^{2A}, wherein R^{2A} is selected from tert-butyl, 2-methoxyethyl, isobutyl, ethyl, isopropyl, benzyl, 2,2-dimethylpropyl, prop-2-yn-1-yl, phenyl, 4-fluorophenyl, 4-methoxyphenyl, 2-fluoro-1-(fluoromethyl)ethyl, (1R)-1-phenylethyl, (1S)-1-phenylethyl, (1S,2R,4R)-bicyclo[2.2.1]hept-2-yl, (1-methylcyclopropyl)methyl, cyclobutyl and 1,3-benzodioxol-5-ylmethyl.

In another embodiment, R^{1A} is C(O)R^{2A}, wherein R^{2A} is selected from tert-butyl, 2-(4-fluorophenyl)ethyl, 4-isopropoxy-phenyl, 3,4-dichlorophenyl, 3-(4-fluorophenyl)propyl, [3-(trifluoromethyl)phenyl]methyl, cyclohexylmethyl, phenyl, 2-methylpropyl, cyclohexyl, 2,2-dimethylpropyl, 2,4-dichlorophenyl, 2,4-difluorophenyl, 2,5-difluorophenyl, 2-fluorophenyl, 3-fluorophenyl, 4-fluorophenyl, 3-methoxyphenyl and 3-chloro-4-methoxyphenyl.

In yet another more preferred subgroup of compounds of Formula (Id), R¹⁰ is independently selected from:
(a) hydrogen,
(b) methyl,
(c) ethyl,
(d) cyclopropyl,
(e) 2-fluoroethyl,
(f) 2,2,2-trifluoroethyl,
(g) isopropyl,
(h) cyclopropylmethyl,
(i) propyl,
(j) 2-hydroxyethyl,
(k) cyanomethyl,
(l) isobutyl,
(m) cyclobutyl,
(n) tetrahydrofuran-2-ylmethyl and
(o) 3,3,3-trifluoropropyl;

Particularly preferred compounds of Formula (Id) are the compounds selected from the group consisting of:
tert-Butyl 4-[({6-[4-(methylsulfonyl)phenyl]pyridin-3-yl}methyl)amino]piperidine-1-carboxylate;
tert-Butyl 4-[methyl({6-[4-(methylsulfonyl)phenyl]pyridin-3-yl}methyl)amino]-piperidine-1-carboxylate;
2-Methoxyethyl 4-[methyl({6-[4-(methylsulfonyl)phenyl]pyridin-3-yl}methyl)-amino]piperidine-1-carboxylate;
Isobutyl 4-[methyl({6-[4-(methylsulfonyl)phenyl]pyridin-3-yl}methyl)amino]-piperidine-1-carboxylate;
Ethyl 4-[methyl({6-[4-(methylsulfonyl)phenyl]pyridin-3-yl}methyl)amino]-piperidine-1-carboxylate;
Isopropyl 4-[methyl({6-[4-(methylsulfonyl)phenyl]pyridin-3-yl}methyl)amino]-piperidine-1-carboxylate;
Benzyl 4-[methyl({6-[4-(methylsulfonyl)phenyl]pyridin-3-yl}methyl)amino]-piperidine-1-carboxylate;
2,2-Dimethylpropyl 4-[methyl({6-[4-(methylsulfonyl)phenyl]pyridin-3-yl}methyl)-amino]piperidine-1-carboxylate;
Prop-2-yn-1-yl 4-[methyl({6-[4-(methylsulfonyl)phenyl]pyridin-3-yl}methyl)-amino]piperidine-1-carboxylate;
Phenyl 4-[methyl({6-[4-(methylsulfonyl)phenyl]pyridin-3-yl}methyl)amino]-piperidine-1-carboxylate;
4-Fluorophenyl 4-[methyl({6-[4-(methylsulfonyl)phenyl]pyridin-3-yl}methyl)-amino]piperidine-1-carboxylate;
4-Methoxyphenyl 4-[methyl({6-[4-(methyl sulfonyl)phenyl]pyridin-3-yl}methyl)-amino]piperidine-1-carboxylate;
2-Fluoro-1-(fluoromethyl)ethyl 4-[methyl({6-[4-(methylsulfonyl)phenyl]pyridin-3-yl}methyl)amino]piperidine-1-carboxylate;
(1R)-1-Phenylethyl 4-[methyl({6-[4-(methylsulfonyl)phenyl]pyridin-3-yl}methyl)-amino]piperidine-1-carboxylate;
(1S)-1-Phenylethyl 4-[methyl({6-[4-(methylsulfonyl)phenyl]pyridin-3-yl}methyl)-amino]piperidine-1-carboxylate;
(1S,2R,4R)-Bicyclo[2.2.1]hept-2-yl 4-[methyl({6-[4-(methylsulfonyl)phenyl]pyridin-3-yl}methyl)amino]piperidine-1-carboxylate;
(1-Methylcyclopropyl)methyl 4-[methyl({6-[4-(methylsulfonyl)phenyl]pyridin-3-yl}methyl)amino]piperidine-1-carboxylate;
Cyclobutyl 4-[methyl({6-[4-(methylsulfonyl)phenyl]pyridin-3-yl}methyl)amino]-piperidine-1-carboxylate;
1,3-Benzodioxol-5-ylmethyl 4-[methyl({6-[4-(methylsulfonyl)phenyl]pyridin-3-yl}-methyl)amino]piperidine-1-carboxylate;
tert-Butyl 4-[(2-fluoroethyl)({6-[4-(methylsulfonyl)phenyl]pyridin-3-yl}methyl)-amino]piperidine-1-carboxylate;
tert-Butyl 4-[(cyclopropylmethyl)({6-[4-(methyl sulfonyl)phenyl]pyridin-3-yl}-methyl)amino]piperidine-1-carboxylate;
tert-Butyl 4-[(2-hydroxyethyl)({6-[4-(methylsulfonyl)phenyl]pyridin-3-yl}methyl)-amino]piperidine-1-carboxylate;
tert-Butyl 4-[(cyanomethyl)({6-[4-(methylsulfonyl)phenyl]pyridin-3-yl}methyl)-amino]piperidine-1-carboxylate;
tert-Butyl 4-[ethyl({6-[4-(methylsulfonyl)phenyl]pyridin-3-yl}methyl)amino]-piperidine-1-carboxylate;
tert-Butyl 4-[cyclobutyl({6-[4-(methylsulfonyl)phenyl]pyridin-3-yl}methyl)amino]-piperidine-1-carboxylate;
tert-Butyl 4-[({6-[4-(methylsulfonyl)phenyl]pyridin-3-yl}methyl)(3,3,3-trifluoropropyl)amino]piperidine-1-carboxylate;
tert-Butyl 4-[({6-[4-(methylsulfonyl)phenyl]pyridin-3-yl}methyl)(2,2,2-trifluoroethyl)amino]piperidine-1-carboxylate;
tert-Butyl 4-[isobutyl({6-[4-(methylsulfonyl)phenyl]pyridin-3-yl}methyl)amino]-piperidine-1-carboxylate;
tert-Butyl 4-[({6-[4-(methylsulfonyl)phenyl]pyridin-3-yl}methyl)(tetrahydrofuran-2-ylmethyl)amino]piperidine-1-carboxylate;
tert-Butyl 4-[isopropyl({6-[4-(methylsulfonyl)phenyl]pyridin-3-yl}methyl)amino]-piperidine-1-carboxylate;
Isopropyl 4-[isopropyl({6-[4-(methylsulfonyl)phenyl]pyridin-3-yl}methyl)amino]-piperidine-1-carboxylate;
tert-Butyl 4-[({6-[4-(methylsulfonyl)phenyl]pyridin-3-yl}methyl)(propyl)amino]-piperidine-1-carboxylate;
Isopropyl 4-[({6-[4-(methylsulfonyl)phenyl]pyridin-3-yl}methyl)(propyl)amino]-piperidine-1-carboxylate;
Isobutyl 4-[({6-[4-(methylsulfonyl)phenyl]pyridin-3-yl}methyl)(propyl)amino]-piperidine-1-carboxylate;
tert-Butyl 4-[cyclopropyl({6-[4-(methylsulfonyl)phenyl]pyridin-3-yl}methyl)-amino]piperidine-1-carboxylate;
Isopropyl 4-[cyclopropyl({6-[4-(methylsulfonyl)phenyl]pyridin-3-yl}methyl)amino]-piperidine-1-carboxylate;
Isobutyl 4-[cyclopropyl({6-[4-(methylsulfonyl)phenyl]pyridin-3-yl}methyl)amino]-piperidine-1-carboxylate;
tert-butyl 4-[cyclopropyl({6-[4-(methylsulfinyl)phenyl]pyridin-3-yl}methyl)amino]-piperidine-1-carboxylate;
tert-butyl 4-{cyclopropyl[(6-{4-[(dimethylamino)carbonyl]phenyl}pyridin-3-yl)-methyl]amino}piperidine-1-carboxylate;
Isopropyl 4-[cyclopropyl({6-[4-(methylsulfinyl)phenyl]pyridin-3-yl}methyl)amino]-piperidine-1-carboxylate;
Isopropyl 4-{cyclopropyl[(6-{4-[(dimethylamino)carbonyl]phenyl}pyridin-3-yl)-methyl]amino}piperidine-1-carboxylate;
tert-Butyl 4-[{[6-(4-cyanophenyl)pyridin-3-yl]methyl}(methyl)amino]piperidine-1-carboxylate;
tert-Butyl 4-[[(6-{4-[(dimethylamino)carbonyl]phenyl}pyridin-3-yl)methyl]-(methyl)amino]piperidine-1-carboxylate;
tert-Butyl 4-[methyl({6-[4-(morpholin-4-ylcarbonyl)phenyl]pyridin-3-yl}methyl)-amino]piperidine-1-carboxylate;
tert-Butyl 4-[({6-[4-(aminocarbonyl)phenyl]pyridin-3-yl}methyl)(3,3,3-trifluoropropyl)amino]piperidine-1-carboxylate;
tert-Butyl 4-[[(6-{4-[(dimethylamino)carbonyl]phenyl}pyridin-3-yl)methyl](3,3,3-trifluoropropyl)amino]piperidine-1-carboxylate;
tert-Butyl 4-[[(6-{4-[(acetylamino)methyl]phenyl}pyridin-3-yl)methyl](3,3,3-trifluoropropyl)amino]piperidine-1-carboxylate;
tert-Butyl 4-[[(6-{3-[(acetylamino)methyl]phenyl}pyridin-3-yl)methyl](3,3,3-trifluoropropyl)amino]piperidine-1-carboxylate;
tert-Butyl 4-[{[6-(3-{[(2-hydroxyethyl)amino]carbonyl}phenyl)pyridin-3-yl]-methyl}(3,3,3-trifluoropropyl)amino]piperidine-1-carboxylate;
tert-Butyl 4-[({6-[3-(aminocarbonyl)phenyl]pyridin-3-yl}methyl)(3,3,3-trifluoropropyl)amino]piperidine-1-carboxylate;
1-(2,2-Dimethylpropanoyl)-N-methyl-N-({6-[4-(methylsulfonyl)phenyl]pyridin-3-yl}methyl)piperidin-4-amine;
tert-Butyl (3R*,4S*)-3-methyl-4-[methyl({6-[4-(methylsulfonyl)phenyl]pyridin-3-yl}methyl)amino]piperidine-1-carboxylate;
tert-Butyl (3S*,4S*)-3-methyl-4-[methyl({6-[4-(methylsulfonyl)phenyl]pyridin-3-yl}methyl)amino]piperidine-1-carboxylate;
1-[3-(4-Fluorophenyl)propanoyl]-N-methyl-N-({6-[4-(methylsulfonyl)phenyl]-pyridin-3-yl}methyl)piperidin-4-amine;
1-(4-Isopropoxybenzoyl)-N-methyl-N-({6-[4-(methylsulfonyl)phenyl]pyridin-3-yl}-methyl)piperidin-4-amine;
1-(3,4-Dichlorobenzoyl)-N-methyl-N-({6-[4-(methylsulfonyl)phenyl]pyridin-3-yl}-methyl)piperidin-4-amine;
1-[4-(4-Fluorophenyl)butanoyl]-N-methyl-N-({6-[4-(methylsulfonyl)phenyl]pyridin-3-yl}methyl)piperidin-4-amine;
N-Methyl-N-({6-[4-(methylsulfonyl)phenyl]pyridin-3-yl}methyl)-1-{[3-(trifluoromethyl)phenyl]acetyl}piperidin-4-amine;
1-(Cyclohexylacetyl)-N-methyl-N-({6-[4-(methylsulfonyl)phenyl]pyridin-3-yl}-methyl)piperidin-4-amine;
1-Benzoyl-N-methyl-N-({6-[4-(methylsulfonyl)phenyl]pyridin-3-yl}methyl)-piperidin-4-amine;
N-Methyl-1-(3-methylbutanoyl)-N-({6-[4-(methylsulfonyl)phenyl]pyridin-3-yl}-methyl)piperidin-4-amine;
1-(Cyclohexylcarbonyl)-N-methyl-N-({6-[4-(methylsulfonyl)phenyl]pyridin-3-yl}-methyl)piperidin-4-amine;
1-(3,3-Dimethylbutanoyl)-N-methyl-N-({6-[4-(methylsulfonyl)phenyl]pyridin-3-yl}-methyl)piperidin-4-amine;
1-(2,4-Dichlorobenzoyl)-N-methyl-N-({6-[4-(methylsulfonyl)phenyl]pyridin-3-yl}-methyl)piperidin-4-amine;
1-(2,4-Difluorobenzoyl)-N-methyl-N-({6-[4-(methylsulfonyl)phenyl]pyridin-3-yl}-methyl)piperidin-4-amine;
1-(2,5-Difluorobenzoyl)-N-methyl-N-({6-[4-(methylsulfonyl)phenyl]pyridin-3-yl}-methyl)piperidin-4-amine;
1-(2-Fluorobenzoyl)-N-methyl-N-({6-[4-(methylsulfonyl)phenyl]pyridin-3-yl}-methyl)piperidin-4-amine;
1-(3-Fluorobenzoyl)-N-methyl-N-({6-[4-(methylsulfonyl)phenyl]pyridin-3-yl}-methyl)piperidin-4-amine;
1-(4-Fluorobenzoyl)-N-methyl-N-({6-[4-(methylsulfonyl)phenyl]pyridin-3-yl}-methyl)piperidin-4-amine;
1-(3-methoxybenzoyl)-N-methyl-N-({6-[4-(methylsulfonyl)phenyl]pyridin-3-yl}-methyl)piperidin-4-amine;
1-(3-Chloro-4-methoxybenzoyl)-N-methyl-N-({6-[4-(methylsulfonyl)phenyl]pyridin-3-yl}methyl)piperidin-4-amine;
tert-Butyl 4-[({6-[4-(methylsulfonyl)phenyl]pyridin-3-yl}amino)methyl]piperidine-1-carboxylate;
tert-Butyl 4-({6-[4-(methylsulfonyl)phenyl]pyridin-3-yl}methoxy)piperidine-1-carboxylate;
tert-Butyl 4-[(6-{4-[(methoxycarbonyl)amino]phenyl}pyridin-3-yl)methoxy]-piperidine-1-carboxylate;
5-[({1-[4-(4-Fluorophenyl)butanoyl]piperidin-4-yl}oxy)methyl]-2-[4-(methylsulfonyl)phenyl]pyridine;
5-({[1-(Cyclohexylacetyl)piperidin-4-yl]oxy}methyl)-2-[4-(methylsulfonyl)phenyl]-pyridine;
tert-Butyl 4-[({6-[4-(methylsulfonyl)phenyl]pyridin-3-yl}oxy)methyl]piperidine-1-carboxylate;
Isobutyl 4-[({6-[4-(methylsulfonyl)phenyl]pyridin-3-yl}oxy)methyl]piperidine-1-carboxylate;
Ethyl 4-[({6-[4-(methylsulfonyl)phenyl]pyridin-3-yl}oxy)methyl]piperidine-1-carboxylate;
tert-Butyl 4-{[(6-{4-[(2-hydroxyethyl)sulfonyl]phenyl}pyridin-3-yl)oxy]methyl}-piperidine-1-carboxylate;
4-(5-{[1-(tert-Butoxycarbonyl)piperidin-4-yl]methoxy}pyridin-2-yl)benzoic acid;
tert-Butyl 4-{[(6-{3-fluoro-4-[(propylamino)carbonyl]phenyl}pyridin-3-yl)oxy]-methyl}piperidine-1-carboxylate;
tert-Butyl 4-{[(6-{4-[(cyclopropylamino)carbonyl]phenyl}pyridin-3-yl)oxy]methyl}-piperidine-1-carboxylate;
tert-Butyl 4-{[(6-{4-[(ethylamino)carbonyl]phenyl}pyridin-3-yl)oxy]methyl}-piperidine-1-carboxylate;
tert-Butyl 4-{[(6-{4-[(methylamino)carbonyl]phenyl}pyridin-3-yl)oxy]methyl}-piperidine-1-carboxylate;
tert-Butyl 4-({[6-(4-{[(2-cyanoethyl)amino]carbonyl}phenyl)pyridin-3-yl]oxy}-methyl)piperidine-1-carboxylate;
tert-Butyl 4-[({6-[4-(5,6-dihydro-4H-1,3-oxazin-2-yl)phenyl]pyridin-3-yl}oxy)-methyl]piperidine-1-carboxylate;
tert-Butyl 4-[({6-[4-(acetylamino)phenyl]pyridin-3-yl}oxy)methyl]piperidine-1-carboxylate;
tert-Butyl 4-({[6-(4-{[(2-methoxyethyl)amino]carbonyl}phenyl)pyridin-3-yl]oxy}-methyl)piperidine-1-carboxylate;
tert-Butyl 4-({[6-(4-{[(2-hydroxyethyl)amino]carbonyl}phenyl)pyridin-3-yl]oxy}-methyl)piperidine-1-carboxylate;
tert-Butyl 4-({[6-(4-{[(2-hydroxybutyl)amino]carbonyl}phenyl)pyridin-3-yl]oxy}-methyl)piperidine-1-carboxylate;
tert-Butyl 4-{[(6-{4-[(acetylamino)methyl]phenyl}pyridin-3-yl)oxy]methyl}-piperidine-1-carboxylate;
tert-Butyl 4-{[(6-{4-[(4-methylpiperazin-1-yl)carbonyl]phenyl}pyridin-3-yl)oxy]-methyl}piperidine-1-carboxylate;
tert-Butyl 4-({[6-(4-{[2-(hydroxymethyl)morpholin-4-yl]carbonyl}phenyl)pyridin-3-yl]oxy}methyl)piperidine-1-carboxylate;
tert-Butyl 4-({[6-(4-{[(2-amino-2-oxoethyl)amino]carbonyl}phenyl)pyridin-3-yl]-oxy}methyl)piperidine-1-carboxylate;
4-{[4-(5-{[1-(tert-Butoxycarbonyl)piperidin-4-yl]methoxy}pyridin-2-yl)phenyl]-amino}-4-oxobutanoic acid;
tert-Butyl 4-[({6-[4-(cyanomethyl)phenyl]pyridin-3-yl}oxy)methyl]piperidine-1-carboxylate;
tert-Butyl 4-[({6-[4-(methylsulfinyl)phenyl]pyridin-3-yl}oxy)methyl]piperidine-1-carboxylate;
tert-Butyl 4-{[(6-{3-[(acetylamino)methyl]phenyl}pyridin-3-yl)oxy]methyl}-piperidine-1-carboxylate;
tert-Butyl 4-[({6-[3-(cyanomethyl)phenyl]pyridin-3-yl}oxy)methyl]piperidine-1-carboxylate;
tert-Butyl 4-[({6-[2-fluoro-4-(methylsulfonyl)phenyl]pyridin-3-yl}oxy)methyl]-piperidine-1-carboxylate;
tert-Butyl 4-[({6-[4-(aminocarbonyl)-3-fluorophenyl]pyridin-3-yl}oxy)methyl]-piperidine-1-carboxylate;
tert-Butyl 4-[({6-[4-(azetidin-1-ylsulfonyl)phenyl]pyridin-3-yl}oxy)methyl]-piperidine-1-carboxylate;
[4-(5-{[1-(tert-Butoxycarbonyl)piperidin-4-yl]methoxy}pyridin-2-yl)phenyl]acetic acid;
tert-Butyl 4-{[(6-{4-[2-(4-hydroxypiperidin-1-yl)-2-oxoethyl]phenyl}pyridin-3-yl)-oxy]methyl}piperidine-1-carboxylate;
tert-Butyl 4-({[6-(4-{2-[2-(hydroxymethyl)morpholin-4-yl]-2-oxoethyl}phenyl)-pyridin-3-yl]oxy}methyl)piperidine-1-carboxylate;
tert-Butyl 4-{[(6-{4-[2-(3-hydroxyazetidin-1-yl)-2-oxoethyl]phenyl}pyridin-3-yl)-oxy]methyl}piperidine-1-carboxylate; and
2-{4-[({6-[4-(Methylsulfonyl)phenyl]pyridin-3-yl}oxy)methyl]piperidin-1-yl}-pyrimidine.

A further preferred group of compounds of the invention are compounds of Formula (Ie): 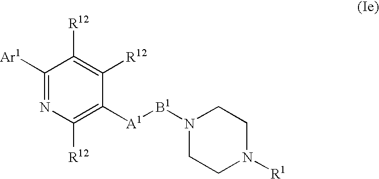
and pharmaceutically acceptable salts, hydrates, geometrical isomers, racemates, tautomers, optical isomers and N-oxides thereof; wherein:
A¹ is CH₂, O or NR¹⁰;
B¹ is CH₂ or C(O);
Z¹, Z², R¹ to R⁷, R⁹ and R¹² are as defined in Formula (Ia), provided that at least two of R¹² are hydrogen;
R⁸ is as defined in Formula (Ic);
R¹⁰ is as defined in Formula (Ib);
Ar¹ is phenyl, which is optionally substituted in one or two positions with a substituent independently selected from the group Z³ as defined in Formula (Ic).

A preferred subgroup of compounds of Formula (Ie) consists of compounds wherein:

A¹ is CH₂;

B¹ is CH₂ or C(O);

Ar¹ is phenyl, which is optionally substituted in one or two positions with a substituent independently selected from the group Z⁴ as defined in Formula (Ic);

Z⁵ is as defined in Formula (Ic);

R¹ is a group R^{1A}, wherein R^{1A} is as defined in Formula (Ic);

R^{2A}, R^{3A}, R^{5A}, R^{7A} and R^{9A} are as defined in Formula (Ic);

R¹² is each hydrogen;

In a more preferred subgroup of compounds of Formula (Ie), Ar¹ is C₁₋₄-alkylsulfonylphenyl. It is especially preferred for Ar¹ to be methylsulfonylphenyl.

In another more preferred subgroup of compounds of Formula (Ie), R^{1A} is selected from C(O)OR^{2A} and C(O)R^{2A}.

In one embodiment, R^{1A} is C(O)OR^{2A} wherein R^{2A} is C₁₋₆-alkyl. Preferably, R^{2A} is selected from tert-butyl and isobutyl.

In another embodiment, R^{1A} is C(O)R^{2A} wherein R^{2A} is phenyl, which is monosubstituted with a substituent selected from methoxy, ethoxy and isopropoxy. Preferably, R^{2A} is 4-isopropoxyphenyl.

Particularly preferred compounds of Formula (Ie) are the compounds selected from the group consisting of:
tert-Butyl 4-({6-[4-(methylsulfonyl)phenyl]pyridin-3-yl}acetyl)piperazine-1-carboxylate;
tert-Butyl 4-(2-{6-[4-(methylsulfonyl)phenyl]pyridin-3-yl}ethyl)piperazine-1-carboxylate;
1-(4-Isopropoxybenzoyl)-4-(2-{6-[4-(methylsulfonyl)phenyl]pyridin-3-yl}ethyl)-piperazine; and
Isobutyl 4-(2-{6-[4-(methylsulfonyl)phenyl]pyridin-3-yl}ethyl)piperazine-1-carboxylate.

All isomeric forms possible (pure enantiomers, diastereomers, tautomers, racemic mixtures and unequal mixtures of two enantiomers) for the compounds delineated are within the scope of the invention. When the compounds described herein contain olefinic double bonds of geometric asymmetry, it is intended to include both trans and cis (E and Z) geometric isomers.

The compounds of the Formula (Ia) to (Ie) may be used as such or, where appropriate, as pharmacologically acceptable salts (acid or base addition salts) thereof. The pharmacologically acceptable addition salts mentioned below are meant to comprise the therapeutically active non-toxic acid and base addition salt forms that the compounds are able to form. Compounds that have basic properties can be converted to their pharmaceutically acceptable acid addition salts by treating the base form with an appropriate acid. Exemplary acids include inorganic acids, such as hydrogen chloride, hydrogen bromide, hydrogen iodide, sulphuric acid, phosphoric acid; and organic acids such as formic acid, acetic acid, propanoic acid, hydroxyacetic acid, lactic acid, pyruvic acid, glycolic acid, maleic acid, malonic acid, oxalic acid, benzenesulphonic acid, toluenesulphonic acid, methanesulphonic acid, trifluoroacetic acid, fumaric acid, succinic acid, malic acid, tartaric acid, citric acid, salicylic acid, p-aminosalicylic acid, pamoic acid, benzoic acid, ascorbic acid and the like. Exemplary base addition salt forms are the sodium, potassium, calcium salts, and salts with pharmaceutically acceptable amines such as, for example, ammonia, alkylamines, benzathine, and amino acids, such as, e.g. arginine and lysine. The term addition salt as used herein also comprises solvates which the compounds and salts thereof are able to form, such as, for example, hydrates, alcoholates and the like.

Another object of the present invention is a compound of Formula (Ia) to (Ie) for use in therapy. The compound can be used in the treatment or prophylaxis of disorders relating to GPR119. Examples of such disorders are Type 1 and Type 2 diabetes, inadequate glucose tolerance, insulin resistance, hyperglycemia, hyperlipidemia, hypercholesterolemia, dyslipidemia, syndrome X, metabolic syndrome, obesity, hypertension, chronic systemic inflammation, retinopathy, neuropathy, nephropathy, atherosclerosis, reduced fibrinolysis, endothelial dysfunction.

Another object of the present invention is a method for the treatment or prophylaxis of disorders related to GPR119, said method comprising administering to a subject (e.g., mammal, human, or animal) in need of such treatment an effective amount of a compound as described above. The GPR119-related disorder is any disorder or symptom wherein GPR119 is involved in the process or presentation of the disorder or the symptom. The GPR119-related disorders include, but are not limited to Type 1 and Type 2 diabetes, inadequate glucose tolerance, insulin resistance, hyperglycemia, hyperlipidemia, hypercholesterolemia, dyslipidemia, syndrome X, metabolic syndrome, obesity, hypertension, chronic systemic inflammation, retinopathy, neuropathy, nephropathy, atherosclerosis, reduced fibrinolysis, endothelial dysfunction.

Another object of the present invention is a method for modulating the GPR119 receptor activity (e.g., agonizing human GPR119), comprising administering to a subject (e.g., mammal, human, or animal) in need thereof an effective amount of a compound as described above or a composition comprising a compound as described above.

Another object of the present invention is the use of a compound as described above in the manufacture of a medicament for use in the treatment or prophylaxis of Type 1 and Type 2 diabetes, inadequate glucose tolerance, insulin resistance, hyperglycemia, hyperlipidemia, hypercholesterolemia, dyslipidemia, syndrome X, metabolic syndrome, obesity, hypertension, chronic systemic inflammation, retinopathy, neuropathy, nephropathy, atherosclerosis, reduced fibrinolysis, endothelial dysfunction.

Another object of the present invention is the use of a compound of Formula (Ia) to (Ie), as described above, in the manufacture of a medicament for use in the treatment or prophylaxis of disorders related to GPR119, said method comprising administering to a subject (e.g., mammal, human, or animal) in need of such treatment an effective amount of a compound as described above. The GPR119-related disorder is any disorder or symptom wherein GPR119 is involved in the process or presentation of the disorder or the symptom. The GPR119-related disorders include, but are not limited to, Type 1 and Type 2 diabetes, inadequate glucose tolerance, insulin resistance, hyperglycemia, hyperlipidemia, hypercholesterolemia, dyslipidemia, syndrome X, metabolic syndrome, obesity, hypertension, chronic systemic inflammation, retinopathy, neuropathy, nephropathy, atherosclerosis, reduced fibrinolysis, endothelial dysfunction.

Methods delineated herein include those wherein the subject is identified as in need of a particular stated treatment. Identifying a subject in need of such treatment can be in the judgment of a subject or a health care professional and can be subjective (e.g. opinion) or objective (e.g. measurable by a test or diagnostic method).

In other aspects, the methods herein include those further comprising monitoring subject response to the treatment administrations. Such monitoring may include periodic sampling of subject tissue, fluids, specimens, cells, proteins, chemical markers, genetic materials, etc. as markers or indicators of the treatment regimen. In other methods, the subject is prescreened or identified as in need of such treatment by assessment for a relevant marker or indicator of suitability for such treatment.

In one embodiment, the invention provides a method of monitoring treatment progress. The method includes the step of determining a level of diagnostic marker (Marker) (e.g., any target or cell type delineated herein modulated by a compound herein) or diagnostic measurement (e.g., screen, assay) in a subject suffering from or susceptible to a disorder or symptoms thereof delineated herein, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

In certain method embodiments, a level of Marker or Marker activity in a subject is determined at least once. Comparison of Marker levels, e.g., to another measurement of Marker level obtained previously or subsequently from the same patient, another patient, or a normal subject, may be useful in determining whether therapy according to the invention is having the desired effect, and thereby permitting adjustment of dosage levels as appropriate. Determination of Marker levels may be performed using any suitable sampling/expression assay method known in the art or described herein. Preferably, a tissue or fluid sample is first removed from a subject. Examples of suitable samples include blood, urine, tissue, mouth or cheek cells, and hair samples containing roots. Other suitable samples would be known to the person skilled in the art. Determination of protein levels and/or mRNA levels (e.g., Marker levels) in the sample can be performed using any suitable technique known in the art, including, but not limited to, enzyme immunoassay, ELISA, radiolabelling/assay techniques, blotting/chemiluminescence methods, real-time PCR, and the like.

For clinical use, the compounds of the invention are formulated into pharmaceutical formulations for oral, rectal, parenteral or other mode of administration. Pharmaceutical formulations are usually prepared by mixing the active substance, or a pharmaceutically acceptable salt thereof, with conventional pharmaceutical excipients. Examples of excipients are water, gelatin, gum arabicum, lactose, microcrystalline cellulose, starch, sodium starch glycolate, calcium hydrogen phosphate, magnesium stearate, talcum, colloidal silicon dioxide, and the like. Such formulations may also contain other pharmacologically active agents, and conventional additives, such as stabilizers, wetting agents, emulsifiers, flavouring agents, buffers, and the like. Usually, the amount of active compounds is between 0.1-95% by weight of the preparation, preferably between 0.2-20% by weight in preparations for parenteral use and more preferably between 1-50% by weight in preparations for oral administration.

The dose level and frequency of dosage of the specific compound will vary depending on a variety of factors including the potency of the specific compound employed, the metabolic stability and length of action of that compound, the patient's age, body weight, general health, sex, diet, mode and time of administration, rate of excretion, drug combination, the severity of the condition to be treated, and the patient undergoing therapy. The daily dosage may, for example, range from about 0.001 mg to about 100 mg per kilo of body weight, administered singly or multiply in doses, e.g. from about 0.01 mg to about 25 mg each. Normally, such a dosage is given orally but parenteral administration may also be chosen.

The formulations can be further prepared by known methods such as granulation, compression, microencapsulation, spray coating, etc. The formulations may be prepared by conventional methods in the dosage form of tablets, capsules, granules, powders, syrups, suspensions, suppositories or injections. Liquid formulations may be prepared by dissolving or suspending the active substance in water or other suitable vehicles. Tablets and granules may be coated in a conventional manner.

The compounds of formula (Ia) to (Ie) may be administered with other active compounds for the treatment of diabetes and/or obesity, for example insulin and insulin analogs, DPP-IV inhibitors, sulfonyl ureas, biguanides, α2 agonists, glitazones, PPAR-γ agonists, mixed PPAR-α/γ agonists, RXR agonists, α-glucosidase inhibitors, PTP1B inhibitors, 11-β-hydroxy steroid dehydrogenase Type 1 inhibitors, phosphodiesterase inhibitors, glycogen phosphorylase inhibitors, MCH-1 antagonists, CB-1 antagonists (or inverse agonists), amylin antagonists, CCK receptor agonists, β₃-agonists, leptin and leptin mimetics, serotonergic/dopaminergic antiobesity drugs, gastric lipase inhibitors, pancreatic lipase inhibitors, fatty acid oxidation inhibitors, lipid lowering agents and thyromimetics.

It is particularly preferred that the compounds of formula (Ia) to (Ie) are administered in combination with a DPP-IV inhibitor. The term “DPP-IV inhibitor” means a compound which inhibits, antagonizes or decreases the activity of dipeptidyl peptidase IV (EC 3.4.14.5). The said DPP-IV inhibitor can e.g. be a compound as disclosed in WO 2005/056003; WO 2005/056013; WO 2005/095343; WO 2005/113510; WO 2005/120494; WO 2005/121131; WO 2005/121089; WO 2006/013104; or WO 2006/076231, including references therein.

In a further aspect the invention relates to methods of making compounds of any of the formulae herein comprising reacting any one or more of the compounds of the formulae delineated herein, including any processes delineated herein. The compounds of the Formula (Ia) to (Ie) above may be prepared by, or in analogy with, conventional methods. The preparation of intermediates and compounds according to the examples of the present invention may in particular be illuminated by the following Schemes 1-5. 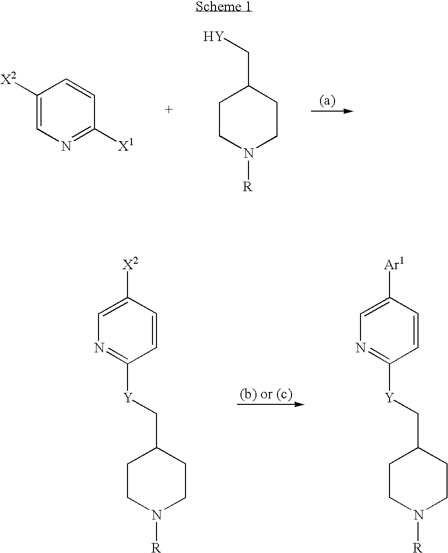
X¹ is Cl, Br;
X² is Cl, Br, I;
Y is O or NH;
R is Boc, CBz or benzyl;
Ar¹ is as defined in Formula (Ia).

Reagents and conditions:
(a) suitable base, such as NaH or t-BuOK; in a suitable solvent, such as DMF, DMSO or THF; at ambient or elevated temperature;
(b) appropriate arylboronic acid; appropriate catalyst, such as Pd(PPh₃)₄; a suitable base, such as K₂CO₃ or NaHCO₃; in a suitable solvent mixture such as 1,4-dioxane and water; at elevated temperature, for example 90° C.;
(c) (i) bis(neopentyl glycolato)diboron; suitable base, such as KOAc; appropriate catalyst, such as PdCl₂(dppf)-DCM; in a suitable solvent, such as DME; at elevated temperature, for example 120° C. (microwaves); (ii) appropriate aryl halide; suitable base, such as NaHCO₃; appropriate catalyst, such as Pd(PPh₃)₄; in a suitable solvent mixture, such as water and DME; at elevated temperature, for example 120° C. (microwaves). 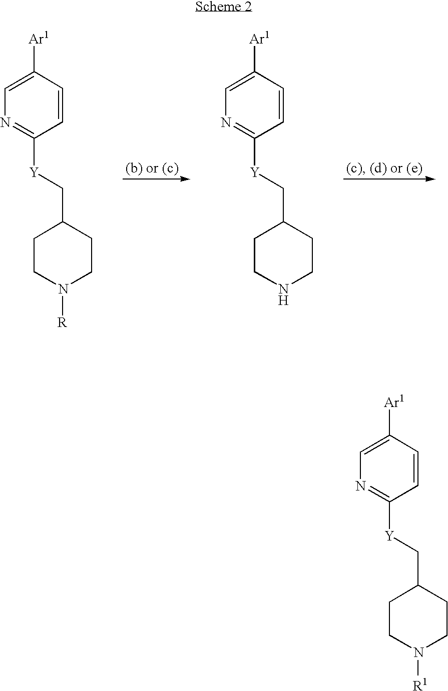
   Y is O or NH;
   Ar¹ is as defined in Formula (Ia);
   R is Boc, CBz or benzyl;
   R¹ is as defined in Formula (Ia).

Reagents and conditions:
(a) suitable deprotecting agent, such as TFA, HCl (g) or aqueous concentrated HCl; in a suitable solvent, such as DCM or ethanol; at ambient or elevated temperature; when R=Boc;
(b) hydrogenolysis, suitable catalyst, such as 10% Pd/C; suitable hydrogen source, such as ammonium formate or H₂ (g); in suitable solvent, such as n-propanol, ethanol, water, or mixtures thereof, at elevated temperature, for example 90° C.; when R=benzyl or CBz;
(c) (i) appropriate carboxylic acid; suitable base, such as triethylamine; in suitable solvent, such as THF, dioxane or DMF; (ii) appropriate coupling reagent, such as HOBT/EDC, propylphosphonic anhydride or TBTU;
(d) appropriate acid chloride or chloroformate; suitable base, such as triethylamine; in suitable solvent, such THF or DMF;
(e) appropriate alcohol; suitable coupling reagents, such as 1,1′-carbonylbis(1H-imidazole); in suitable solvent, such DCM, acetonitrile or DCM/THF; at elevated temperature. 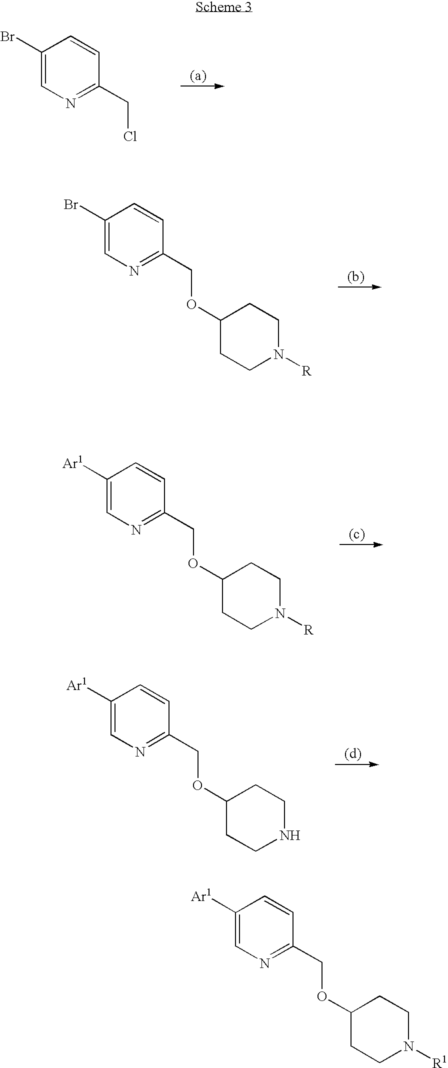
   Ar¹ is as defined in Formula (Ia);
   R is Boc;
   R₁ is as defined in Formula (Ia);

Reagents and conditions:
(a) tert-butyl 4-hydroxypiperidine-1-carboxylate; suitable base, such as potassium tert-butoxide or NaH; in a suitable solvent, such as THF or DMF; at elevated temperature, for example 60° C.;
(b) appropriate arylboronic acid; appropriate catalyst, such as Pd(PPh₃)₄; a suitable base, such as K₂CO₃ or NaHCO₃; in a suitable solvent mixture, such as 1,4-dioxane and water; at elevated temperature, for example 90° C.;
(c) (i) suitable deprotecting agent, such as TFA, HCl (g) or aqueous concentrated HCl; in a suitable solvent, such as DCM or ethanol; at ambient or elevated temperature; (ii) suitable base, such as 2 M NaOH;
(d) (i) appropriate carboxylic acid; suitable base, such as triethylamine; in suitable solvent, such as THF, dioxane or DMF; (ii) appropriate coupling reagent, such as HOBT/EDC, propylphosphonic anhydride or TBTU. 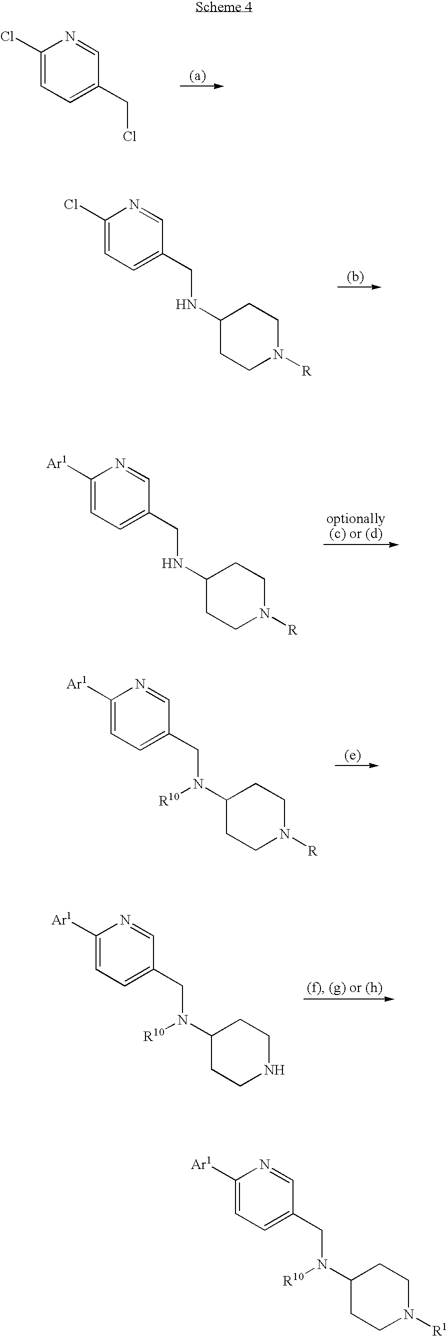
   Ar¹ is as defined in Formula (Ia);
   R is Boc;
   R¹ is as defined in Formula (Ia);
   R¹⁰ is as defined in Formula (Ia);

Reagents and conditions:
(a) 4-amino-piperidine-1-carboxylic acid tert-butyl ester, suitable base, such as N,N-diisopropylethylamine or triethylamine; in a suitable solvent, such as DMF; at elevated temperature;
(b) appropriate arylboronic acid; appropriate catalyst, such as Pd(PPh₃)₄; a suitable base, such as K₂CO₃ or NaHCO₃; in a suitable solvent mixture such as 1,4-dioxane and water; at elevated temperature, for example 90° C.;
(c) appropriate aldehyde or ketone corresponding to R¹⁰; appropriate reducing agents, e.g., NaBH(OAc)₃ or NaBH₃CN; in a suitable solvent, such as 1,2-dichloroethane, DCM, or in a solvent mixture such as methanol/water; at ambient or elevated temperature;
(d) appropriate alkylating agent corresponding to R¹⁰, such as alkylhalide, alkyltriflate; suitable base, such N,N-diisopropylethyl amine or triethylamine; in a suitable solvent, such as THF or DMF; at elevated temperature;
(e) (i) suitable deprotecting agent, such as TFA, HCl (g) or aqueous concentrated HCl; in a suitable solvent, such as DCM or ethanol; at ambient or elevated temperature; (ii) suitable base, such as 2 M NaOH;
(f) appropriate carboxylic acid; suitable base, such as triethylamine; suitable coupling reagents such as TBTU; in a suitable solvent such as DMF; at ambient temperature;
(g) appropriate acid chloride or chloroformate, suitable base such as triethylamine, in a suitable solvent, such as DCM or DMF;
(h) appropriate alcohol; suitable coupling reagents, such as 1,1′-carbonylbis(1H-imidazole); in suitable solvent, such DCM, acetonitrile or DCM/THF; at elevated temperature. 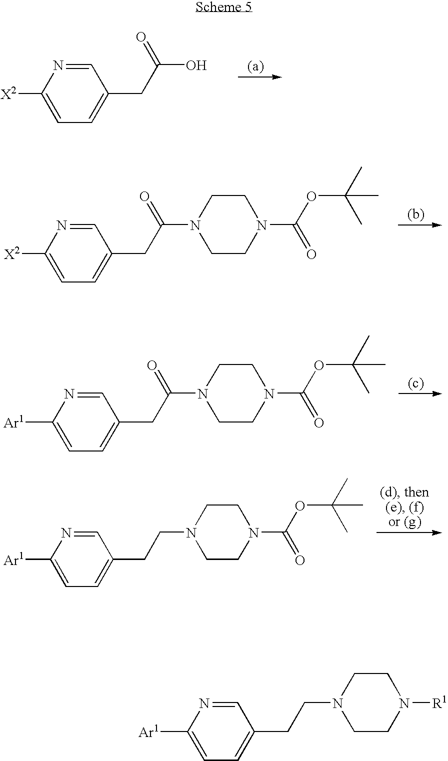
   X² is Cl, Br, I;
   Ar¹ is as defined in Formula (Ia);
   R¹ is as defined in Formula (Ia);

Reagents and conditions:
(a) appropriate coupling reagent, such as HOBT/EDC or propylphosphonic anhydride; suitable base, such as triethylamine; in a suitable solvent mixture, such as THF/MeOH; at ambient temperature;
(b) appropriate arylboronic acid, appropriate catalyst, such as Pd(PPh₃)₄; a suitable base, such as K₂CO₃ or NaHCO₃; in a suitable solvent mixture such as 1,4-dioxane and water; at elevated temperature, for example 90° C.;
(c) appropriate reducing agent, such as borane-methyl sulfide complex; in a suitable solvent, such as THF; at elevated temperature.
(d) (i) suitable deprotecting agent, such as TFA, HCl (g) or aqueous concentrated HCl; in a suitable solvent, such as DCM or ethanol; at ambient or elevated temperature; (ii) suitable base, such as 2 M NaOH;
(e) appropriate carboxylic acid; suitable base, such as triethylamine; suitable coupling reagents such as TBTU; in a suitable solvent such as DMF; at ambient temperature;
(f) appropriate acid chloride or chloroformate; suitable base such as triethylamine; in a suitable solvent, such as DCM or DMF;
(g) appropriate alcohol; suitable coupling reagents, such as 1,1′-carbonylbis(1H-imidazole); in suitable solvent, such DCM, acetonitrile or DCM/THF; at elevated temperature.

Definitions of variables in the structures in the schemes herein are commensurate with those of corresponding positions in the formulae delineated herein.

The processes described below in the example section may be carried out to give a compound of the invention in the form of a free base or as an acid addition salt. A pharmaceutically acceptable acid addition salt may be obtained by dissolving the free base in a suitable organic solvent and treating the solution with an acid, in accordance with conventional procedures for preparing acid addition salts from base compounds. Examples of addition salt forming acids are mentioned above.

The compounds of Formula (Ia) to (Ie) may possess one or more chiral carbon atoms, and they may therefore be obtained in the form of optical isomers, e.g. as a pure enantiomer, or as a mixture of enantiomers (racemate) or as a mixture containing diastereomers. The separation of mixtures of optical isomers to obtain pure enantiomers is well known in the art and may, for example, be achieved by fractional crystallization of salts with optically active (chiral) acids or by chromatographic separation on chiral columns.

The chemicals used in the synthetic routes delineated herein may include, for example, solvents, reagents, catalysts, and protecting group and deprotecting group reagents. The methods described above may also additionally include steps, either before or after the steps described specifically herein, to add or remove suitable protecting groups in order to ultimately allow synthesis of the compounds. In addition, various synthetic steps may be performed in an alternate sequence or order to give the desired compounds. Synthetic chemistry transformations and protecting group methodologies (protection and deprotection) useful in synthesizing applicable compounds are known in the art and include, for example, those described in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

The necessary starting materials for preparing the compounds of Formula (Ia) to (Ie) and other compounds herein are either known or may be prepared in analogy with the preparation of known compounds.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

The invention will now be further illustrated by the following non-limiting Examples. The specific examples below are to be construed as merely illustrative, and not limitative of the remainder of the disclosure in any way whatsoever. Without further elaboration, it is believed that one skilled in the art can, based on the description herein, utilize the present invention to its fullest extent. All references and publications cited herein are hereby incorporated by reference in their entirety.

### EXAMPLES AND INTERMEDIATE COMPOUNDS

### Experimental Methods

¹H Nuclear magnetic resonance (NMR) and ¹³C NMR were recorded on a Bruker Advance DPX 400 spectrometer at 400.1 MHz and 100.6 MHz, respectively. All spectra were recorded using residual solvent or tetramethylsilane (TMS) as internal standard. Low-resolution electrospray ionization mass spectra (LRESIMS) were obtained using an Agilent MSD mass spectrometer or a Waters ZQ mass spectrometer. High-resolution electrospray ionization mass spectra (HRESIMS) were obtained on an Agilent LC/MSD TOF connected to an Agilent 1100 LC-system, on Source: ESI, Ion polarity: pos, Data: profile mode, Scan range: 100-1100 Da, MS parameters; Fragmentor 215V, Skimmer 560V och OCT RF (octpole rods) 250 V.; Reference Masses 121.050873 and 922.009798 (Agilent reference Mix); LC: A 15 mM ammonium acetate; B 100 MeCN; flow 400 μL/min isocratic. Flash chromatography was performed on Merck silica gel 60 (230-400 mesh). Microwave irraditions were carried out using the Smith Creator or Optimizer (Personal Chemistry) using 0.5-2 mL or 2-5 mL Smith Process vials fitted with aluminum caps and septa. The compounds were automatically named using ACD 6.0.

Analytical LCMS data were obtained with:

System A: Agilent MSD mass spectrometer; Agilent 1100 system; ACE 3 C8 column (50×3.0 mm); Water containing 0.1% TFA and acetonitrile were used as mobile phases at a flow rate of 1 mL/min with gradient times of 3.0 min (gradient 10-97% acetonitrile); or

System B: Agilent MSD mass spectrometer; Agilent 1100 system; YMC ODS-AQ column (33×3.0 mm); Water containing 0.1% TFA and acetonitrile were used as mobile phases at a flow rate of 1 mL/min with gradient times of 3.0 min (gradient 10-97% acetonitrile); or

System C: Waters ZQ mass spectrometer; Waters 996 PDA detector (DAD 215-395 nm); ACE C8 (3 μm) column (30×3.0 mm) (from ACT); Water containing 10 mM ammonium acetate (pH=7) and acetonitrile were used as mobile phases at a flow rate of 1 mL/min with gradient times of 3.2 min (gradient 5-100% acetonitrile).

Preparative HPLC was performed on Gilson system equipped with:

System D: ACE C8 5 μm (21.2×50 mm) column. Water containing 0.1% TFA and acetonitrile were used as mobile phases at a flow rate of 25 mL/min with gradient times of 6 min.; or

System E: XTerra Prep MS C18 5 μm (19×50 mm) column. Water containing 50 mM NH₄HCO₃ (pH=10) and acetonitrile were used as mobile phases at a flow rate of 25 mL/min with gradient times of 6 min; or Xterra MS C18 5 μm (30×100 mm) column. Water containing 50 mM NH₄HCO₃ (pH=10) and acetonitrile were used as mobile phases at a flow rate of 40 mL/min with gradient times of 8.5 min.

Methods for Preparation

General Method A: Preparation of Carbamates and Amides (from Chloroformates or Acid Chlorides).

N-Methyl-N-({6-[4-(methylsulfonyl)phenyl]pyridin-3-yl}methyl)piperidin-4-amine (327 mg, 13×0.07 mmol; Intermediate B2) was dissolved in DCM (9.1 mL) and distributed into 13 vials (4 mL each). Triethylamine (0.025 mL, 18 mg, 0.18 mmol) was added to each vial followed by the appropriate chloroformates or acid chlorides (0.1 mmol) in DCM (0.7 mL). The reaction mixtures were stirred at r.t. and the progress was monitored by analytical LC-MS. When the reaction was completed NH₄OAc in MeOH (0.5 mL) was added and the mixture was evaporated under reduced pressure. The crude product was purified by preparative HPLC (System D) to give the desired products.

General Method B: Preparation of Amides from Carboxylic Acids Using TBTU as Coupling Reagent.

N-Methyl-N-({6-[4-(methylsulfonyl)phenyl]pyridin-3-yl}methyl)piperidin-4-amine (276 mg, 0.77 mmol; Intermediate B2) was dissolved in DMF (7.7 mL) and triethylamine (0.215 mL, 155 mg, 1.54 mmol) was added. The solution was distributed into 11 vials containing the appropriate carboxylic acids (0.084 mmol). TBTU (27 mg, 0.084 mmol) was added to each vial. The solutions were stirred at room temperature overnight and then concentrated under reduced pressure. The crude products were purified by preparative HPLC (System D).

General Method C: Palladium-Catalyzed Suzuki-Type Coupling Reaction.

tert-Butyl 4-{[(6-chloropyridin-3-yl)methyl]amino}piperidine-1-carboxylate (65 mg, 0.2 mmol; Intermediate B1) and the appropriate arylboronic acid (0.24 mmol) were mixed in dioxane (1.6 mL). K₂CO₃ (69 mg, 0.5 mmol) in water (0.4 mL) and Pd(PPh₃)₄ (0.12 mg, 0.01 mmol) were added to the mixture. The reaction mixture was heated to 80° C. and the progress was monitored by analytical LC-MS. The solvent was removed under reduced pressure and the residue was partitioned between 10% aqueous Na₂CO₃ (0.8 mL) and DCM (7 mL). The organic phase was separated and concentrated under reduced pressure. The crude product was purified by preparative HPLC (System E).

General Method D: Reductive Amination with 3,3,3-trifluoropropanal.

The starting amine in Example B26 (tert-butyl 4-[({6-[4-(methylsulfonyl)phenyl]pyridin-3-yl}methyl)amino]piperidine-1-carboxylate, 0.024 mmol) was dissolved in THF (1 mL). 3,3,3-Trifluoropropanal (58 mg, 0.045 mL, 0.5 mmol) and NaBH(OAc)₃ (76 mg, 0.36 mmol) were added and the mixture was stirred at r.t. overnight. Work-up was performed by addition of 1 mL 10% aqueous Na₂CO₃ and extraction with DCM (8 mL). After evaporation the residue was purified by preparative HPLC (System D, water containing 5 mM NH₄OAc and acetonitrile were used as mobile phase, gradient 40-75% acetonitrile). The starting amines in Examples B45, B46, B47, B48, B49 and B50 were subjected to the same procedure.

### Intermediate A1

### 2-[(1-Benzylpiperidin-4-yl)methoxy]-5-bromopyridine

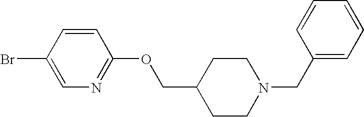

To a solution of 2,5-dibromopyridine (1.0 g, 4.2 mmol) in DMF (25 mL) was added sodium, hydride (0.21 g, 0.0063 mol; 60% dispersion in mineral oil). The mixture was cooled to 0° C. and treated slowly with (1-benzylpiperidin-4-yl)methanol (0.86 g, 4.2 mmol). The reaction was allowed to reach room temperature and then stirred for 72 hours. The reaction mixture was concentrated under reduced pressure. Brine (75 mL) was added and the mixture was extracted with chloroform (3×100 mL). The combined organic layers were concentrated. The crude residue was purified by flash chromatography on silica with gradient elution (first 10% EtOAc in DCM, then 30% EtOAc in DCM) to give the title compound as a light brown solid. Yield: 1.13 g (74%); Analytical HPLC: purity 99% (System A and B); LRESIMS for C₁₈H₂₁BrN₂O m/z 361 (M+H)⁺.

### Intermediate A2

### tert-Butyl 4-{[(5-bromopyridin-2-yl)oxy]methyl}piperidine-1-carboxylate

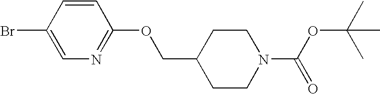

To a solution of 2,5-dibromopyridine (3.0 g, 0.0127 mol) in DMF (75 mL) was added sodium hydride (0.64 g, 0.0190 mol; 60% dispersion in mineral oil). The mixture was cooled to 0° C. and treated slowly with tert-butyl 4-(hydroxymethyl)piperidine-1-carboxylate (2.73 g, 0.0127 mol). The reaction was allowed to reach room temperature and then stirred overnight. The reaction mixture was concentrated under reduced pressure. Brine (75 mL) was added and the mixture was extracted with chloroform (3×100 mL). The combined organic layers were dried with MgSO₄, filtered and evaporated to give the title compound as a light brown solid. Yield 4.54 g (96%), Analytical HPLC: purity 100% (System A and B); ¹H NMR (400 MHz, CDCl₃) δ ppm 1.19-1.35 (m, 3H) 1.48 (s, 9H) 1.75-1.85 (m, 2H) 1.88-2.03 (m, 1H) 2.68-2.81 (m, 2H) 4.13 (d, J=6.5 Hz, 2H) 4.14-4.21 (m, 1H) 6.64-6.68 (m, 1H) 7.65 (m, 1H) 8.17-8.19 (m, 1H); LRESIMS for C₁₆H₂₃BrN₂O₃ m/z 371 (M+H)⁺.

### Intermediate A3

### 2-[(1-Benzylpiperidin-4-yl)methoxy]-5-[4-(methylsulfonyl)phenyl]pyridine

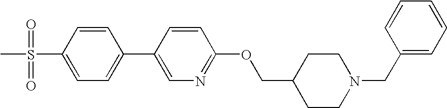

A mixture of 2-[(1-benzylpiperidin-4-yl)methoxy]-5-bromopyridine (700 mg, 1.94 mmol; Intermediate A1), [4-(methylsulfonyl)phenyl]boronic acid (426 mg, 2.13 mmol), Pd(PPh₃)₄ (112 mg, 0.097 mmol), potassium carbonate (670 mg, 4.85 mmol), 1,4-dioxane (15 mL) and water (5 mL) was stirred in a sealed flask for 16 h at 90° C. (STEM block). The reaction mixture was concentrated under reduced pressure. Water (50 mL) was added and the mixture was extracted with chloroform (2×75 mL). The combined organic layers were concentrated and the residue was purified by flash chromatography on silica (gradient 20% n-heptane in DCM→40% EtOAc in DCM) to give the title compound as a white solid. Yield: 661 mg (78%); Analytical HPLC: purity 100% (System A and B); ¹H NMR (400 MHz, CDCl₃) δ ppm 1.39-1.55 (m, 2H) 1.80-1.93 (m, 3H) 1.98-2.09 (m, 2H) 2.91-3.01 (m, 2H) 3.11 (s, 3H) 3.54 (s, 2H) 4.23 (d, J=6.3 Hz, 2H) 6.83-6.89 (m, 1H) 7.24-7.39 (m, 5H) 7.69-7.76 (m, 2H) 7.80-7.86 (m, 1H) 7.99-8.06 (m, 2H) 8.40-8.43 (m, 1H); LRESIMS for C₂₅H₂₈N₂O₃S m/z=437 (M+H)⁺.

### Example A1

### tert-Butyl 4-[({5-[4-(methylsulfonyl)phenyl]pyridin-2-yl}oxy)methyl]piperidine-1-carboxylate

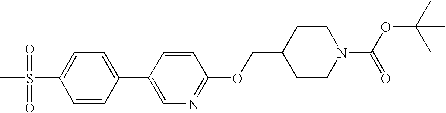

A mixture of tert-butyl 4-{[(5-bromopyridin-2-yl)oxy]methyl}piperidine-1-carboxylate (2.00 g, 0.0054 mol; Intermediate A2), (4-methylsulfonylphenyl)boronic acid (1.18 g, 0.0059 mol), Pd(PPh₃)₄ (312 mg, 0.00027 mmol), potassium carbonate (1.87 g, 0.014 mol), 1,4-dioxane (40 mL) and water (10 mL) was stirred in a sealed flask for 16 h at 90° C. (STEM block). The reaction mixture was concentrated and the residue was purified by flash chromatography on silica with gradient elution (20% heptane in DCM, then 100% DCM and finally 20% EtOAc in DCM) to give the title compound as a white solid. Yield: 1.77 mg (73%), Analytical HPLC: purity 99% (System A and B); ¹H NMR (400 MHz, CDCl₃) δ ppm 1.23-1.40 (m, 2H) 1.49 (s, 9H) 1.79-1.90 (m, 2H) 1.96-2.09 (m, 1H) 2.68-2.85 (m, 2H) 3.12 (s, 3H) 4.12-4.23 (m, 2H) 4.25 (d, J=6.5 Hz, 2H) 6.86-6.90 (m, 1H) 7.70-7.76 (m, 2H) 7.83-7.88 (m, 1H) 8.00-8.06 (m, 2H) 8.40-8.44 (m, 1H); LRESIMS for C₂₃H₃₀N₂O₅S m/z=447 (M+H)⁺; HRESIMS, calc. monoiso mass (Da): 446.1875, found monoiso mass (Da): 446.1869.

### Intermediate A4

### 5-[4-(Methylsulfonyl)phenyl]-2-(piperidin-4-ylmethoxy)pyridine

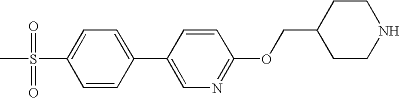

To a stirred suspension of 2-[(1-benzylpiperidin-4-yl)methoxy]-5-[4-(methylsulfonyl)-phenyl]pyridine (150 mg, 0.344 mmol; Intermediate A3) and 10% Pd/C in propanol (10 mL) was added a solution of ammonium formate (65 mg, 1.03 mmol) in water (3 mL). The suspension was heated at 90° C. overnight, filtered through Celite and evaporated. The residue was partitioned between saturated aqueous NaHCO₃ (15 mL) and DCM (15 mL). The water phase was extracted with an additional portion of DCM (15 mL). The organic layers were combined and evaporated. The crude product was used without further purification in subsequent experiments. Yield: 74 mg (62%). LRESIMS for C₁₈H₂₂N₂O₃S m/z=347 (M+H)⁺.

### Intermediate A5

### 5-[4-(Methylsulfonyl)phenyl]-2-(piperidin-4-ylmethoxy)pyridine, trifluoroacetate

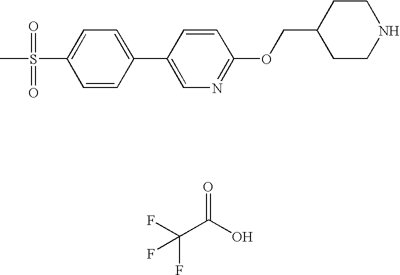

To a flask containing tert-butyl 4-[({5-[4-(methylsulfonyl)phenyl]pyridin-2-yl}oxy)-methyl]piperidine-1-carboxylate (850 mg, 1.903 mmol; obtained in Example A1) were added DCM (50 mL) and TFA (5 mL) The reaction mixture was stirred for 90 minutes at room temperature and then concentrated under reduced pressure to give the title compound as an yellow oil. Yield: 870 mg (99%); Analytical HPLC: purity 100% (System A and B); LRESIMS for C₁₈H₂₂N₂O₃S m/z=347 (M+H)⁺.

### Example A2

### Benzyl 4-[({5-[4-(methylsulfonyl)phenyl]pyridin-2-yl}oxy)methyl]piperidine-1-carboxylate

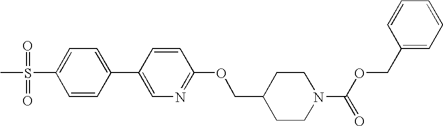

To a vial containing benzyl chloroformate (9 mg, 0.052 mmol) were added a solution of 5-[4-(methylsulfonyl)phenyl]-2-(piperidin-4-ylmethoxy)pyridine (15 mg, 0.043 mmol; Intermediate A4) in dry THF (2 mL) and triethylamine (12 μL, 0.086 mmol). The reaction mixture was shaken overnight and evaporated. The residue was purified by preparative HPLC (System D, gradient 25-70%). Yield: 2.1 mg (10%); Analytical HPLC: purity 99% (System A and B); ¹H NMR (400 MHz, CDCl₃) δ ppm 1.26-1.45 (m, 3H) 1.82-1.95 (m, 2H) 2.02-2.16 (m, 1H) 2.80-2.95 (m, 1H) 3.12 (s, 3H) 4.23 (d, J=6.3 Hz, 2H) 4.25-4.34 (m, 2H) 5.17 (s, 2H) 6.93 (d, J=8.5 Hz, 1H) 7.32-7.42 (m, 5H) 7.72-7.77 (m, 2H) 7.93 (m, 1H) 8.03-8.07 (m, 2H) 8.46-8.49 (m, 1H); LRESIMS for C₂₆H₂₈N₂O₅S m/z=481 (M+H)⁺; HRESIMS, calc. monoiso mass (Da): 480.1719, found monoiso mass (Da): 480.1707.

### Example A3

### 2-({1-[3-(3-Chloro-4-methoxyphenyl)propanoyl]piperidin-4-yl}methoxy)-5-[4-(methylsulfonyl)phenyl]pyridine

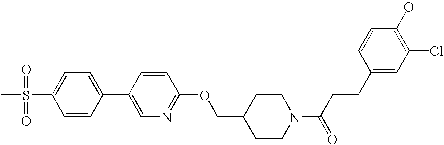

To a vial containing 3-(3-chloro-4-methoxyphenyl)propanoic acid (6.86 mg, 0.032 mmol) were added a solution of 5-[4-(methylsulfonyl)phenyl]-2-(piperidin-4-ylmethoxy)pyridine (10 mg, 0.029 mmol; Intermediate A4) in dry THF (2 mL) and triethylamine (16 μL, 0.12 mmol). Then HOBT (8 mg, 0.058 mmol) and EDC (11 mg, 0.058 mmol) were added to the solution. The mixture was shaken overnight, evaporated and then purified by preparative HPLC (System D). Yield: 2.0 mg (13%); Analytical HPLC: purity 98% (System A and B); ¹H NMR (400 MHz, CDCl₃) δ ppm 1.10-1.37 (m, 3H) 1.85-1.96 (m, 2H) 2.05-2.18 (m, 1H) 2.61-2.73 (m, 2H) 2.89-2.96 (m, 2H) 2.99-3.09 (m, 1H) 3.12 (s, 3H) 3.83-3.89 (m, 1H) 3.90 (s, 3H) 4.23 (d, J=6.5 Hz, 2H) 4.67-4.78 (m, 1H) 6.85-6.94 (m, 2H) 7.11 (m, 1H) 7.23-7.25 (m, 1H) 7.72-7.76 (m, 2H) 7.88-7.93 (m, 1H) 8.03-8.07 (m, 2H) 8.44-8.47 (m, 1H); LRESIMS for C₂₈H₃₁ClN₂O₅S m/z=543 (M+H)⁺; HRESIMS, calc. monoiso mass (Da): 542.1642, found monoiso mass (Da): 542.1630.

### Example A4

### 2-{[1-(Bicyclo[2.2.1]hept-2-ylcarbonyl)piperidin-4-yl]methoxy}-5-[4-(methylsulfonyl)-phenyl]pyridine

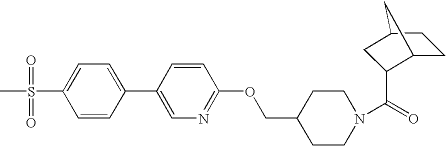

The title compound was prepared from Intermediate A4 (0.029 mmol) and bicyclo[2.2.1]heptane-2-carboxylic acid in accordance with the procedure described for Example A3. Yield: 2.6 mg (19%); Analytical HPLC: purity 99% (System A and B); LRESIMS for C₂₆H₃₂N₂O₄S m/z=469 (M+H)⁺; HRESIMS, calc. monoiso mass (Da): 468.2083, found monoiso mass (Da): 468.2084.

### Example A5

### 2-{[1-(Cyclohexylacetyl)piperidin-4-yl]methoxy}-5-[4-(methylsulfonyl)phenyl]-pyridine

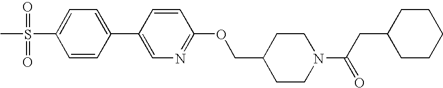

The title compound was prepared from Intermediate A4 (0.029 mmol) and cyclohexylacetic acid in accordance with the procedure described for Example A3. Yield: 3.1 mg (23%); Analytical HPLC: purity 99% (System A and B); LRESIMS for C₂₆H₃₄N₂O₄S m/z=471 (M+H)⁺; HRESIMS, calc. monoiso mass (Da): 470.2239, found monoiso mass (Da): 470.2238.

### Example A6

### 5-isopropoxy-2-({4-[({5-[4-(methylsulfonyl)phenyl]pyridin-2-yl}oxy)methyl]piperidin-1-yl}carbonyl)pyridine

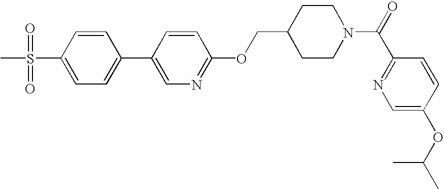

The title compound was prepared from Intermediate A4 (0.065 mmol) and 5-isopropoxypyridine-2-carboxylic acid in accordance with the procedure described for Example A3. Yield: 12.3 mg (37%); Analytical HPLC: purity 96% (System A and B); LRESIMS for C₂₇H₃₁N₃O₅S m/z=510 (M+H)⁺; HRESIMS, calc. monoiso mass (Da): 509.1984, found monoiso mass (Da): 509.1989.

### Example A7

### 2-{[1-(Cyclohexylcarbonyl)piperidin-4-yl]methoxy}-5-[4-(methylsulfonyl)phenyl]-pyridine

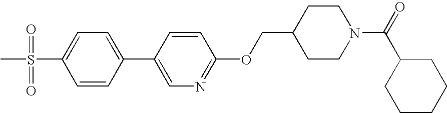

The title compound was prepared from Intermediate A4 (0.029 mmol) and cyclohexanecarboxylic acid in accordance with the procedure described for Example A3. Yield: 25.5 mg (86%); Analytical HPLC: purity 99% (System A and B); ¹H NMR (400 MHz, CDCl₃) δ ppm 1.18-1.38 (m, 5H) 1.46-1.62 (m, 2H) 1.65-2.02 (m, 7H) 2.03-2.18 (m, 1H) 2.44-2.70 (m, 2H) 2.97-3.14 (m, 4H) 3.91-4.07 (m, 1H) 4.20-4.30 (m, 2H) 4.62-4.79 (m, 1H) 6.88 (m, 1H) 7.70-7.75 (m, 2H) 7.86 (m, 1H) 8.00-8.05 (m, 2H) 8.40-8.43 (m, 1H); LRESIMS for C₂₅H₃₂N₂O₄S m/z=457 (M+H)⁺; HRESIMS, calc. monoiso mass (Da): 456.2083, found monoiso mass (Da): 456.2085.

### Example A8

### 2-({1-[(4-Methoxycyclohexyl)carbonyl]piperidin-4-yl}methoxy)-5-[4-(methylsulfonyl)-phenyl]pyridine

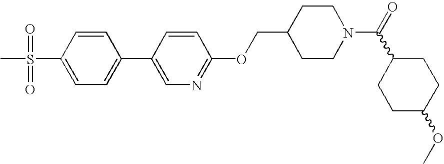

The title compound was prepared from Intermediate A4 (0.065 mmol) and 4-methoxycyclohexanecarboxylic acid (mixture of cis/trans isomers) in accordance with the procedure described for Example A3. Yield: 6.4 mg (20%); Analytical HPLC: purity 100% (System A and B); ¹H NMR (400 MHz, CDCl₃) δ ppm 1.12-1.42 (m, 4H) 1.50-1.73 (m, 2H) 1.77-2.05 (m, 4H) 2.08-2.25 (m, 3H) 2.42-2.54 (m, 1H) 2.54-2.71 (m, 1H) 3.01-3.24 (m, 5H) 3.38 (s, 3H) 3.91-4.06 (m, 1H) 4.27 (m, 2H) 4.63-4.77 (m, 1H) 6.87-6.91 (m, 1H) 7.71-7.76 (m, 2H) 7.87 (m, 1H) 8.01-8.06 (m, 2H) 8.41-8.44 (m, 1H); LRESIMS for C₂₆H₃₄N₂O₅S m/z=487 (M+H)⁺; HRESIMS, calc. monoiso mass (Da): 486.2188, found monoiso mass (Da): 486.2190.

### Example A9

### 3-({4-[({5-[4-(Methylsulfonyl)phenyl]pyridin-2-yl}oxy)methyl]piperidin-1-yl}-carbonyl)benzonitrile

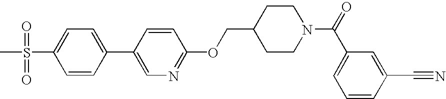

The title compound was prepared from Intermediate A4 (0.065 mmol) and 3-cyanobenzoic acid in accordance with the procedure described for Example A3. Yield: 20.7 mg (67%); Analytical HPLC: purity 100% (System A and B, R_{TA}=2.15 min, R_{TB}=2.11 min); ¹H NMR (400 MHz, CDCl₃) δ ppm 1.29-1.59 (m, 2H) 1.78-2.12 (m, 2H) 2.10-2.27 (m, 1H) 2.76-2.99 (m, 1H) 3.04-3.23 (m, 4H) 3.60-3.84 (m, 1H) 4.30 (d, J=6.3 Hz, 2H) 4.70-4.87 (m, 1H) 6.89 (m, 1H) 7.53-7.60 (m, 1H) 7.65-7.70 (m, 1H) 7.70-7.76 (m, 4H) 7.84-7.90 (m, 1H) 8.01-8.07 (m, 2H) 8.40-8.44 (m, 1H); LRESIMS for C₂₆H₂₅N₃O₄S m/z=476 (M+H)⁺; HRESIMS, calc. monoiso mass (Da): 475.1566, found monoiso mass (Da): 475.1568.

### Example A10

### 2-Methyl-4-{4-[({5-[4-(methylsulfonyl)phenyl]pyridin-2-yl}oxy)methyl]piperidin-1-yl}-4-oxobutan-2-ol

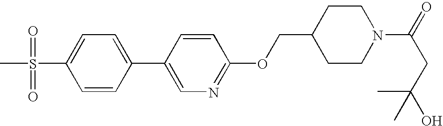

The title compound was prepared from Intermediate A4 (0.065 mmol) and 3-hydroxy-3-methylbutanoic acid in accordance with the procedure described for Example A3. Yield: 5.5 mg (19%); Analytical HPLC: purity 100% (System A and B, R_{TA}=1.92 min, R_{TB}=1.84 min); ¹H NMR (400 MHz, CDCl₃) δ ppm 1.14-1.30 (m, 8H) 1.78-1.91 (m, 2H) 1.98-2.11 (m, 1H) 2.38 (s, 2H) 2.49-2.61 (m, 1H) 2.94-3.05 (m, 4H) 3.78-3.88 (m, 1H) 4.18 (d, J=6.3 Hz, 2H) 4.58-4.68 (m, 1H) 6.80 (m, 1H) 7.59-7.66 (m, 2H) 7.78 (m, 1H) 7.91-7.97 (m, 2H) 8.31-8.34 (m, 1H); LRESIMS for C₂₃H₃₀N₂O₅S m/z=447 (M+H)⁺; HRESIMS, calc. monoiso mass (Da): 446.1875, found monoiso mass (Da): 446.1875.

### Example A11

### 1,1,1-Trifluoro-2-methyl-4-{4-[({5-[4-(methylsulfonyl)phenyl]pyridin-2-yl}oxy)-methyl]piperidin-1-yl}-4-oxobutan-2-ol

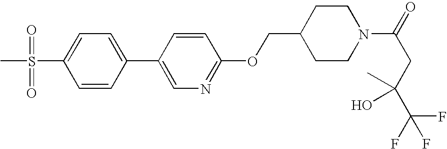

The title compound was prepared from Intermediate A4 (0.065 mmol) and 4,4,4-trifluoro-3-hydroxy-3-methylbutanoic acid in accordance with the procedure described for Example A3. Yield: 8.0 mg (25%); Analytical HPLC: purity 100% (System A and B, R_{TA}=2.24 min, R_{TB}=2.22 min); ¹H NMR (400 MHz, CDCl₃) δ ppm 1.25-1.43 (m, 2H) 1.44-1.48 (m, 3H) 1.89-2.05 (m, 2H) 2.10-2.24 (m, 1H) 2.41-2.53 (m, 1H) 2.63-2.77 (m, 1H) 2.82-2.92 (m, 1H) 3.12 (s, 3H) 3.13-3.21 (m, 1H) 3.89-4.00 (m, 1H) 4.25-4.31 (m, 2H) 4.65-4.79 (m, 1H) 6.90 (m, 1H) 7.70-7.77 (m, 2H) 7.88 (m, 1H) 8.02-8.07 (m, 2H) 8.41-8.44 (m, 1H); LRESIMS for C₂₃H₂₇F₃N₂O₅S m/z=501 (M+H)⁺; HRESIMS, calc. monoiso mass (Da): 500.1593, found monoiso mass (Da): 500.1586.

### Example A12

### 1-[3-({4-[({5-[4-(Methylsulfonyl)phenyl]pyridin-2-yl}oxy)methyl]piperidin-1-yl}-carbonyl)phenyl]ethanone

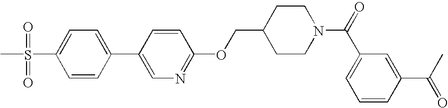

The title compound was prepared from Intermediate A4 (0.434 mmol) and 3-acetylbenzoic acid in accordance with the procedure described for Example A3. Yield: 61 mg (29%); Analytical HPLC: purity 100% (System A and B, R_{TA}=2.112 min, R_{TB}=2.07 min); ¹H NMR (400 MHz, CDCl₃) δ ppm 1.26-1.57 (m, 2H) 1.76-2.08 (m, 2H) 2.09-2.24 (m, 1H) 2.63 (s, 3H) 2.79-2.97 (m, 1H) 3.02-3.21 (m, 4H) 3.69-3.86 (m, 1H) 4.28 (d, J=6.5 Hz, 2H) 4.72-4.87 (m, 1H) 6.88 (m, 1H) 7.48-7.57 (m, 1H) 7.59-7.65 (m, 1H) 7.69-7.75 (m, 2H) 7.86 (m, 1H) 7.98-8.04 (m, 4H) 8.39-8.42 (m, 1H); LRESIMS for C₂₇H₂₈N₂O₅S m/z=493 (M+H)⁺; HRESIMS, calc. monoiso mass (Da): 492.1719, found monoiso mass (Da): 492.1715.

### Example A13

### tert-Butyl 4-({[5-(4-cyanophenyl)pyridin-2-yl]oxy}methyl)piperidine-1-carboxylate

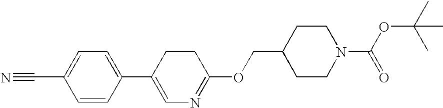

The title compound was prepared from Intermediate A2 (0.135 mmol) and (4-cyanophenyl)boronic acid in accordance with the procedure described for Example A1. Yield: 1.5 mg (3%); Analytical HPLC: purity 91% (System A and B, R_{TA}=2.89 min, R_{TB}=2.92 min); LRESIMS for C₂₃H₂₇N₃O₃ m/z=394 (M+H)⁺; HRESIMS, calc. monoiso mass (Da): 393.2052, found monoiso mass (Da): 393.2052.

### Example A14

### tert-Butyl 4-[({5-[4-(morpholin-4-ylsulfonyl)phenyl]pyridin-2-yl}oxy)methyl]-piperidine-1-carboxylate

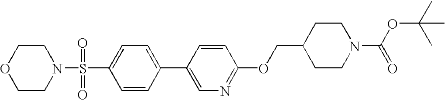

The title compound was prepared from Intermediate A2 (0.135 mmol) and [4-(morpholin-4-ylsulfonyl)phenyl]boronic acid in accordance with the procedure described for Example A1. Yield: 26.6 mg (38%); Analytical HPLC: purity 100% (System A and B, R_{TA}=2.80 min, R_{TB}=2.78 min); ¹H NMR (400 MHz, CDCl₃) δ ppm 1.22-1.41 (m, 2H) 1.48 (s, 9H) 1.79-1.90 (m, 2H) 1.95-2.10 (m, 1H) 2.70-2.84 (m, 2H) 3.01-3.10 (m, 4H) 3.72-3.82 (m, 4H) 4.12-4.23 (m, 2H) 4.27 (d, J=6.5 Hz, 2H) 6.90-6.94 (m, 1H) 7.68-7.73 (m, 2H) 7.81-7.87 (m, 2H) 7.90 (m, 1H) 8.42-8.45 (m, 1H); LRESIMS for C₂₆H₃₅N₃O₆S m/z=518 (M+H)⁺; HRESIMS, calc. monoiso mass (Da): 517.2247, found monoiso mass (Da): 517.2261.

### Example A15

### 2-[(1-Benzoylpiperidin-4-yl)methoxy]-5-[4-(methylsulfonyl)phenyl]pyridine

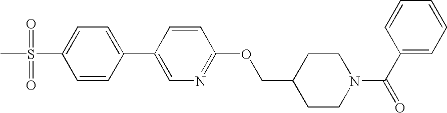

The title compound was prepared from Intermediate A4 (0.065 mmol) and benzoic acid in accordance with the procedure described for Example A3. Yield: 3.5 mg (11%); Analytical HPLC: purity 100% (System A and B); ¹H NMR (400 MHz, CDCl₃) δ ppm 1.31-1.60 (m, 2H) 1.79-2.08 (m, 2H) 2.14-2.26 (m, 1H) 2.80-3.11 (m, 2H) 3.15 (s, 3H) 3.78-3.99 (m, 1H) 4.35 (d, J=6.5 Hz, 2H) 4.77-4.94 (m, 1H) 6.94 (m, 1H) 7.43-7.49 (m, 5H) 7.74-7.80 (m, 2H) 7.92 (m, 1H) 8.05-8.10 (m, 2H) 8.46-8.49 (m, 1H); LRESIMS C₂₅H₂₆N₂O₄S m/z=451 (M+H)⁺; HRESIMS, calc. monoiso mass (Da): 450.1613, found monoiso mass (Da): 450.1609.

### Example A16

### N,N-Dimethyl-3-({4-[({5-[4-(methylsulfonyl)phenyl]pyridin-2-yl}oxy)methyl]-piperidin-1-yl}carbonyl)aniline, trifluoroacetate

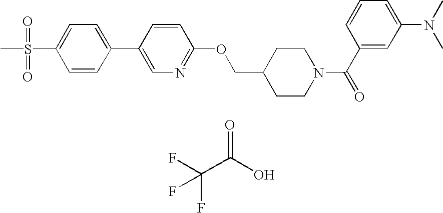

The title compound was prepared from Intermediate A4 (0.065 mmol) and 3-(dimethylamino)benzoic acid in accordance with the procedure described for Example A3. Yield: 4.2 mg (10%); Analytical HPLC: purity 98% (System A and B); LRESIMS C₂₇H₃₁N₃O₄S m/z=494 (M+H)⁺; HRESIMS, calc. monoiso mass (Da): 493.2035, found monoiso mass (Da): 493.2035.

### Example A17

### 4-{4-[({5-[4-(Methylsulfonyl)phenyl]pyridin-2-yl}oxy)methyl]piperidin-1-yl}-4-oxo-1-phenylbutan-1-one

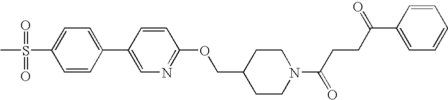

The title compound was prepared from Intermediate A4 (0.065 mmol) and 4-oxo-4-phenylbutanoic acid in accordance with the procedure described for Example A3. Yield: 29.5 mg (66%); Analytical HPLC: purity 100% (System A and B); LRESIMS C₂₈H₃₀N₂O₅S m/z=507 (M+H)⁺; HRESIMS, calc. monoiso mass (Da): 506.1875, found monoiso mass (Da): 506.1870.

### Example A18

### 5-[4-(Methylsulfonyl)phenyl]-2-{[1-(pyridin-2-ylcarbonyl)piperidin-4-yl]methoxy}-pyridine

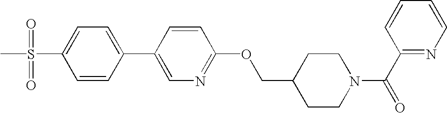

The title compound was prepared from Intermediate A4 (0.065 mmol) and pyridine-2-carboxylic acid in accordance with the procedure described for Example A3. Yield: 20.9 mg (64%); Analytical HPLC: purity 100% (System A and B); ¹H NMR (400 MHz, CDCl₃) δ ppm 1.40-1.62 (m, 2H) 1.79-1.91 (m, 1H) 1.97-2.05 (m, 1H) 2.11-2.25 (m, 1H) 2.85-2.98 (m, 1H) 3.11 (s, 3H) 3.17-3.29 (m, 1H) 3.85-3.95 (m, 1H) 4.29 (d, J=6.5 Hz, 2H) 4.75-4.87 (m, 1H) 6.87-6.91 (m, 1H) 7.42-7.48 (m, 1H) 7.66-7.75 (m, 3H) 7.86 (m, 1H) 7.89-7.95 (m, 1H) 8.00-8.05 (m, 2H) 8.40-8.43 (m, 1H) 8.62-8.66 (m, 1H); LRESIMS C₂₄H₂₅N₃O₄S m/z=452 (M+H)⁺; HRESIMS, calc. monoiso mass (Da): 451.1566, found monoiso mass (Da): 451.1565.

### Example A19

### 2-({4-[({5-[4-(Methylsulfonyl)phenyl]pyridin-2-yl}oxy)methyl]piperidin-1-yl}-carbonyl)pyridin-3-ol

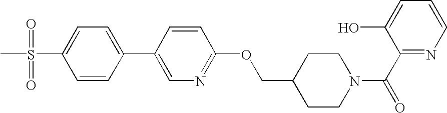

The title compound was prepared from Intermediate A4 (0.065 mmol) and 3-hydroxypyridine-2-carboxylic acid in accordance with the procedure described for Example A3. Yield: 2.7 mg (8%); Analytical HPLC: purity 90% (System A and B); LRESIMS for C₂₄H₂₅N₃O₅S m/z=468 (M+H)⁺; HRESIMS, calc. monoiso mass (Da): 467.1515, found monoiso mass (Da): 467.1512.

### Example A20

### 2-{[1-(4-Isopropoxybenzoyl)piperidin-4-yl]methoxy}-5-[4-(methylsulfonyl)phenyl]-pyridine

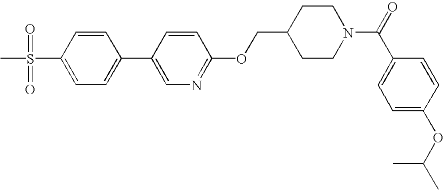

The title compound was prepared from Intermediate A4 (0.065 mmol) and 4-isopropoxybenzoic acid in accordance with the procedure described for Example A3. Yield 10.2 mg (31%); Analytical HPLC: purity 98% (System A, R_{T}=2.46 min); ¹H NMR (400 MHz, CDCl₃) δ ppm 1.18-1.34 (m, 2H) 1.37 (d, J=6.0 Hz, 6H) 1.39-1.49 (m, 2H) 1.85-1.99 (m, 2H) 2.09-2.22 (m, 1H) 2.83-3.08 (m, 2H) 3.12 (s, 3H) 4.30 (d, J=6.3 Hz, 2H) 4.55-4.66 (m, 1H) 6.87-6.93 (m, 2H) 7.36-7.42 (m, 3H) 7.70-7.77 (m, 2H) 7.88 (m, 1H) 8.02-8.07 (m, 2H) 8.42-8.45 (m, 1H); LRESIMS for C₂₈H₃₂N₂O₅S m/z 509 (M+H)⁺; HRESIMS, calc. monoiso mass (Da): 508.2032, found monoiso mass (Da): 508.2047.

### Intermediate A6

### tert-Butyl 4-{[(5-bromo-3-nitropyridin-2-yl)oxy]methyl}piperidine-1-carboxylate

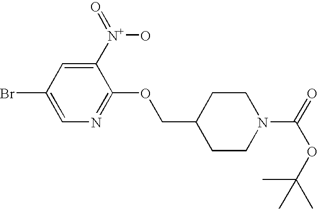

KOH (672 mg, 12 mmol) and K₂CO₃ (414 mg, 3 mmol) were mixed with toluene (15 mL). 4-Hydroxymethyl-piperidine-1-carboxylic acid tert-butyl ester (968 mg, 4.5 mmol) dissolved in toluene (5 mL) was added, followed by 2-chloro-3-nitro-5-bromopyridine (711 mg, 3.00 mmol). The resulting mixture was stirred for 2 min and tris[2-(2-methoxyethoxy)ethyl]amine (100 μL, 0.3 mmol) was added. The mixture was stirred for 3 h at r.t. The reaction mixture was filtered through Celite and evaporated. The residue was purified by flash chromatography on silica gel (40 g) using 10% EtOAc/toluene as eluent to give the title compound. Yield: 383 mg (31%); Analytical HPLC: purity 92% (System A, R_{T}=2.78 min), purity 96% (System B, R_{T}=2.87 min); ¹H NMR (400 MHz, CDCl₃) δ ppm 1.27 (m, 2H), 1.45 (s, 9H), 1.81 (m, 2H), 2.00 (m, 1H), 2.73 (m, 2H), 4.15 (m, 2H), 4.30 (d, J=6.5 Hz, 1H), 8.37 (m, 1H), 8.40 (m, 1H); LRESIMS for C₁₆H₂₂BrN₃O₅ m/z 318, 316 (M+H−t-Boc)⁺.

### Example A21

### tert-Butyl 4-[({5-[4-(methylsulfonyl)phenyl]-3-nitropyridin-2-yl}oxy)methyl]-piperidine-1-carboxylate

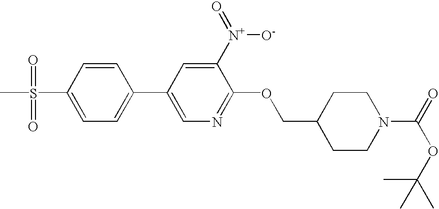

tert-Butyl 4-{[(5-bromo-3-nitropyridin-2-yl)oxy]methyl}piperidine-1-carboxylate (410 mg, 0.99 mmol; Intermediate A6), (4-methylsulfonyl)phenylboronic acid (220 mg, 1.10 mmol), Pd(PPh₃)₄ (58 mg. 0.05 mmol), K₂CO₃ (345 mg, 2.5 mmol) were mixed with dioxane (8 mL) and water (2 mL). The mixture was heated to 90° C. for 2 h and then cooled and filtered through a pad of Celite. The filtrate was evaporated and the residue was extracted with DCM and washed with 5% aqueous NaHCO₃ and brine. Concentration of the organic phase gave 547 mg of the crude product. A mixture of 25% EtOAc in toluene was added and the precipitate* formed was filtered off. Flash chromatography of the filtrate on silica gel using 25-30% EtOAc in toluene as eluent gave the title compound. Yield 82 mg (17%). Analytical HPLC: purity 100% (System A and B, R_{TA}=2.57 min, R_{TB}=2.61 min); ¹H NMR (400 MHz, CDCl₃) δ ppm 1.24-1.38 (m, 2H), 1.46 (s, 9H), 1.80-1.90 (m, 2H), 2.05 (m, 1H), 2.75 (m, 2H), 3.10 (s, 3H), 4.17 (br s, 2H), 4.39 (d, J=6.6 Hz, 2H), 7.75 (m, 2H), 8.07 (m, 2H), 8.50 (m, 1H), 8.62 (m, 1H); LRESIMS for C₂₃H₂₉N₃O₇S m/z=392 (M+H−t-Boc)⁺.
The precipitate (325 mg) was analyzed and showed the same HPLC, MS and NMR as the purified product. Total yield 407 mg (83%). Analytical HPLC: purity 97% (System A and B, R_{TA}=2.57 min, R_{TB}=2.61 min); HRESIMS, calc. monoiso mass (Da): 491.1726, found monoiso mass (Da): 491.1743.

### Intermediate A7

### 5-[4-(Methylsulfonyl)phenyl]-3-nitro-2-(piperidin-4-ylmethoxy)pyridine

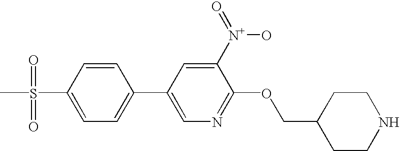

tert-Butyl 4-[({5-[4-(methylsulfonyl)phenyl]-3-nitropyridin-2-yl}oxy)methyl]piperidine-1-carboxylate (325 mg, 0.66 mmol; obtained in Example A21) was dissolved in DCM (3 mL) and treated with TFA (0.75 mL) over 0.5 h. The mixture was concentrated under reduced pressure and the residue was dissolved in CHCl₃ and washed with 2 M NaOH. The organic phase was dried (Na₂SO₄) and evaporation of the solvent gave the title product. Yield 260 mg (100%). Analytical HPLC: purity 97% (System A and B, R_{TA}=1.50 min, R_{TB}=1.36 min); ¹H NMR (400 MHz, CDCl₃) δ ppm 1.27-1.43 (m, 2H), 1.75-1.94 (m), 2.68 m, 2H), 3.12-3.21 (m, 2H), 4.38 (d, J=6.7 Hz, 2H), 7.75 (m, 2H), 8.07 (m, 2H), 8.49 (m, 1H), 8.61 (m, 1H); LRESIMS for C₁₈H₂₁N₃O₅S m/z 392 (M+H)⁺.

### Example A22

### 2-{[1-(Cyclohexylacetyl)piperidin-4-yl]methoxy}-5-[4-(methylsulfonyl)phenyl]-3-nitropyridine

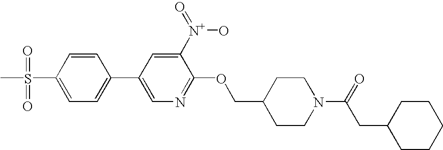

5-[4-(Methylsulfonyl)phenyl]-3-nitro-2-(piperidin-4-ylmethoxy)pyridine (27 mg, 0.07 mmol; Intermediate A7) and cyclohexylacetic acid (12 mg, 0.084 mmol) were mixed with DMF (0.7 mL) and Et₃N (0.02 mL). TBTU (27 mg, 0.084 mmol) was added and the reaction mixture was stirred at room temp overnight and then concentrated under reduced pressure. The residue was purified by preparative HPLC (System D). Yield 9 mg (25%). Analytical HPLC: purity 100% (System A, R_{T}=2.58 min); ¹H NMR (400 MHz, DMSO-d₆) δ ppm 0.82-0.98 (m, 2H), 1.01-1.32 (m, 6H), 1.53-1.85 (m, 9H), 2.00-2.14 (m, 1H), 2.14-2.21 (m, 2H), 2.95-3.06 (m, 1H), 3.26 (s, 3H), 3.86-3.99 (m, 1H), 4.31-4.50 (m, 3H), 7.98-8.09 (m, 4H), 8.81 (m, 1H), 8.92 (m, 1H); LRESIMS for C₂₆H₃₃N₃O₆S m/z 516 (M+H)⁺; HRESIMS, calc. monoiso mass (Da): 515.2090, found monoiso mass (Da): 515.2102.

### Example A23

### 2-{[1-(Bicyclo[2.2.1]hept-2-ylcarbonyl)piperidin-4-yl]methoxy}-5-[4-(methylsulfonyl)-phenyl]-3-nitropyridine

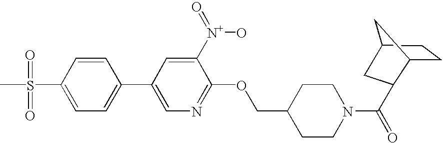

The title compound was prepared from bicyclo[2.2.1]heptane-2-carboxylic acid in accordance with the procedure described for Example A22. The product was purified by preparative HPLC (System D). Yield 21 mg (58%). Analytical HPLC: purity 100% (System A, R_{T}=2.57 min); ¹H NMR (400 MHz, DMSO-d₆) δ ppm 1.01-1.53 (m, 9H), 1.66-1.91 (m, 3H), 2.01-2.40 (m, 4H), 2.92-3.08 (m, 2H), 3.26 (s, 3H), 4.00-4.10 (m, 1H), 4.32-4.55 (m, 3H), 7.99-8.08 (m, 4H), 8.81 (m, 1H), 8.91 (m, 1H); LRESIMS for C₂₆H₃₁N₃O₆S m/z 514 (M+H)⁺; HRESIMS, calc. monoiso mass (Da): 513.1934, found monoiso mass (Da): 513.1938.

### Intermediate A8

### tert-Butyl 4-{[(5-bromopyridin-2-yl)amino]methyl}piperidine-1-carboxylate

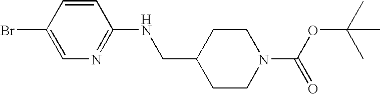

A solution of 2,5-dibromopyridine (2.00 g, 0.00844 mol), 4-aminomethyl-piperidine-1-carboxylic acid tert-butyl ester (18.0 g, 0.0844 mol) and pyridine (2 mL) was heated in a Stemblock at 150° C. overnight. The solvent was evaporated and the residue purified by flash chromatography (10% EtOAc in DCM). Yield 2.65 g (85%); Analytical HPLC: purity 95% (System A, R_{T}=1.85 min); ¹H NMR (400 MHz, CDCl₃) δ ppm 1.08-1.32 (m, 2H) 1.47 (s, 9H) 1.61-2.02 (m, 3H) 2.63-2.80 (m, 2H) 2.86-3.64 (m, 2H) 4.06-4.23 (m, 2H) 6.70-6.82 (m, 1H) 7.80-7.96 (m, 1H) 8.36-8.57 (m, 1H); LRESIMS for C₁₆H₂₄BrN₃O₂ m/z 370 (M+H)⁺.

### Example A24

### tert-Butyl 4-[({5-[4-(methylsulfonyl)phenyl]pyridin-2-yl}amino)methyl]piperidine-1-carboxylate

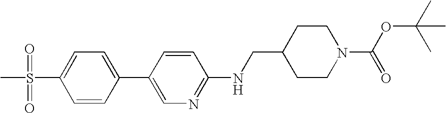

The title compound was prepared from tert-butyl 4-{[(5-bromopyridin-2-yl)amino]-methyl}piperidine-1-carboxylate (Intermediate A8; 5.4 mmol) and [4-(methylsulfonyl)phenyl]boronic acid in accordance with the procedure described for Example A1. Yield 1.33 g (55%); Analytical HPLC: purity 98% (System A, R_{T}=1.78 min); LRESMS for C₂₃H₃₁N₃O₄S m/z 446 (M+H)⁺; HRESIMS, calc. monoiso mass (Da): 445.2035, found monoiso mass (Da): 445.2038.

### Intermediate A9

### 5-[4-(Methylsulfonyl)phenyl]-N-(piperidin-4-ylmethyl)pyridin-2-amine, trifluoroacetate

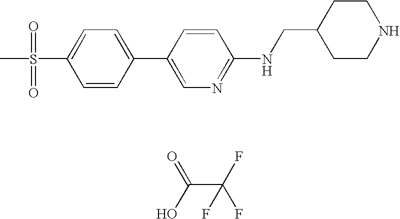

The title compound was prepared from tert-butyl 4-[({5-[4-(methylsulfonyl)phenyl]-pyridin-2-yl}amino)methyl]piperidine-1-carboxylate (1.41 mmol, obtained in Example A24) in accordance with the procedure described for Intermediate A5. Yield 636 mg (98%); Analytical HPLC: purity 98% (System A, R_{T}=0.92 min); LRESIMS for C₁₈H₂₃N₃O₂S m/z 346 (M+H)⁺.

### Example A25

### Isobutyl 4-[({5-[4-(methylsulfonyl)phenyl]pyridin-2-yl}amino)methyl]piperidine-1-carboxylate, trifluoroacetate

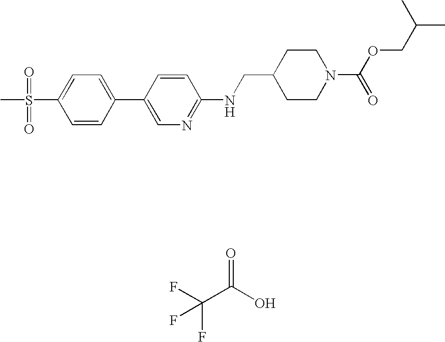

The title compound was prepared from 5-[4-(methylsulfonyl)phenyl]-N-(piperidin-4-ylmethyl)pyridin-2-amine (0.065 mmol; free base of Intermediate A9) and isobutyl chlorodicarbonate in accordance with the procedure described for Example A2. Yield 9 mg (25%); Analytical HPLC: purity 100% (System A, R_{T}=1.82 min); ¹H NMR (400 MHz, CDCl₃) δ ppm 0.95 (d, J=6.8 Hz, 6H) 1.18-1.33 (m, 2H) 1.81-2.02 (m, 4H) 2.75-2.89 (m, 2H) 3.12 (s, 3H) 3.22-3.30 (m, 2H) 3.88 (d, J=6.5 Hz, 2H) 4.17-4.31 (m, 2H) 6.93 (m, 1H) 7.64-7.70 (m, 2H) 8.04-8.15 (m, 4H); LRESIMS for C₂₃H₃₁N₃O₄S m/z 446 (M+H)⁺; HRESIMS, calc. monoiso mass (Da): 445.2035, found monoiso mass (Da): 445.2038.

### Example A26

### Benzyl 4-[({5-[4-(methylsulfonyl)phenyl]pyridin-2-yl}amino)methyl]piperidine-1-carboxylate, trifluoroacetate

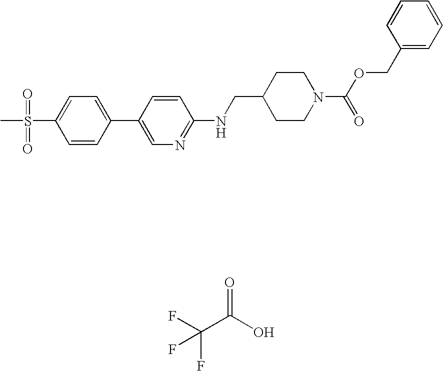

The title compound was prepared from 5-[4-(methylsulfonyl)phenyl]-N-(piperidin-4-ylmethyl)pyridin-2-amine (0.065 mmol; free base of Intermediate A9) and benzyl chlorodicarbonate in accordance with the procedure described for Example A2. Yield 2.6 mg (7%); Analytical HPLC: purity 100% (System A, R_{T}=1.86 min); LRESIMS for C₂₆H₂₉N₃O₄S m/z 480 (M+H)⁺; HRESIMS, calc. monoiso mass (Da): 479.1879, found monoiso mass (Da): 479.1881.

### Example A27

### Ethyl 4-[({5-[4-(methylsulfonyl)phenyl]pyridin-2-yl}amino)methyl]piperidine-1-carboxylate, trifluoroacetate

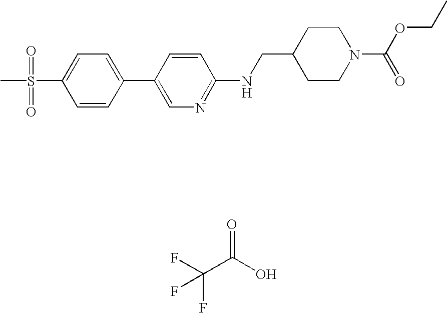

The title compound was prepared from 5-[4-(methylsulfonyl)phenyl]-N-(piperidin-4-ylmethyl)pyridin-2-amine (0.065 mmol; free base of Intermediate A9) and ethyl chloroformate in accordance with the procedure described for Example A2. Yield 6.6 mg (19%); Analytical HPLC: purity 99% (System A, R_{T}=1.56 min); LRESIMS for C₂₁H₂₇N₃O₄S m/z 418 (M+H)⁺; HRESIMS, calc. monoiso mass (Da): 417.1722, found monoiso mass (Da): 417.1725.

### Example A28

### N-{[1-(Cyclohexylcarbonyl)piperidin-4-yl]methyl}-5-[4-(methylsulfonyl)phenyl]-pyridin-2-amine, trifluoroacetate

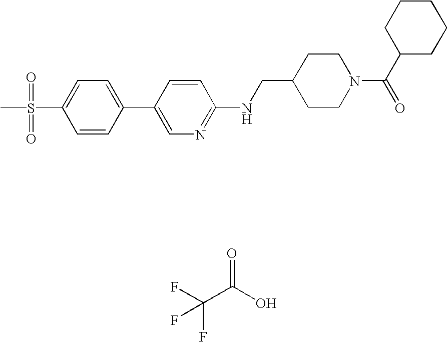

The title compound was prepared from 5-[4-(methylsulfonyl)phenyl]-N-(piperidin-4-ylmethyl)pyridin-2-amine (0.065 mmol; free base of Intermediate A9) and cyclohexanecarbonyl chloride in accordance with the procedure described for Example A2. Yield 8.8 mg (24%); Analytical HPLC: purity 99% (System A, R_{T}=1.73 min); LRESIMS for C₂₅H₃₃N₃O₃S m/z 456 (M+H)⁺; HRESIMS, calc. monoiso mass (Da): 455.2243, found monoiso mass (Da): 455.2244.

### Example A29

### N-{[1-(Cyclohexylacetyl)piperidin-4-yl]methyl}-5-[4-(methylsulfonyl)phenyl]pyridin-2-amine, trifluoroacetate

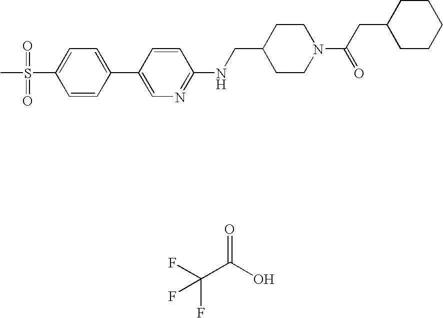

The title compound was prepared from 5-[4-(methylsulfonyl)phenyl]-N-(piperidin-4-ylmethyl)pyridin-2-amine (0.065 mmol; free base of Intermediate A9) and cyclohexylacetic acid in accordance with the procedure described for Example A3. Yield 11.5 mg (30%); Analytical HPLC: purity 99% (System A, R_{T}=1.83 min); LRESIMS for C₂₆H₃₅N₃O₃S m/z 470 (M+H)⁺; HRESIMS, calc. monoiso mass (Da): 469.2399, found monoiso mass (Da): 469.2397.

### Example A30

### N-{[1-(3-Cyclopentylpropanoyl)piperidin-4-yl]methyl}-5-[4-(methylsulfonyl)phenyl]-pyridin-2-amine, trifluoroacetate

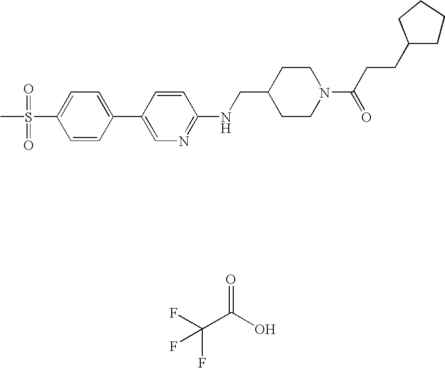

The title compound was prepared from 5-[4-(methylsulfonyl)phenyl]-N-(piperidin-4-ylmethyl)pyridin-2-amine (0.065 mmol; free base of Intermediate A9) and 3-cyclopentylpropanoyl chloride in accordance the procedure described for Example A2. Yield 5.2 mg (14%); Analytical HPLC: purity 99% (System A, R_{T}=1.85 min); ¹H NMR (400 MHz, CDCl₃) δ ppm 1.04-1.38 (m, 4H) 1.46-1.71 (m, 6H) 1.73-1.93 (m, 4H) 1.97-2.11 (m, 2H) 2.31-2.44 (m, 2H) 2.53-2.69 (m, 1H) 3.05-3.10 (m, 1H) 3.12 (s, 3H) 3.18-3.36 (m, 2H) 3.91-4.00 (m, 1H) 4.66-4.78 (m, 1H) 6.93 (m, 1H) 7.63-7.71 (m, 2H) 8.04-8.16 (m, 4H); LRESIMS for C₂₆H₃₅N₃O₃S m/z 470 (M+H)⁺; HRESIMS, calc. monoiso mass (Da): 469.2399, found monoiso mass (Da): 469.2399.

### Example A31

### 5-[4-(Methylsulfonyl)phenyl]-N-({1-[(2,3,6-trifluorophenyl)acetyl]piperidin-4-yl}-methyl)pyridin-2-amine, trifluoroacetate

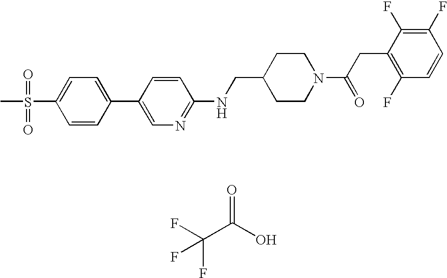

The title compound was prepared from 5-[4-(methylsulfonyl)phenyl]-N-(piperidin-4-ylmethyl)pyridin-2-amine (0.065 mmol; free base of Intermediate A9) and (2,3,6-trifluorophenyl)acetic acid in accordance with the procedure described for Example A3. Yield 9.3 mg (23%); Analytical HPLC: purity 100% (System A, R_{T}=1.74 min); ¹H NMR (400 MHz, CDCl₃) δ ppm 1.21-1.43 (m, 2H) 1.82-1.95 (m, 1H) 2.00-2.16 (m, 2H) 2.63-2.75 (m, 1H) 3.13 (s, 3H) 3.18-3.38 (m, 3H) 3.75-3.79 (m, 2H) 4.03-4.13 (m, 1H) 4.66-4.75 (m, 1H) 6.80-6.90 (m, 1H) 6.91-6.97 (m, 1H) 7.03-7.14 (m, 1H) 7.64-7.71 (m, 2H) 8.04-8.14 (m, 4H); LRESIMS for C₂₆H₂₆F₃N₃O₃S m/z 518 (M+H)⁺; HRESIMS, calc. monoiso mass (Da): 517.1647, found monoiso mass (Da): 517.1646.

### Example A32

### 5-[4-(Methylsulfonyl)phenyl]-N-[(1-pentanoylpiperidin-4-yl)methyl]pyridin-2-amine, trifluoroacetate

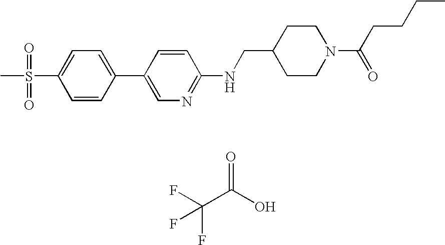

The title compound was prepared from 5-[4-(methylsulfonyl)phenyl]-N-(piperidin-4-ylmethyl)pyridin-2-amine (0.065 mmol; free base of Intermediate A9) and pentanoyl chloride in accordance with the procedure described for Example A2. Yield 5 mg (14%); Analytical HPLC: purity 100% (System A, R_{T}=1.60 min); ¹H NMR (400 MHz, CDCl₃) δ ppm 0.95 (t, J=7.3 Hz, 3H) 1.17-1.32 (m, 2H) 1.33-1.45 (m, 2H) 1.56-1.69 (m, 2H) 1.82-1.92 (m, 1H) 1.96-2.11 (m, 2H) 2.34-2.43 (m, 2H) 2.56-2.68 (m, 1H) 3.04-3.11 (m, 1H) 3.13 (s, 3H) 3.20-3.35 (m, 2H) 3.91-3.99 (m, 1H) 4.67-4.79 (m, 1H) 6.93 (m, 1H) 7.64-7.71 (m, 2H) 8.05-8.14 (m, 4H); LRESIMS for C₂₃H₃₁N₃O₃S m/z 430 (M+H)⁺; HRESIMS, calc. monoiso mass (Da): 429.2086, found monoiso mass (Da): 429.2088.

### Example A33

### tert-Butyl 4-[(methyl{5-[4-(methylsulfonyl)phenyl]pyridin-2-yl}amino)methyl]-piperidine-1-carboxylate

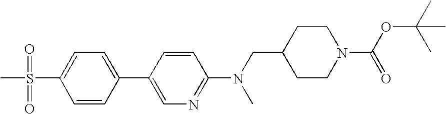

A mixture of tert-butyl 4-[({5-[4-(methylsulfonyl)phenyl]pyridin-2-yl}amino)methyl]-piperidine-1-carboxylate (50 mg, 0.112 mmol; obtained in Example A24), acetonitrile (1.5 mL), 37% formalin (25 μL, 27 mg, 0.560 mmol) and sodium cyanoborohydride (11 mg, 0.179 mmol) was stirred at r.t. for 15 minutes. Then acetic acid (200 μL) was added and stirring continued for 48 hours before evaporation. The residue was partitioned between water (10 mL) and chloroform (3×10 mL). The organic layers were combined, evaporated and the residue was purified by flash chromatography (20% EtOAc in DCM). Yield 42 mg (82%); Analytical HPLC: purity 97% (System A, R_{T}=1.89 min); ¹H NMR (400 MHz, CDCl₃) δ ppm 1.13-1.33 (m, 2H) 1.47 (s, 9H) 1.62-1.78 (m, 3H) 1.90-2.01 (m, 1H) 2.61-2.77 (m, 2H) 3.10 (s, 3H) 3.13 (s, 3H) 3.48-3.55 (m, 2H) 4.06-4.20 (m, 1H) 6.58 (m, 1H) 7.67-7.76 (m, 3H) 7.94-8.00 (m, 2H) 8.45-8.49 (m, 1H); LRESIMS for C₂₄H₃₃N₃O₄S m/z 460 (M+H)⁺; HRESIMS, calc. monoiso mass (Da): 459.2192, found monoiso mass (Da): 459.2193.

### Intermediate A10

### 5-[4-(Methylsulfonyl)phenyl]-2-[(piperidin-4-yloxy)methyl]pyridine, trifluoroacetate

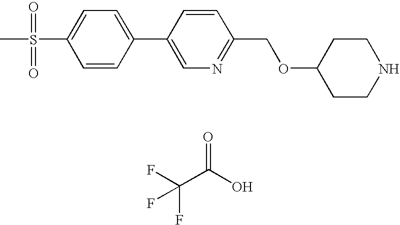

Thionyl chloride (0.949 g, 7.98 mmol) was added to an ice-cold solution of (5-bromopyridin-2-yl)-methanol (1.00 g, 5.32 mmol) in DCM (10 mL) giving a milky mixture. After the addition was completed, the ice-bath was removed and the mixture was stirred for 1.5 hour at room temperature. The mixture was washed with saturated aqueous NaHCO₃ and the organic phase was dried over Na₂SO₄ and concentrated under reduced pressure to give 5-bromo-2-(chloromethyl)pyridine (1.11 g) as an oil.

Potassium tert-butoxide (0.656 g, 5.86 mmol) was added to tert-butyl 4-hydroxy-1-piperidinecarboxylate (1.07 g, 5.32 mmol) in THF (10 mL) and the mixture was stirred for 5 minutes. The mixture was added to 5-bromo-2-(chloromethyl)pyridine (1.11 g) in THF (10 mL) and stirred at 60° C. over night. The solvent was evaporated and the mixture was taken up in DCM and washed with brine. The organic phase was dried over Na₂SO₄, filtered and the solvent was evaporated. The residue was purified by flash chromatography gradient eluting with 2→5% acetone in DCM giving crude tert-butyl 4-[(5-bromopyridin-2-yl)methoxy]piperidine-1-carboxylate (0.44 g).

A mixture of tert-butyl 4-[(5-bromopyridin-2-yl)methoxy]piperidine-1-carboxylate (0.44 g), (4-methylsulphonylphenyl)boronic acid (0.018 g, 0.10 mmol), Pd(PPh₃)₄ (0.034 g, 0.03 mmol), K₂CO₃ (0.20 g, 1.48 mmol), 1,4-dioxane (2.4 mL) and water (0.6 mL) was exposed to microwave irradiation (130° C.) for 20 minutes. Solid material was filtered off and the filtrate was evaporated. The residue was dissolved in EtOAc (30 mL) and HCl (g) was bubbled through the solution for 5 minutes to remove the N-t-Boc protecting group. The solvent was evaporated and the residue was purified by preparative HPLC (System D) to give the title compound. Yield: 120 mg; Analytical HPLC: purity 75% (System A), purity 80% (System B); ¹H NMR (400 MHz, CDCl₃) δ ppm 2.02-2.12 (m, 4H) 3.09 (s, 3H) 3.15 (m, 2H) 3.35 (m, 2H) 3.86 (m, 1H) 4.71 (s, 2H) 7.56 (m, 1H) 7.74-7.79 (m, 2H) 7.94 (m, 1H) 8.05 (m, 2H) 8.79 (m, 1H); LRESIMS for C₁₈H₂₂N₂O₃S m/z/347 (M+H)⁺.

### Example A34

### tert-Butyl 4-({5-[4-(methylsulfonyl)phenyl]pyridin-2-yl}methoxy)piperidine-1-carboxylate

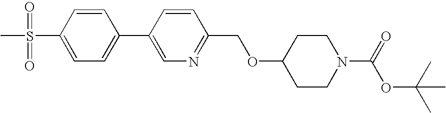

A mixture of 5-bromo-2-(chloromethyl)pyridine (100 mg, 0.48 mmol), prepared from 5-bromopyridin-2-yl)methanol in accordance with the literature procedure (Heuvel et al., J. Org. Chem. 2004, 69(2), 250-262), tert-butyl 4-hydroxypiperidine-1-carboxylate (97.5 mg, 0.48 mmol) and potassium tert-butoxide (65.2 mg, 0.58 mmol) in dry THF (2 mL) was heated at 60° C. for 16 h. The reaction mixture was filtered and the filtrate was concentrated under reduced pressure. (4-Methylsulfonylphenyl)boronic acid (106.5 mg, 0.53 mmol), K₂CO₃ (80.3 mg, 0.58 mmol) and Pd(PPh₃)₄ (55.9 mg, 0.048 mmol) in dioxane (5 mL) and water (1 mL) were heated at 90° C. for 16 h. The reaction mixture was filtered and the filtrate was concentrated under reduced pressure. The product was purified by preparative HPLC (System D). The product-containing fractions were combined and diluted with ethyl acetate and washed with saturated aqueous NaHCO₃. The organic phase was collected and the solvent was removed under reduced pressure to give the title compound. Yield: 68 mg (31%); Analytical HPLC: purity 96%, (System A, R_{T}=1.94 min); ¹H NMR (400 MHz, CD₃OD) δ ppm 1.43 (s, 9H) 1.51-1.69 (m, 2H) 1.81-2.03 (m, 2H) 3.14 (s, 3H) 3.19 (s, 2H) 3.60-3.87 (m, 3H) 4.71 (s, 2H) 7.67 (m, 1H) 7.85-7.98 (m, 2H) 7.99-8.10 (m, 2H) 8.17 (m, 1H) 8.80 (m, 1H); LRESIMS for C₂₃H₃₀N₂O₅S m/z 447 (M+H)⁺; HRESIMS, calc. monoiso mass (Da): 446.1875, found monoiso mass (Da): 446.1884.

### Example A35

### 4-Methoxyphenyl 4-({5-[4-(methylsulfonyl)phenyl]pyridin-2-yl}methoxy)piperidine-1-carboxylate

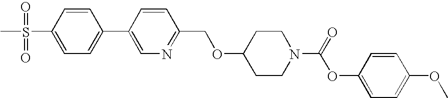

Concentrated HCl (4.3 μL, 0.052 mmol) was added to a solution of tert-butyl 4-({5-[4-(methylsulfonyl)phenyl]pyridin-2-yl}methoxy)piperidine-1-carboxylate (63.0 mg, 0.14 mmol; obtained in Example A34) in DCM (2 mL). The reaction mixture was stirred at r.t. for 16 h, after which time additional concentrated HCl (43 μL, 10 equiv, 0.52 mmol) was added. After 17 h, the reaction mixture was distributed equally to 4 vials. Triethylamine (50.7 μL, 0.36 mmol), 4-methoxyphenyl chlorodicarbonate (5.4 μL, 0.036 mmol) and DCM (1 mL) were added to one of the vials. This mixture was stirred at ambient temperature for 16 h. The solvent was removed under reduced pressure and the product was purified by preparative HLPC (System E) to give the title compound. Yield: 1.4 mg (8%); Analytical HPLC: purity 100%, (System A, R_{T}=2.49 min); LRESIMS for C₂₆H₂₈N₂O₆S m/z 497 (M+H)⁺; HRESIMS, calc. monoiso mass (Da): 496.1668, found monoiso mass (Da): 496.1672.

### Example A36

### Prop-2-yn-1-yl 4-({5-[4-(methylsulfonyl)phenyl]pyridin-2-yl}methoxy)piperidine-1-carboxylate

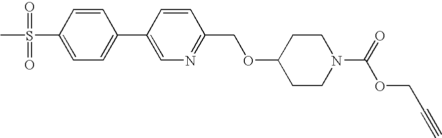

Concentrated HCl HCl (4.3 μL, 0.052 mmol) was added to a solution of tert-butyl 4-({5-[4-(methylsulfonyl)phenyl]pyridin-2-yl}methoxy)piperidine-1-carboxylate (63.0 mg, 0.14 mmol; obtained in Example A34) in DCM (2 mL). The reaction mixture was stirred at r.t. for 16 h, after which time additional concentrated HCl (43 μL, 10 equiv, 0.52 mmol) was added. After 17 h, the reaction mixture was distributed equally into 4 vials. Triethylamine (50.7 μL, 0.36 mmol), prop-2-yn-1-yl chlorodicarbonate (4.3 mg, 0.036 mmol) and DCM (1 mL) were added to one of the vials. This mixture was stirred at ambient temperature for 16 h. The solvent was removed under reduced pressure and the product was purified by preparative HLPC (System E) to give the title compound. Yield 0.6 mg (4%); Analytical HPLC: purity 100% (System A and B, R_{TA}=2.49 min, R_{TB}=2.29 min); LRESIMS for C₂₂H₂₄N₂O₅S m/z 429 (M+H)⁺; HRESIMS, calc. monoiso mass (Da): 428.1406, found monoiso mass (Da): 428.1405.

### Example A37

### 2-({[1-(Bicyclo[2.2.1]hept-2-ylcarbonyl)piperidin-4-yl]oxy}methyl)-5-[4-(methylsulfonyl)phenyl]pyridine, trifluoroacetate

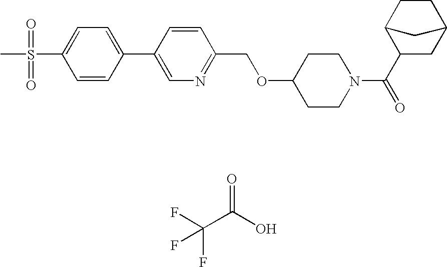

A mixture of norbornane-2-carboxylic acid (11 mg, 0.080 mmol), propylphosphonic anhydride (1.57 M solution in EtOAc, 212 μL, 0.33 mmol) in DMF (0.3 mL) containing triethylamine (28 μL, 0.20 mmol) was stirred for 10 minutes. 5-[4-(Methylsulfonyl)-phenyl]-2-[(piperidin-4-yloxy)methyl]pyridine (23 mg, 0.066 mmol; Intermediate A10) was added and the mixture was stirred over night at r.t. The product was purified by preparative HPLC (System D) giving the title compound. Yield: 10 mg (27%), Analytical HPLC: purity 97% (System A), purity 94% (System B); ¹H NMR (400 MHz, CDCl₃) δ ppm 1.13-2.13 (m, 12H) 2.25-2.46 (m, 2H) 2.91-2.99 (m, 1H) 3.11 (s, 3H) 3.30-3.55 (m, 2H) 3.80-4.10 (m 2H) 4.98 (br s, 2H) 7.83 (m, 2H) 7.99 (m, 1H) 8.13 (m, 2H) 8.44 (m, 1H) 9.10 (br s, 1H); LRESIMS for C₂₆H₃₂N₂O₄S m/z 469 (M+H)⁺; HRESIMS, calc. monoiso mass (Da): 468.2083, found monoiso mass (Da): 468.2080.

### Example A38

### Isopropyl 4-({5-[4-(methylsulfonyl)phenyl]pyridin-2-yl}methoxy)piperidine-1-carboxylate, trifluoroacetate

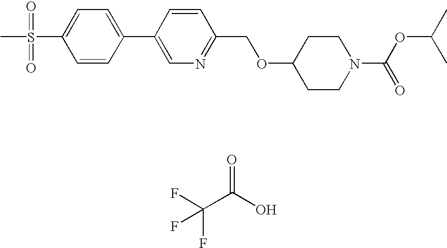

Isopropyl chloroformate 1 M solution in toluene (80 μL, 0.080 mmol) was added to a solution of 5-[4-(methylsulfonyl)phenyl]-2-[(piperidin-4-yloxy)methyl]pyridine (23 mg, 0.066 mmol; Intermediate A10) in DMF (0.3 mL) containing triethylamine (28 μL, 0.20 mmol). The mixture was stirred for 20 minutes at r.t. and the solvent was evaporated. The product was purified by preparative HPLC (System D) giving the title compound. Yield: 7 mg (20%); Analytical HPLC: purity 97% (System A), purity 94% (System B); ¹H NMR (400 MHz, CDCl₃) δ ppm 1.24 (d, J=6.4 Hz, 6H) 1.37-1.45 (m, 1H) 1.60-1.71 (m, 2H) 1.91-2.00 (m, 2H) 3.11 (s, 3H) 3.17-3.26 (m, 2H) 3.70-3.87 (m, 3H) 4.88-4.97 (m, 2H) 7.81 (m, 2H) 7.92 (m, 1H) 8.11 (m, 2H) 8.31 (m, 1H) 9.04 (m, 1H). LRESIMS for C₂₂H₂₈N₂O₅S m/z 433 (M+H)⁺; HRESIMS, calc. monoiso mass (Da): 432.1719, found monoiso mass (Da): found 432.1717.

### Intermediate A11

### tert-Butyl 4-{[(5-bromopyridin-2-yl)methyl]amino}piperidine-1-carboxylate

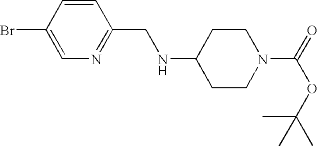

Thionyl chloride (1.90 g, 15.95 mmol) was added to an ice-cold solution of (5-bromopyridin-2-yl)-methanol (2.00 g, 10.64 mmol) in DCM (20 mL) giving a milky mixture. After addition the ice-bath was removed and the mixture was stirred for 1.5 hour at room-temperature. The mixture was washed with saturated aqueous NaHCO₃ and the organic phase was dried over Na₂SO₄ giving (2.16 g) 5-bromo-2-(chloromethyl)pyridine as an oil. This intermediate was dissolved in DMF (10 mL) and N,N-diisopropylethylamine (5.41 mL, 31.38 mmol) was added followed by tert-butyl 4-aminopiperidine-1-carboxylate (2.30 g, 11.51 mmol). The mixture was stirred at 70° C. for 3 hours, diluted with EtOAc and then washed with water containing brine. The organic phase was dried over Na₂SO₄, filtered and the solvent was evaporated. The residue was purified by flash chromatography using EtOAc/n-pentane/25% aqueous NH₃ (800:195:5) as eluent to give the title compound. Yield: 2.57 g (65%), Analytical HPLC: purity 97% (System A), purity 100% (System B); ¹H NMR (400 MHz, CDCl₃) δ ppm 1.24-1.36 (m, 2H) 1.44 (s, 9H) 1.70 (s, 2H) 1.81-1.88 (m, 2H) 2.64 (m, 1H) 2.78 (s, 2H) 3.89 (s, 2H) 3.94-4.10 (m, 2H) 7.23 (m, 1H) 7.75 (m, 1H) 8.59 (m, 1H); LRESIMS for C₁₆H₂₄BrN₃O₂ m/z 371 (M+H)⁺

### Intermediate A12

### tert-Butyl 4-[({5-[4-(methylsulfonyl)phenyl]pyridin-2-yl}methyl)amino]piperidine-1-carboxylate

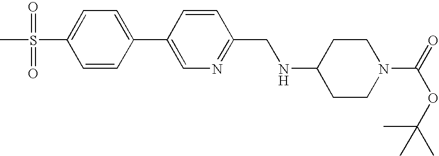

A mixture of tert-butyl 4-{[(5-bromopyridin-2-yl)methyl]amino}piperidine-1-carboxylate (1.50 g, 4.05 mmol; Intermediate A11), (4-methylsulfonylphenyl)boronic acid (0.89 g, 4.45 mmol), Pd(PPh₃)₄ (0.23 g, 0.20 mmol), K₂CO₃ (1.39 g, 10.13 mmol), 1,4-dioxane (40 mL) and water (10 mL) was stirred over night at 90° C. The mixture was diluted with DCM and washed with water. The organic phase was dried over Na₂SO₄, filtered and the solvent was evaporated to give the title compound as a crude oil. Yield 2.35 g. This intermediate was used without further purification in the synthesis of Example A39.

### Example A39

### tert-Butyl 4-[methyl({5-[4-(methylsulfonyl)phenyl]pyridin-2-yl}methyl)amino]-piperidine-1-carboxylate

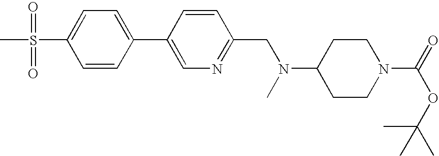

Sodium cyanoborohydride (0.257 g, 4.08 mmol) was added to a solution of tert-butyl 4-[({5-[4-(methylsulfonyl)phenyl]pyridin-2-yl}methyl)amino]piperidine-1-carboxylate (1.30 g, 2.92 mmol; Intermediate A12) in MeOH (130 mL), formaldehyde 37 wt. % sol. in water (0.526 g, 17.50 mmol) and 5 M HCl in MeOH (0.23 mL, 1.17 mmol). The mixture was stirred over night at r.t. Saturated aqueous NaHCO₃, water and brine were added and the mixture was extracted with DCM. The organic phase was dried over Na₂SO₄ giving (1.30 g) crude product. 20 mg of this material was purified by preparative HPLC (System D). Pure fractions were combined, saturated aqueous NaHCO₃ was added and the resulting mixture was extracted with DCM. The organic phase was dried over Na₂SO₄, filtered and the solvent was evaporated giving (14 mg) solid product. Analytical HPLC: purity 96% (System A), purity 99% (System B); ¹H NMR (400 MHz, CDCl₃) δ ppm 1.45 (s, 9H) 1.46-1.87 (m, 5H) 2.28 (s, 3H) 2.57-2.74 (m, 2H) 3.79 (s, 2H) 4.16 (br s, 2H) 7.56 (m, 1H) 7.65-7.69 (m, 2H) 7.73-7.77 (m, 2H) 7.85 (m, 1H) 8.75 (m, 1H); LRESIMS for C₂₄H₃₃N₃O₄S m/z 460 (M+H)⁺ HRESIMS, calc. monoiso mass (Da): 459.2192, found monoiso mass (Da): 459.2192.

### Intermediate A13

### N-Methyl-N-({5-[4-(methylsulfonyl)phenyl]pyridin-2-yl}methyl)piperidin-4-amine

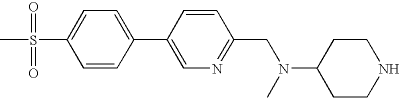

tert-Butyl 4-[methyl({5-[4-(methylsulfonyl)phenyl]pyridin-2-yl}methyl)amino]piperidine-1-carboxylate (1.28 g, 2.83 mmol, obtained in Example A39) was dissolved in EtOAc (75 mL) and HCl (g) was bubbled through the solution for 3 minutes immediately giving a precipitate. The mixture was stirred for 45 minutes at r.t. and the solid was collected by filtration. The solid was treated with saturated aqueous NaHCO₃ and the product was extracted with CHCl₃. The organic phase was dried over Na₂SO₄, filtered and the filtrate was concentrated giving the title compound. Yield: 803 mg (79%); Analytical HPLC: purity 90% (System A); ¹H NMR (400 MHz, CDCl₃) δ ppm 1.47-1.58 (m, 2H) 1.65 (m, 3H) 1.87 (m, 2H) 2.30 (s, 3H) 2.53-2.63 (m, 3H) 3.09 (s, 3H) 3.13-3.19 (m, 2H) 3.81 (s, 2H) 7.58 (m, 1H) 7.76 (m, 2H) 7.87 (m, 1H) 8.04 (m, 2H) 8.77 (m, 1H); LRESIMS for C₁₉H₂₅N₃O₂S m/z 360 (M+H)⁺.

### Example A40

### (1S,2R,4R)-bicyclo[2.2.1]hept-2-yl 4-[methyl({5-[4-(methylsulfonyl)phenyl]pyridin-2-yl}methyl)amino]piperidine-1-carboxylate

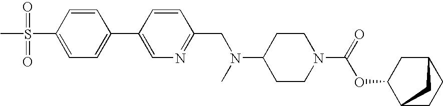

To a solution of (1S,2R,4R)-bicyclo[2.2.1]heptan-2-ol (7.0 mg, 0.062 mmol) in DCM (0.3 mL) at r.t. under N₂ (g) was added 1,1′-carbonylbis(1H-imidazole) (11.2 mg, 0.062 mmol) in DCM (0.4 mL). The mixture was stirred for 1 h after which N-methyl-N-({5-[4-(methylsulfonyl)phenyl]pyridin-2-yl}methyl)piperidin-4-amine (11.2 mg, 0.031 mmol; Intermediate A13) was added, and the reaction was stirred for 3 days at 35° C. The solvent was removed, and the residue was purified by preparative HPLC (System D) to give the title compound. Yield: 11.7 mg (75%). Analytical HPLC: purity 100% (System A and B); ¹H NMR (400 MHz, CDCl₃) δ ppm 0.93-1.01 (m, 1H) 1.27-1.32 (m, 2H) 1.35-1.43 (m, 2H) 1.53-1.83 (m, 4H) 1.93-2.04 (m, 1H) 2.18-2.24 (m, 1H) 2.34-2.47 (m, 3H) 2.66-2.76 (m, 2H) 2.77 (s, 3H) 3.10 (s, 3H) 3.36-3.49 (m, 1H) 4.24-4.41 (m, 2H) 4.44 (s, 2H) 4.85-4.93 (m, 1H) 7.76-7.81 (m, 2H) 8.06-8.11 (m, 3H) 8.32-8.38 (m, 1H) 8.81-8.86 (m, 1H); LRESIMS for C₂₇H₃₅N₃O₄S m/z 498 (M+H)⁺; HRESIMS, calc. monoiso mass (Da): 497.2348, found monoiso mass (Da): 497.2347.

### Example A41

### (3-Methyloxetan-3-yl)methyl 4-[methyl({5-[4-(methylsulfonyl)phenyl]pyridin-2-yl}-methyl)amino]piperidine-1-carboxylate

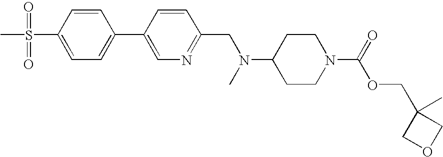

To a solution of (3-methyloxetan-3-yl)methanol (28.0 mg, 0.274 mmol) in acetonitrile (0.3 mL) in a Smith Process vial at r.t. under N₂ (g) was slowly added 1,1′-carbonylbis(1H-imidazole) (44.5 mg, 0.274 mmol) in acetonitrile (0.4 mL). The mixture was stirred for 20 min after which N-methyl-N-({5-[4-(methylsulfonyl)phenyl]pyridin-2-yl}methyl)piperidin-4-amine (9.9 mg, 0.027 mmol; Intermediate A13) was added, and the reaction was heated by microwave irradiation at 100° C. for 10 min. The mixture was purified by preparative HPLC (System D) to give the title compound. Yield: 6.4 mg (48%); Analytical HPLC: purity 100% (System A and B); ¹H NMR (400 MHz, CDCl₃) δ ppm 1.32 (s, 3H) 1.74-1.86 (m, 2H) 2.35-2.50 (m, 2H) 2.66-2.91 (m, 2H) 2.77 (s, 3H) 3.10 (s, 3H) 3.37-3.48 (m, 1H) 4.15 (s, 2H) 4.25-4.47 (m, 6H) 4.52 (d, J=6.0 Hz, 2H) 7.78 (m, 2H) 8.04 (m, 1H) 8.08 (m, 2H) 8.28 (m, 1H) 8.82 (m, 1H); LRESIMS for C₁₋₅H₃₃N₃O₅S m/z 488 (M+H)⁺; HRESIMS, calc. monoiso mass (Da): 487.2141, found monoiso mass (Da): 487.2137.

### Example A42

### (1-Methylcyclopropyl)methyl 4-[methyl({5-[4-(methylsulfonyl)phenyl]pyridin-2-yl}-methyl)amino]piperidine-1-carboxylate

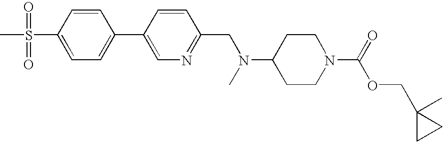

To a solution of (1-methylcyclopropyl)methanol (25.0 mg, 0.290 mmol) in acetonitrile (0.3 mL) in a Smith Process vial at r.t. under N₂ (g) was slowly added 1,1′-carbonylbis(1H-imidazole) (47.1 mg, 0.290 mmol) in acetonitrile (0.4 mL). The mixture was stirred for 30 min after which N-methyl-N-({5-[4-(methylsulfonyl)phenyl]pyridin-2-yl}methyl)piperidin-4-amine (10.4 mg, 0.029 mmol; Intermediate A13) was added, and the reaction was heated by microwave irradiation at 100° C. for 10 min. The residue was purified by preparative HPLC (System D) to give the title compound. Yield: 9.2 mg (67%); Analytical HPLC: purity 100% (System A and B); ¹H NMR (400 MHz, CDCl₃) δ ppm 0.32-0.38 (m, 2H) 0.44-0.49 (m, 2H) 1.11 (s, 3H) 1.70-1.84 (m, 2H) 2.34-2.47 (m, 2H) 2.67-2.77 (m, 2H) 2.77 (s, 3H) 3.10 (s, 3H) 3.36-3.46 (m, 1H) 3.87 (s, 2H) 4.30-4.39 (m, 2H) 4.39 (s, 2H) 7.76-7.79 (m, 2H) 8.04 (m, 1H) 8.06-8.10 (m, 2H) 8.27-8.32 (m, 1H) 8.82 (m, 1H); LRESIMS for C₂₅H₃₃N₃O₄S m/1472 (M+H)⁺; HRESIMS, calc. monoiso mass (Da): 471.2192, found monoiso mass (Da): 471.2181.

### Intermediate A14

### tert-butyl 4-{[(5-bromopyridin-2-yl)methyl](methyl)amino}piperidine-1-carboxylate

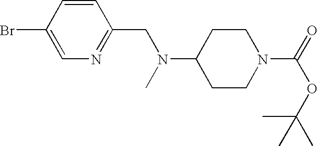

Sodium cyanoborohydride (0.119 g, 1.89 mmol) was added to a solution of tert-butyl 4-{[(5-bromopyridin-2-yl)methyl]amino}piperidine-1-carboxylate (0.50 g, 1.35 mmol; Intermediate A11) in MeOH (50 mL), formaldehyde 37 wt. % solution in water (0.243 g, 8.10 mmol) and 5 M HCl in MeOH (0.108 mL, 0.54 mmol). The mixture was stirred at r.t. for 0.5 h. Saturated aqueous NaHCO₃, water and brine were added and the mixture was extracted with DCM. The organic phase was dried over Na₂SO₄ filtered and the solvent was evaporated. Yield 0.49 g (94%) crude product.

### Example A43

### tert-Butyl 4-[{[5-(4-cyanophenyl)pyridin-2-yl]methyl}(methyl)amino]piperidine-1-carboxylate

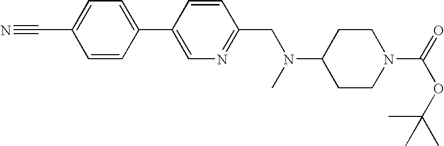

A mixture of tert-butyl 4-{[(5-bromopyridin-2-yl)methyl](methyl)amino}piperidine-1-carboxylate (0.03 g, 0.08 mmol; Intermediate A14), (4-cyanophenyl)boronic acid (0.013 g, 0.09 mmol), Pd(PPh₃)₄ (0.005 g, 0.004 mmol), K₂CO₃ (0.027 g, 0.20 mmol), 1,4-dioxane (0.8 mL) and water (0.2 mL) was heated by microwave irradiation at 130° C. for 20 minutes. Solid material was filtered off and the filtrate was subjected to purification by preparative HPLC (System D). Pure fractions were combined, saturated aqueous NaHCO₃ was added and the resulting mixture was extracted with DCM. The organic phase was dried over Na₂SO₄, filtered and the solvent was evaporated giving the title compound. Yield: 14 mg (43%); Analytical HPLC: purity 99% (System A), purity 100% (System B); ¹H NMR (400 MHz, CDCl₃) δ ppm 1.45 (s, 9H) 1.46-1.56 (m, 2H) 1.71-1.79 (m, 1H) 1.79-1.88 (m, 2H) 2.28 (s, 3H) 2.57-2.74 (m, 2H) 3.79 (s, 2H) 4.16 (m, 2H) 7.56 (m, 1H) 7.65-7.69 (m, 2H) 7.73-7.77 (m, 2H) 7.85 (m, 1H) 8.75 (m, 1H); LRESIMS for C₂₄H₃₀N₄O₂ m/z 407 (M+H)⁺; HRESIMS, calc. monoiso mass (Da): 406.2369, found monoiso mass (Da): 406.2371.

### Example A44

### Isobutyl 4-[methyl({5-[4-(methylsulfonyl)phenyl]pyridin-2-yl}methyl)amino]-piperidine-1-carboxylate, trifluoroacetate

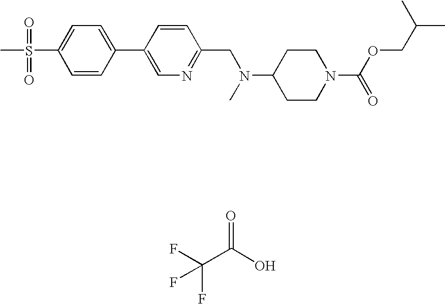

Isobutyl chloroformate (13 μL, 0.10 mmol) was added to a solution of N-methyl-N-({5-[4-(methylsulfonyl)phenyl]pyridin-2-yl}methyl)piperidin-4-amine (30 mg, 0.083 mmol; Intermediate A13) in DMF (0.3 mL) containing triethylamine (35 μL, 0.25 mmol) and the mixture was stirred for 15 minutes at room temperature. The crude mixture was purified by preparative HPLC (System D) to give the trifluoroacetate salt of title compound. Yield: 36 mg (76%); Analytical HPLC: purity 100% (System A), purity 100% (System B); ¹H NMR (400 MHz, CDCl₃) δ ppm 0.93 (d, J=6.8 Hz, 6H) 1.73 (m, 2H) 1.88-1.98 (m, 1H) 2.18-2.26 (m, 2H) 2.73-2.83 (m, 2H) 2.85 (s, 3H) 3.11 (s, 3H) 3.64-3.75 (m, 1H) 3.87 (d, J=6.5 Hz, 2H) 4.37 (m, 1H) 4.48 (s, 3H) 7.74-7.80 (m, 3H) 8.04 (m, 1H) 8.08 (m, 2H) 8.85 (m, 1H); LRESIMS for C₂₄H₃₃N₃O₄S m/z 460 (M+H)⁺; HRESIMS, calc. monoiso mass (Da): 459.2192, found monoiso mass (Da): 459.2194.

### Example A45

### Cyclobutyl 4-[methyl({5-[4-(methylsulfonyl)phenyl]pyridin-2-yl}methyl)amino]-piperidine-1-carboxylate

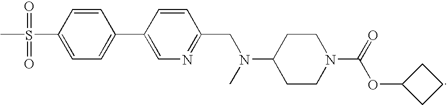

To a solution of cyclobutanol (5.0 mg, 0.069 mmol) in DCM (0.3 mL) at r.t. under N₂ (g) was added 1,1′-carbonylbis(1H-imidazole) (11.2 mg, 0.069 mmol) in DCM (0.4 mL). The mixture was stirred for 1 h after which N-methyl-N-({5-[4-(methylsulfonyl)phenyl]pyridin-2-yl}methyl)piperidin-4-amine (12.5 mg, 0.035 mmol; Intermediate A13) was added, and the reaction was stirred for 3 days at 35° C. The solvent was removed, and the residue was purified by preparative HPLC (System D) to give the title compound as a white solid. Yield: 11 mg (71%); Analytical HPLC: purity 100% (System A and B); ¹H NMR (400 MHz, CDCl₃) δ ppm 1.52-1.81 (m, 4H) 1.98-2.10 (m, 2H) 2.12-2.25 (m, 2H) 2.27-2.38 (m, 2H) 2.60 (br s, 4H) 2.67-2.84 (m, 3H) 3.10 (s, 3H) 4.07-4.21 (m, 1H) 4.28 (br s, 2H) 4.91 (quint, J=7.5 Hz, 1H) 7.73-7.80 (m, 2H) 7.90-8.02 (m, 2H) 8.02-8.09 (m, 2H) 8.80 (s, 1H); LRESIMS for C₂₄H₃₁N₃O₄S m/z 458 (M+H)⁺; HRESIMS, calc. monoiso mass (Da): 457.2035, found monoiso mass (Da): 457.2037.

### Example A46

### 1-Cyclopropylethyl 4-[methyl({5-[4-(methylsulfonyl)phenyl]pyridin-2-yl}methyl)-amino]piperidine-1-carboxylate

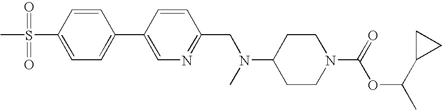

To a solution of 1-cyclopropylethanol (6.0 mg, 0.070 mmol) in DCM (0.3 mL) at r.t. under N₂ (g) was added 1,1′-carbonylbis(1H-imidazole) (11.3 mg, 0.070 mmol) in DCM (0.4 mL). The mixture was stirred for 2 h after which N-methyl-N-({5-[4-(methylsulfonyl)phenyl]pyridin-2-yl}methyl)piperidin-4-amine (10.0 mg, 0.028 mmol; Intermediate A13) was added, and the reaction was stirred for 3 days at 35° C. The solvent was removed, and the residue was purified by preparative HPLC (System D) to give the title compound as a white solid. Yield: 7.4 mg (56%); Analytical HPLC: purity 100% (System A and B); ¹H NMR (400 MHz, CDCl₃) δ ppm 0.18-0.27 (m, 1H) 0.34-0.42 (m, 1H) 0.43-0.56 (m, 2H) 0.91-1.01 (m, 1H) 1.26-1.40 (m, 5H) 1.69-1.82 (m, 2H) 2.26-2.53 (m, 3H) 2.66-2.76 (m, 2H) 2.77 (s, 3H) 3.35-3.47 (m, 1H) 4.18-4.28 (m, 1H) 4.29-4.40 (m, 2H) 4.42 (s, 2H) 7.75-7.81 (m, 2H) 8.04-8.11 (m, 3H) 8.35 (m, 1H) 8.80-8.86 (m, 1H); LRESIMS for C₂₅H₃₃N₃O₄S m/z 472 (M+H)⁺; HRESIMS, calc. monoiso mass (Da): 471.2192, found monoiso mass (Da): 471.2192.

### Example A47

### Isopropyl 4-[methyl({5-[4-(methylsulfonyl)phenyl]pyridin-2-yl}methyl)amino]-piperidine-1-carboxylate, trifluoroacetate

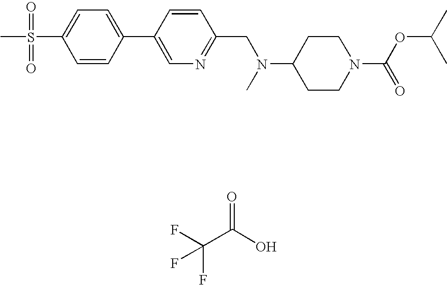

Isopropyl chloroformate 1 M in toluene (100 μL, 0.1 mmol) was added to a solution of N-methyl-N-({5-[4-(methylsulfonyl)phenyl]pyridin-2-yl}-methyl)piperidin-4-amine (30 mg, 0.083 mmol; Intermediate A13) in DMF (0.3 mL) containing triethylamine (35 μL, 0.25 mmol) and the mixture was stirred for 15 minutes at room temperature. The crude mixture was subjected to purification by preparative HPLC (System D) to give the trifluoroacetate salt of title compound. Yield: 38 mg (81%); Analytical HPLC: purity 100% (System A), purity 100% (System B); ¹H NMR (400 MHz, CDCl₃) δ ppm 1.24 (d, J=6.3 Hz, 6H) 1.72 (m, 2H) 2.20 (m, 2H) 2.74-2.84 (m, 2H) 2.85 (s, 3H) 3.10 (s, 3H) 3.68 (m, 1H) 4.37 (m, 2H) 4.49 (s, 2H) 4.86-4.94 (m, 1H) 7.74-7.79 (m, 3H) 8.04 (m, 1H) 8.06-8.09 (m, 2H) 8.84 (m, 1H); LRESIMS for C₂₃H₃₁N₃O₄S m/z 446 (M+H)⁺; HRESIMS, calc. monoiso mass (Da): 445.2035, found monoiso mass (Da): 445.2043.

### Example A48

### 3-Hydroxy-3-methylbutyl 4-[methyl({5-[4-(methylsulfonyl)phenyl]pyridin-2-yl}-methyl)amino]piperidine-1-carboxylate

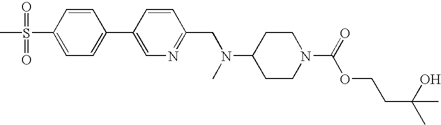

To a solution of 3-methylbutane-1,3-diol (28.0 mg, 0.269 mmol) in acetonitrile (0.3 mL) in a Smith Process vial at r.t. under N₂ (g) was slowly added 1,1′-carbonylbis(1H-imidazole) (43.6 mg, 0.269 mmol) in acetonitrile (0.4 mL). The mixture was stirred for 30 min after which N-methyl-N-({5-[4-(methylsulfonyl)phenyl]pyridin-2-yl}methyl)piperidin-4-amine (9.7 mg, 0.027 mmol; Intermediate A13) was added, and the reaction was heated by microwave irradiation at 100° C. for 10 min. The mixture was purified by preparative HPLC (System D) to give the title compound as a white solid. Yield: 9.4 mg (71%); Analytical HPLC: purity 100% (System A and B); ¹H NMR (400 MHz, CDCl₃) δ ppm 1.25 (s, 6H) 1.69-1.80 (m, 2H) 1.83 (t, J=6.7 Hz, 2H) 2.29-2.44 (m, 2H) 2.64-2.86 (m, 5H) 3.10 (s, 3H) 3.29-3.47 (m, 1H) 4.26 (t, J=6.7 Hz, 2H) 4.29-4.48 (m, 4H) 7.75-7.79 (m, 2H) 8.02 (m, 1H) 8.05-8.09 (m, 2H) 8.14-8.31 (m, 1H) 8.81 (m, 1H); LRESIMS for C₂₅H₃₅N₃O₅S m/z 490 (M+H)⁺; HRESIMS, calc. monoiso mass (Da): 489.2297, found monoiso mass (Da): 489.2291.

### Intermediate B1

### tert-Butyl 4-{[(6-chloropyridin-3-yl)methyl]amino}piperidine-1-carboxylate

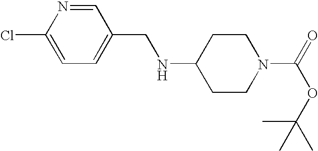

2-Chloro-5-(chloromethyl)pyridine (0.81 g, 5 mmol), 4-amino-piperidine-1-carboxylic acid tert-butyl ester (1.00 g, 5 mmol) and N,N-diisopropylethylamine (1.74 mL, 10 mmol) were mixed with DMF (10 mL) and heated to 50° C. overnight. The mixture was concentrated under reduced pressure. To the residue were added DCM and 10% aqueous Na₂CO₃. The organic phase was separated, dried (Na₂SO₄), filtered and concentrated. Flash chromatography on silica gel with EtOAc gave the title compound. Yield: 1.15 g (71%); Analytical HPLC: purity 92% (System A and B, R_{TA}=1.53 min, R_{TB}=1.30 min); ¹H NMR (400 MHz, CDCl₃) δ ppm 1.20-1.36 (m, 2H), 1.44 (s, 9H), 1.77-1.91 (m, 2H), 2.64 (m, 1H), 2.78 (m, 2H), 3.81 (s, 2H), 4.00 (br s, 2H), 7.29 (d, 1H), 7.67 (m, 1H), 8.32 (s, 1H); LRESIMS for C₁₆H₂₄ClN₃O₂ m/z=270 (M+H−t-Bu)⁺.

### Example B1

### tert-Butyl 4-[({6-[4-(methylsulfonyl)phenyl]pyridin-3-yl}methyl)amino]piperidine-1-carboxylate

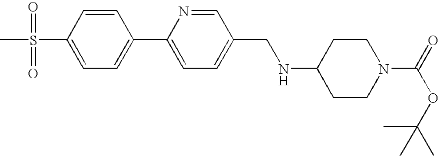

tert-Butyl 4-{[(6-chloropyridin-3-yl)methyl]amino}piperidine-1-carboxylate (812 mg, 2.5 mmol; Intermediate B1), (4-methylsulfonyl)phenylboronic acid (550 mg, 2.75 mmol), Pd(PPh₃)₄ (145 mg, 0.125 mmol) and K₂CO₃ (863 mg, 6.25 mmol) were mixed with dioxane (20 mL) and water (5 mL) and heated to 90° C. overnight. The mixture was filtered and concentrated under reduced pressure. The residue was extracted with DCM and the organic phase was washed with 5% aqueous NaHCO₃ and brine and then concentrated. Flash chromatography of the residue on silica gel using ammonia-saturated CHCl₃/MeOH (97:3) as eluent gave the title compound. Yield: 765 mg (69%); Analytical HPLC: purity 97% (System A and B, R_{TA}=1.60 min, R_{TB}=1.40 min); ¹H NMR (400 MHz, CDCl₃) δ ppm 1.25-1.40 (m, 2H), 1.40-1.48 (s, 9H), 1.82-1.95 (m, 2H), 2.65-2.87 (m, 3H), 3.08 (s, 3H), 3.91 (s, 2H), 3.94-4.01 (m, 2H), 7.72-7.78 (m, 1H), 7.81-7.87 (m, 1H), 7.98-8.06 (m, 2H), 8.14-8.71 (m, 2H), 8.67 (s, 1H); LRESIMS for C₂₃H₃₁N₃O₄S m/z=446 (M+H)⁺.

### Example B2

### tert-Butyl 4-[methyl({6-[4-(methylsulfonyl)phenyl]pyridin-3-yl}methyl)amino]-piperidine-1-carboxylate

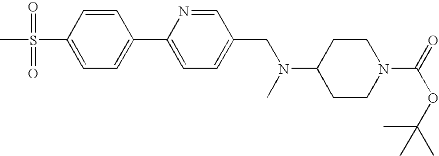

The reductive amination reaction was carried by using similar conditions reported in the literature (J. Org. Chem. 1996, 61, 3849-3862). tert-Butyl 4-[({6-[4-(methylsulfonyl)-phenyl]pyridin-3-yl}methyl)amino]piperidine-1-carboxylate (760 mg, 1.7 mmol; obtained in Example B1) was dissolved in 1,2-dichloroethane (10 mL) and 37% formalin (0.225 mL, 276 mg, 9.2 mmol) and NaBH(OAc)₃ (1.44 g, 6.8 mmol) were added. The mixture was stirred at room temperature overnight. 1 M NaOH was added and the mixture was extracted with DCM. Concentration under reduced pressure gave the title compound. Yield: 776 mg (99%); Analytical HPLC: purity 100%, (System A and B, R_{TA}=1.70 min, R_{T}=1.49 min); ¹H NMR (400 MHz, DMSO-d₆) δ ppm 1.30-1.44 (m, 11H), 1.69-1.81 (m, 2H), 2.12 (s, 3H), 2.53-2.82 (m, 3H), 3.25 (s, 3H), 3.63 (s, 2H) 3.92-4.05 (m, 2H), 7.80-7.87 (m, 1H), 7.98-8.08 (m, 3H), 8.30-8.36 (m, 2H), 8.62 (m, 1H); LRESIMS for C₂₄H₃₃N₃O₄S m/z=460 (M+H)⁺; HRESIMS, calc. monoiso mass (Da): 459.2192, found monoiso mass (Da): 459.2200.

### Intermediate B2

### N-Methyl-N-({6-[4-(methylsulfonyl)phenyl]pyridin-3-yl}methyl)piperidin-4-amine

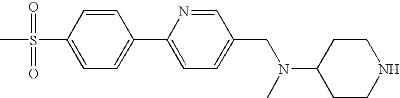

tert-Butyl 4-[methyl({6-[4-(methylsulfonyl)phenyl]pyridin-3-yl}methyl)amino]piperidine-1-carboxylate (460 mg, 1.00 mmol; obtained in Example B2) was dissolved in DCM (6 mL) and TFA (1.5 mL) was added. The mixture was evaporated after 45 min. To the residue was added 2 M NaOH and the resulting mixture was extracted with CHCl₃. The extract was concentrated under reduced pressure to give the title compound. Yield 355 mg (99%); Analytical HPLC: purity 100% (System A, R_{T}=1.31 min, 5-60% MeCN over 3 min), purity 100% (System B, R_{T}=1.17 min, 5-60% MeCN over 3 min); ¹H NMR (400 MHz, DMSO-d₆) δ ppm 1.29-1.43 (m, 2H), 1.64-1.74 (m, 2H), 2.12 (s, 3H), 2.33-2.43 (m, 2H), 2.91-3.01 (m, 3H), 3.25 (s, 3H), 3.62 (s, 2H), 7.79-7.87 (m, 1H), 7.98-8.07 (m, 3H), 8.29-8.36 (m, 2H), 8.62 (m, 1H); LRESIMS for C₁₉H₂₅N₃O₂S m/z=360 (M+H)⁺.

### Example B3

### 2-Methoxyethyl 4-[methyl({6-[4-(methylsulfonyl)phenyl]pyridin-3-yl}methyl)amino]-piperidine-1-carboxylate, trifluoroacetate

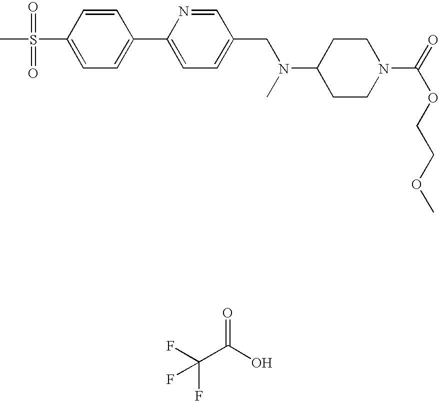

The title compound was prepared from Intermediate B2 and 2-methoxyethyl chloroformate in accordance with general method A. Yield 26 mg (65%); Analytical HPLC: purity 100% (System A, R_{T}=1.28 min); ¹H NMR (400 MHz, CDCl₃) δ ppm 1.66-1.82 (m, 2H), 2.09-2.19 (m, 2H), 2.71 (s, 3H), 2.80 (br s, 2H), 3.10 (s, 3H), 3.38 (s, 3H), 3.50-3.63 (m, 3H), 4.25 (m, 2H), 4.28-4.51 (m, 4H), 7.91 (m, 1H), 8.07 (m, 2H), 8.13-8.22 (m, 3H), 8.72 (s, 1H); LRESIMS for C₂₃H₃₁N₃O₅S m/z 462 (M+H)⁺; HRESIMS, calc. monoiso mass (Da): 461.1984, found monoiso mass (Da): 461.1983.

### Example B4

### Isobutyl 4-[methyl({6-[4-(methylsulfonyl)phenyl]pyridin-3-yl}methyl)amino]-piperidine-1-carboxylate, trifluoroacetate

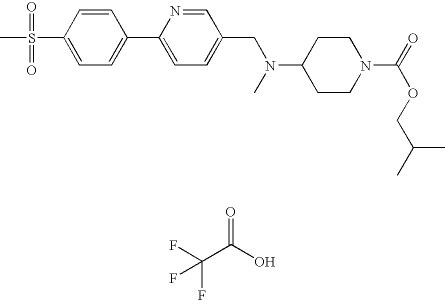

The title compound was prepared from Intermediate B2 and isobutyl chloroformate in accordance with general method A. Yield 39 mg (97%); Analytical HPLC: purity 100% (System A, R_{T}=1.69 min); ¹H NMR (400 MHz, CDCl₃) δ ppm 0.93 (d, J=6.5 Hz, 6H), 1.66-1.81 (m, 2H), 1.93 (m, 1H), 2.10-2.20 (m, 2H), 2.72 (s, 3H), 2.83 (br s, 2H), 3.10 (s, 3H), 3.60 (m, 1H), 3.88 (d, 2H), 4.24-4.54 (m, 5H), 7.92 (m, 1H), 8.08 (m, 2H), 8.12-8.24 (m, 3H), 8.79 (m, 1H); LRESIMS for C₂₄H₃₃N₃O₄S m/z 460 (M+H)⁺; HRESIMS, calc. monoiso mass (Da): 459.2192, found monoiso mass (Da): 459.2193.

### Example B5

### Ethyl 4-[methyl({6-[4-(methylsulfonyl)phenyl]pyridin-3-yl}methyl)amino]piperidine-1-carboxylate, trifluoroacetate

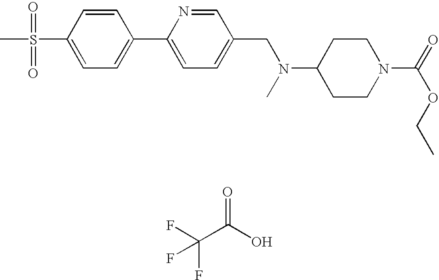

The title compound was prepared from Intermediate B2 and ethyl chloroformate in accordance with general method A. Yield 33 mg (86%); Analytical HPLC: purity 100% (System A, R_{T}=1.37 min); ¹H NMR (400 MHz, CDCl₃) δ ppm 1.26 (t, J=7.3 Hz, 3H), 1.66-1.80 (m, 2H), 2.09-2.19 (m, 2H), 2.71 (s, 3H), 2.80 (m, 2H), 3.10 (s, 3H), 3.56 (m, 1H), 4.15 (q, J=7.0 Hz, 2H), 4.25-4.50 (m, 4H), 7.91 (m, 1H), 8.07 (m, 2H), 8.14-8.22 (m, 3H), 8.72 (m, 1H); LRESIMS for C₂₂H₂₉N₃O₄S m/z 432 (M+H)⁺; HRESIMS, calc. monoiso mass (Da): 431.1879, found monoiso mass (Da): 431.1879.

### Example B6

### Isopropyl 4-[methyl({6-[4-(methylsulfonyl)phenyl]pyridin-3-yl}methyl)amino]-piperidine-1-carboxylate, trifluoroacetate

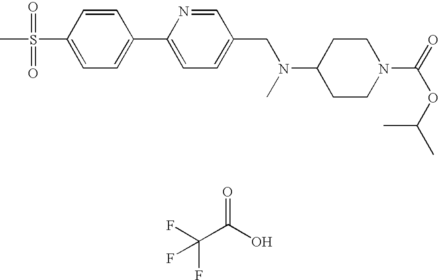

The title compound was prepared from Intermediate B2 and isopropyl chloroformate in accordance with general method A. Yield 33 mg (84%); Analytical HPLC: purity 100% (System A, R_{T}=1.51 min); ¹H NMR (400 MHz, CDCl₃) δ ppm 1.24 (d, J=6.3 Hz, 6H), 1.64-1.80 (m, 2H), 2.09-2.19 (m, 2H), 2.71 (s, 3H), 2.77 (m, 2H), 3.10 (S, 3H), 3.55 (m, 1H), 4.24-4.49 (m, 4H), 4.91 (m, 1H), 7.91 (m, 1H), 8.07 (m, 2H), 8.14-8.22 (m, 3H), 8.72 (m, 1H); LRESIMS for C₂₃H₃₁N₃O₄S m/z 446 (M+H)⁺; HRESIMS, calc. monoiso mass (Da): 445.2035, found monoiso mass (Da): 445.2036.

### Example B7

### Benzyl 4-[methyl({6-[4-(methylsulfonyl)phenyl]pyridin-3-yl}methyl)amino]-piperidine-1-carboxylate

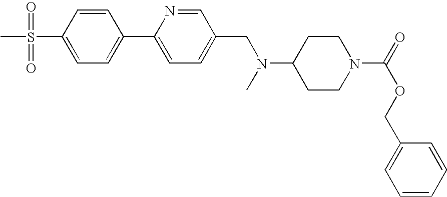

The title compound was prepared from Intermediate B2 and benzyl chloroformate in accordance with general method A (but without the preparative HPLC). Yield 33 mg (81%); Analytical HPLC: purity 100% (System A, R_{T}=1.78 min); ¹H NMR (400 MHz, CDCl₃) δ ppm 1.43-1.64 (m, 2H), 1.81 (m, 2H), 2.21 (s, 3H), 2.63 (m, 1H), 2.78 (m, 2H), 3.08 (s, 3H), 3.64 (s, 2H), 4.26 (br s, 2H), 5.12 (s, 2H), 7.27-7.40 (m, 5H), 7.71-7.83 (m, 2H), 8.03 (m, 2H), 8.19 (m, 2H), 8.63 (m, 1H); LRESIMS for C₂₇H₃₁N₃O₄S m/z 494 (M+H)⁺; HRESIMS, calc. monoiso mass (Da): 493.2035, found monoiso mass (Da): 493.2034.

### Example B8

### 2,2-Dimethylpropyl 4-[methyl({6-[4-(methylsulfonyl)phenyl]pyridin-3-yl}methyl)-amino]piperidine-1-carboxylate, trifluoroacetate

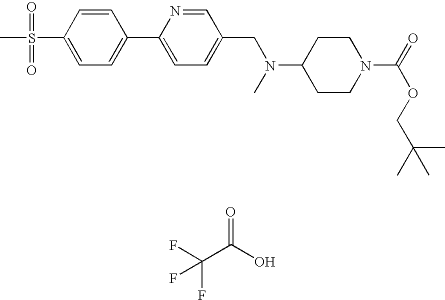

The title compound was prepared from Intermediate B2 and neopentyl chloroformate in accordance with general method A. Yield 19 mg (46%); Analytical HPLC: purity 100% (System A, R_{T}=1.79 min); ¹H NMR (400 MHz, CD₃OD) δ ppm 0.97 (s, 9H), 1.74-1.89 (m, 2H), 2.20 (m, 2H), 2.80 (s, 3H), 2.96-3.08 (m, 2H), 3.17 (s, 3H), 3.64 (m, 1H), 3.80 (s, 2H), 4.37 (m, 2H), 8.05-8.16 (m, 4H), 8.34 (m, 2H), 8.85 (m, 1H); LRESIMS for C₂₅H₃₅N₃O₄S m/z 474 (M+H)⁺; HRESIMS, calc. monoiso mass (Da): 473.2348, found monoiso mass (Da): 473.2343.

### Example B9

### Prop-2-yn-1-yl 4-[methyl({6-[4-(methylsulfonyl)phenyl]pyridin-3-yl}methyl)amino]-piperidine-1-carboxylate

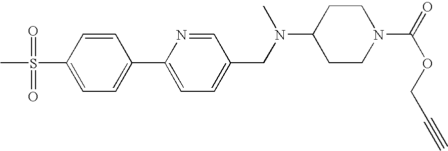

Intermediate B2 (25 mg, 0.07 mmol) was dissolved in DCM (0.8 mL) and triethylamine (0.025 mL, 18 mg, 0.18 mmol) was added. Propargyl chloroformate (0.01 mL, 0.1 mmol) dissolved in DCM (0.4 mL) was added to the solution. The mixture was stirred at r.t. overnight. More propargyl chloroformate (0.01 mL, 0.1 mmol) was added and after 3 h 2 M NH₃ in MeOH was added and the mixture was concentrated under reduced pressure. Flash chromatography of the residue using 2 M NH₃ in MeOH/CHCl₃ (2.5:97.5) as eluent gave the title compound: Yield 27 mg (87%). Analytical HPLC: purity 99% (System A, R_{T}=1.37 min); ¹H NMR (400 MHz, CDCl₃) δ ppm 1.44-1.63 (m, 2H), 1.76-1.92 (m, 2H), 2.21 (s, 3H), 2.45 (s, 1H), 2.63 (m, 1H), 2.79 (br s, 2H), 3.07 (s, 3H), 3.64 (s, 2H), 4.23 (br s, 2H), 4.68 (s, 2H), 7.69-7.82 (m, 2H), 8.01 (m, 2H), 8.18 (m, 2H), 8.62 (m, 1H); LRESIMS for C₂₃H₂₇N₃O₄S m/z 442 (M+H)⁺; HRESIMS, calc. monoiso mass (Da): 441.1722, found monoiso mass (Da): 441.1723.

### Example B10

### Phenyl 4-[methyl({6-[4-(methylsulfonyl)phenyl]pyridin-3-yl}methyl)amino]-piperidine-1-carboxylate

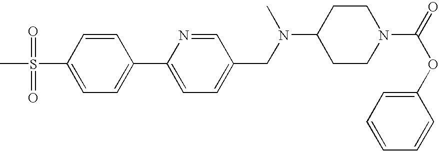

The title compound was prepared from Intermediate B2 and phenyl chloroformate in accordance with general method A with the exception that the product precipitated from MeOH (1 mL) and was collected by filtration (no preparative chromatography was used). Yield 27 mg (80%). Analytical HPLC: purity 100% (System A, R_{T}=1.65 min); ¹H NMR (400 MHz, CDCl₃) δ ppm 1.59-1.73 (m, 2H), 1.91 (m, 2H), 2.26 (s, 3H), 2.70 (m, 1H), 2.78-3.05 (m, 2H), 3.08 (s, 3H), 3.68 (s, 2H), 4.37 (m, 2H), 7.06-7.14 (m, 2H), 7.19 (m, 1H), 7.30-7.39 (m, 2H), 8.00-8.07 (m, 2H), 8.03 (m, 2H), 8.20 (m, 2H), 8.66 (m, 1H); LRESIMS for C₂₆H₂₉N₃O₄S m/z 480 (M+H)⁺; HRESIMS, calc. monoiso mass (Da): 479.1879, found monoiso mass (Da): 479.1880.

### Example B11

### 4-Fluorophenyl 4-[methyl({6-[4-(methylsulfonyl)phenyl]pyridin-3-yl}methyl)amino]-piperidine-1-carboxylate

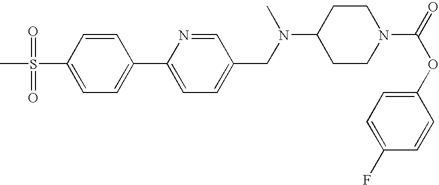

The title compound was prepared from Intermediate B2 and 4-fluorophenyl chloroformate in accordance with general method A with the exception that the product was purified by flash chromatography (instead of preparative HPLC) using 2 M NH₃ in MeOH/CHCl₃ (3:97) as eluent. Yield 37 mg (100%). Analytical HPLC: purity 97% (System A, R_{T}=1.71 min); ¹H NMR (400 MHz, CDCl₃) δ ppm 1.58-1.73 (m, 2H), 1.91 (m, 2H), 2.25 (s, 3H), 2.70 (m, 1H), 2.78-3.05 (m, 2H), 3.08 (s, 3H), 3.68 (s, 2H), 4.34 (m, 2H), 6.96-7.11 (m, 4H), 7.19 (m, 1H), 7.73-7.85 (m, 2H), 8.03 (m, 2H), 8.20 (m, 2H), 8.66 (m, 1H); LRESIMS for C₂₆H₂₈FN₃O₄S m/z 498 (M+H)⁺; HRESIMS, calc. monoiso mass (Da): 497.1785, found monoiso mass (Da): 497.1782.

### Example B12

### 4-Methoxyphenyl 4-[methyl({6-[4-(methylsulfonyl)phenyl]pyridin-3-yl}methyl)-amino]piperidine-1-carboxylate

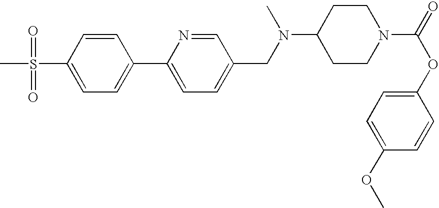

The title compound was prepared from Intermediate B2 and 4-methoxyphenyl chloroformate in accordance with general method A with the exception that the product was purified by flash chromatography (instead of preparative HPLC) using 2 M NH₃ in MeOH/CHCl₃ (3:97) as eluent. Yield 8 mg (22%). Analytical HPLC: purity 98% (System A, R_{T}=1.66 min); ¹H NMR (400 MHz, CDCl₃) δ ppm 1.58-1.73 (m, 2H), 1.91 (m, 2H), 2.25 (s, 3H), 2.69 (m, 1H), 2.76-3.04 (m, 2H), 3.08 (s, 3H), 3.68 (s, 2H), 3.78 (s, 3H), 4.35 (m, 2H), 6.86 (m, 2H), 7.01 (m, 2H), 7.99-8.09 (m, 2H), 8.03 (m, 2H), 8.20 (m, 2H), 8.65 (m, 1H); LRESIMS for C₂₇H₃₁N₃O₅S m/z 510 (M+H)⁺; HRESIMS, calc. monoiso mass (Da): 509.1984, found monoiso mass (Da): 509.1984.

### Example B13

### 2-Fluoro-1-(fluoromethyl)ethyl 4-[methyl({6-[4-(methylsulfonyl)phenyl]pyridin-3-yl}-methyl)amino]piperidine-1-carboxylate

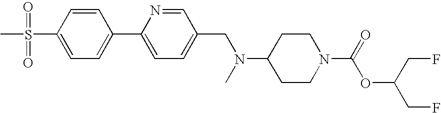

To a solution of 1,3-difluoropropan-2-ol (7.1 mg, 0.074 mmol) in DCM (0.4 mL) was added 1,1′-carbonylbis(1H-imidazole) (14.4 mg, 0.089 mmol) in DCM/THF (1:1; 0.8 mL) over 5 min. The mixture was stirred overnight at r.t. N-Methyl-N-({6-[4-(methylsulfonyl)-phenyl]pyridin-3-yl}methyl)piperidin-4-amine (20.5 mg, 0.057 mmol; Intermediate B2) in DCM (0.4 mL) was added, and the reaction mixture was stirred at r.t. for 24 h. The solvent was removed, and the residue was purified by preparative HPLC (System D) to give the title compound. Yield: 5.9 mg (22%); Analytical HPLC: purity 91% (System A and B); ¹H NMR (400 MHz, CDCl₃) δ ppm 1.51-1.72 (m, 4H) 1.82-2.01 (m, 2H) 2.20-2.39 (m, 2H) 2.81 (br s, 3H) 3.08 (s, 3H) 3.68-3.81 (m, 1H) 4.15-4.40 (m, 2H) 4.55 (app d, 2H) 4.67 (app d, 2H) 5.03-5.21 (m, 1H) 7.74-7.82 (m, 1H) 7.81-7.90 (m, 1H) 7.99-8.07 (m, 2H) 8.15-8.24 (m, 2H) 8.64 (m, 1H); LRESIMS for C₂₃H₂₉F₂N₃O₄S m/z 482 (M+H)⁺; HRESIMS, calc. monoiso mass (Da): 481.1847, found monoiso mass (Da): 481.1847.

### Example B14

### (1R)-1-Phenylethyl 4-[methyl({6-[4-(methylsulfonyl)phenyl]pyridin-3-yl}methyl)-amino]piperidine-1-carboxylate

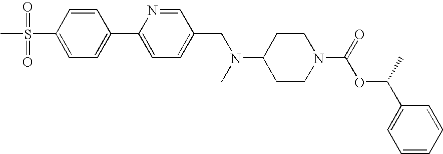

To a solution of 1,1′-carbonylbis(1H-imidazole) (21.9 mg, 0.135 mmol) in DCM (0.4 mL) at r.t. under N₂ (g) was added (1R)-1-phenylethanol (11.0 mg, 0.090 mmol) in DCM (0.4 mL). The mixture was stirred for 40 min after which N-methyl-N-({6-[4-(methylsulfonyl)phenyl]pyridin-3-yl}methyl)piperidin-4-amine (16.2 mg, 0.045 mmol; Intermediate B2) in DCM (0.4 mL) was added, and the reaction was stirred at r.t. overnight. Additional N-methyl-N-({6-[4-(methylsulfonyl)phenyl]pyridin-3-yl}methyl)-piperidin-4-amine (7 mg, 0.019 mmol) was added, and the mixture was stirred for 24 h at rt. The solvent was removed, and the residue was purified by preparative HPLC (System D) to give the title compound as a white solid. Yield: 9.8 mg (46%); Analytical HPLC: purity 99% (System A and B); ¹H NMR (400 MHz, CDCl₃) δ ppm 1.53 (d, J=6.6 Hz, 3H) 1.55-1.86 (m, 2H) 1.90-2.24 (m, 2H) 2.23-2.64 (m, 4H) 2.77 (br s, 3H) 3.08 (s, 3H) 3.84-4.05 (m, 1H) 4.29-4.38 (m, 2H) 5.79 (q, J=6.6 Hz, 1H) 7.25-7.38 (m, 6H) 7.81-7.90 (m, 1H) 7.99-8.07 (m, 2H) 8.15-8.23 (m, 2H) 8.66 (s, 1H); LRESIMS for C₂₈H₃₃N₃O₄S m/z 508 (M+H)⁺; HRESIMS, calc. monoiso mass (Da): 507.2192, found monoiso mass (Da): 507.2189.

### Example B15

### (1S)-1-Phenylethyl 4-[methyl({6-[4-(methylsulfonyl)phenyl]pyridin-3-yl}methyl)-amino]piperidine-1-carboxylate

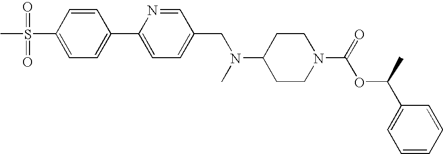

To a solution of (1S)-1-phenylethanol (12.5 mg, 0.102 mmol) in DCM (0.4 mL) at r.t. under N₂ (g) was added 1,1′-carbonylbis(1H-imidazole) (16.6 mg, 0.102 mmol) in DCM (0.4 mL). The mixture was stirred for 1.5 h after which N-methyl-N-({6-[4-(methylsulfonyl)phenyl]pyridin-3-yl}methyl)piperidin-4-amine (18.4 mg, 0.051 mmol; Intermediate B2) in DCM (0.4 mL) was added, and the reaction was stirred at r.t. for 2 days. The solvent was removed, and the residue was purified by preparative HPLC (System D) to give the title compound as a white solid. Yield: 4.3 mg (17%); Analytical HPLC: purity 96% (System A and B); ¹H NMR (400 MHz, CDCl₃) δ ppm 1.54 (d, J=6.6 Hz, 3H) 1.66-1.81 (m, 2H) 2.16-2.37 (m, 2H) 2.46-2.57 (m, 2H) 2.68 (br s, 3H) 2.74-2.85 (m, 2H) 3.09 (s, 3H) 3.36-3.50 (m, 1H) 4.36-4.47 (m, 2H) 5.79 (q, J=6.5 Hz, 1H) 7.26-7.39 (m, 6H) 7.96-8.03 (m, 1H) 8.04-8.12 (m, 2H) 8.17-8.27 (m, 2H) 8.90-9.02 (m, 1H); LRESIMS for C₂₈H₃₃N₃O₄S m/z 508 (M+H)⁺; HRESIMS, calc. monoiso mass (Da): 507.2192, found monoiso mass (Da): 507.2197.

### Example B16

### (1S,2R,4R)-Bicyclo[2.2.1]hept-2-yl 4-[methyl({6-[4-(methylsulfonyl)phenyl]pyridin-3-yl}methyl)amino]piperidine-1-carboxylate

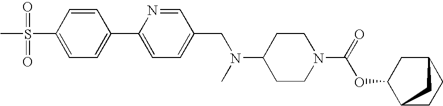

To a solution of (2R)-(+)-endo-norborneol (31 mg, 0.28 mmol) in CH₃CN (0.3 mL) was 1,1′-carbonylbis(1H-imidazole) (45 mg, 0.28 mmol) in CH₃CN (0.4 mL) dropwise added at room temperature. The mixture was stirred for 10 minutes. Solid N-methyl-N-({6-[4-(methylsulfonyl)phenyl]pyridin-3-yl}methyl)piperidin-4-amine (10 mg, 0.028 mmol; intermediate B2) was added and the mixture was heated by microwave irradiation at 100° C. for 10 minutes. The crude mixture was purified by preparative HPLC (System D). Pure fractions were combined, saturated aqueous NaHCO₃ was added and the resulting mixture was extracted with EtOAc. The organic phase was dried over Na₂SO₄, filtered and the solvent was evaporated to give the title compound. Yield: 2 mg (15%); Analytical HPLC: purity 90% (System A), purity 90% (System B); ¹H NMR (400 MHz, CDCl₃) δ ppm 0.95-1.03 (m, 1H) 1.23-1.41 (m, 4H) 1.49-1.60 (m, 9H) 1.69-1.88 (m, 2H) 1.94-2.05 (m, 1H) 2.19-2.25 (m, 3H) 2.48 (m, 1H) 2.57-2.82 (m, 2H) 3.08 (s, 3H) 3.65 (s, 2H) 4.91 (m, 1H) 7.74-7.82 (m, 2H) 8.00-8.07 (m, 2H) 8.17-8.22 (m, 2H) 8.64 (m, 1H); LRESIMS for C₂₇H₃₅N₃O₄S m/z 498 (M+H)⁺; HRESIMS, calc. monoiso mass (Da): 497.2348, found monoiso mass (Da): 497.2362.

### Example B17

### (1-Methylcyclopropyl)methyl 4-[methyl({6-[4-(methylsulfonyl)phenyl]pyridin-3-yl}-methyl)amino]piperidine-1-carboxylate

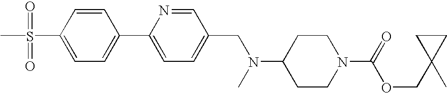

To a solution of (1-methyl-cyclopropyl)-methanol (23 mg, 0.28 mmol) in CH₃CN (0.3 mL) was 1,1′-carbonylbis(1H-imidazole) (45 mg, 0.28 mmol) in CH₃CN (0.4 mL) dropwise added at room-temperature. The mixture was stirred for 10 minutes. Solid N-methyl-N-({6-[4-(methylsulfonyl)phenyl]pyridin-3-yl}methyl)piperidin-4-amine (10 mg, 0.028 mmol; Intermediate B2) was added and the mixture was exposed to microwave irradiation (100° C.) for 10 minutes. The crude was purified by preparative HPLC (System D). Pure fractions were combined, saturated aqueous NaHCO₃ was added and the resulting mixture was extracted with EtOAc. The organic phase was dried over Na₂SO₄, filtered and the solvent was evaporated to give the title compound. Yield: 5 mg (38%); Analytical HPLC: purity 90% (System A), purity 90% (System B); ¹H NMR (400 MHz, CDCl₃) δ ppm 0.31-0.37 (m, 2H) 0.45-0.51 (m, 2H) 1.13 (s, 3H) 1.22-1.29 (m, 1H) 1.49-1.61 (m, 3H) 1.80-1.88 (m, 2H) 2.23 (s, 2H) 2.58-2.85 (m, 2H) 3.08 (s, 3H) 3.65 (s, 2H) 3.87 (s, 2H) 4.20-4.30 (m, 2H) 7.72-7.82 (m, 2H) 8.00-8.06 (m, 2H) 8.17-8.22 (m, 2H) 8.19 (m, 1H); LRESIMS for C₂₅H₃₃N₃O₄S m/z 472 (M+H)⁺; HRESIMS, calc. monoiso mass (Da): 471.2192, found monoiso mass (Da): 471.2210.

### Example B18

### Cyclobutyl 4-[methyl({6-[4-(methylsulfonyl)phenyl]pyridin-3-yl}methyl)amino]-piperidine-1-carboxylate

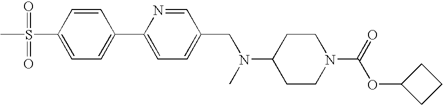

To a solution of cyclobutanol (20 mg, 0.28 mmol) in CH₃CN (0.3 mL) was 1,1′-carbonylbis(1H-imidazole) (45 mg, 0.28 mmol) in CH₃CN (0.4 mL) dropwise added at room-temperature. The mixture was stirred for 10 minutes. Solid N-methyl-N-({6-[4-(methyl sulfonyl)phenyl]pyridin-3-yl}methyl)piperidin-4-amine (10 mg, 0.028 mmol; Intermediate B2) was added and the mixture was exposed to microwave irradiation (100° C.) for 10 minutes. The crude product was purified by preparative HPLC (System D). Pure fractions were combined, saturated aqueous NaHCO₃ was added and the resulting mixture was extracted with EtOAc. The organic phase was dried over Na₂SO₄, filtered and the solvent was evaporated to give the title compound. Yield: 2 mg (15%); Analytical HPLC: purity 100% (System A), purity 97% (System B); ¹H NMR (400 MHz, CDCl₃) δ ppm 1.22-1.29 (m, 1H) 1.50-1.63 (m, 4H) 1.69-1.87 (m, 2H) 1.99-2.09 (m, 2H) 2.22 (s, 3H) 2.33 (m, 2H) 2.57-2.82 (m, 2H) 3.08 (s, 3H) 3.65 (s, 2H) 4.20 (m, 2H) 4.92 (m, 1H) 7.73-7.82 (m, 2H) 8.00-8.05 (m, 2H) 8.17-8.22 (m, 2H) 8.63 (m, 1H); LRESIMS for C₂₄H₃₁N₃O₄5 m/z 456 (M+H)⁺; HRESIMS, calc. monoiso mass (Da): 457.2035, found monoiso mass (Da): 457.2048.

### Example B19

### 1,3-Benzodioxol-5-ylmethyl 4-[methyl({6-[4-(methylsulfonyl)phenyl]pyridin-3-yl}-methyl)amino]piperidine-1-carboxylate

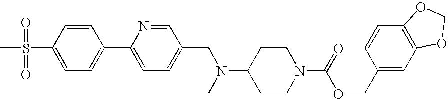

To a solution of 3,4-(methylenedioxy)-benzyl alcohol (43 mg, 0.28 mmol) in CH₃CN (0.3 mL) was 1,1′-carbonylbis(1H-imidazole) (45 mg, 0.28 mmol) in CH₃CN (0.4 mL) dropwise added at room-temperature. The mixture was stirred for 10 minutes. Solid N-methyl-N-({6-[4-(methylsulfonyl)phenyl]pyridin-3-yl}methyl)piperidin-4-amine (10 mg, 0.028 mmol; Intermediate B2) was added and the mixture was exposed to microwave irradiation (100° C.) for 10 minutes. The crude was purified by preparative HPLC (System D). Pure fractions were combined, saturated aqueous NaHCO₃ was added and the resulting mixture was extracted with EtOAc. The organic phase was dried over Na₂SO₄, filtered and the solvent was evaporated to give the title compound. Yield: 12 mg (90%); Analytical HPLC: purity 100% (System A), purity 91% (System B); ¹H NMR (400 MHz, CDCl₃) δ ppm 1.54-1.89 (m, 4H) 2.21 (s, 3H) 2.57-2.85 (m, 3H) 3.08 (s, 3H) 3.64 (s, 2H) 4.13-4.33 (m, 2H) 5.01 (s, 2H) 5.95 (s, 2H) 6.75-6.87 (m, 3H) 7.72-7.81 (m, 2H) 8.00-8.06 (m, 2H) 8.16-8.21 (m, 2H) 8.63 (m, 1H); LRESIMS for C₂₈H₃₁N₃O₆S m/z 538 (M+H)⁺; HRESIMS, calc. monoiso mass (Da): 537.1934, found monoiso mass (Da): 537.1942.

### Example B20

### tert-Butyl 4-[(2-fluoroethyl)({6-[4-(methylsulfonyl)phenyl]pyridin-3-yl}methyl)-amino]piperidine-1-carboxylate

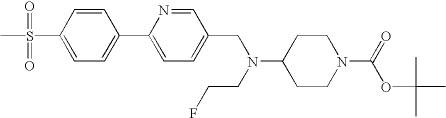

A mixture of tert-butyl 4-[({6-[4-(methylsulfonyl)phenyl]pyridin-3-yl}methyl)amino]-piperidine-1-carboxylate (90 mg, 0.20 mmol; obtained in Example B1), 1-fluoro-2-iodoethane (52 mg, 0.3 mmol), (iPr)₂EtN (0.052 mL, 0.30 mmol) in CH₃CN (1 mL) was heated to 85° C. for 3 days. The mixture was concentrated under reduced pressure. Flash chromatography of the residue using 2 M NH₃ in MeOH/CHCl₃ (3:97) as eluent gave the title product. Yield 10 mg. Analytical HPLC: purity 93%, R_{T}=1.72 min (System A); ¹H NMR (400 MHz, CDCl₃) δ ppm 1.39-1.52 (m, 11H); 1.78 (m, 2H), 2.55-2.75 (m, 3H), 2.83 (m, 1H), 2.89 (m, 1H), 3.08 (s, 3H), 3.79 (s, 2H), 4.17 (br s, 2H), 4.33 (m, 1H), 4.45 (m, 1H), 7.75 (m, 1H), 7.83 (m, 1H), 8.03 (m, 2H), 8.19 (m, 2H), 8.65 (m, 1H); LRESIMS for C₂₅H₃₄FN₃O₄S m/z 492 (M+H)⁺; HRESIMS, calc. monoiso mass (Da): 491.2254, found monoiso mass (Da): 491.2252.

### Example B21

### tert-Butyl 4-[(cyclopropylmethyl)({6-[4-(methylsulfonyl)phenyl]pyridin-3-yl}methyl)-amino]piperidine-1-carboxylate

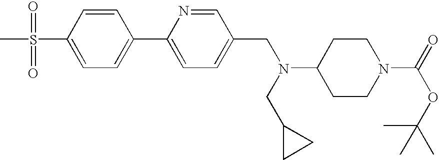

tert-Butyl 4-[({6-[4-(methylsulfonyl)phenyl]pyridin-3-yl}methyl)amino]piperidine-1-carboxylate (45 mg, 0.10 mmol; obtained in Example B1) was dissolved in DCE (1 mL) and cyclopropane carboxaldehyde (0.015 mL, 0.2 mmol) followed by NaBH(OAc)₃ (42 mg, 0.2 mmol) were added. The mixture was stirred for 3 days at r.t. The solvent was evaporated and the residue was partitioned between DCM and 1 M NaOH. The organic phase was evaporated. Flash chromatography of the residue using 2 M NH₃ in MeOH/CHCl₃ (1.5:98.5) as eluent gave the title compound. Yield 53 mg (94%). Analytical HPLC: purity 100% (System A, R_{T}=1.83 min); ¹H NMR (400 MHz, CDCl₃) δ ppm 0.02-0.07 (m, 2H), 0.38-0.47 (m, 2H), 0.78 (m, 1H), 1.35-1.52 (m, 11H), 1.76 (m, 2H), 2.41 (d, J=6.5 Hz, 2H), 2.64 (m, 2H), 2.82 (m, 1H), 3.08 (s, 3H), 3.75 (s, 2H), 4.17 (br s, 2H), 7.73 (m, 1H), 7.82 (m, 1H), 8.03 (m, 2H), 8.22 (m, 2H), 8.69 (m, 1H); LRESIMS for C₂₇H₃₇N₃O₄S m/z 500 (M+H)⁺; HRESIMS, calc. monoiso mass (Da): 499.2505, found monoiso mass (Da): 499.2506.

### Example B22

### tert-Butyl 4-[(2-hydroxyethyl)({6-[4-(methylsulfonyl)phenyl]pyridin-3-yl}methyl)-amino]piperidine-1-carboxylate

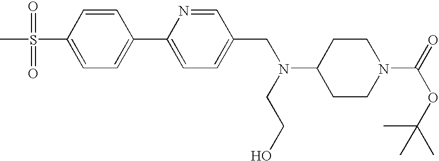

tert-Butyl 4-[({6-[4-(methylsulfonyl)phenyl]pyridin-3-yl}methyl)amino]piperidine-1-carboxylate (45 mg, 0.10 mmol; obtained in Example B1) was dissolved in DCE (1 mL) and glycolaldehyde (12 mg, 0.2 mmol) followed by NaBH(OAc)₃ (42 mg, 0.2 mmol) were added. The mixture was stirred at r.t. overnight. The solvent was evaporated and the residue was partitioned between DCM and 1 M NaOH. The organic phase was evaporated. Flash chromatography of the residue using 2 M NH₃ in MeOH/CHCl₃ (1.5:98.5) as eluent gave the title compound. Yield 49 mg (100%). Analytical HPLC: purity 96% (System A, R_{T}=1.62 min); ¹H NMR (400 MHz, CDCl₃) δ ppm 1.44 (s, 9H), 1.47-1.53 (m, 2H), 1.74 (m, 2H), 2.52-2.70 (m, 3H), 2.73 (m, 2H), 3.08 (s, 3H), 3.52 (m, 2H), 3.74 (s, 2H), 4.19 (br s, 2H), 7.73-7.79 (m, 2H), 8.03 (m, 2H), 8.19 (m, 2H), 8.63 (m, 1H); LRESIMS for C₂₅H₃₅N₃O₅S m/z 490 (M+H)⁺; HRESIMS, calc. monoiso mass (Da): 489.2297, found monoiso mass (Da): 489.2291.

### Example B23

### tert-Butyl 4-[(cyanomethyl)({6-[4-(methylsulfonyl)phenyl]pyridin-3-yl}methyl)-amino]piperidine-1-carboxylate

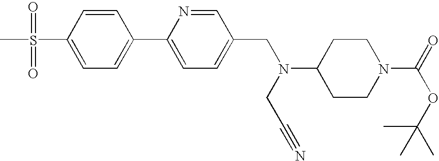

tert-Butyl 4-[({6-[4-(methylsulfonyl)phenyl]pyridin-3-yl}methyl)amino]piperidine-1-carboxylate (45 mg, 0.10 mmol; obtained in Example B1) was dissolved in CH₃CN (0.5 mL) and (iPr)₂EtN (0.026 mL, 0.15 mmol). Iodoacetonitrile (25 mg, 0.15 mmol) was added. After 40 min at r.t. the solution was heated to 85° C. over 2 h. The mixture was concentrated under reduced pressure. Flash chromatography of the residue using 2 M NH₃ in MeOH/CHCl₃ (3:97) as eluent gave the title product (33 mg, 68% yield). Analytical HPLC: purity 98% (System A, R_{T}=2.22 min); ¹H NMR (400 MHz, CDCl₃) δ ppm 1.46 (s, 9H), 1.89-2.04 (m, 2H), 1.95 (m, 2H), 2.79 (m, 3H), 3.09 (s, 3H), 3.49 (s, 2H), 3.89 (s, 2H), 4.18 (br s, 2H), 7.78 (s, 2H), 8.04 (m, 2H), 8.20 (m, 2H), 8.68 (m, 1H); LRESIMS for C₂₅H₃₂N₄O₄S m/z 485 (M+H)⁺; HRESIMS, calc. monoiso mass (Da): 484.2144, found monoiso mass (Da): 484.2143.

### Example B24

### tert-Butyl 4-[ethyl({6-[4-(methylsulfonyl)phenyl]pyridin-3-yl}methyl)amino]-piperidine-1-carboxylate

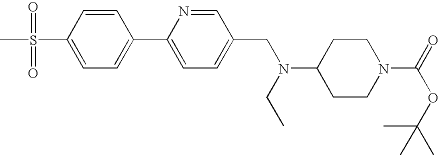

tert-Butyl 4-[({6-[4-(methylsulfonyl)phenyl]pyridin-3-yl}methyl)amino]piperidine-1-carboxylate (45 mg, 0.10 mmol; obtained in Example B1) was dissolved in CH₃CN (0.5 mL) and (iPr)₂EtN (0.026 mL, 0.15 mmol). Iodoethane (23 mg, 0.15 mmol) was added. After 40 min at r.t. the solution was heated to 85° C. over 2 h. The reaction mixture was concentrated under reduced pressure. Flash chromatography of the residue using 2 M NH₃ in MeOH/CHCl₃ (3:97) as eluent gave the title product (39 mg, 82% yield). Analytical HPLC: purity 100% (System A, R_{T}=1.71 min); ¹H NMR (400 MHz, CDCl₃) δ ppm 1.02 (t, J=7.1 Hz, 3H), 1.39-1.52 (m, 11H), 1.74 (m, 2H), 2.53-2.73 (m, 5H), 3.08 (s, 3H), 3.68 (s, 2H), 4.16 (br s, 2H), 7.73 (m, 1H), 7.80 (m, 1H), 8.03 (m, 2H), 8.19 (m, 2H), 8.67 (s, 1H); LRESIMS for C₂₅H₃₅N₃O₄S m/z 474 (M+H)⁺; HRESIMS, calc. monoiso mass (Da): 473.2348, found monoiso mass (Da): 473.2345.

### Example B25

### tert-Butyl 4-[cyclobutyl({6-[4-(methylsulfonyl)phenyl]pyridin-3-yl}methyl)amino]-piperidine-1-carboxylate

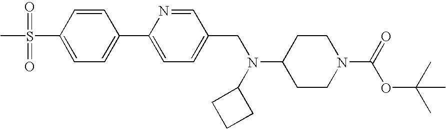

tert-Butyl 4-[({6-[4-(methylsulfonyl)phenyl]pyridin-3-yl}methyl)amino]piperidine-1-carboxylate (223 mg, 0.50 mmol; obtained in Example B1) was mixed with THF (0.83 mL) and water (0.008 mL). HOAc (0.092 mL) and cyclobutanone (0.056 mL, 0.75 mmol) were added. NaCNBH₃ (0.75 mL of a 1 M solution in THF, 0.75 mmol) was added and the mixture was heated to 60° C. overnight. The solvent was evaporated and the residue acidified with 1 M HCl. 10% aqueous Na₂CO₃ was added and the product was extracted with EtOAc and concentrated. Flash chromatography of the residue using 2 M NH₃ in MeOH/CHCl₃ (3:97) as eluent gave the title compound. Yield 153 mg (61%). Analytical HPLC: purity 98% (System A, R_{T}=1.74 min); ¹H NMR (400 MHz, CDCl₃) δ ppm 1.34-1.48 (m, 11H), 1.49-1.70 (m, obscured by solvent signal), 1.83 (m, 2H), 1.93 (m, 2H), 2.49-2.71 (m, 3H), 3.08 (s, 3H), 3.44 (m, 1H), 3.68 (s, 2H), 4.14 (br s, 2H), 7.72 (m, 1H), 7.82 (m, 1H), 8.02 (m, 2H), 8.19 (m, 2H), 8.67 (m, 1H); LRESIMS for C₂₇H₃₇N₃O₄S m/z 500 (M+H)⁺; HRESIMS, calc. monoiso mass (Da): 499.2505, found monoiso mass (Da): 499.2505.

### Example B26

### tert-Butyl 4-[({6-[4-(methylsulfonyl)phenyl]pyridin-3-yl}methyl)(3,3,3-trifluoropropyl)amino]piperidine-1-carboxylate*

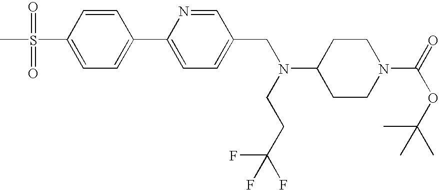

tert-butyl 4-[({6-[4-(methylsulfonyl)phenyl]pyridin-3-yl}methyl)amino]piperidine-1-carboxylate (92 mg, 0.2 mmol; obtained in Example B1) was dissolved in DCE (1 mL) and 3,3,3-trifluoropropionaldehyde (45 mg, 0.4 mmol) followed by NaBH(OAc)₃ (84 mg, 0.4 mmol) were added. The mixture was stirred for 5 h at r.t. More aldehyde (0.03 mL) and NaBH(OAc)₃ (42 mg, 0.2 mmol) were added and the mixture was stirred overnight. DCM (10 mL) and 1 M NaOH (2 mL) were added. The aqueous phase was extracted once more with DCM and the combined organic phases were dried (Na₂SO₄), filtered and evaporated. Flash chromatography of the residue using MeOH/CHCl₃ (1:99) as eluent gave the title compound. Yield 77 mg (71%). Analytical HPLC: purity 100%, R_{T}=1.99 min (System A); ¹H NMR (400 MHz, CDCl₃) δ ppm 1.37-1.53 (m, 11H), 1.76 (m, 2H), 2.21 (m, 2H), 2.62 (m, 3H), 2.79 (m, 2H), 3.08 (s, 3H), 3.73 (s, 2H), 4.19 (br s, 2H), 7.72-7.81 (m, 2H), 8.03 (m, 2H), 8.20 (m, 2H), 8.65 (m, 1H); LRESIMS for C₂₆H₃₄F₃N₃O₄S m/z 542 (M+H)⁺; HRESIMS, calc. monoiso mass (Da): 541.2222, found monoiso mass (Da): 541.2218.
* This compound could also be prepared in accordance with general method D.

### Example B27

### tert-Butyl 4-[({6-[4-(methylsulfonyl)phenyl]pyridin-3-yl}methyl)(2,2,2-trifluoroethyl)-amino]piperidine-1-carboxylate

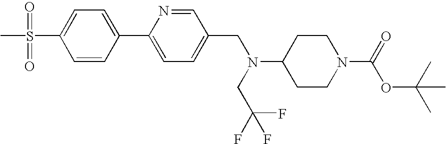

A mixture of tert-butyl 4-[({6-[4-(methylsulfonyl)phenyl]pyridin-3-yl}methyl)amino]-piperidine-1-carboxylate (30 mg, 0.07 mmol; obtained in Example B1), trifluoromethanesulfonic acid 2,2,2-trifluoroethyl ester (47 mg, 0.20 mmol) and N,N-diisopropylethyl amine (26 mg, 0.20 mmol) in THF (0.5 mL) was exposed to microwave irradiation (150° C.) for 1 hour. The reaction mixture was purified by preparative HPLC (System E) to give the title compound. Yield: 1 mg (2.7%); Analytical HPLC: purity 100% (System A), purity 100% (System B); ¹H NMR (400 MHz, CDCl₃) δ ppm 0.78-0.90 (m, 1H) 1.24 (m, 2H) 1.34-1.42 (m, 1H) 1.44 (s, 9H) 1.75-1.84 (m, 2H) 2.55-2.66 (m, 3H) 3.08 (s, 3H) 3.10-3.17 (m, 1H) 3.93 (s, 2H) 4.19 (m, 1H) 7.75-7.86 (m, 1H) 8.04 (m, 1H) 8.18-8.22 (m, 1H); LRESIMS for C₂₅H₃₂F₃N₃O₄S m/z 528; HRESIMS, calc. monoiso mass (Da): 527.2066, found monoiso mass (Da): 527.2066.

### Example B28

### tert-Butyl 4-[isobutyl({6-[4-(methylsulfonyl)phenyl]pyridin-3-yl}methyl)amino]-piperidine-1-carboxylate

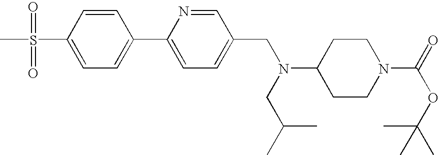

To a stirred solution of tert-butyl 4-[({6-[4-(methylsulfonyl)phenyl]pyridin-3-yl}methyl)-amino]piperidine-1-carboxylate (46 mg, 0.1 mmol; obtained in Example B1) in DCE (1 mL) were added isobutyraldehyde (14 mg, 0.19 mmol) and NaBH(OAc)₃ (42 mg, 0.2 mmol). DCM and 1 M NaOH were added after 2 days of stirring at r.t. The organic phase was separated and dried (Na₂SO₄). Evaporation gave the title compound. Yield 48 mg (95%). Analytical HPLC: purity 100%, R_{T}=1.88 min (System A); ¹H NMR (400 MHz, CDCl₃) δ 0.85 (d, J=6.5 Hz, 6H), 1.37-1.50 (m, 11H), 1.58-1.78 (m, 3H), 2.25 (d, J=7.0 Hz, 2H), 2.57 (m, 3H), 3.08 (s, 3H), 3.67 (s, 2H), 4.16 (br s, 2H), 7.70-7.81 (m, 2H), 8.03 (m, 2H), 8.20 (m, 2H), 8.66 (m, 1H); LRESIMS for C₂₇H₃₉N₃O₄S m/z 502 (M+H)⁺; HRESIMS, calc. monoiso mass (Da): 501.2661, found monoiso mass (Da): 501.2655.

### Example B29

### tert-Butyl 4-[({6-[4-(methylsulfonyl)phenyl]pyridin-3-yl}methyl)(tetrahydrofuran-2-ylmethyl)amino]piperidine-1-carboxylate

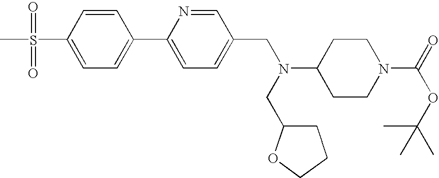

To a stirred solution of tert-butyl 4-[({6-[4-(methylsulfonyl)phenyl]pyridin-3-yl}methyl)-amino]piperidine-1-carboxylate (46 mg, 0.1 mmol; obtained in Example B1) in THF (0.5 mL) were added tetrahydrofuran-2-carboxaldehyde (40 mg, 50% aqueous solution, 0.2 mmol) and HOAc (0.02 mL) and then 1 M NaBH₃CN in THF (0.2 mL, 0.2 mmol). After one day at r.t. NaBH₃CN (0.2 mmol) was added and the mixture was stirred overnight. The reaction mixture was concentrated, acidified with 1 M HCl and then alkalinized with 10% aqueous Na₂CO₃. The alkaline mixture was extracted with EtOAc. Flash chromatography on silica using 2 M NH₃ in MeOH/CHCl₃ (2:98) as eluent gave the title compound. Yield 30 mg (56%). Analytical HPLC: purity 100%, R_{T}=1.72 min (System A); ¹H NMR (400 MHz, CDCl₃) δ ppm 1.37-1.60 (m, 12H), 1.63-1.81 (m, 2H), 1.87-1.98 (m, 1H), 2.27-2.40 (m, 1H), 2.41-2.67 (m, 5H), 3.07 (s, 3H), 3.41-3.49 (m, 1H), 3.61-3.80 (m, 5H), 4.16 (br s, 2H), 7.70-7.81 (m, 2H), 8.02 (m, 2H), 8.19 (m, 2H), 8.64 (m, 1H); LRESIMS for C₂₈H₃₉N₃O₅S m/z 530 (M+H)⁺; HRESIMS, calc. monoiso mass (Da): 529.2610, found monoiso mass (Da): 529.2605.

### Example B30

### tert-Butyl 4-[isopropyl({6-[4-(methylsulfonyl)phenyl]pyridin-3-yl}methyl)amino]-piperidine-1-carboxylate

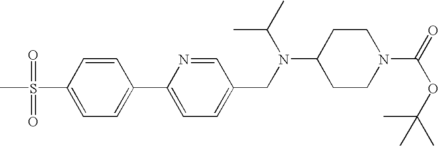

To a stirred solution of tert-butyl 4-[({6-[4-(methylsulfonyl)phenyl]pyridin-3-yl}methyl)-amino]piperidine-1-carboxylate (336 mg, 0.75 mmol; obtained in Example B1) in EtOH (4.5 mL) and acetone (2.45 mL) were added NaBH₃CN (186 mg, 3.0 mmol) and HOAc (0.135 mL). The reaction mixture was heated to reflux for 3 days. The mixture was concentrated and acidified with 1 M HCl (5 mL). 10% aqueous Na₂CO₃ (5 mL) was added and the mixture was extracted with DCM (2×20 mL). Flash chromatography on silica using 2 M NH₃ in MeOH/CHCl₃ (2:98) as eluent gave the title compound as a solid. Yield 244 mg (67%). Analytical HPLC: purity 98%, R_{T}=1.72 min (System A), LRESIMS for C₂₆H₃₇N₃O₄S m/z 488 (M+H)⁺.

### Intermediate B3

### N-isopropyl-N-({6-[4-(methylsulfonyl)phenyl]pyridin-3-yl}methyl)piperidin-4-amine

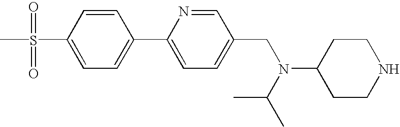

tert-Butyl 4-[isopropyl({6-[4-(methylsulfonyl)phenyl]pyridin-3-yl}methyl)amino]-piperidine-1-carboxylate (210 mg, 0.430 mmol; obtained in Example B30) was dissolved in DCM (2 mL) and TFA (0.5 mL) was added. After being stirred at room temp for 1 h, the mixture was concentrated under reduced pressure. The residue was partitioned between DCM (80 mL) and 1 M NaOH (8 mL). The DCM phase was separated, dried (Na₂SO₄) and evaporated to give the title compound. Yield 136 mg (81%); Analytical HPLC: purity 97%, R_{T}=0.97 min (System A); LRESIMS for C₂₁H₂₉N₃O₂S m/z 388 (M+H)⁺.

### Example B31

### Isopropyl 4-[isopropyl({6-[4-(methylsulfonyl)phenyl]pyridin-3-yl}methyl)amino]-piperidine-1-carboxylate, trifluoroacetate

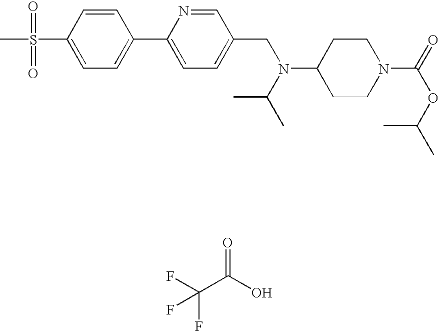

N-isopropyl-N-({6-[4-(methylsulfonyl)phenyl]pyridin-3-yl}methyl)piperidin-4-amine (79 mg, 0.20 mmol; Intermediate B3) was dissolved in DCM (2.5 mL) and Et₃N (0.075 mL, 0.54 mmol) was added. Isopropyl chloroformate (1 M solution in toluene, 0.4 mL, 0.4 mmol) was added. The reaction mixture was stirred overnight, concentrated under reduced pressure, and purified by preparative HPLC (System D). Yield 29 mg (25%). Analytical HPLC: purity 97%, R_{T}=1.59 min (System A); ¹H NMR (400 MHz, CDCl₃) δ ppm 1.24 (d, J=6.3 Hz, 6H), 1.49 (br s, 6H), 1.82 (br s, 2H), 2.18 (m, 2H), 3.17 (s, 3H), 3.89 (m, 1H), 3.93 (m, 1H), 3.97 (s, 2H), 4.29 (m, 2H), 4.60 (s, 2H), 8.05-8.15 (m, 4H), 8.34 (m, 2H), 8.85 (m, 1H); LRESIMS for C₂₅H₃₅N₃O₄S m/z 474 (M+H)⁺; HRESIMS, calc. monoiso mass (Da): 473.2348, found monoiso mass (Da): 473.2351.

### Example B32

### tert-Butyl 4-[({6-[4-(methylsulfonyl)phenyl]pyridin-3-yl}methyl)(propyl)amino]-piperidine-1-carboxylate

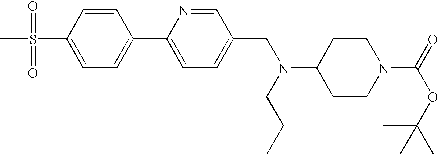

tert-Butyl 4-[({6-[4-(methylsulfonyl)phenyl]pyridin-3-yl}methyl)amino]piperidine-1-carboxylate (45 mg, 0.10 mmol; obtained in Example B1) was dissolved in DCE (1 mL) and propionaldehyde (0.015 mL, 0.2 mmol) and NaBH(OAc)₃ (42 mg, 0.2 mmol) were added. The mixture was stirred at r.t. overnight. The solvent was evaporated in vacuo and the residue was partitioned between DCM and 1 M NaOH. The organic phase was evaporated. Flash chromatography of the residue using 2 M NH₃ in MeOH/CHCl₃ (1.5:98.5) as eluent gave the title compound. Yield 50 mg (98%). Analytical HPLC: purity 95% (System A, R_{T}=1.81 min); ¹H NMR (400 MHz, CDCl₃) δ ppm 0.84 (t, J=7.2 Hz, 3H), 0.88-0.99 (m, 2H), 1.35-1.51 (m, 14H), 1.73 (m, 2H), 2.46 (t, J=7.3 Hz, 2H), 2.63 (m, 3H), 3.08 (s, 3H), 3.68 (s, 2H), 4.16 (br s, 2H), 7.70-7.82 (m, 2H), 8.03 (m, 2H), 8.19 (m, 2H), 8.66 (m, 1H); LRESIMS for C₂₆H₃₇N₃O₄S m/z 488 (M+H)⁺; HRESIMS, calc. monoiso mass (Da): 487.2505, found monoiso mass (Da): 487.2502.

### Intermediate B4

### N-({6-[4-(methylsulfonyl)phenyl]pyridin-3-yl}methyl)-N-propylpiperidin-4-amine

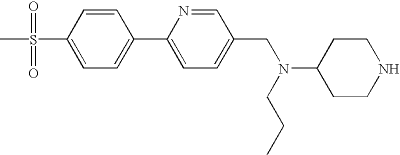

tert-Butyl 4-[({6-[4-(methylsulfonyl)phenyl]pyridin-3-yl}methyl)(propyl)amino]-piperidine-1-carboxylate (199 mg, 408 mmol; obtained in Example B32) was dissolved in DCM (2 mL) and TFA (0.5 mL) was added. The reaction mixture was stirred at room temp for 1 h and concentrated. The residue partitioned between DCM (80 mL) and 1 M NaOH (8 mL). The DCM phase was separated, dried (Na₂SO₄) and evaporated to give the title compound. Yield 138 mg (87%). Analytical HPLC: purity 92%, R_{T}=1.01 min (System A); LRESIMS for C₂₁H₂₉N₃O₂S m/z 388 (M+H)⁺.

### Example B33

### Isopropyl 4-[({6-[4-(methylsulfonyl)phenyl]pyridin-3-yl}methyl)(propyl)amino]-piperidine-1-carboxylate, trifluoroacetate

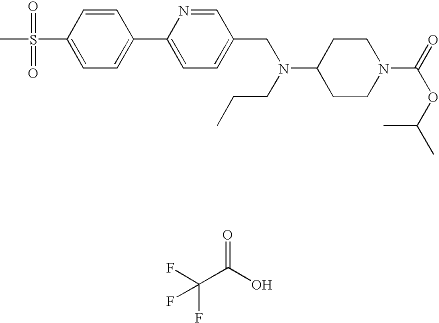

N-({6-[4-(methylsulfonyl)phenyl]pyridin-3-yl}methyl)-N-propylpiperidin-4-amine (80 mg, 0.206 mmol; Intermediate B4) was dissolved in DCM (2.5 mL) and Et₃N (0.075 mL, 0.54 mmol). Isopropyl chloroformate (1 M solution in toluene, 0.4 mL, 0.4 mmol) was added. The mixture was stirred at r.t. overnight, concentrated under reduced pressure, and purified by preparative HPLC (System D). Yield 54 mg (46%). Analytical HPLC: purity 100%, R_{T}=1.64 min (System A), ¹H NMR (400 MHz, CDCl₃) δ ppm 0.96 (t, J=7.3 Hz, 3H), 1.26 (d, J=6.3 Hz, 6H), 1.59-1.93 (m, 4H), 2.14 (m, 2H), 2.91 (m, 2H), 3.17 (s, 3H), 3.64 (m, 1H), 3.97 (s, 2H), 4.32 (m, 2H), 4.40-4.74 (m, 2H), 4.88 (m, 1H) partially obscured by solvent peak), 8.06-8.17 (m, 4H), 8.35 (m, 2H), 8.85 (m, 1H); LRESIMS for C₂₅H₃₅N₃O₄S m/z 474 (M+H)⁺; HRESIMS, calc. monoiso mass (Da): 473.2348, found monoiso mass (Da): 473.2352.

### Example B34

### Isobutyl 4-[({6-[4-(methylsulfonyl)phenyl]pyridin-3-yl}methyl)(propyl)amino]-piperidine-1-carboxylate, trifluoroacetate

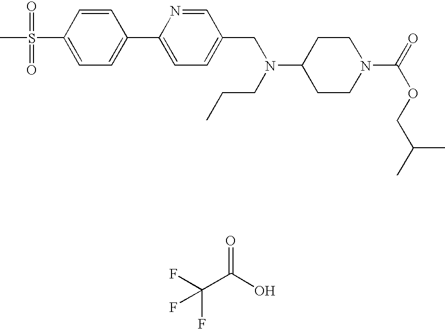

N-({6-[4-(methylsulfonyl)phenyl]pyridin-3-yl}methyl)-N-propylpiperidin-4-amine (73 mg, 0.188 mmol; Intermediate B4) was dissolved in DCM (2.5 mL) and Et₃N (0.075 mL, 0.54 mmol). Isobutyl chloroformate (0.04 mL, 0.3 mmol) dissolved in DCM (0.46 mL) was added. The reaction mixture was stirred overnight at r.t., evaporated in vacuo and purified by preparative HPLC (System D). Yield 55 mg (46%). Analytical HPLC: purity 100%, R_{T}=1.79 min (System A); ¹H NMR (400 MHz, CD₃OD) Selected peaks: δ ppm 0.91-1.00 (m, 9H), 1.59-2.00 (m, 5H), 2.82-3.04 (m, 2H), 3.17 (s, 3H), 3.64 (m, 1H), 3.88 (m, 2H), 3.97 (s, 2H), 4.30-4.39 (m, 2H), 4.40-4.74 (m, 2H), 8.05-8.17 (m, 4H), 8.35 (m, 2H), 8.85 (m, 1H); LRESIMS for C₂₆H₃₇N₃O₄S m/z 488 (M+H)⁺; HRESIMS, calc. monoiso mass (Da): 487.2505, found monoiso mass (Da): 487.2509.

### Intermediate B5

### tert-Butyl 4-[[(6-chloropyridin-3-yl)methyl](cyclopropyl)amino]piperidine-1-carboxylate

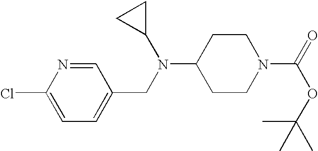

tert-Butyl 4-{[(6-chloropyridin-3-yl)methyl]amino}piperidine-1-carboxylate (1470 mg, 4.5 mmol; Intermediate B1) was dissolved in MeOH (18 mL) and HOAc (2.7 mL, 10 equiv). First, [(1-ethoxycyclopropyl)oxy]trimethylsilane (3.66 g, 4.2 mL, 21 mmol) was added and then NaBH₃CN (1.13 g, 18 mmol). The reaction mixture was heated at reflux overnight. The mixture was concentrated under reduced pressure and the residue was acidified with 1 M HCl. 10% aqueous Na₂CO₃ was added and the mixture was extracted with EtOAc. Flash chromatography on silica gel using MeOH/CHCl₃ (2:98) as eluent gave the title compound as an oil. Yield 1.815 g. Analytical HPLC: purity 98%, R_{T}=1.64 min (System A), HPLC 100%, R_{T}=2.90 min (System B); ¹H NMR (400 MHz, CDCl₃) δ ppm 0.20-0.29 (m, 2H), 0.40-0.49 (m, 2H), 1.44 (s, 9H), 1.47-1.61 (m, 3H), 1.66-1.80 (m, 2H), 1.92-2.01 (m, 1H), 2.51-2.69 (m, 3H), 3.76 (s, 2H), 4.00-4.35 (m, 3H), 7.22 (1H), 7.52-7.59 (m, 1H), 8.25 (m, 1H); LRESIMS for C₁₉H₂₈ClN₃O₂ m/z 366 (M+H)⁺.

### Intermediate B6

### N-cyclopropyl-N-({6-[4-(methylsulfonyl)phenyl]pyridin-3-yl}methyl)piperidin-4-amine

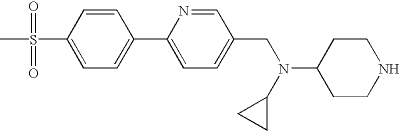

tert-Butyl 4-[cyclopropyl({6-[4-(methylsulfonyl)phenyl]pyridin-3-yl}methyl)amino]-piperidine-1-carboxylate (392 mg, 0.807 mmol; obtained in Example B35) was dissolved in DCM (3.3 mL) and TFA (0.825 mL) and the reaction mixture was stirred at r.t. for 1 h. The mixture was concentrated under reduced pressure and the residue was partitioned between DCM (120 mL) and 1 M NaOH (12 mL). The DCM phase was separated, dried (Na₂SO₄), filtered and concentrated to give the title compound. Yield 262 mg (84%). Analytical HPLC: purity 99%, R_{T}=0.95 min (System A); LRESIMS for C₂₁H₂₇N₃O₂S m/z 386 (M+H)⁺.

### Intermediate B7

### Isopropyl 4-[[(6-chloropyridin-3-yl)methyl](cyclopropyl)amino]piperidine-1-carboxylate

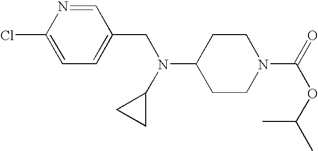

tert-Butyl 4-[[(6-chloropyridin-3-yl)methyl](cyclopropyl)amino]piperidine-1-carboxylate (1.54 g, 4.2 mmol; Intermediate B5) was dissolved in DCM (18 mL) and TFA (4.4 mL, 57 mmol) was added. The reaction mixture was stirred at r.t. for 80 min and then concentrated in vacuo. DCM (100 mL) and 1 M NaOH (20 mL) were added to the crude mixture. The aqueous solution was extracted with DCM (50 mL) and the combined DCM layers were dried (Na₂SO₄), filtered and concentrated under reduced pressure to give (6-chloro-pyridin-3-ylmethyl)-cyclopropyl-piperidin-4-yl-amine as an oil. This intermediate (998 mg, 3.75 mmol) was dissolved in DCM (45 mL) and Et₃N (1.38 mL, 1.0 g, 10 mmol). Isopropyl chloroformate (1 M solution in toluene, 7.5 mL, 7.5 mmol) was added. The reaction mixture was stirred at r.t. for two days and then concentrated under reduced pressure. EtOAc (50 mL) and 10% aqueous Na₂CO₃ (10 mL) were added and the organic phase was separated, dried (Na₂SO₄), filtered and concentrated under reduced pressure to give the title compound. Yield 1.35 g (91%). Analytical HPLC: purity 97%, R_{T}=1.49 min (System A); LRESIMS for C₁₈H₂₆ClN₃O₂: m/z 352 (M+H)⁺.

### Example B35

### tert-Butyl 4-[cyclopropyl({6-[4-(methylsulfonyl)phenyl]pyridin-3-yl}methyl)amino]-piperidine-1-carboxylate

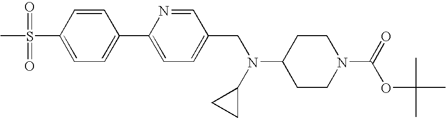

tert-Butyl 4-[({6-[4-(methylsulfonyl)phenyl]pyridin-3-yl}methyl)amino]piperidine-1-carboxylate (223 mg, 0.50 mmol; obtained in Example B1) was dissolved in MeOH (2 mL) and HOAc (0.285 mL, 10 equiv). First, [1(1-ethoxycyclopropyl)oxy]trimethylsilane (0.50 mL, 2.5 mmol) was added and then NaBH₃CN (126 mg, 2.0 mmol). The mixture stirred at reflux overnight. The solvent was evaporated and the residue acidified with 1 M HCl. 10% aqueous Na₂CO₃ was added and the mixture was extracted with EtOAc. Flash chromatography using 2 M NH₃ in MeOH/CHCl₃ (3:97) as eluent gave the title compound. Yield 212 mg (87%). Analytical HPLC: purity 97% (System A, R_{T}=1.74 min); ¹H NMR (400 MHz, CDCl₃) δ ppm 0.27-0.35 (m, 2H), 0.43-0.51 (m, 2H), 1.44 (s, 9H), 1.50-1.64 (m, 2H), 1.79 (m, 2H), 2.02 (m, 1H), 2.52-2.74 (m, 3H), 3.08 (s, 3H), 3.86 (s, 2H), 4.16 (br s, 2H), 7.72 (s, 2H), 8.03 (m, 2H), 8.19 (m, 2H), 8.61 (m, 1H); LRESIMS for C₂₆H₃₅N₃O₄S m/z 486 (M+H)⁺; HRESIMS, calc. monoiso mass (Da): 485.2348, found monoiso mass (Da): 485.2352.

### Example B36

### Isopropyl 4-[cyclopropyl({6-[4-(methylsulfonyl)phenyl]pyridin-3-yl}methyl)amino]-piperidine-1-carboxylate, trifluoroacetate

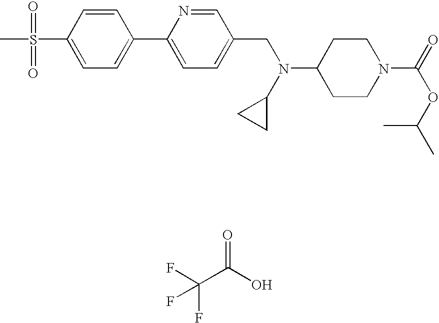

N-Cyclopropyl-N-({6-[4-(methylsulfonyl)phenyl]pyridin-3-yl}methyl)piperidin-4-amine (75 mg, 0.195 mmol; Intermediate B6) was dissolved in DCM (2.5 mL) and Et₃N (0.075 mL, 0.54 mmol). Isopropyl chloroformate (1 M solution in toluene, 0.4 mL, 0.4 mmol) was added. The mixture was stirred at r.t. overnight, evaporated in vacuo and purified by preparative HPLC (System D). Yield 60 mg (66%). Analytical HPLC: purity 100%, R_{T}=1.58 min (System A); ¹H NMR (400 MHz, CD₃OD) δ ppm 0.67-0.87 (m, 1H), 0.90-1.02 (m, 2H), 1.26 (d, J=6.3 Hz, 6H), 1.82-1.97 (m, 2H), 2.23-2.33 (m, 2H), 2.82-2.98 (m, 3H), 3.17 (s, 3H), 3.62-3.71 (m, 1H), 3.97 (s, 2H), 4.29-4.39 (m, 2H), 4.65 (s, 2H), 4.83-4.93 (m, 1H, partially obscured by solvent peak), 8.05-8.14 (m, 4H), 8.34 (m, 2H), 8.85-8.87 (m, 1H); LRESIMS for C₂₅H₃₃N₃O₄S m/z 472 (M+H)⁺; HRESIMS, calc. monoiso mass (Da): 471.2192, found monoiso mass (Da): 471.2195.

### Example B37

### Isobutyl 4-[cyclopropyl({6-[4-(methylsulfonyl)phenyl]pyridin-3-yl}methyl)amino]-piperidine-1-carboxylate, trifluoroacetate

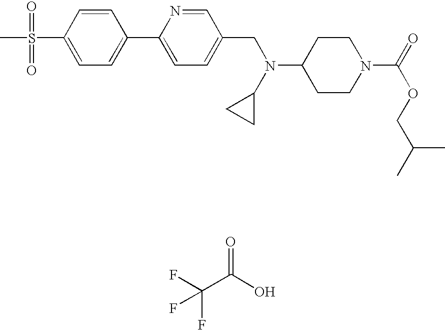

N-Cyclopropyl-N-({6-[4-(methylsulfonyl)phenyl]pyridin-3-yl}methyl)piperidin-4-amine (73 mg, 0.189 mmol; Intermediate B6) was dissolved in DCM (2.5 mL) and Et₃N (0.075 mL, 0.54 mmol). Isobutyl chloroformate (0.04 mL, 0.3 mmol) dissolved in DCM (0.46 mL) was added. The mixture was stirred overnight at r.t., concentrated under reduced pressure and purified by preparative HPLC (System D). Yield 68 mg (56%). Analytical HPLC: purity 100%, R_{T}=1.73 min (System A); ¹H NMR (400 MHz, CDCl₃) δ ppm 0.77 (m, 2H), 0.96 (d, J=6.8 Hz, 6H), 1.83-2.00 (m, 3H), 2.27 (m, 2H), 2.83-3.04 (m, 3H), 3.17 (s. 3H), 3.67 (m, 1H), 3.88 (d, J=6.5 Hz, 2H), 3.97 (s, 2H), 4.36 (m, 2H), 4.65 (s, 2H), 8.06-8.16 (m, 4H), 8.34 (m, 2H), 8.85 (m, 1H); LRESIMS for C₂₆H₃₅N₃O₄S m/z 486 (M+H)⁺; HRESIMS, calc. monoiso mass (Da): 485.2348, found monoiso mass (Da): 485.2352.

### Example B38

### tert-Butyl 4-[cyclopropyl({6-[4-(methylsulfinyl)phenyl]pyridin-3-yl}methyl)amino]-piperidine-1-carboxylate

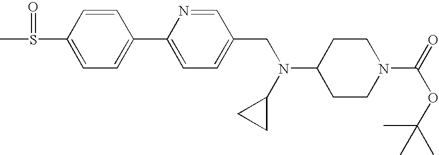

tert-Butyl 4-[[(6-chloropyridin-3-yl)methyl](cyclopropyl)amino]piperidine-1-carboxylate, (73 mg, 0.20 mmol; Intermediate B5) was mixed with 4-(methanesulfinyl)benzeneboronic acid (44 mg, 0.24 mmol) and dioxane (1.6 mL). First, K₂CO₃ (69 mg, 0.5 mmol) dissolved in water (0.4 mL) was added and then Pd(PPh₃)₄ (12 mg, 0.01 mmol). The mixture was stirred at 85° C. for 5 h. EtOH was added and the mixture was concentrated under reduced pressure. 10% aqueous Na₂CO₃ (0.8 mL) and DCM (8 mL) were added to the crude mixture. The organic phase was filtered and evaporated. The crude product was purified by preparative HPLC (System E, gradient 36-65% MeCN). Yield 10 mg. Analytical HPLC: purity 99%, R_{T}=1.57 min (System A); ¹H NMR (400 MHz, CDCl₃) δ ppm 0.27-0.34 (m, 2H), 0.46-0.53 (m, 2H), 1.44 (s, 9H), 1.50-1.64 (m, 2H), 1.82-1.92 (m, 2H), 2.06 (m, 1H), 2.59-2.82 (m, 3H), 2.84 (s, 3H), 3.92 (s, 2H), 4.13 (m, 2H), 7.82 (m, 2H), 7.87 (m, 2H), 8.18 (m, 2H), 8.57 (m, 1H); LRESIMS for C₂₆H₃₅N₃O₃S m/z 470 (M+H)⁺; HRESIMS, calc. monoiso mass (Da): 469.2399, found monoiso mass (Da): 469.2408.

### Example B39

### tert-Butyl 4-{cyclopropyl[(6-{4-[(dimethylamino)carbonyl]phenyl}pyridin-3-yl)-methyl]amino}piperidine-1-carboxylate

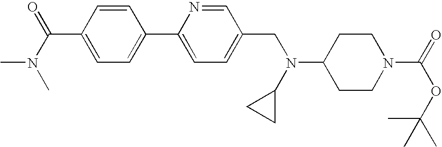

To a stirred solution of tert-butyl 4-[[(6-chloropyridin-3-yl)methyl](cyclopropyl)amino]-piperidine-1-carboxylate (73 mg, 0.20 mmol; Intermediate B5) in dioxane (1.6 mL) were added [4-(N,N-dimethylaminocarbonyl)phenyl]boronic acid (46 mg, 0.24 mmol), K₂CO₃ (69 mg, 0.5 mmol) dissolved in water (0.4 mL), and Pd(PPh₃)₄ (12 mg, 0.01 mmol). The mixture was stirred at 85° C. for 5 h. EtOH was added and the mixture was concentrated under reduced pressure. 10% aqueous Na₂CO₃ (0.8 mL) and DCM (8 mL) were added. The organic phase was separated, dried (Na₂SO₄), filtered and concentrated under reduced pressure. The crude product was purified by preparative HPLC (System E, gradient 40-70% MeCN). Yield 2 mg. Analytical HPLC: purity 99%, R_{T}=1.65 min (System A); ¹H NMR (400 MHz, CDCl₃) δ ppm 0.27-0.34 (m, 2H), 0.46-0.53 (m, 2H), 1.44 (s, 9H), 1.50-1.64 (m, 2H), 1.82-1.92 (m, 2H), 2.07 (m, 1H), 2.59-2.81 (m, 3H), 3.04 (s, 3H), 3.12 (s, 3H), 3.91 (s, 2H), 4.13 (m, 2H), 7.54 (m, 2H), 7.84 (s, 2H), 8.04 (m, 2H), 8.55 (m, 1H); LRESIMS for C₂₈H₃₈N₄O₃ m/z 479 (M+H)⁺; HRESIMS, calc. monoiso mass (Da): 478.2944, found monoiso mass (Da): 478.2954.

### Example B40

### Isopropyl 4-[cyclopropyl({6-[4-(methylsulfinyl)phenyl]pyridin-3-yl}methyl)amino]-piperidine-1-carboxylate, trifluoroacetate

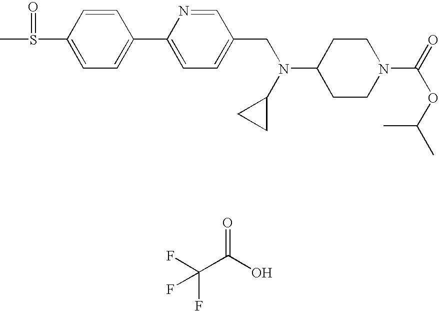

Isopropyl 4-[[(6-chloropyridin-3-yl)methyl](cyclopropyl)amino]piperidine-1-carboxylate (35 mg, 0.1 mmol; Intermediate B7) was dissolved in 80% aqueous dioxane (0.8 mL) and added to a vial containing 4-(methanesulfinyl)benzeneboronic acid (22 mg, 0.12 mmol). NaHCO₃ (21 mg, 0.25 mmol) and Pd(PPh₃)₄ (6 mg, 0.005 mmol) were added. The reaction mixture was heated to 85° C. overnight. The mixture was filtered and the solvent evaporated. Purification of the residue by preparative HPLC (System D) gave the title compound as its TFA-salt. Yield 11 mg (19%). Analytical HPLC: purity 91%, R_{T}=1.44 min (System A); LRESIMS for C₂₅H₃₃N₃O₃S m/z 456 (M+H)⁺; HRESIMS, calc. monoiso mass (Da): 455.2243, found monoiso mass (Da): 455.2246.

### Example B41

### Isopropyl 4-{cyclopropyl[(6-{4-[(dimethylamino)carbonyl]phenyl}pyridin-3-yl)-methyl]amino}piperidine-1-carboxylate, trifluoroacetate

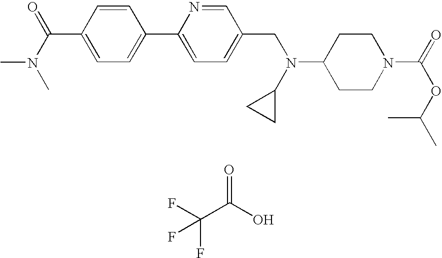

Isopropyl 4-[[(6-chloropyridin-3-yl)methyl](cyclopropyl)amino]piperidine-1-carboxylate (35 mg, 0.1 mmol; Intermediate B7) was dissolved in 80% aqueous dioxane (0.8 mL) and added to a vial containing [4-(N,N-dimethylaminocarbonyl)phenyl]boronic acid (23 mg, 0.12 mmol). NaHCO₃ (21 mg, 0.25 mmol) and Pd(PPh₃)₄ (6 mg, 0.005 mmol) were added. The reaction mixture was heated to 85° C. and stirred overnight. The mixture was allowed to cool and then filtered. The filtrate was concentrated under reduced pressure. Purification of the residue by preparative HPLC (System D) gave the title compound as its TFA-salt. Yield 3.6 mg (6%). Analytical HPLC: purity 97%, R_{T}=1.53 min (System A); LRESIMS for C₂₇H₃₆N₄O₃ m/z 465 (M+H)⁺; HRESIMS, calc. monoiso mass (Da): 464.2787, found monoiso mass (Da): 464.2789.

### Intermediate B8

### tert-Butyl 4-{[(6-chloropyridin-3-yl)methyl](methyl)amino}piperidine-1-carboxylate

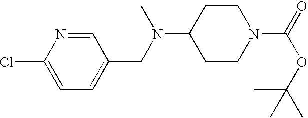

Sodium cyanoborohydride (0.135 g, 2.15 mmol) was added to a solution of tert-butyl 4-{[(6-chloropyridin-3-yl)methyl]amino}piperidine-1-carboxylate (0.50 g, 1.53 mmol; Intermediate B1) in MeOH (50 mL). Formaldehyde 37 wt. % solution in water (0.274 g, 3.37 mmol) and 5 M HCl in MeOH (0.123 mL, 0.61 mmol) were added and the mixture was stirred for 0.5 h at r.t. Saturated aqueous NaHCO₃ and water were added and the product was extracted with DCM. The organic phase was dried over Na₂SO₄, filtered and concentrated to give the title compound. Yield 0.49 g (94%).

### Example B42

### tert-Butyl 4-[{[6-(4-cyanophenyl)pyridin-3-yl]methyl}(methyl)amino]piperidine-1-carboxylate

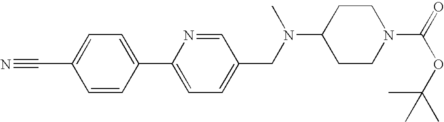

A mixture of tert-butyl 4-{[(6-chloropyridin-3-yl)methyl](methyl)amino}piperidine-1-carboxylate (0.03 g, 0.09 mmol; Intermediate B8), (4-cyanophenyl)boronic acid (0.014 g, 0.10 mmol), Pd(PPh₃)₄ (0.005 g, 0.004 mmol) and K₂CO₃ (0.030 g, 0.22 mmol) in a solvent mixture of 1,4-dioxane (0.8 mL) and water (0.2 mL) was exposed to microwave irradiation (130° C.) for 20 minutes. Solid material was filtered off and the filtrate was concentrated under reduced pressure and purified by preparative HPLC (System D). Pure fractions were combined, saturated aqueous NaHCO₃ was added and the mixture was extracted with EtOAc. The organic phase was dried over Na₂SO₄, filtered and the solvent was evaporated to give the title compound. Yield: 16 mg (44%); Analytical HPLC: purity 96% (System A and B); LRESIMS for C₂₄H₃₀N₄O₂ m/z 407 (M+H)⁺; HRESIMS, calc. monoiso mass (Da): 406.2369, found monoiso mass (Da): 406.2372.

### Example B43

### tert-Butyl 4-[[(6-{4-[(dimethylamino)carbonyl]phenyl}pyridin-3-yl)methyl](methyl)-amino]piperidine-1-carboxylate

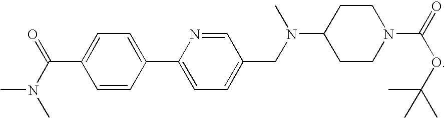

A mixture of tert-butyl 4-{[(6-chloropyridin-3-yl)methyl](methyl)amino}piperidine-1-carboxylate (0.03 g, 0.09 mmol; Intermediate B8), 4-(N,N-dimethylaminocarbonyl)-phenyl)boronic acid (0.018 g, 0.10 mmol), Pd(PPh₃)₄ (0.005 g, 0.004 mmol) and K₂CO₃ (0.030 g, 0.22 mmol) in a solvent mixture of 1,4-dioxane (0.8 mL) and water (0.2 mL) was exposed to microwave irradiation (130° C.) for 20 minutes. Solid material was filtered off and the filtrate was concentrated under reduced pressure and purified by preparative HPLC (System D). Pure fractions were combined, saturated aqueous NaHCO₃ was added and the mixture was extracted with EtOAc. The organic phase was dried over Na₂SO₄, filtered and the solvent was evaporated to give the title compound. Yield: 17 mg (42%); Analytical HPLC: purity 98% (System A), purity 96% (System B); ¹H NMR (400 MHz, CDCl₃) δ ppm 1.45 (s, 9H) 1.47-1.85 (m, 5H) 2.21 (s, 3H) 2.54-2.75 (m, 2H) 3.00 (s, 3H) 3.12 (s, 3H) 3.62 (s, 2H) 4.10-4.21 (m, 2H) 7.51 (m, 2H) 7.68-7.75 (m, 2H) 8.01 (m, 2H) 8.59 (m, 1H); LRESIMS for C₂₆H₃₆N₄O₃ m/z 453 (M+H)⁺; HRESIMS, calc. monoiso mass (Da): 452.2787, found monoiso mass (Da): 452.2791.

### Example B44

### tert-Butyl 4-[methyl({6-[4-(morpholin-4-ylcarbonyl)phenyl]pyridin-3-yl}methyl)-amino]piperidine-1-carboxylate

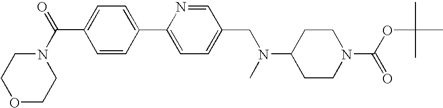

tert-Butyl 4-{[(6-chloropyridin-3-yl)methyl]amino}piperidine-1-carboxylate (163 mg, 0.50 mmol; Intermediate B1) and [4-(morpholine-4-carbonyl)phenyl]boronic acid (141 mg, 0.60 mmol) were dissolved in dioxane (4 mL). K₂CO₃ (173 mg, 1.25 mmol) in water (1 mL) and Pd(PPh₃)₄ (29 mg, 0.02 mmol) were added. The mixture was stirred at 80° C. overnight. The solvent was evaporated in vacuo and the residue taken up in DCM. Flash chromatography on silica gel using 2M NH₃ in MeOH/CHCl₃ (5:95) as eluent gave the intermediate tert-butyl 4-[({6-[4-(morpholin-4-ylcarbonyl)phenyl]pyridin-3-yl}methyl)-amino]piperidine-1-carboxylate. Yield 162 mg (67%).

Part of this material (100 mg, 0.21 mmol) was dissolved in 1,2-dichloroethane (1.5 mL) and formalin (0.04 mL) and NaBH(OAc)₃ (141 mg, 0.66 mmol) were added. The mixture was stirred overnight at r.t. The solvent was evaporated in vacuo and 10% aqueous Na₂CO₃ (2 mL) was added. The mixture was extracted with CHCl₃ (2×25 mL) and the combined organic phases were concentrated under reduced pressure. The residue was purified by preparative HPLC (System E) to give the title compound. Analytical HPLC: purity 99%, R_{T}=1.55 min (System A); ¹H NMR (400 MHz, CDCl₃) δ ppm 1.41-1.57 (m, 11H), 1.83-1.93 (m, 2H), 2.24 (s, 3H), 2.61-2.85 (m, 3H), 3.42-3.86 (m, 10H), 4.10-4.19 (m, 2H), 7.51-7.59 (m, 2H), 7.85-7.92 (m, 2H), 8.03-8.10 (m, 2H), 8.58 (m, 1H); LRESIMS for C₂₈H₃₈N₄O₄ m/z 495 (M+H)⁺; HRESIMS, calc. monoiso mass (Da): 494.2893, found monoiso mass (Da): 494.2908.

### Example B45

### tert-Butyl 4-[({6-[4-(aminocarbonyl)phenyl]pyridin-3-yl}methyl)(3,3,3-trifluoropropyl)amino]piperidine-1-carboxylate

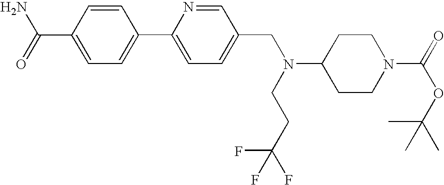

tert-Butyl 4-[({6-[4-(aminocarbonyl)phenyl]pyridin-3-yl}methyl)amino]piperidine 1-carboxylate was prepared from 4-aminocarbonylphenylboronic acid and tert-butyl 4-{[(6-chloropyridin-3-yl)methyl]amino}piperidine-1-carboxylate (Intermediate B1) by the general method C. Yield 10 mg, 12%. Reductive amination of tert-butyl 4-[({6-[4-(aminocarbonyl)phenyl]pyridin-3-yl}methyl)amino]piperidine-1-carboxylate was done by the general method D. Analytical HPLC: purity 97%, R_{T}=1.80 min (System A); LRESIMS for C₂₆H₃₃F₃N₄O₃ m/z 507 (M+H)⁺; HRESIMS, calc. monoiso mass (Da): 506.2505, found monoiso mass (Da): 506.2500.

### Example B46

### tert-Butyl 4-[[(6-{4-[(dimethylamino)carbonyl]phenyl}pyridin-3-yl)methyl](3,3,3-trifluoropropyl)amino]piperidine-1-carboxylate

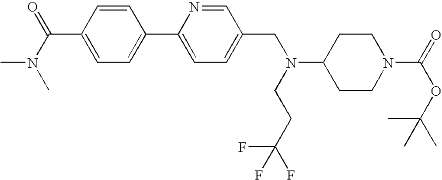

tert-Butyl 4-{[(6-{4-[(dimethylamino)carbonyl]phenyl}pyridin-3-yl)methyl]amino}-piperidine-1-carboxylate was prepared from [4-(N,N-dimethylaminocarbonyl)phenyl]-boronic acid and tert-butyl 4-{[(6-chloropyridin-3-yl)methyl]amino}piperidine-1-carboxylate (Intermediate B1), by the general method C with the exception that flash chromatography was used instead of preparative HPLC. Yield 45 mg (51%). Reductive amination of tert-butyl 4-{[(6-{4-[(dimethylamino)carbonyl]-phenyl}3 pyridin-3-yl)methyl]amino}piperidine-1-carboxylate was done by the general method D. Analytical HPLC: purity 91%, R_{T}=1.91 min (System A); LRESIMS for C₂₈H₃₇F₃N₄O₃ m/z 535 (M+H)⁺.

### Example B47

### tert-Butyl 4-[[(6-{4-[(acetylamino)methyl]phenyl}pyridin-3-yl)methyl](3,3,3-trifluoropropyl)amino]piperidine-1-carboxylate

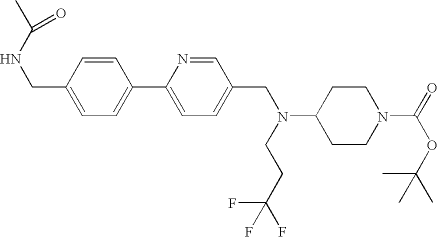

4-({6-[4-(Acetylamino-methyl)phenyl]pyridin-3-ylmethyl}amino)piperidine-1-carboxylic acid tert-butyl ester was prepared from 4-acetamidomethylphenylboronic acid and tert-butyl 4-{[(6-chloropyridin-3-yl)methyl]amino}piperidine-1-carboxylate (Intermediate B1) by the general method C. Yield 47 mg (54%). Reductive amination of 4-({6-[4-(acetylamino-methyl)-phenyl]-pyridin-3-ylmethyl}-amino)-piperidine-1-carboxylic acid tert-butyl ester (41 mg, 0.093 mmol) was done by the general method D. Analytical HPLC: purity 90%, R_{T}=1.88 min (System A), LRESIMS for C₂₈H₃₇F₃N₄O₃ m/z 535 (M+H)⁺; HRESIMS, calc. monoiso mass (Da): 534.2818, found monoiso mass (Da): 534.2811.

### Example B48

### tert-Butyl 4-[[(6-{3-[(acetylamino)methyl]phenyl}pyridin-3-yl)methyl](3,3,3-trifluoropropyl)amino]piperidine-1-carboxylate

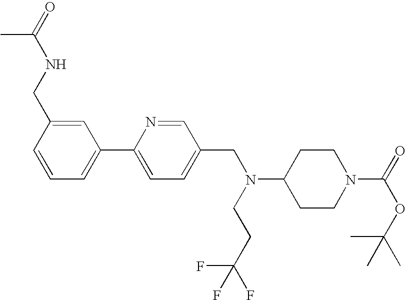

4-({6-[3-(Acetylamino-methyl)phenyl]pyridin-3-ylmethyl}amino)piperidine-1-carboxylic acid tert-butyl ester was prepared from 3-acetamidomethylphenylboronic acid and tert-butyl 4-{[(6-chloropyridin-3-yl)methyl]amino}piperidine-1-carboxylate (Intermediate B1) by the general method C. Yield 32 mg (36%). Reductive amination of 4-({6-[3-(acetylamino-methyl)phenyl]pyridin-3-ylmethyl}amino)piperidine-1-carboxylic acid tert-butyl ester (22 mg, 0.05 mmol) was done by the general method D. Analytical HPLC: purity 95%, R_{T}=1.89 min (System A), LRESIMS for C₂₈H₃₇F₃N₄O₃ m/z 535 (M+H)⁺. HRESIMS, calc. monoiso mass (Da): 534.2818, found monoiso mass (Da): 534.2813.

### Example B49

### tert-Butyl 4-[{[6-(3-{[(2-hydroxyethyl)amino]carbonyl}phenyl)pyridin-3-yl]methyl}-(3,3,3-trifluoropropyl)amino]piperidine-1-carboxylate

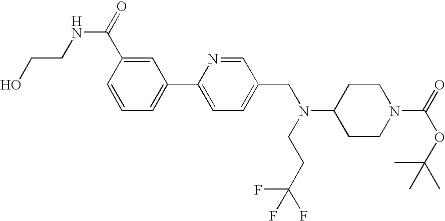

tert-Butyl 4-({[6-(3-{[(2-hydroxyethyl)amino]carbonyl}phenyl)pyridin-3-yl]methyl}-amino)piperidine-1-carboxylate was prepared from N-[2-hydroxyethyl]benzamide-3-boronic acid and tert-butyl 4-{[(6-chloropyridin-3-yl)methyl]amino}piperidine-1-carboxylate (Intermediate B1) by the general method C. Yield 24 mg, 26%. Reductive amination of tert-butyl 4-({[6-(3-{[(2-hydroxyethyl)amino]carbonyl}-phenyl)pyridin-3-yl]methyl}amino)piperidine-1-carboxylate was done by the general method D. Analytical HPLC: purity 92%, R_{T}=1.79 min (System A); LRESIMS for C₂₈H₃₇F₃N₄O₄ m/z 551 (M+H)⁺; HRESIMS, calc. monoiso mass (Da): 550.2767, found monoiso mass (Da): 550.2765.

### Example B50

### tert-Butyl 4-[({6-[3-(aminocarbonyl)phenyl]pyridin-3-yl}methyl)(3,3,3-trifluoropropyl)amino]piperidine-1-carboxylate

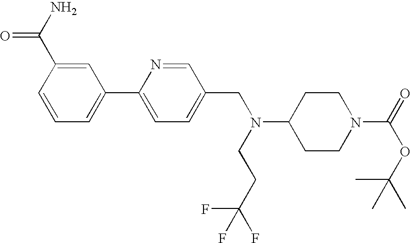

tert-Butyl 4-[({6-[3-(aminocarbonyl)phenyl]pyridin-3-yl}methyl)amino]piperidine-1-carboxylate was prepared from 3-aminocarbonylphenylboronic acid and tert-butyl 4-{[(6-chloropyridin-3-yl)methyl]amino}piperidine-1-carboxylate (Intermediate B1) by the general method C. Yield 26 mg (12%). Reductive amination of tert-butyl 4-[({6-[3-(aminocarbonyl)phenyl]pyridin-3-yl}methyl)amino]piperidine-1-carboxylate was done by the general method D. Analytical HPLC: purity 90%, R_{T}=1.81 min (System A); LRESIMS for C₂₆H₃₃F₃N₄O₃ m/z 451 (M+H−t-Bu)⁺; HRESIMS, calc. monoiso mass (Da): 506.2505, found monoiso mass (Da): 506.2499.

### Example B51

### 1-(2,2-Dimethylpropanoyl)-N-methyl-N-({6-[4-(methylsulfonyl)phenyl]pyridin-3-yl}-methyl)piperidin-4-amine, trifluoroacetate

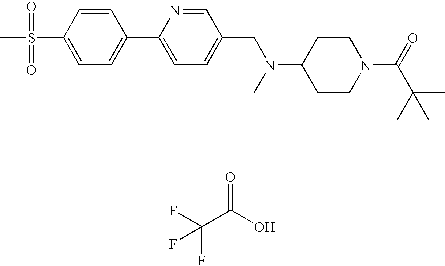

The title compound was prepared from Intermediate B2 and 2,2-dimethyl-propionyl chloride in accordance with general method A. Yield 35 mg (90%); Analytical HPLC: purity 100% (System A, R_{T}=1.46 min); ¹H NMR (400 MHz, CDCl₃) δ ppm 1.28 (s, 9H), 1.64-1.78 (m, 2H), 2.20 (m, 2H), 2.70 (s, 3H), 2.82 (m, 2H), 3.10 (s, 3H), 3.63 (m, 1H), 4.32 (br s, 2H), 4.65 (m, 2H), 7.91 (m, 1H), 8.07 (m, 2H), 8.13-8.23 (m, 3H), 8.72 (m, 1H); LRESIMS for C₂₄H₃₃N₃O₃S m/z 444 (M+H)⁺; HRESIMS, calc. monoiso mass (Da): 443.2243, found monoiso mass (Da): 443.2242.

### Intermediate B9

### Benzyl 4-{[(6-chloropyridin-3-yl)methyl]amino}-3-methylpiperidine-1-carboxylate

### Cis/Trans Mixture

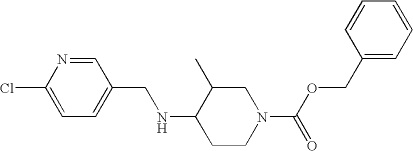

A suspension of 5-aminomethyl-2-chloropyridine (1.08 g, 7.56 mmol), 3-methyl-4-oxopiperidine-1-carboxylic acid benzyl ester (1.87 g, 7.56 mmol) and sodium cyanoborohydride (0.950 g, 15.12 mmol) in methanol (50 mL) and acetic acid (15 mL) was stirred overnight. After evaporation the residue was purified by preparative HPLC (System E, gradient 30-60% MeCN). This intermediate was used without further characterization in the preparation of Intermediate B10. Yield 502 mg (18%).

### Intermediate B10

### Benzyl 3-methyl-4-[({6-[4-(methylsulfonyl)phenyl]pyridin-3-yl}methyl)amino]-piperidine-1-carboxylate

### Cis/Trans Mixture

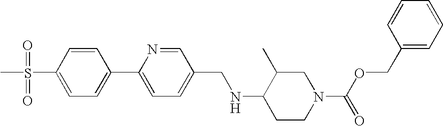

The title compound was prepared from [4-(methylsulfonyl)phenyl]boronic acid and Intermediate B9 in accordance with the procedure described for Example A1. This intermediate was used directly in the preparation of Intermediate B11. LRESIMS for C₂₇H₃₁N₃O₄S m/z 494 (M+H)⁺;

### Intermediate B11

### 3-Methyl-N-({6-[4-methylsulfonyl)phenyl]pyridin-3-yl}methyl)piperidin-4-amine

### Cis/Trans Mixture

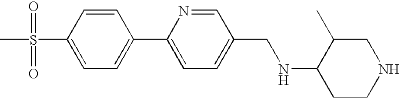

A crude mixture containing a cis- and trans-mixture of benzyl 3-methyl-4-[({6-[4-(methylsulfonyl)phenyl]pyridin-3-yl}methyl)amino]piperidine-1-carboxylate (Intermediate B10) was heated at 90° C. in a mixture of ethanol (15 mL) and 30% aqueous NaOH (10 mL) overnight and then concentrated under reduced pressure. This intermediate was used directly in the preparation of Intermediate B12.

### Intermediate B12

### tert-Butyl 3-methyl-4-[({6-[4-(methylsulfonyl)phenyl]pyridin-3-yl}methyl)amino]-piperidine-1-carboxylate

### Cis/Trans Mixture

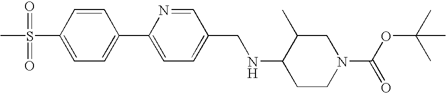

To a reaction tube containing crude 3-methyl-N-({6-[4-(methylsulfonyl)phenyl]pyridin-3-yl}methyl)piperidin-4-amine (Intermediate B11) was added methanol (5 mL), di-tert-butyl dicarbonate (1.17 g, 5.37 mmol), 2M NaOH (0.5 mL) and 4-dimethylaminopyridine (spatula tip). The mixture was stirred overnight, concentrated and then purified by preparative HPLC (System E, gradient 30-60% MeCN). The combined fractions were concentrated under reduced pressure to give 130 mg of the title compound. This intermediate was used directly in the preparation of Example B52 and Example B53.

### Example B52

### tert-Butyl (3R*,4S*)-3-methyl-4-[methyl({6-[4-(methylsulfonyl)phenyl]pyridin-3-yl}-methyl)amino]piperidine-1-carboxylate

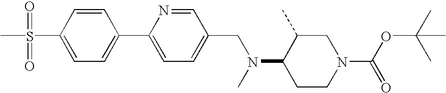

The title compound was prepared from tert-butyl 3-methyl-4-[({6-[4-(methylsulfonyl)-phenyl]pyridin-3-yl}methyl)amino]piperidine-1-carboxylate (Intermediate B12) in accordance with the procedure described for Example A33. Yield 7 mg (5%); Analytical HPLC: purity 100% (System A, R_{T}=1.71 min); LRESIMS for C₂₅H₃₅N₃O₄S m/z 474 (M+H)⁺; HRESIMS, calc. monoiso mass (Da): 473.2348, found monoiso mass (Da): 473.2360.

### Example B53

### tert-Butyl (3S*,4S*)-3-methyl-4-[methyl({6-[4-(methylsulfonyl)phenyl]pyridin-3-yl}-methyl)amino]piperidine-1-carboxylate

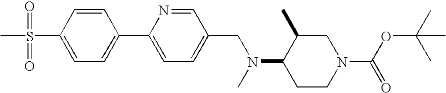

The title compound was prepared from tert-butyl 3-methyl-4-[({6-[4-(methylsulfonyl)-phenyl]pyridin-3-yl}methyl)amino]piperidine-1-carboxylate (Intermediate B12) in accordance with the procedure described for Example A33. Yield 11 mg (8%); Analytical HPLC: purity 98% (System A, R_{T}=1.67 min); LRESIMS for C₁₅H₃₅N₃O₄S m/z 474 (M+H)⁺; HRESIMS, calc. monoiso mass (Da): 473.2348, found monoiso mass (Da): 473.2343.

The relative stereochemistry of Example B52 and Example B53 was determined by NMR analysis.

### Example B54

### 1-[3-(4-Fluorophenyl)propanoyl]-N-methyl-N-({6-[4-(methylsulfonyl)phenyl]pyridin-3-yl}methyl)piperidin-4-amine, trifluoroacetate

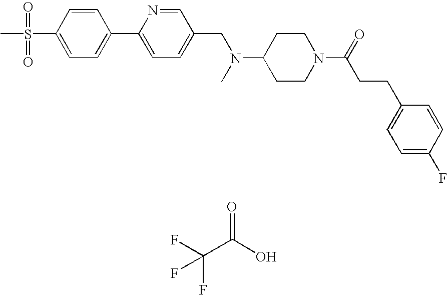

The title compound was prepared from intermediate B2 and 3-(4-fluoro-phenyl)-propionic acid in accordance with general method B. Yield 40 mg (91%); Analytical HPLC: purity 100% (System A, R_{T}=1.73 min); ¹H NMR (400 MHz, CD₃OD) δ ppm Selected peaks: 1.49-1.75 (m, 2H), 2.15 (m, 2H), 2.56-2.70 (m, 1H), 2.75 (s, 3H), 2.92 (m, 2H), 3.08 (m, 1H), 3.17 (s, 3H), 3.66 (m, 1H), 4.06-4.19 (m, 1H), 4.72-4.83 (m, 1H), 7.01 (m, 2H), 7.25 (m, 1H), 8.03-8.19 (m, 4H), 8.29-8.38 (m, 2H), 8.83 (m, 1H); LRESIMS for C₂₈H₃₂FN₃O₃S m/z 510 (M+H)⁺; HRESIMS, calc. monoiso mass (Da): 509.2148, found monoiso mass (Da): 509.2152.

### Example B55

### 1-(4-Isopropoxybenzoyl)-N-methyl-N-({6-[4-(methylsulfonyl)phenyl]pyridin-3-yl}-methyl)piperidin-4-amine, trifluoroacetate

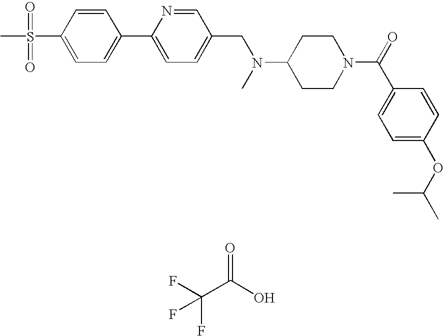

The title compound was prepared from intermediate B2 and 4-isopropoxy-benzoic acid in accordance with general method B. Yield 42 mg (94%); Analytical HPLC: purity 100% (System A, R_{T}=1.78 min); ¹H NMR (400 MHz, CD₃OD) δ ppm Selected peaks: 1.33 (d, 6H), 1.90 (m, 2H), 2.21 (m, 2H), 2.82 (s, 3H), 3.17 (s, 3H), 3.74 (m, 1H), 4.67 (m, 2H), 6.88-7.04 (m, 2H), 7.31-7.50 (m, 2H), 8.01-8.20 (m, 4H), 8.26-8.41 (m, 2H), 8.85 (m, 1H); LRESIMS for C₂₉H₃₅N₃O₄S m/z 522 (M+H)⁺; HRESIMS, calc. monoiso mass (Da): 521.2348, found monoiso mass (Da): 521.2368.

### Example B56

### 1-(3,4-Dichlorobenzoyl)-N-methyl-N-({6-[4-(methylsulfonyl)phenyl]pyridin-3-yl}-methyl)piperidin-4-amine, trifluoroacetate

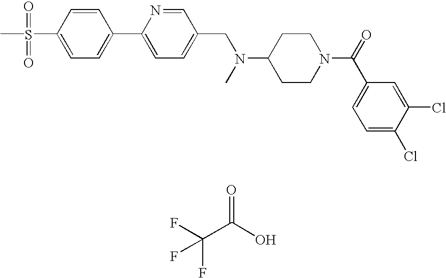

The title compound was prepared from intermediate B2 and 3,4-dichlorobenzoic acid in accordance with general method B. Yield 35 mg (77%); Analytical HPLC: purity 100% (System A, R_{T}=1.84 min); ¹H NMR (400 MHz, CD₃OD) δ ppm Selected peaks: 2.82 (s, 3H), 3.17 (s, 3H), 3.67-3.81 (m, 1H), 7.36-7.43 (m, 1H), 7.62-7.70 (m, 2H), 8.05-8.18 (m, 4H), 8.30-8.38 (m, 2H), 8.85 (m, 1H); LRESIMS for C₂₆H₂₇Cl₂N₃O₃S m/z 532 (M+H)⁺; HRESIMS, calc. monoiso mass (Da): 531.1150, found monoiso mass (Da): 531.1148.

### Example B57

### 1-[4-(4-Fluorophenyl)butanoyl]-N-methyl-N-({6-[4-(methylsulfonyl)phenyl]pyridin-3-yl}methyl)piperidin-4-amine, trifluoroacetate

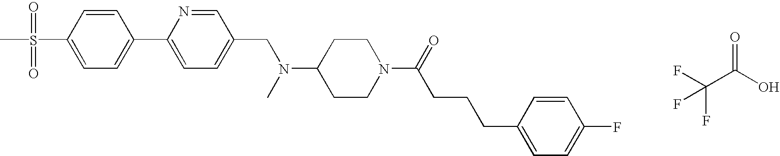

The title compound was prepared from intermediate B2 and 4-(4-fluoro-phenyl)-butyric acid in accordance with general method B. Yield 41 mg (91%); Analytical HPLC: purity 100% (System A, R_{T}=1.85 min); ¹H NMR (400 MHz, CD₃OD) δ ppm Selected peaks: 1.65-1.96 (m, 4H), 2.14-2.26 (m, 2H), 2.39-2.48 (m, 2H), 2.66 (m, 3H), 2.79 (s, 3H), 3.09-3.21 (m, 4H), 3.68 (m, 1H), 4.06-4.18 (m, 1H), 4.73-4.83 (m, 1H), 6.94-7.03 (m, 2H), 7.16-7.25 (m, 2H), 8.06-8.17 (m, 4H), 8.30-8.38 (m, 2H), 8.84 (m, 1H); LRESIMS for C₂₉H₃₄FN₃O₃S m/z 524 (M+H)⁺; HRESIMS, calc. monoiso mass (Da): 523.2305, found monoiso mass (Da): 523.2304.

### Example B58

### N-Methyl-N-({6-[4-(methylsulfonyl)phenyl]pyridin-3-yl}methyl)-1-{[3-(trifluoromethyl)phenyl]acetyl}piperidin-4-amine, trifluoroacetate

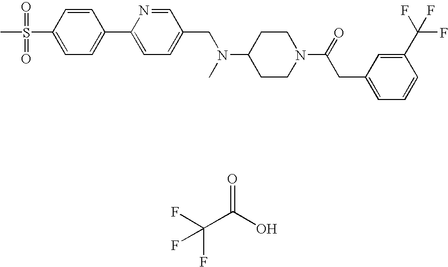

The title compound was prepared from intermediate B2 and (3-trifluoromethyl-phenyl)-acetic acid in accordance with general method B. Yield 48 mg (100%); Analytical HPLC: purity 100% (System A, R_{T}=1.86 min); ¹H NMR (400 MHz, CD₃OD) δ ppm Selected peaks: 1.65-1.83 (m, 2H), 2.20 (m, 2H), 2.67-2.80 (m, 4H), 3.14-3.25 (m, 4H), 3.70 (s, 1H), 3.94 (s, 2H), 4.22-4.33 (m, 1H), 4.75-4.83 (m, 1H), 7.50-7.62 (m, 4H), 8.05-8.17 (m, 4H), 8.30-8.38 (m, 2H), 8.83 (m, 1H); LRESIMS for m/z C₂₈H₃₀F₃N₃O₃S m/z 546 (M+H)⁺; HRESIMS, calc. monoiso mass (Da): 545.1960, found monoiso mass (Da): 545.1960.

### Example B59

### 1-(Cyclohexylacetyl)-N-methyl-N-({6-[4-(methylsulfonyl)phenyl]pyridin-3-yl}methyl)-piperidin-4-amine, trifluoroacetate

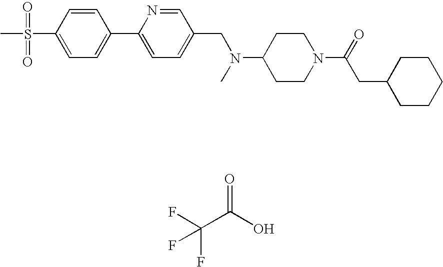

The title compound was prepared from intermediate B2 and cyclohexyl-acetic acid in accordance with general method B. Yield 14 mg (33%); Analytical HPLC: purity 100% (System A, R_{T}=1.78 min); ¹H NMR (400 MHz, CD₃OD) δ ppm 0.93-1.38 (m, 5H), 1.61-1.91 (m, 8H), 2.16-2.41 (m, 4H), 2.68 (m, 1H), 2.80 (s, 3H), 3.17 (s, 3H), 3.69 (m, 1H), 4.17-4.27 (m, 1H), 4.28-4.78 (m, 2H) 4.74-4.83 (m, 1H), 8.05-8.38 (m, 4H), 8.30-8.38 (m, 2H), 8.84 (m, 1H); LRESIMS for C₂₇H₃₇N₃O₃S m/z 484 (M+H)⁺; HRESIMS, calc. monoiso mass (Da): 483.2556, found monoiso mass (Da): 483.2562.

### Example B60

### 1-Benzoyl-N-methyl-N-({6-[4-(methylsulfonyl)phenyl]pyridin-3-yl}methyl)piperidin-4-amine, trifluoroacetate

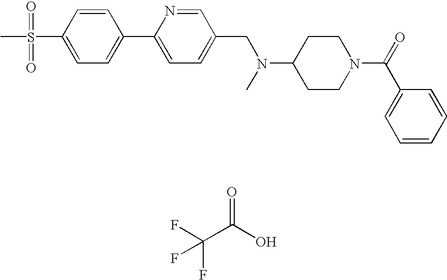

The title compound was prepared from intermediate B2 and benzoic acid in accordance with general method B. Yield 26 mg (64%). Analytical HPLC: purity 100%, R_{T}=1.48 (System A); ¹H NMR (400 MHz, CD₃OD) Selected peaks: δ ppm 2.82 (s, 3H), 3.17 (s, 3H), 7.43-7.54 (m, 5H), 8.08-8.18 (m, 4H), 8.34 (m, 2H), 8.85 (s, 1H); LRESIMS for C₂₆H₂₉N₃O₃S m/z 464 (M+H)⁺; HRESIMS, calc. monoiso mass (Da): 463.1930, found monoiso mass (Da): 463.1933.

### Example B61

### N-Methyl-1-(3-methylbutanoyl)-N-({6-[4-(methylsulfonyl)phenyl]pyridin-3-yl}-methyl)piperidin-4-amine, trifluoroacetate

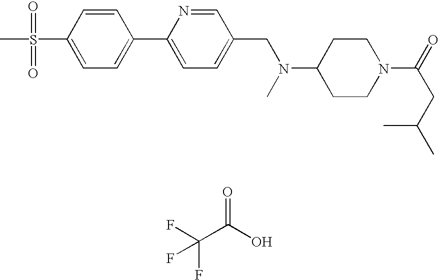

The title compound was prepared from intermediate B2 and isovaleryl chloride in accordance with general method A. Yield 33 mg (84%); Analytical HPLC: purity 100% (System A, R_{T}=1.43 min); ¹H NMR (400 MHz, CDCl₃) δ ppm 0.96 (m, 6H), 1.71 (m, 2H), 2.01-2.33 (m, 5H), 2.57 (m, 1H), 2.70 (s, 3H), 3.03-3.16 (m, 4H), 3.62 (m, 1H), 4.01-4.14 (m, 1H), 4.33 (br s, 2H), 4.93 (m, 1H), 7.91 (m, 1H), 8.06 (m, 2H), 8.12-8.22 (m, 3H), 8.72 (m, 1H); LRESIMS for C₂₄H₃₃N₃O₃S m/z 444 (M+H)⁺; HRESIMS, calc. monoiso mass (Da): 443.2243, found monoiso mass (Da): 443.2243.

### Example B62

### 1-(Cyclohexylcarbonyl)-N-methyl-N-({6-[4-(methylsulfonyl)phenyl]pyridin-3-yl}-methyl)piperidin-4-amine, trifluoroacetate

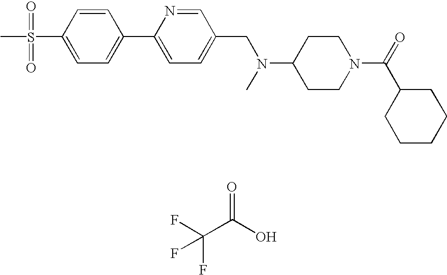

The title compound was prepared from intermediate B2 and cyclohexanecarbonyl chloride in accordance with general method A. Yield 37 mg (90%); Analytical HPLC: purity 100% (System A, R_{T}=1.61 min); ¹H NMR (400 MHz, CDCl₃) δ ppm 1.16-1.85 (m, 13H), 2.06-2.61 (m, 4H), 2.70 (s, 3H), 3.02-3.14 (m, 4H), 3.62 (m, 1H), 4.13 (m, 1H), 4.33 (br s, 2H), 4.91 (m, 1H), 7.91 (m, 1H), 8.07 (m, 2H), 8.12-8.22 (m, 3H), 8.71 (m, 1H); LRESIMS for C₂₆H₃₅N₃O₃S m/z 470 (M+H)⁺; HRESIMS, calc. monoiso mass (Da): 469.2400, found monoiso mass (Da): 469.2396.

### Example B63

### 1-(3,3-Dimethylbutanoyl)-N-methyl-N-({6-[4-(methylsulfonyl)phenyl]pyridin-3-yl}-methyl)piperidin-4-amine, trifluoroacetate

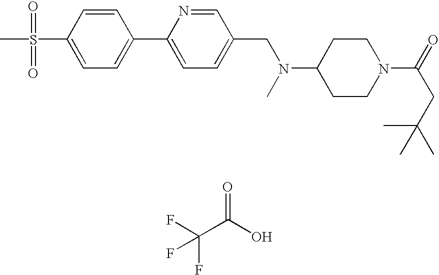

The title compound was prepared from intermediate B2 and 3,3-dimethyl-butyryl chloride in accordance with general method A. Yield 43 mg (100%); Analytical HPLC: purity 100% (System A, R_{T}=1.55 min); LRESIMS for C₂₅H₃₅N₃O₃S m/z 458 (M+H)⁺; HRESIMS, calc. monoiso mass (Da): 457.2400, found monoiso mass (Da): 457.2399.

### Example B64

### 1-(2,4-Dichlorobenzoyl)-N-methyl-N-({6-[4-(methylsulfonyl)phenyl]pyridin-3-yl}-methyl)piperidin-4-amine, trifluoroacetate

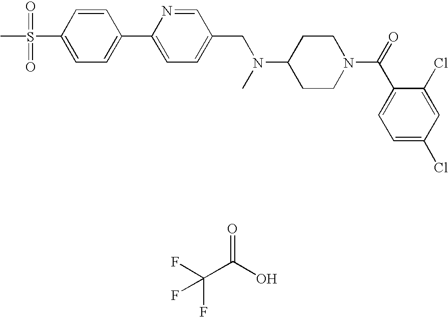

The title compound was prepared from intermediate B2 and 2,4-dichlorobenzoic acid in accordance with general method B. Yield 6.8 mg (15%); Analytical HPLC: purity 100% (System A, R_{T}=1.73 min); ¹H NMR (400 MHz, CD₃OD) Selected peaks: δ ppm 1.73-2.01 (m, 2H), 2.06-2.25 (m, 1H), 2.33 (m, 1H), 2.81 (s, 3H), 2.95 (m, 1H), 3.17 (s, 3H), 3.20-3.28 (m, obscured by solvent signal) 3.60 (m, 1H), 3.72 (m, 1H), 4.52 (br s, 2H), 4.90 (m, 1H) 7.34 (d, 0.5H), 7.42-7.51 (m, 1.5H), 7.62 (m, 1H), 8.04-8.17 (m, 4H), 8.34 (m, 2H), 8.84 (m, 1H); LRESIMS for C₂₆H₂₇Cl₂N₃O₃S m/z 532 (M+H)⁺; HRESIMS, calc. monoiso mass (Da): 531.1150, found monoiso mass (Da): 531.1155.

### Example B65

### 1-(2,4-Difluorobenzoyl)-N-methyl-N-({6-[4-(methylsulfonyl)phenyl]pyridin-3-yl}-methyl)piperidin-4-amine, trifluoroacetate

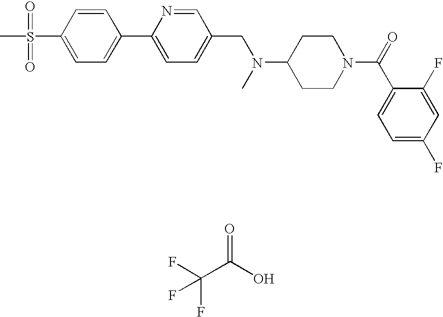

The title compound was prepared from intermediate B2 and 2,4-difluorobenzoic acid in accordance with general method B. Yield 41 mg (95%); Analytical HPLC: purity 100% (System A, R_{T}=1.53 min; ¹H NMR (400 MHz, CD₃OD) Selected peaks: δ ppm 1.74-2.01 (m, 2H), 2.08-2.25 (m, 1H), 2.27-2.40 (m, 1H), 2.81 (s, 3H), 2.91 (m, 1H), 3.17 (s, 3H), 3.75 (m, 2H), 4.54 (br s, 2H), 6.98-7.18 (m, 2H), 7.50 (m, 1H), 8.05-8.17 (m, 4H), 8.33 (m, 2H), 8.84 (m, 1H); LRESIMS for C₂₆H₂₇F₂N₃O₃S m/z 500 (M+H)⁺; HRESIMS, calc. monoiso mass (Da): 499.1741, found monoiso mass (Da): 499.1745.

### Example B66

### 1-(2,5-Difluorobenzoyl)-N-methyl-N-({6-[4-(methylsulfonyl)phenyl]pyridin-3-yl}-methyl)piperidin-4-amine, trifluoroacetate

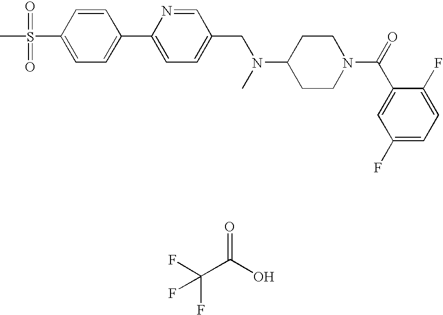

The title compound was prepared from intermediate B2 and 2,5-difluorobenzoic acid in accordance with general method B. Yield 10.7 mg (25%); Analytical HPLC: purity 100% (System A, R_{T}=1.54 min); ¹H NMR (400 MHz, CD₃OD) Selected peaks: δ ppm 1.77-2.00 (m, 2H), 2.10-2.25 (m, 1H), 2.26-2.40 (m, 1H), 2.82 (s, 3H), 2.95 (m, 1H), 3.17 (s, 3H), 3.75 (m, 2H), 4.54 (br s, 2H), 7.17-7.39 (m, 4H), 8.05-8.17 (m, 4H), 8.34 (m, 2H), 8.85 (m, 1H); LRESIMS for C₂₆H₂₇F₂N₃O₃S m/z 500 (M+H)⁺; HRESIMS, calc. monoiso mass (Da): 499.1741, found monoiso mass (Da): 499.1746.

### Example B67

### 1-(2-Fluorobenzoyl)-N-methyl-N-({6-[4-(methylsulfonyl)phenyl]pyridin-3-yl}methyl)-piperidin-4-amine, trifluoroacetate

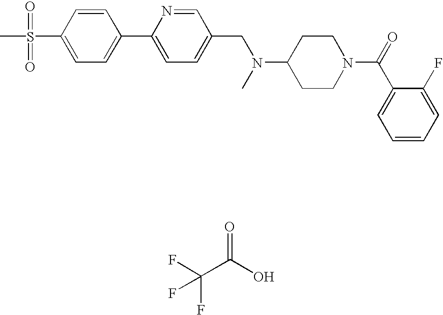

The title compound was prepared from intermediate B2 and 2-fluorobenzoic acid in accordance with general method B. Yield 32 mg (77%); Analytical HPLC: purity 97% (System A, R_{T}=1.47 min); ¹H NMR (400 MHz, CD₃OD) δ ppm 1.76-2.00 (m, 2H), 2.10-2.25 (m, 1H), 2.27-2.41 (m, 1H), 2.82 (s, 3H), 2.85 (s, 0.5H), 2.95 (m, 1H), 2.98 (s, 0.5H), 3.17 (s, 3H), 3.74 (m, 2H), 4.54 (br s, 2H), 4.84-4.97 (m, obscured by solvent signal), 7.20-7.36 (m, 2H), 7.44 (br s, 1H), 7.49-7.63 (m, 1H), 8.05-8.17 (m, 4H), 8.34 (m, 2H), 8.85 (m, 1H); LRESIMS for C₂₆H₈FN₃O₃S m/z 482 (M+H)⁺; HRESIMS, calc. monoiso mass (Da): 481.1835, found monoiso mass (Da): 481.1842.

### Example B68

### 1-(3-Fluorobenzoyl)-N-methyl-N-({6-[4-(methylsulfonyl)phenyl]pyridin-3-yl}methyl)-piperidin-4-amine, trifluoroacetate

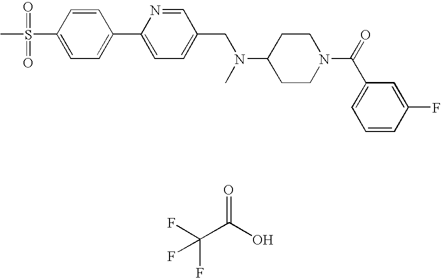

The title compound was prepared from intermediate B2 and 3-fluorobenzoic acid in accordance with general method B. Yield 27 mg (65%); Analytical HPLC: purity 100% (System A, R_{T}=1.52 min); ¹H NMR (400 MHz, CD₃OD) δ ppm 1.76-2.02 (m, 2H), 2.03-2.43 (m, 2H), 2.82 (s, 3H), 2.96 (m, 1H), 3.17 (s, 3H), 3.18-3.28 (m, obscured in part by solvent signal), 3.74 (m, 1H), 3.91 (m, 1H), 3.97 (s, 1H), 4.54 (br s, 2H), 7.20-7.32 (m, 3H), 7.50 (m, 1H), 8.04-8.17 (m, 4H), 8.34 (m, 2H), 8.85 (m, 1H); LRESIMS for C₂₆H₂₈FN₃O₃S m/z 482 (M+H)⁺; HRESIMS, calc monoiso mass (Da): 481.1835, found found monoiso mass (Da): 481.1837.

### Example B69

### 1-(4-Fluorobenzoyl)-N-methyl-N-({6-[4-(methylsulfonyl)phenyl]pyridin-3-yl}methyl)-piperidin-4-amine, trifluoroacetate

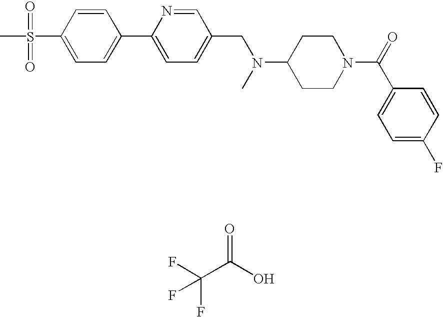

The title compound was prepared from intermediate B2 and 4-fluorobenzoic acid in accordance with general method B. Yield: 3.3 mg (8%); Analytical HPLC: purity 99% (System A, R_{T}=1.49 min); ¹H NMR (400 MHz, CD₃OD) Selected peaks: δ ppm 1.82-2.00 (m, 2H), 2.06-2.39 (m, 2H), 2.82 (s, 3H), 3.17 (s, 3H), 3.74 (m, 1H), 4.34-4.78 (m, 2H), 7.14-7.28 (m, 2H), 7.50 (m, 2H), 8.03-8.19 (m, 4H), 8.34 (m, 2H), 8.85 (m, 1H); LRESIMS for C₂₆H₂₈FN₃O₃S m/z 482 (M+H)⁺; HRESIMS, calc. monoiso mass (Da): 481.1835, found monoiso mass (Da): 481.1833.

### Example B70

### 1-(3-Methoxybenzoyl)-N-methyl-N-({6-[4-(methylsulfonyl)phenyl]pyridin-3-yl}-methyl)piperidin-4-amine, trifluoroacetate

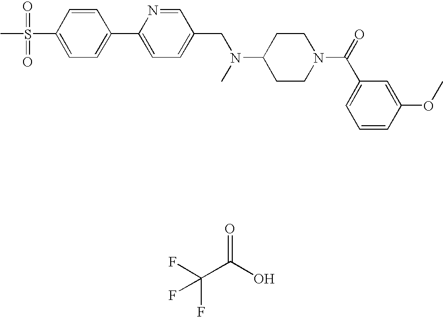

The title compound was prepared from intermediate B2 and 3-methoxybenzoic acid in accordance with general method B. Yield: 5.2 mg (12%); Analytical HPLC: purity 100% (System A, R_{T}=1.49 min); ¹H NMR (400 MHz, CD₃OD) Selected peaks: δ ppm 1.82-2.00 (m, 2H), 2.05-2.38 (m, 2H), 2.82 (s, 3H), 2.86-3.04 (m, 1H), 3.11-3.27 (m, obscured by solvent signal), 3.17 (s, 3H), 3.74 (m, 1H), 3.84 (s, 3H) 3.90-4.08 (m, 1H) 4.33-4.74 (m, 2H, obscured in part by solvent signal) 7.14-7.28 (m, 2H), 7.50 (m, 2H), 8.03-8.19 (m, 4H), 8.34 (m, 2H), 8.85 (m, 1H); LRESIMS for C₂₇H₃₁N₃O₄S m/z 494 (M+H)⁺; HRESIMS, calc. monoiso mass (Da): 493.2035, found monoiso mass (Da): 493.2031.

### Example B71

### 1-(3-Chloro-4-methoxybenzoyl)-N-methyl-N-({6-[4-(methylsulfonyl)phenyl]pyridin-3-yl}methyl)piperidin-4-amine, trifluoroacetate

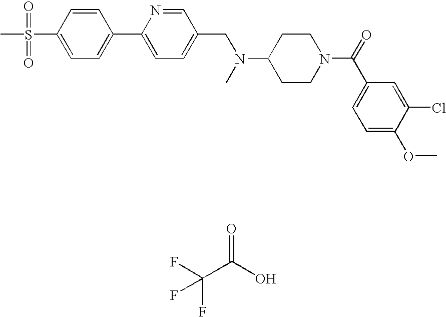

The title compound was prepared from intermediate B2 and 3-chloro-4-methoxybenzoic acid in accordance with general method B. Yield: 4.6 mg (10%); Analytical HPLC: purity 98% (System A, R_{T}=1.61 min); ¹H NMR (400 MHz, CD₃OD) Selected peaks: δ ppm 1.83-1.99 (m, 2H), 2.11-2.31 (m, 2H), 2.82 (s, 3H), 3.17 (s, 3H), 3.67-3.81 (m, 1H), 3.94 (s, 3H), 4.32-4.76 (m, 2H) 7.16 (m, 1H), 7.43 (m, 8.5 Hz, 1H), 7.53 (m, 1H), 8.06-8.17 (m, 4H), 8.36 (m, 2H), 8.85 (m, 1H); LRESIMS for C₂₇H₃₀ClN₃O₄S m/z 528 (M+H)⁺; HRESIMS, calc. monoiso mass (Da): 527.1646, found monoiso mass (Da): 527.1643.

### Intermediate B13

### tert-butyl 4-{[(6-chloropyridin-3-yl)amino]methyl}piperidine-1-carboxylate

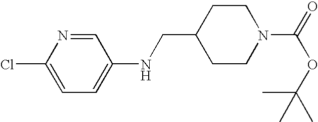

N-Boc-4-formylpiperidine (0.42 g, 2.0 mmol) was dissolved in MeOH:HOAc (9:1; 9 mL) and 2-chloro-5-aminopyridine (0.26 g, 2.0 mmol) was added. NaBH₃CN (251 mg, 4.0 mmol) was added and the mixture was stirred at r.t. for 35 min. The solvent was evaporated in vacuo and 5% aqueous NaHCO₃ was added. The mixture was extracted with EtOAc, washed with 5% aqueous NaHCO₃ and brine, and concentrated under reduced pressure. Flash chromatography of the residue using EtOAc/toluene (2:3) as eluent gave the title compound. Yield 394 mg (60%). Analytical HPLC: purity 96% (System A, R_{T}=2.37 min); ¹H NMR (400 MHz, CDCl₃) δ ppm 1.09-1.25 (m, 2H), 1.45 (s, 9H), 1.65-1.79 (m, 3H), 2.68 (m, 2H), 3.00 (d, J=6.3 Hz, 2H), 4.13 (s, 2H), 6.84 (m, 1H), 7.07 (m, 1H), 7.75 (m, 1H); LRESIMS for C₁₆H₂₄ClN₃O₂ m/z 270 (M+H−t-Bu)⁺.

### Example B72

### tert-Butyl 4-[({6-[4-(methylsulfonyl)phenyl]pyridin-3-yl}amino)methyl]piperidine-1-carboxylate

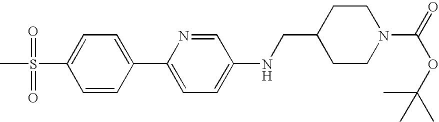

tert-Butyl 4-{[(6-chloropyridin-3-yl)amino]methyl}piperidine-1-carboxylate (325 mg, 1.0 mmol; Intermediate B13), (4-methylsulfonyl)phenylboronic acid (220 mg, 1.1 mmol), Pd(PPh₃)₄ (58 mg, 0.05 mmol), NaHCO₃ (210 mg, 2.5 mmol) were mixed with 80% aqueous dioxane (5 mL) and heated to 85° C. overnight. The reaction mixture was partitioned between DCM and 5% aqueous NaHCO₃. The organic phase was washed with 5% aqueous NaHCO₃ and brine. Flash chromatography using EtOAc/toluene (2:3) followed by MeOH/CHCl₃ (2:98) as eluents gave the title compound. Yield: 28 mg (6%); Analytical HPLC: purity 100% (System A, R_{T}=1.87 min); ¹H NMR (400 MHz, CDCl₃) δ ppm 1.13-1.28 (m, 2H), 1.45 (s, 9H), 1.78 (m, 3H), 2.70 (m, 2H), 3.06 (s, 3H), 3.08-3.15 (m, 2H), 4.16 (br s, 2H), 6.95 (m, 1H), 7.62 (m, 1H), 7.98 (m, 2H), 8.05-8.15 (m, 3H); LRESIMS for C₂₃H₃₁N₃O₄S m/z 446 (M+H)⁺; HRESIMS, calc. monoiso mass (Da): 445.2035, found monoiso mass (Da): 445.2035.

### Intermediate B14

### tert-Butyl 4-[(6-chloropyridin-3-yl)methoxy]piperidine-1-carboxylate

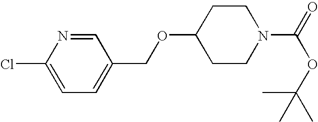

4-Hydroxy-piperidine-1-carboxylic acid tert-butyl ester (2.01 g, 10 mmol) was dissolved in THF (30 mL). NaH (60%, 0.48 g, 12 mmol) was added and the mixture was stirred for 0.5 h. 2-Chloro-5-(chloromethyl)pyridine (1.62 g, 10 mmol) was added. The mixture was stirred at room temperature overnight. The reaction mixture was concentrated under reduced pressure and EtOAc and water was added. The organic phase was washed with brine and dried (Na₂SO₄). Evaporation of the solvent gave 3.72 g crude product (yellow oil). Flash chromatography on silica gel (40 g) using n-heptane with 25% EtOAc as eluent gave the title compound. Yield: 815 mg (25%); Analytical HPLC: purity 97% (System A, R_{T}=2.46 min); ¹H NMR (400 MHz, CD₃OD) δ ppm 1.45 (s, 9H), 1.48-1.62 (m, 2H), 1.81-1.94 (m, 2H), 3.17 (m, 2H), 3.60-3.78 (m, 3H), 4.60 (s, 2H), 7.44 (m, 1H), 7.81 (m, 1H), 8.33 (m, 1H); LRESIMS for C₁₆H₂₃ClN₂O₃ m/z 271 (M+H−t-Bu)⁺.

### Example B73

### tert-Butyl 4-({6-[4-(methylsulfonyl)phenyl]pyridin-3-yl}methoxy)piperidine-1-carboxylate

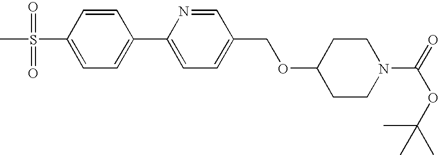

tert-Butyl 4-[(6-chloropyridin-3-yl)methoxy]piperidine-1-carboxylate (774 mg, 2.36 mmol; Intermediate B14), (4-methylsulfonyl)phenylboronic acid (519 mg, 2.60 mmol), Pd(PPh₃)₄ (136 mg, 0.118 mmol), K₂CO₃ (814 mg, 5.9 mmol) were mixed with dioxane (20 mL) and water (5 mL) and heated overnight to 90° C. The mixture was filtered through Celite and concentrated under reduced pressure. EtOAc (50 mL) was added to the crude residue. The organic phase was washed with 5% aqueous NaHCO₃ and brine. Flash chromatography of the crude product (1.42 g) on silica gel (40 g) using EtOAc/toluene (1:1) gave the title compound. Yield: 896 mg (85%); Analytical HPLC: purity 93% (System A, R_{T}=2.16 min); ¹H NMR (400 MHz, CDCl₃) δ ppm 1.45 (s, 9H), 1.55-1.68 (m, 2H), 1.84-1.95 (m, 2H), 3.08 (s, 3H), 3.09-3.18 (m, 2H), 3.62 (m, 1H), 3.72-3.84 (m, 2H), 4.64 (s, 2H), 7.79-7.91 (m, 2H), 8.02-8.08 (m, 2H), 8.19 (m, 2H), 8.72 (m, 1H); LRESIMS for C₂₃H₃₀N₂O₅S m/z 447 (M+H)⁺; HRESIMS, calc. monoiso mass (Da): 446.1875, found monoiso mass (Da): 446.1870.

### Intermediate B15

### 2-[4-(Methylsulfonyl)phenyl]-5-[(piperidin-4-yloxy)methyl]pyridine

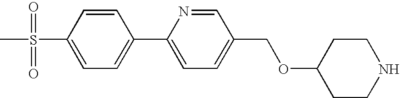

tert-Butyl 4-({6-[4-(methylsulfonyl)phenyl]pyridin-3-yl}methoxy)piperidine-1-carboxylate (448 mg, 1.0 mmol; Example B73) was dissolved in DCM (6 mL) and TFA (1.5 mL) was added. After being stirred for 45 min at r.t., the mixture was concentrated. The residue was mixed with CHCl₃ (40 mL) and washed with 10% aqueous Na₂CO₃ (4 mL) followed by 2 M NaOH (2 mL). Evaporation of the solvent gave the title compound as a white solid. Yield: 325 mg (93%); Analytical HPLC: purity 96% (System A, R_{T}=1.10 min); ¹H NMR (400 MHz, CDCl₃) δ ppm 1.58-1.72 (m, 2H), 1.98-2.08 (m, 2H), 2.76 (m, 2H), 3.08 (s, 3H), 3.12-3.22 (m, 2H), 3.59 (m, 1H), 4.63 (s, 2H), 7.73-7.85 (m, 2H), 8.03 (m, 2H), 8.19 (m, 2H), 8.68 (m, 1H); LRESIMS for C₁₈H₂₂N₂O₃S m/z 347 (M+H)⁺.

### Example B74

### tert-Butyl 4-[(6-{4-[(methoxycarbonyl)amino]phenyl}pyridin-3-yl)methoxy]-piperidine-1-carboxylate

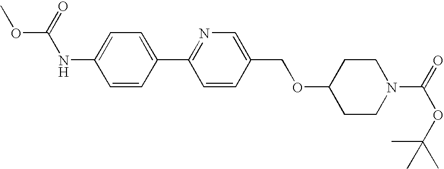

A mixture of tert-butyl 4-[(6-chloropyridin-3-yl)methoxy]piperidine-1-carboxylate (10 mg, 0.03 mmol), 4-(methoxycarbonylamino)phenylboronic acid (7 mg, 0.03 mmol), tetrakis(triphenylphosphine)palladium (2 mg, 0.001 mmol), K₂CO₃ (10 mg, 0.08 mmol) in dioxane (0.4 mL) and water (0.1 mL) was exposed to microwave irradiation (130° C.) for 20 minutes. The crude mixture was purified by preparative HPLC (System D). Pure fractions were combined, saturated aqueous NaHCO₃ was added and the resulting mixture was extracted with EtOAc. The organic phase was dried over Na₂SO₄, filtered and the solvent was evaporated giving the title compound. Yield: 8 mg (60%); Analytical HPLC: purity 97% (System A), purity 96% (System B); ¹H NMR (400 MHz, CDCl₃) δ ppm 1.45 (s, 9H) 1.54-1.67 (m, 2H) 1.86 (m, 2H) 3.06-3.16 (m, 2H) 3.53-3.64 (m, 1H) 3.72-3.83 (m, 5H) 4.58 (s, 2H) 7.48 (m, 2H) 7.64-7.75 (m, 2H) 7.95 (m, 1H) 7.95 (m, 1H) 8.60 (m, 1H); LRESIMS for C₂₄H₃₁N₃O₅ m/z 442 (M+H)⁺; HRESIMS, calc. monoiso mass (Da): 441.2264, found monoiso mass (Da): 441.2258.

### Example B75

### 5-[({1-[4-(4-Fluorophenyl)butanoyl]piperidin-4-yl}oxy)methyl]-2-[4-(methylsulfonyl)-phenyl]pyridine, trifluoroacetate

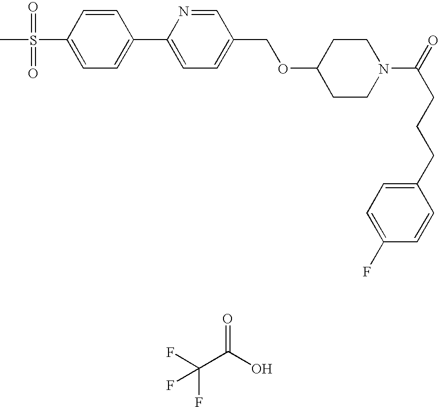

2-[4-(Methylsulfonyl)phenyl]-5-[(piperidin-4-yloxy)methyl]pyridine (24 mg, 0.07 mmol; Intermediate B15) and 4-(4-fluorophenyl)butanoic acid (15 mg, 0.084 mmol) were dissolved in DMF (0.7 mL) and triethylamine (0.02 mL, 0.14 mmol). TBTU (27 mg, 0.084 mmol) was added and the mixture was stirred at room temperature for 4 h. The reaction mixture was concentrated under reduced pressure. Preparative HPLC (System D) gave the title compound as its TFA-salt. Yield 27.5 mg (63%). Analytical HPLC: purity 99% (System A, R_{T}=2.21 min); ¹H NMR (400 MHz, CDCl₃) δ ppm 1.72 (m, 2H), 1.86-1.99 (m, 4H), 2.33-2.44 (m, 2H), 2.65 (m, 2H), 3.11 (s, 3H), 3.20-3.96 (m, 5H), 4.74 (m, 2H), 6.96 (m, 2H), 7.12 (m, 2H), 7.94 (m, 1H), 8.01-8.17 (m, 4H), 8.22 (m, 1H), 8.99 (m, 1H); LRESIMS for C₂₈H₃₁FN₂O₄S m/z 511 (M+H)⁺; HRESIMS, calc. monoiso mass (Da): 510.1989, found monoiso mass (Da): 510.1987.

### Example B76

### 5-({[1-(Cyclohexylacetyl)piperidin-4-yl]oxy}methyl)-2-[4-(methylsulfonyl)phenyl]-pyridine, trifluoroacetate

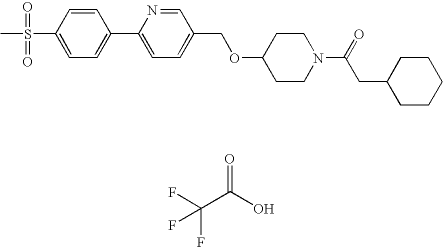

2-[4-(Methylsulfonyl)phenyl]-5-[(piperidin-4-yloxy)methyl]pyridine (24 mg, 0.07 mmol; Intermediate B15) and cyclohexylacetic acid (12 mg, 0.084 mmol) were dissolved in DMF (0.7 mL) and triethylamine (0.02 mL, 0.14 mmol). TBTU (27 mg, 0.084 mmol) was added and the mixture was stirred at room temperature for 4 h. The mixture was concentrated under reduced pressure. Preparative HPLC (System D) gave the final compound as its TFA-salt. Yield 35 mg (85%). Analytical HPLC: purity 99% (System A, R_{T}=2.21 min); ¹H NMR (400 MHz, CDCl₃) δ ppm 0.89-1.33 (m, 5H), 1.58-1.81 (m, 8H), 1.84-2.01 (m, 2H), 2.31 (d, 2H), 3.11 (s, 3H), 3.30-3.55 (m, 2H), 3.66-4.00 (m, 3H), 4.72 (m, 2H), 7.90 (m, 1H), 8.03-8.17 (m, 5H), 8.93 (m, 1H); LRESIMS for C₂₆H₃₄N₂O₄S m/z 471 (M+H)⁺; HRESIMS, calc. monoiso mass (Da): 470.2239, found monoiso mass (Da): 470.2243.

### Intermediate B16

### tert-Butyl 4-{[(6-chloropyridin-3-yl)oxy]methyl}piperidine-1-carboxylate

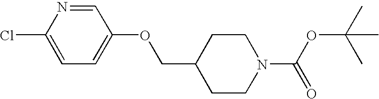

To a suspension of 2-chloro-5-hydroxypyridine (1.95 g, 15 mmol), tert-butyl 4-(hydroxymethyl)piperidine-1-carboxylate (3.23 g, 15 mmol), triphenylphosphine (3.93 g, 15 mmol) and THF (85 mL) was added 1,1′-azobis(N,N-dimethylformamide) (2.58 g, 15 mmol). The mixture was stirred at r.t. over the weekend and then filtered and evaporated. Purification of the residue was made by flash chromatography (gradient: 100% toluene→10% EtOAc/toluene). Yield 2.3 g (47%); Analytical HPLC: purity 99% (System A, R_{T}=2.64 min); LRESIMS for C₁₆H₂₃ClN₂O₃ m/z 327 (M+H)⁺.

### Example B77

### tert-Butyl 4-[({6-[4-(methylsulfonyl)phenyl]pyridin-3-yl}oxy)methyl]piperidine-1-carboxylate

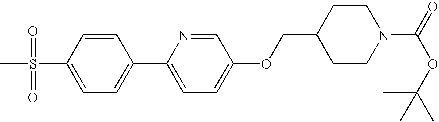

tert-Butyl 4-{[(6-chloropyridin-3-yl)oxy]methyl}piperidine-1-carboxylate (325 mg, 1.0 mmol; Intermediate B16), (4-methylsulfonyl)phenylboronic acid (220 mg, 1.1 mmol), Pd(PPh₃)₄ (58 mg. 0.05 mmol), K₂CO₃ (345 mg, 2.5 mmol) were mixed with dioxane (8 mL) and water (2 mL) and heated to 80° C. overnight. The mixture was filtered and the filtrate concentrated under reduced pressure. DCM and 5% aqueous NaHCO₃ were added to the residue and the organic phase was separated and dried. Flash chromatography on silica using MeOH/CHCl₃ (1:99) as eluent gave a fraction (48 mg, 94% pure) which was further purified by preparative HPLC (System E) affording the title compound. Yield: 11 mg (2%); Analytical HPLC: purity 100%, R_{T}=2.32 min (System A); ¹H NMR (400 MHz, CDCl₃) δ ppm 1.30 (m, 2H), 1.46 (s, 9H), 1.84 (m, 2H), 2.01 (m, 1H), 2.76 (m, 2H), 3.07 (m, 3H), 3.91 (d, J=6.5 Hz, 2H), 4.17 (br s, 2H), 7.29 (m, 1H), 7.72 (m, 1H), 8.00 (m, 2H), 8.13 (m, 2H), 8.40 (m, 1H); LRESIMS for C₂₃H₃₀N₂O₅S m/z 447 (M+H)⁺; HRESIMS, calc. monoiso mass (Da): 446.1875, found monoiso mass (Da): 446.1872.

### Intermediate B17

### 2-[4-(Methylsulfonyl)phenyl]-5-(piperidin-4-ylmethoxy)pyridine, trifluoroacetate

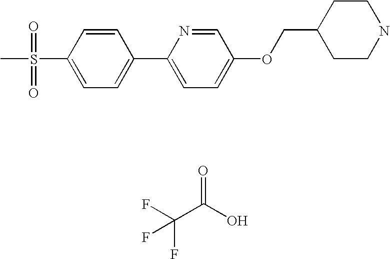

The title compound was prepared from tert-butyl 4-[({6-[4-(methylsulfonyl)phenyl]-pyridin-3-yl}oxy)methyl]piperidine-1-carboxylate (250 mg, 0.560 mmol) in accordance with the procedure described for Intermediate A5. Yield 250 mg (97%); Analytical HPLC: purity 100% (System A, R_{T}=1.20 min); ¹H NMR (400 MHz, CD₃OD) δ ppm 1.49-1.63 (m, 2H) 1.97-2.06 (m, 2H) 2.06-2.19 (m, 1H) 2.92-3.02 (m, 2H) 3.07 (s, 3H) 3.33-3.42 (m, 2H) 3.98 (d, J=6.0 Hz, 2H) 7.51 (m, 1H) 7.88 (m, 1H) 7.92-7.98 (m, 2H) 8.05-8.10 (m, 2H) 8.32 (m, 1H); LRESIMS for C₁₈H₂₂N₂O₃S m/z 347 (M+H)⁺; HRESIMS, calc. monoiso mass (Da): 346.1351, found monoiso mass (Da): 346.1346.

### Example B78

### Isobutyl 4-[({6-[4-(methylsulfonyl)phenyl]pyridin-3-yl}oxy)methyl]piperidine-1-carboxylate

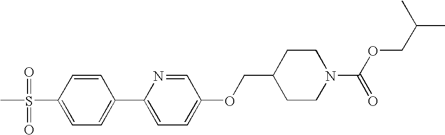

The title compound was prepared from Intermediate B17 and isobutyl chlorodicarbonate in accordance with the procedure described for Example A2. Yield 3.1 mg (16%); Analytical HPLC: purity 94% (System A, R_{T}=2.32 min); ¹H NMR (400 MHz, CDCl₃) δ ppm 0.97 (d, J=6.78 Hz, 6H) 1.30-1.43 (m, 2H) 1.86-1.93 (m, 2H) 1.93-2.01 (m, 1H) 2.01-2.13 (m, 1H) 2.78-2.93 (m, 2H) 3.11 (s, 3H) 3.90 (d, J=6.5 Hz, 2H) 3.94 (d, J=6.3 Hz, 2H) 4.17-4.37 (m, 2H) 7.31 (m, 1H) 7.76 (m, 1H) 8.00-8.06 (m, 2H) 8.13-8.19 (m, 2H) 8.43 (m, 1H); LRESIMS for C₂₃H₃₀N₂O₅S m/z 447 (M+H)⁺; HRESIMS, calc. monoiso mass (Da): 446.1875, found monoiso mass (Da): 446.1872.

### Example B79

### Ethyl 4-[({6-[4-(methylsulfonyl)phenyl]pyridin-3-yl}oxy)methyl]piperidine-1-carboxylate

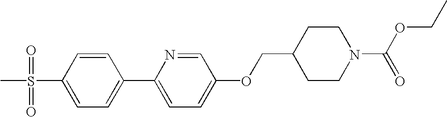

The title compound was prepared from Intermediate B17 and ethyl chlorodicarbonate in accordance with the procedure described for Example A2. Yield 4.4 mg (24%); Analytical HPLC: purity 92% (System A, R_{T}=2.04 min); ¹H NMR (400 MHz, CDCl₃) δ ppm 1.29 (t, J=7.0 Hz, 3H) 1.32-1.42 (m, 2H) 1.85-1.93 (m, 2H) 1.99-2.13 (m, 1H) 2.78-2.91 (m, 2H) 3.11 (s, 3H) 3.94 (d, J=6.3 Hz, 2H) 4.17 (q, J=7.1 Hz, 2H) 4.21-4.34 (m, 2H) 7.31 (m, 1H) 7.74-7.78 (m, 1H) 8.00-8.06 (m, 2H) 8.13-8.19 (m, 2H) 8.43 (m, 1H); LRESIMS for C₂₁H₂₆N₂O₅S m/z 419 (M+H)⁺; HRESIMS, calc. monoiso mass (Da): 418.1562, found monoiso mass (Da): 418.1559.

### Example B80

### tert-Butyl 4-{[(6-{4-[(2-hydroxyethyl)sulfonyl]phenyl}pyridin-3-yl)oxy]methyl}-piperidine-1-carboxylate

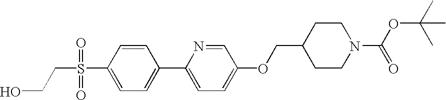

A suspension of 2-[(4-bromophenyl)sulfonyl]ethanol (prepared using similar conditions as described in Verhart, C. G. J et al., New base-labile amino-protective groups for peptide synthesis; Rec. Trav. Chim. Pays-Bas. 1988, 107(11), 621-6) (27 mg, 0.1 mmol), bis(neopentyl glycolato)diboron (34 mg, 0.15 mmol), potassium acetate (29 mg, 0.3 mmol), PdCl₂(dppf).DCM (4 mg, 0.005 mmol) and DME (2 mL) was heated at 90° C. for 1 hour. To the mixture were then added sodium hydrogen carbonate (27 mg, 0.2 mmol), Pd(PPh₃)₄ (12 mg, 0.01 mmol), tert-butyl 4-{[(6-chloropyridin-3-yl)oxy]methyl}-piperidine-1-carboxylate (32 mg, 0.1 mmol, Intermediate B16) and water (1 mL). The mixture was heated at 90° C. overnight, concentrated and purified by preparative HPLC (System E, gradient 30-60% MeCN). Yield 9 mg (19%); Analytical HPLC: purity 99% (System A, R_{T}=2.24 min); ¹H NMR (400 MHz, CDCl₃) δ ppm 1.27-1.41 (m, 2H) 1.50 (s, 9H) 1.83-1.91 (m, 2H) 1.98-2.10 (m, 1H) 2.73-2.86 (m, 2H) 3.38-3.43 (m, 2H) 3.94 (d, J=6.3 Hz, 2H) 4.02-4.07 (m, 2H) 4.14-4.28 (m, 2H) 7.31 (m, 1H) 7.76 (m, 1H) 8.02 (m, 2H) 8.18 (m, 2H) 8.43 (m, 1H); LRESIMS for C₂₄H₃₂N₂O₆S m/z 477 (M+H)⁺; HRESIMS, calc. monoiso mass (Da): 476.1981, found monoiso mass (Da): 476.1976.

### Example B81

### 4-(5-{[1-(tert-Butoxycarbonyl)piperidin-4-yl]methoxy}pyridin-2-yl)benzoic acid, ammoniate

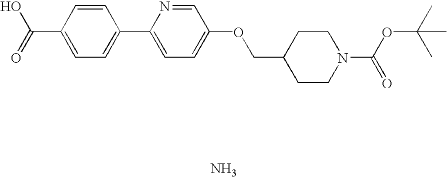

The title compound was prepared from Intermediate B16 (1.84 mmol) and 4-(4,4,5,5-tetramethyl-1,3,2-dioxaborolan-2-yl)benzoic acid in accordance with the procedure described for Example B88. Yield: 243 mg (31%); Analytical HPLC: purity 99% (System A, R_{T}=2.16 min); ¹H NMR (400 MHz, CD₃OD) δ ppm 1.13-1.28 (m, 2H) 1.31-1.40 (m, 9H) 1.72-1.81 (m, 2H) 1.88-2.02 (m, 1H) 2.62-2.83 (m, 2H) 3.88 (d, J=6.3 Hz, 2H) 4.04 (d, J=13.3 Hz, 2H) 7.37 (m, 1H) 7.72 (m, 1H) 7.76-7.82 (m, 2H) 7.90-7.96 (m, 2H) 8.18-8.21 (m, 1H); LRESIMS for C₂₃H₂₈N₂O₅ m/z 413 (M+H)⁺; HRESIMS, calc. monoiso mass (Da): 412.1998, found monoiso mass (Da): 412.1998.

### Example B82

### tert-Butyl 4-{[(6-{3-fluoro-4-[(propylamino)carbonyl]phenyl}pyridin-3-yl)oxy]-methyl}piperidine-1-carboxylate

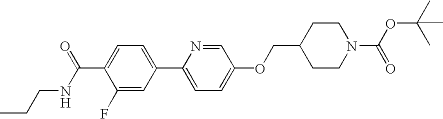

The title compound was prepared from Intermediate B16 (0.092 mmol) and {3-fluoro-4-[(propylamino)carbonyl]phenyl}boronic acid in accordance with the procedure described for Example A1. Yield 6.2 mg (14%); Analytical HPLC: purity 100% (System A, R_{T}=2.59 min); ¹H NMR (400 MHz, CDCl₃) δ ppm 1.03 (t, J=7.4 Hz, 3H) 1.26-1.40 (m, 2H) 1.49 (s, 9H) 1.64-1.75 (m, 2H) 1.82-1.91 (m, 2H) 1.96-2.10 (m, 1H) 2.72-2.86 (m, 2H) 3.45-3.53 (m, 2H) 3.93 (d, J=6.3 Hz, 2H) 4.13-4.30 (m, 2H) 6.76-6.87 (m, 1H) 7.28 (m, 1H) 7.72 (m, 1H) 7.78-7.80 (m, 1H) 7.80-7.84 (m, 1H) 8.16-8.22 (m, 1H) 8.40 (m, 1H); LRESIMS for C₂₆H₃₄FN₃O₄ m/z 472 (M+H)⁺; HRESIMS, calc. monoiso mass (Da): 471.2533, found monoiso mass (Da): 471.2538.

### Example B83

### tert-Butyl 4-{[(6-{4-[(cyclopropylamino)carbonyl]phenyl}pyridin-3-yl)oxy]methyl}-piperidine-1-carboxylate

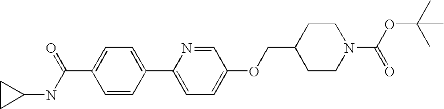

The title compound was prepared in from Intermediate B16 (0.061 mmol) and {4-[(cyclopropylamino)carbonyl]phenyl}boronic acid accordance with the procedure described for Example A1. Yield 3.3 mg (12%); Analytical HPLC: purity 100% (System A, R_{T}=2.22 min); ¹H NMR (400 MHz, CDCl₃) δ ppm 0.54-0.62 (m, 2H) 0.78-0.86 (m, 2H) 1.15-1.31 (m, 2H) 1.37-1.43 (m, 9H) 1.73-1.83 (m, 2H) 1.87-2.00 (m, 1H) 2.63-2.76 (m, 2H) 2.81-2.91 (m, 1H) 3.83 (d, J=6.3 Hz, 2H) 4.04-4.18 (m, 2H) 7.16-7.22 (m, 1H) 7.63 (m, 1H) 7.74 (m, 2H) 7.92 (m, 2H) 8.31 (m, 1H); LRESIMS for C₂₆H₃₃N₃O₄ m/z 452 (M+H)⁺; HRESIMS, calc. monoiso mass (Da): 451.2471, found monoiso mass (Da): 451.2497

### Example B84

### tert-Butyl 4-{[(6-{4-[(ethylamino)carbonyl]phenyl}pyridin-3-yl)oxy]methyl}-piperidine-1-carboxylate

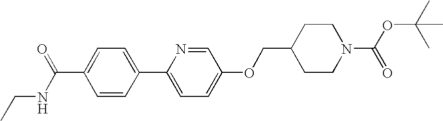

The title compound was prepared from Intermediate B16 (0.061 mmol) and {4-[(ethylamino)carbonyl]phenyl}boronic acid in accordance with the procedure described for Example A1. Yield 3.9 mg (15%); Analytical HPLC: purity 100% (System A, R_{T}=2.20 min); ¹H NMR (400 MHz, CDCl₃) δ ppm 1.21 (t, J=7.3 Hz, 3H) 1.21-1.30 (m, 2H) 1.40 (s, 9H) 1.74-1.82 (m, 2H) 1.87-2.00 (m, 1H) 2.63-2.76 (m, 2H) 3.41-3.50 (m, 2H) 3.83 (d, J=6.27 Hz, 2H) 4.04-4.20 (m, 2H) 7.19 (m, 1H) 7.63 (m, 1H) 7.77 (m, 2H) 7.93 (m, 2H) 8.31 (m, 1H); LRESIMS for C₂₅H₃₃N₃O₄ m/z 440 (M+H)⁺; HRESIMS, calc. monoiso mass (Da): 439.2471, found monoiso mass (Da): 439.2472.

### Example B85

### tert-Butyl 4-{[(6-{4-[(methylamino)carbonyl]phenyl}pyridin-3-yl)oxy]methyl}-piperidine-1-carboxylate

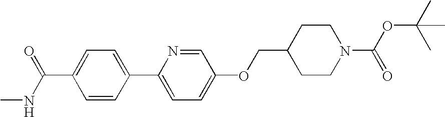

The title compound was prepared from Intermediate B16 (0.061 mmol) and {4-[(methylamino)carbonyl]phenyl}boronic acid in accordance with the procedure described for Example A1. Yield 6.4 mg (25%); Analytical HPLC: purity 100% (System A, R_{T}=2.10 min); LRESIMS for C₂₄H₃₁N₃O₄ m/z 426 (M+H)⁺; HRESIMS, calc. monoiso mass (Da): 425.2315, found monoiso mass (Da): 425.2323.

### Example B86

### tert-Butyl 4-({[6-(4-{[(2-cyanoethyl)amino]carbonyl}phenyl)pyridin-3-yl]oxy}methyl)-piperidine-1-carboxylate

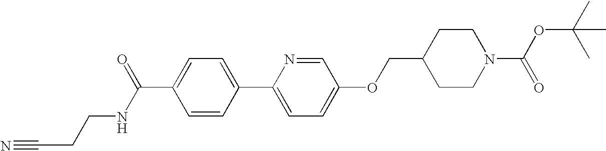

The title compound was prepared from Intermediate B16 (0.061 mmol) and (4-{[(2-cyanoethyl)amino]carbonyl}phenyl)boronic acid in accordance with the procedure described for Example A1. Yield 7 mg (25%); Analytical HPLC: purity 100% (System A, R_{T}=2.15 min); LRESIMS for C₂₆H₃₂N₄O₄ m/z 465 (M+H)⁺; HRESIMS, calc. monoiso mass (Da): 464.2424, found monoiso mass (Da): 464.2439.

### Example B87

### tert-Butyl 4-[({6-[4-(5,6-dihydro-4H-1,3-oxazin-2-yl)phenyl]pyridin-3-yl}oxy)methyl]-piperidine-1-carboxylate

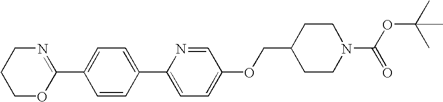

The title compound was prepared from Intermediate B16 (0.061 mmol) and [4-(5,6-dihydro-4H-1,3-oxazin-2-yl)phenyl]boronic acid in accordance with the procedure described for Example A1. Yield 1.3 mg (5%); Analytical HPLC: purity 94% (System A, R_{T}=2.13 min); LRESIMS for C₂₆H₃₃N₃O₄ m/z 452 (M+H)⁺; HRESIMS, calc. monoiso mass (Da): 451.2471, found monoiso mass (Da): 451.2479

### Example B88

### tert-Butyl 4-[({6-[4-(acetylamino)phenyl]pyridin-3-yl}oxy)methyl]piperidine-1-carboxylate

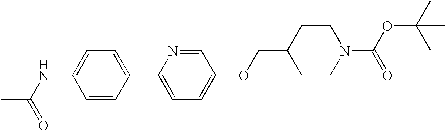

To a reaction tube containing tert-butyl 4-{[(6-chloropyridin-3-yl)oxy]methyl}piperidine-1-carboxylate (20 mg, 0.061 mmol; Intermediate B16) was added N-[4-(4,4,5,5-tetramethyl-1,3,2-dioxaborolan-2-yl)phenyl]acetamide (19.3 mg, 0.074 mmol), Pd(PPh₃)₄ (7.0 mg, 0.0061 mmol), potassium carbonate (21 mg, 0.15 mmol), 1,4-dioxane (2.5 mL) and water (1 mL). The suspension was heated in a Stemblock at 95° C. overnight. The reaction mixture was filtered and concentrated under reduced pressure. The crude product was purified by preparative HPLC (System E, gradient 30-60% MeCN). Yield 20 mg (77%); Analytical HPLC: purity 100% (System A, R_{T}=1.99 min); ¹H NMR (400 MHz, CD₃OD) δ ppm 1.25-1.41 (m, 2H) 1.49 (s, 9H) 1.84-1.92 (m, 2H) 1.99-2.13 (m, 1H) 2.16 (s, 3H) 2.77-2.93 (m, 2H) 3.99 (d, J=6.3 Hz, 2H) 4.11-4.21 (m, 2H) 7.47 (m, 1H) 7.67 (m, 2H) 7.77 (m, 1H) 7.85 (m, 2H) 8.28 (m, 1H); LRESIMS for C₂₄H₃₁N₃O₄ m/z 426 (M+H)⁺; HRESIMS, calc. monoiso mass (Da): 425.2315, found monoiso mass (Da): 425.2312.

### Example B89

### tert-Butyl 4-({[6-(4-{[(2-methoxyethyl)amino]carbonyl}phenyl)pyridin-3-yl]oxy}-methyl)piperidine-1-carboxylate

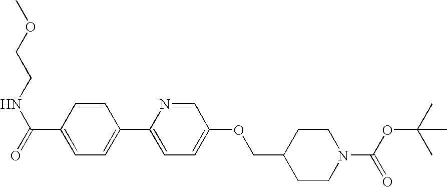

A solution of 4-(5-{[1-(tert-butoxycarbonyl)piperidin-4-yl]methoxy}pyridin-2-yl)benzoic acid ammoniate (20 mg, 0.047 mmol; obtained in Example B81), (2-methoxyethyl)amine (4.8 μL, 0.056 mmol), HOBT (13 mg, 0.094 mmol) and EDC (11 mg, 0.094 mmol), triethylamine (25 μL, 0.19 mmol) in methanol (1.5 mL) was stirred at r.t. over weekend. Additional (2-methoxyethyl)amine (4.8 μL, 0.056 mmol), HOBT (13 mg, 0.094 mmol) EDC (11 mg, 0.094 mmol) and triethylamine (25 μL, 0.19 mmol) were added. The reaction mixture was stirred at r.t. overnight. The mixture was concentrated under reduced pressure and the residue was purified by preparative HPLC (System D). Yield 6.3 mg (24%); Analytical HPLC: purity 100% (System A, R_{T}=2.07 min); ¹H NMR (400 MHz, CDCl₃) δ ppm 1.25-1.40 (m, 2H) 1.49 (s, 9H) 1.82-1.91 (m, 2H) 1.96-2.09 (m, 1H) 2.72-2.85 (m, 2H) 3.43 (s, 3H) 3.58-3.64 (m, 2H) 3.67-3.73 (m, 2H) 3.92 (d, J=6.3 Hz, 2H) 4.12-4.28 (m, 2H) 6.55-6.61 (m, 1H) 7.25-7.31 (m, 1H) 7.72 (m, 1H) 7.86-7.92 (m, 2H) 8.00-8.05 (m, 2H) 8.41 (m, 1H); LRESIMS for C₂₆H₃₅N₃O₅ m/z 470 (M+H)⁺; HRESIMS, calc. monoiso mass (Da): 469.2577, found monoiso mass (Da): 469.2588.

### Example B90

### tert-Butyl 4-({[6-(4-{[(2-hydroxyethyl)amino]carbonyl}phenyl)pyridin-3-yl]oxy}-methyl)piperidine-1-carboxylate

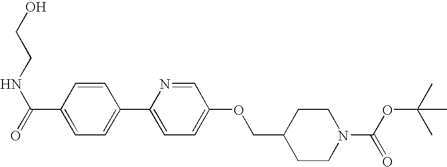

The title compound was prepared from Example B81 (0.047 mmol) and 2-aminoethanol in accordance with the procedure described for Example B89. Yield 1.2 mg (5%); Analytical HPLC: purity 94% (System A, R_{T}=1.90 min); LRESIMS for C₂₅H₃₃N₃O₅ m/z 456 (M+H)⁺; HRESIMS, calc. monoiso mass (Da): 455.2420, found monoiso mass (Da): 455.2428.

### Example B91

### tert-Butyl 4-({[6-(4-{[(2-hydroxybutyl)amino]carbonyl}phenyl)pyridin-3-yl]oxy}-methyl)piperidine-1-carboxylate

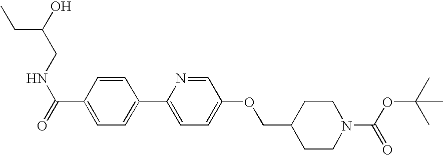

The title compound was prepared from Example B81 (0.047 mmol) and 1-aminobutan-2-ol in accordance with the procedure described for Example B89. Yield 1.3 mg (5%); Analytical HPLC: purity 100% (System A, R_{T}=2.04 min); LRESIMS for C₂₇H₃₇N₃O₅ m/z 484 (M+H)⁺; HRESIMS, calc. monoiso mass (Da): 483.2733, found monoiso mass (Da): 483.2737.

### Example B92

### tert-Butyl 4-{[(6-{4-[(acetylamino)methyl]phenyl}pyridin-3-yl)oxy]methyl}piperidine-1-carboxylate

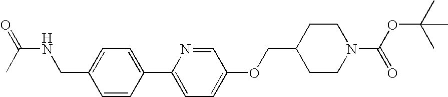

The title compound was prepared from Intermediate B16 (0.061 mmol) and {4-[(acetylamino)methyl]phenyl}boronic acid in accordance with the procedure described for Example A1. Yield 8.1 mg (30%); Analytical HPLC: purity 100% (System A, R_{T}=1.96 min); ¹H NMR (400 MHz, CDCl₃) δ ppm 1.24-1.39 (m, 2H) 1.49 (s, 9H) 1.82-1.90 (m, 2H) 1.96-2.04 (m, 1H) 2.06 (s, 3H) 2.71-2.85 (m, 2H) 3.91 (d, J=6.3 Hz, 2H) 4.12-4.27 (m, 2H) 4.50 (d, J=5.8 Hz, 2H) 7.26 (m, 1H) 7.38 (m, 2H) 7.66 (m, 1H) 7.91 (m, 2H) 8.37 (m, 1H); LRESIMS for C₂₅H₃₃N₃O₄ m/z 440 (M+H)⁺; HRESIMS, calc. monoiso mass (Da): 439.2471, found monoiso mass (Da): 439.2464.

### Example B93

### tert-Butyl 4-{[(6-{4-[(4-methylpiperazin-1-yl)carbonyl]phenyl}pyridin-3-yl)oxy]-methyl}piperidine-1-carboxylate

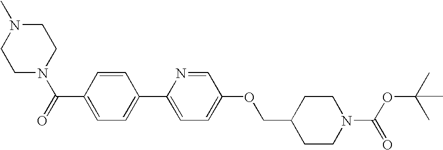

The title compound was prepared from Example B81 (0.047 mmol) and 1-methylpiperazine in accordance with the procedure described for Example B89. Yield 2.1 mg (8%); Analytical HPLC: purity 100% (System A, R_{T}=1.82 min); ¹H NMR (400 MHz, CDCl₃) δ ppm 1.14-1.32 (m, 2H) 1.40 (s, 9H) 1.72-1.83 (m, 2H) 1.86-2.00 (m, 1H) 2.21-2.51 (m, 7H) 2.62-2.77 (m, 2H) 3.35-3.52 (m, 2H) 3.65-3.80 (m, 2H) 3.83 (d, J=6.3 Hz, 2H) 4.03-4.19 (m, 2H) 7.15-7.22 (m, 1H) 7.39-7.45 (m, 2H) 7.60 (m, 1H) 7.86-7.92 (m, 2H) 8.31 (m, 1H); LRESIMS for C₂₈H₃₈N₄O₄ m/z 495 (M+H)⁺; HRESIMS, calc. monoiso mass (Da): 494.2893, found monoiso mass (Da): 494.2898.

### Example B94

### tert-Butyl 4-({[6-(4-{[2-(hydroxymethyl)morpholin-4-yl]carbonyl}phenyl)pyridin-3-yl]oxy}methyl)piperidine-1-carboxylate

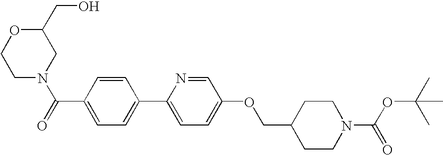

The title compound was prepared from Example B81 (0.047 mmol) and morpholin-2-ylmethanol in accordance with the procedure described for Example B89. Yield 5 mg (10%); Analytical HPLC: purity 100% (System A, R_{T}=1.98 min); ¹H NMR (400 MHz, CDCl₃) δ ppm 1.16-1.31 (m, 2H) 1.40 (s, 9H) 1.72-1.82 (m, 2H) 1.87-1.99 (m, 1H) 2.63-2.77 (m, 2H) 2.82-3.26 (m, 2H) 3.39-3.69 (m, 6H) 3.83 (d, J=6.3 Hz, 2H) 4.04-4.19 (m, 2H) 4.34-4.63 (m, 1H) 7.17-7.22 (m, 1H) 7.40-7.44 (m, 2H) 7.58-7.62 (m, 1H) 7.88-7.93 (m, 2H) 8.31 (m, 1H); LRESIMS for C₂₈H₃₇N₃O₆ m/z 512 (M+H)⁺; HRESIMS, calc. monoiso mass (Da): 511.2682, found monoiso mass (Da): 511.2700.

### Example B95

### tert-Butyl 4-({[6-(4-{[(2-amino-2-oxoethyl)amino]carbonyl}phenyl)pyridin-3-yl]oxy}-methyl)piperidine-1-carboxylate

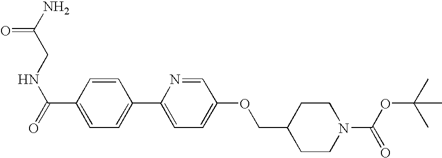

The title compound was prepared from Example B81 (0.047 mmol) and glycinamide in accordance with the procedure described for Example B89. Yield 2 mg (8%); Analytical HPLC: purity 100% (System A, R_{T}=1.86 min); LRESIMS for C₂₅H₃₂N₄O₅ m/z 469 (M+H)⁺; HRESIMS, calc. monoiso mass (Da): 468.2373, found monoiso mass (Da): 468.2393.

### Example B96

### 4-{[4-(5-{[1-(tert-Butoxycarbonyl)piperidin-4-yl]methoxy}pyridin-2-yl)phenyl]-amino}-4-oxobutanoic acid

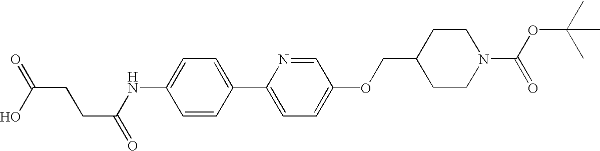

The title compound was prepared from Intermediate B16 (0.061 mmol) and 4-{[4-(dihydroxyboryl)phenyl]amino}-4-oxobutanoic acid in accordance with the procedure described for Example A1. Yield 12 mg (41%); Analytical HPLC: purity 100% (System A, R_{T}=1.94 min); ¹H NMR (400 MHz, CD₃OD) δ ppm 1.24-1.39 (m, 2H) 1.49 (s, 9H) 1.83-1.93 (m, 2H) 1.99-2.13 (m, 1H) 2.58-2.73 (m, 4H) 2.76-2.93 (m, 2H) 3.98 (d, J=6.3 Hz, 2H) 4.10-4.21 (m, 2H) 7.46 (m, 1H) 7.68 (m, 2H) 7.76 (m, 1H) 7.84 (m, 2H) 8.27 (m, 1H); LRESIMS for C₂₆H₃₃N₃O₆ m/z 484 (M+H)⁺; HRESIMS, calc. monoiso mass (Da): 483.2369, found monoiso mass (Da): 483.2364.

### Example B97

### tert-Butyl 4-[({6-[4-(cyanomethyl)phenyl]pyridin-3-yl}oxy)methyl]piperidine-1-carboxylate

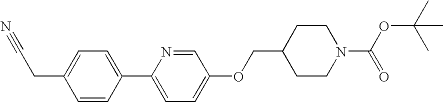

The title compound was prepared from Intermediate B16 (0.061 mmol) and [4-(cyanomethyl)phenyl]boronic acid in accordance with the procedure described for Example A1. Yield 2.5 mg (10%); Analytical HPLC: purity 96% (System A, R_{T}=2.30 min); LRESIMS for C₂₄H₂₉N₃O₃ m/z 408 (M+H)⁺; HRESIMS, calc. monoiso mass (Da): 407.2209, found monoiso mass (Da): 407.2211.

### Example B98

### tert-Butyl 4-[({6-[4-(methylsulfinyl)phenyl]pyridin-3-yl}oxy)methyl]piperidine-1-carboxylate

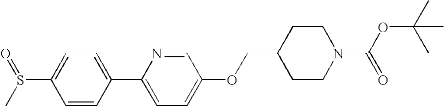

The title compound was prepared from Intermediate B16 (0.061 mmol) and [4-(methylsulfinyl)phenyl]boronic acid in accordance with the procedure described for Example A1. Yield 5.6 mg (21%); Analytical HPLC: purity 100% (System A, R_{T}=2.17 min); ¹H NMR (400 MHz, CDCl₃) δ ppm 1.18-1.31 (m, 2H) 1.40 (s, 9H) 1.51-1.54 (m, 1H) 1.74-1.82 (m, 2H) 1.89-1.99 (m, 1H) 2.64-2.75 (m, 1H) 2.69 (s, 3H) 3.84 (d, J=6.3 Hz, 2H) 4.03-4.19 (m, 2H) 7.18-7.22 (m, 1H) 7.61-7.68 (m, 3H) 8.02 (m, 2H) 8.32 (m, 1H); LRESIMS for C₂₃H₃₀N₂O₄S m/z 431 (M+H)⁺; HRESIMS, calc. monoiso mass (Da): 430.1926, found monoiso mass (Da): 430.1924.

### Example B99

### tert-Butyl 4-{[(6-{3-[(acetylamino)methyl]phenyl}pyridin-3-yl)oxy]methyl}piperidine-1-carboxylate

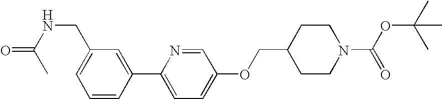

The title compound was prepared from Intermediate B16 (0.061 mmol) and {3-[(acetylamino)methyl]phenyl}boronic acid in accordance with the procedure described for Example A1. Yield 8.5 mg (32%); Analytical HPLC: purity 96% (System A, R_{T}=2.00 min); LRESIMS for C₂₅H₃₃N₃O₄ m/z 440 (M+H)⁺; HRESIMS, calc. monoiso mass (Da): 439.2471, found monoiso mass (Da): 439.2473.

### Example B100

### tert-Butyl 4-[({6-[3-(cyanomethyl)phenyl]pyridin-3-yl}oxy)methyl]piperidine-1-carboxylate

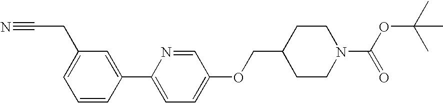

The title compound was prepared from Intermediate B16 (0.061 mmol) and [3-(cyanomethyl)phenyl]boronic acid in accordance with the procedure described for Example A1. Yield 4.1 mg (33%); Analytical HPLC: purity 100% (System A, R_{T}=2.36 min); LRESIMS for C₂₄H₂₉N₃O₃ m/z 408 (M+H)⁺; HRESIMS, calc. monoiso mass (Da): 407.2209, found monoiso mass (Da): 407.2208.

### Example B100

### tert-Butyl 4-[({6-[2-fluoro-4-(methylsulfonyl)phenyl]pyridin-3-yl}oxy)methyl]-piperidine-1-carboxylate

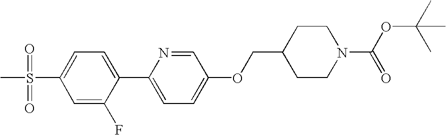

The title compound from Intermediate B16 (0.092 mmol) and [2-fluoro-4-(methylsulfonyl)phenyl]boronic acid was prepared in accordance with the procedure described for Example A1. Yield 1.8 mg (4%); Analytical HPLC: purity 98% (System A, R_{T}=2.47 min); LRESIMS for C₂₃H₂₉FN₂O₅S m/z 465 (M+H)⁺; HRESIMS, calc. monoiso mass (Da): 464.1781, found monoiso mass (Da): 464.1774.

### Example B102

### tert-Butyl 4-[({6-[4-(aminocarbonyl)-3-fluorophenyl]pyridin-3-yl}oxy)methyl]-piperidine-1-carboxylate

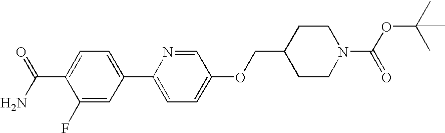

The title compound was prepared from Intermediate B16 (0.092 mmol) and [4-(aminocarbonyl)-3-fluorophenyl]boronic acid in accordance with the procedure described for Example A1. Yield 0.9 mg (2%); Analytical HPLC: purity 100% (System A, R_{T}=2.27 min); LRESIMS for C₂₃H₂₈FN₃O₄ m/z 430 (M+H)⁺; HRESIMS, calc. monoiso mass (Da): 429.2064, found monoiso mass (Da): 429.2069.

### Example B103

### tert-Butyl 4-[({6-[4-(azetidin-1-ylsulfonyl)phenyl]pyridin-3-yl}oxy)methyl]piperidine-1-carboxylate

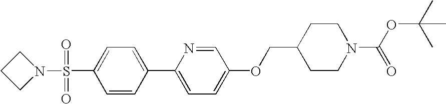

The title compound was prepared from Intermediate B16 (0.10 mmol) and 1-[(4-bromophenyl)sulfonyl]azetidine in accordance with the procedure described for Example B80. Yield 1.2 mg (2%); Analytical HPLC: purity 98% (System A, R_{T}=2.53 min); LRESIMS for C₂₅H₃₃N₃O₅S m/z 488 (M+H)⁺; HRESIMS, calc. monoiso mass (Da): 487.2141, found monoiso mass (Da): 487.2142.

### Example B104

### [4-(5-{[1-(tert-Butoxycarbonyl)piperidin-4-yl]methoxy}pyridin-2-yl)phenyl]acetic acid

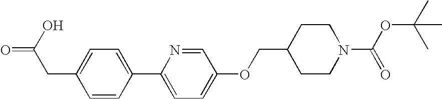

The title compound was prepared from Intermediate B16 (1.00 mmol) and (4-bromophenyl)acetic acid in accordance with the procedure described for Example B80. Yield 201 mg (47%); Analytical HPLC: purity 100% (System A, R_{T}=2.01 min); ¹H NMR (400 MHz, CD₃OD) δ ppm 1.25-1.39 (m, 2H) 1.49 (s, 9H) 1.84-1.93 (m, 2H) 2.00-2.13 (m, 1H) 2.77-2.93 (m, 2H) 3.56 (s, 2H) 3.99 (d, J=6.3 Hz, 2H) 4.11-4.21 (m, 2H) 7.42 (m, 2H) 7.47 (m, 1H) 7.75-7.83 (m, 3H) 8.27 (m, 1H); LRESIMS for C₂₄H₃₀N₂O₅ m/z 427 (M+H)⁺; HRESIMS, calc. monoiso mass (Da): 426.2155, found monoiso mass (Da): 426.2142.

### Example B105

### tert-Butyl 4-{[(6-{4-[2-(4-hydroxypiperidin-1-yl)-2-oxoethyl]phenyl}pyridin-3-yl)oxy]-methyl}piperidine-1-carboxylate

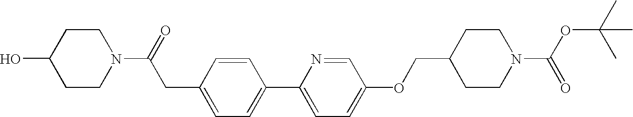

The title compound was prepared from piperidin-4-ol and [4-(5-{[1-(tert-butoxycarbonyl)-piperidin-4-yl]methoxy}pyridin-2-yl)phenyl]acetic acid (Example B104; 0.070 mmol) using similar conditions as described for Example B89. Yield 2.1 mg (6%); Analytical HPLC: purity 99% (System A, R_{T}=1.92 min); LRESIMS for C₂₉H₃₉N₃O₅ m/z 510 (M+H)⁺; HRESIMS, calc. monoiso mass (Da): 509.2890, found monoiso mass (Da): 509.2892.

### Example B106

### tert-Butyl 4-({[6-(4-{2-[2-(hydroxymethyl)morpholin-4-yl]-2-oxoethyl}phenyl)pyridin-3-yl]oxy}methyl)piperidine-1-carboxylate

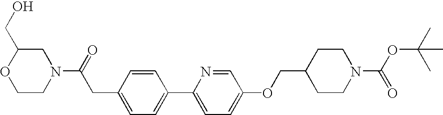

The title compound was prepared from morpholin-2-ylmethanol and [4-(5-{[1-(tertbutoxycarbonyl)piperidin-4-yl]methoxy}pyridin-2-yl)phenyl]acetic acid (Example B104; 0.070 mmol) using similar conditions as described for Example B89. Yield 1.8 mg (5%); Analytical HPLC: purity 98% (System A, R_{T}=1.90 min); LRESIMS for C₂₉H₃₉N₃O₆ m/z 526 (M+H)⁺; HRESIMS, calc. monoiso mass (Da): 525.2839, found monoiso mass (Da): 525.2850.

### Example B107

### tert-Butyl 4-{[(6-{4-[2-(3-hydroxyazetidin-1-yl)-2-oxoethyl]phenyl}pyridin-3-yl)oxy]-methyl}piperidine-1-carboxylate

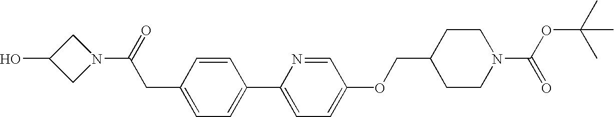

The title compound was prepared from azetidin-3-ol hydrochloride and [4-(5-{[1-(tertbutoxycarbonyl)piperidin-4-yl]methoxy}pyridin-2-yl)phenyl]acetic acid (Example B104; 0.070 mmol) using similar conditions as described for Example B89. Yield 0.6 mg (2%); Analytical HPLC: purity 100% (System A, R_{T}=1.88 min); LRESIMS for C₂₇H₃₅N₃O₅ M/Z 482 (M+H)⁺; HRESIMS, calc. monoiso mass (Da): 481.2577, found monoiso mass (Da): 481.2571.

### Intermediate B18

### 2-(4-{[(6-Chloropyridin-3-yl)oxy]methyl}piperidin-1-yl)pyrimidine

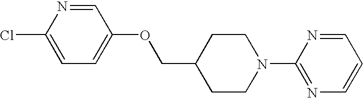

To a stirred suspension of 2-chloro-5-hydroxypyridine (194 mg, 1.5 mmol), [1-(2-pyrimidinyl)-4-piperidinyl]methanol (290 mg, 1.5 mmol) and triphenylphosphine (393 mg, 1.5 mmol) in THF (7 mL) was added 1,1′-azobis(N,N-dimethylformamide) (258 mg, 1.5 mmol). After 72 hours, the reaction mixture was filtered and concentrated under reduced pressure. The residue was purified by preparative HPLC (System E). Yield: 12 mg (3%); Analytical HPLC: purity 99% (System A, R_{T}=1.42 min); LRESIMS C₁₅H₁₇ClN₄O m/z=305 (M+H)⁺.

### Example B108

### 2-{4-[({6-[4-(Methylsulfonyl)phenyl]pyridin-3-yl}oxy)methyl]piperidin-1-yl}-pyrimidine

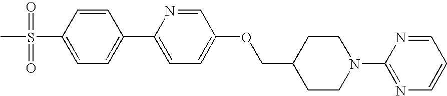

To a reaction tube containing 2-(4-{[6-chloropyridin-3-yl)oxy]methyl}piperidin-1-yl)-pyrimidine (7 mg, 0.023 mmol) was added (4-methylsulfonyl)phenylboronic acid (5 mg, 0.025 mmol), Pd(PPh₃)₄ (cat. amount), potassium carbonate (8 mg, 0.058 mmol), 1,4-dioxane (1 mL) and water (0.5 mL). The reaction mixture was heated in a Stemblock at 95° C. for 3 hours. The reaction mixture was concentrated under reduced pressure and the resulting residue was purified by preparative HPLC (System E). Yield: 1.5 mg (15%); Analytical HPLC: purity 99% (System A, R_{T}=1.70 min); LRESIMS C₂₂H₂₄N₄O₃S m/z=425 (M+H)⁺; HRESIMS, calc. monoiso mass (Da): 424.1569, found monoiso mass (Da): 424.1552.

### Example C1

### tert-Butyl 4-({6-[4-(methylsulfonyl)phenyl]pyridin-3-yl}acetyl)piperazine-1-carboxylate

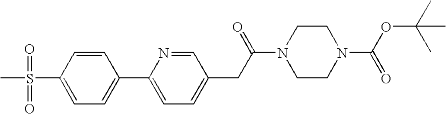

A mixture of (6-chloro-pyridin-3-yl)acetic acid (2.0 g, 11.66 mmol), tert-butyl piperazine-1-carboxylate (2.17 g, 11.66 mmol), HOBT (3.15 g, 23.31 mmol) and EDC (4.47 g, 23.31 mmol), in THF/MeOH (100 mL; 1:1) and triethylamine (8 mL) was stirred at r.t. overnight. The solvents were evaporated and the residue was partitioned between 1M HCl (75 mL) and chloroform (100 mL). The layers were separated and the water phase was extracted with chloroform (2×100 mL). The organic layers were combined, dried with MgSO₄ and evaporated to yield 3.81 g off-white solid. Part of the obtained intermediate (tert-butyl 4-[(6-chloropyridin-3-yl)acetyl]piperazine-1-carboxylate; 2.0 g, 5.89 mmol) was then heated for 3 hours at 95° C. with (4-methylsulfonylphenyl)boronic acid (1.24 g, 6.18 mmol), Pd(PPh₃)₄ (340 mg, 0.295 mmol), potassium carbonate (2.03 g, 14.7 mmol) in 1,4-dioxane (20 mL) and water (5 mL). The solvents were evaporated and the residue was partitioned between water (100 mL) and chloroform (100 mL). The layers were separated and the water phase was extracted with chloroform (2×100 mL). The combined organic layers were evaporated and the residue was purified by flash chromatography (gradient 100% DCM→50% EtOAc in DCM). Yield 1.71 g (61%); Analytical HPLC: purity 99% (System A, R_{T}=1.83 min); LRESIMS for C₂₃H₂₉N₃O₅S m/z 460 (M+H)⁺; HRESIMS, calc. monoiso mass (Da): 459.1828, found monoiso mass (Da): 459.1831.

### Example C2

### tert-Butyl 4-(2-{6-[4-(methylsulfonyl)phenyl]pyridin-3-yl}ethyl)piperazine-1-carboxylate

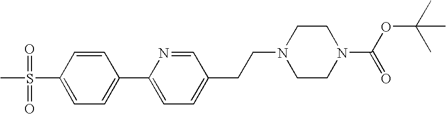

To a reaction tube containing tert-butyl 4-({6-[4-(methylsulfonyl)phenyl]pyridin-3-yl}-acetyl)piperazine-1-carboxylate (400 mg, 0.87 mmol; Example C1) and THF (5 mL) was added borane-methyl sulfide complex (1 mL of a 2M solution in THF, 2 mmol). The mixture was then heated at 70° C. for 1.5 h. Upon evaporation, methanol (10 mL) was added to the residue and heating was continued for 45 min at 80° C. The mixture was concentrated under reduced pressure and the resulting residue was purified by preparative HPLC (System E, gradient 30-70% MeCN). Yield 314 mg (81%); Analytical HPLC: purity 100% (System A, R_{T}=1.55 min); ¹H NMR (400 MHz, CDCl₃) δ ppm 1.40 (s, 9H) 2.37-2.47 (m, 4H) 2.54-2.63 (m, 2H) 2.76-2.84 (m, 2H) 3.02 (s, 3H) 3.36-3.45 (m, 4H) 7.57-7.63 (m, 1H) 7.64-7.69 (m, 1H) 7.94-7.99 (m, 2H) 8.09-8.14 (m, 2H) 8.51-8.54 (m, 1H); LRESIMS for C₂₃H₃₁N₃O₄S m/z 446 (M+H)⁺; HRESIMS, calc. monoiso mass (Da): 445.2035, found monoiso mass (Da): 445.2033.

### Intermediate C1

### 1-(2-{6-[4-(Methylsulfonyl)phenyl]pyridin-3-yl}ethyl)piperazine, bis(trifluoroacetate)

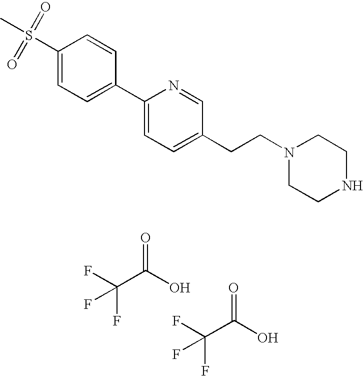

The title compound was prepared from tert-butyl 4-(2-{6-[4-(methylsulfonyl)phenyl]-pyridin-3-yl}ethyl)piperazine-1-carboxylate (obtained in Example C2) in accordance with the procedure described for Intermediate A5. Intermediate C1 was used without further purification in the synthesis of Example C3 and Example C4.

### Example C3

### 1-(4-Isopropoxybenzoyl)-4-(2-{6-[4-(methylsulfonyl)phenyl]pyridin-3-yl}ethyl)-piperazine, trifluoroacetate

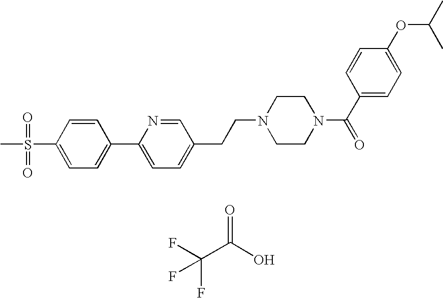

The title compound was prepared from Intermediate C1 (0.063 mmol) and 4-isopropoxybenzoic acid in accordance with the procedure described for Example A3. Yield 4.2 mg (11%); Analytical HPLC: purity 97% (System A, R_{T}=1.67 min); ¹H NMR (400 MHz, CD₃OD) δ ppm 1.35 (d, J=6.3 Hz, 6H) 3.19 (s, 3H) 3.21-3.28 (m, 4H) 3.34-3.43 (m, 4H) 3.48-3.58 (m, 4H) 4.67-4.75 (m, 1H) 7.00-7.05 (m, 2H) 7.46-7.51 (m, 2H) 7.95-7.99 (m, 1H) 8.02-8.06 (m, 1H) 8.08-8.12 (m, 2H) 8.25-8.29 (m, 2H) 8.69-8.71 (m, 1H); LRESIMS for C₂₈H₃₃N₃O₄S m/z 508 (M+H)⁺; HRESIMS, calc. monoiso mass (Da): 507.2192, found monoiso mass (Da): 507.2195.

### Example C4

### Isobutyl 4-(2-{6-[4-(methylsulfonyl)phenyl]pyridin-3-yl}ethyl)piperazine-1-carboxylate, trifluoroacetate

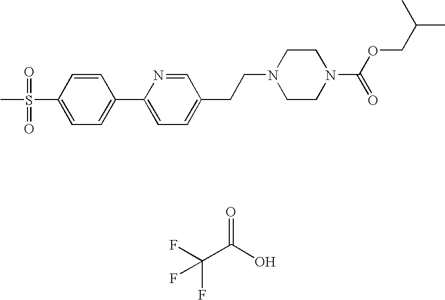

The title compound was prepared from Intermediate C1 (0.063 mmol) and isobutyl chlorodicarbonate in accordance with the procedure described for Example A2. Yield 16.6 mg (47%); Analytical HPLC: purity 100% (System A, R_{T}=1.57 min); ¹H NMR (400 MHz, CD₃OD) δ ppm 0.98 (d, J=6.8 Hz, 6H) 1.92-2.04 (m, 1H) 3.20 (s, 3H) 3.24-3.31 (m, 4H) 3.37-3.49 (m, 4H) 3.50-3.58 (m, 4H) 3.94 (d, J=6.53 Hz, 2H) 8.06-8.14 (m, 4H) 8.22-8.27 (m, 2H) 8.72-8.75 (m, 1H); LRESIMS for C₂₃H₃₁N₃O₄S m/z 446 (M+H)⁺; HRESIMS, calc. monoiso mass (Da): 445.2035, found monoiso mass (Da): 445.2037.

Biological Tests

Human GPR119 Activity Assay

Agonists to the human GPR119 receptor were characterized by measuring human GPR119 receptor-mediated stimulation of cyclic AMP (cAMP) in HEK 293 cells expressing the human GPR119 receptor.

Briefly, cAMP content was determined using a cAMP kit based on HTRF technology (Homogeneous Time-Resolved Fluorescence, Cisbio Cat. no. 62AM2PEC). HEK293 cells stably expressing the human GPR119 receptor (HEK293-hGPR119 cells) were cultured in DMEM (Gibco # 31966-021) supplemented with 10% Bovine Calf Serum (Hyclone # SH30072.03), and, 500 μg/mL Hygromycin B (Roche Diagnostics 843555). At 80% confluency, cells were detached using Trypsine and aliquoted at a density of 5×10⁶ cells/mL in freezing medium (DMEM (Gibco # 31966-021), 20% BCS (Hyclone # SH30072.03), 10% DMSO (Sigma #D2650) and stored at −135° C. On the experimental day, HEK293-hGPR119 cells were thawn and diluted to 0.4×10⁶ cells/mL in assay buffer (1×HBSS (Gibco Cat. no. 14025-049), 20 mM Hepes (Gibco Cat. no. 15630-056), 0.1% BSA, pH 7.4) and incubated with test substances for 20 min at room temperature. After addition of HTRF reagents diluted in lysis buffer, the 96- or 384-well plates were incubated 1 hour, followed by measuring the fluorescence ratio at 665 nm/620 nm. Test substances was diluted in compound buffer (1×HBSS (Gibco Cat. no. 14025-049), 20 mM Hepes (Gibco Cat. no. 15630-056), 0.1% BSA, 2 mM IBMX (Sigma-Aldrich Cat. No. 17018, pH 7.4). The potency of the agonist was quantified by determine the concentration that cause 50% activation of hGPR119 evoked increase in cAMP, EC₅₀.

Compounds of the invention showed a concentration-dependant increase in intracellular cAMP level and generally had an EC₅₀ value of <5 μM. Obtained EC₅₀ values for representative compounds of the present invention are shown in Table A.

TABLE A
Agonist potency at the human GPR119.

| Compound | EC₅₀ (μM) |
|---|---|
| EXAMPLE A2 | 0.110 |
| EXAMPLE A39 | 0.139 |
| EXAMPLE B2 | 0.022 |

Effects of GPR119 Modulators on Glucose-Stimulated Insulin Release
In Vitro Experiments

The effect of GPR119 modulators on glucose-stimulated insulin release is determined in isolated pancreatic islets from Wistar rats and diabetic rat models, e.g. GK rat. Briefly, islets are isolated from the rats by digestion with collagenase according to standard protocol. The islets are cultured for 24 h in RPMI-1640 medium supplemented with 11.1 mM glucose and 10% (vol/vol) fetal calf serum. On the experimental day, batches of three islets are preincubated in KRB (Krebs-Ringer bicarbonate) buffer and 3.3 mM glucose for 30 min, 37° C. Thereafter the batches with islets are incubated in 16.7 mM glucose and KRB buffer supplemented with vehicle or test compounds for 60 min at 37° C. Aliquots of the medium will be frozen for measurement of insulin using a radioimmunoassay with rabbit ant-porcine insulin antibodies.

In Vivo Experiments

The effects of GPR119 modulators on glucose stimulated insulin release is determined in diabetic mice models (e.g. Lep^{ob/ob} or diet-induced obese (DIO) mice) undergoing an oral glucose tolerance test. Briefly, overnight fasted mice is given either vehicle or test compound at desired doses via oral gavage. Based on the pharmacokinetic of the test compounds, a glucose boluse dose is delivered via oral gavage 30 min-2 hrs following the test compound. Plasma glucose and insulin levels are determined at desired time points over a 2 hour period using blood collection from tail nick. Plasma glucose is determined using a Glucometer and plasma insulin is determined using an insulin ELISA following blood collection in heparinated tubes and centrifugation.

Effects of GPR119 Modulators on GLP-1 Secretion and Body Weight

In Vivo Experiments

The effect of GPR119 modulators on body weight is determined in diabetic and obese mice models, e.g. Lep^{ob/ob} or diet-induced obese (DIO) mice. The food intake and body weight gain is measured during subchronic treatment with vehicle or test compound via oral gavage. At the end of the experiment, vena cava blood is collected and e.g. HbA1c, GLP-1, insulin, ALAT, ASAT are measured.

1. A compound of Formula (Ia) 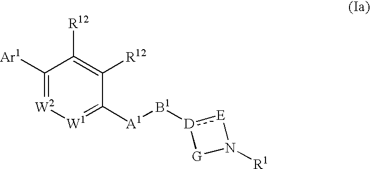
including pharmaceutically acceptable salts, hydrates, geometrical isomers, racemates, tautomers, optical isomers, and N-oxides thereof; wherein:
one of W¹ and W² is N and the other is CR¹²;
A¹ is CH₂, O, NR¹⁰, S, S(O) or S(O)₂;
B¹ is CH₂, O, NR¹⁰, S, S(O), S(O)₂, C(O) or CONR¹⁰, provided that when B¹ is O, NR¹⁰, S(O), S(O)₂, C(O) or CONR¹⁰, then A¹ is CH₂;
D is N, C or CR¹¹, provided that D must be CR¹¹ and said R¹¹ must be hydrogen or methyl when B¹ is selected from O, NR¹⁰, S, S(O), S(O)₂, and CONR¹⁰; [symbol]
is a single bond when D is N or CR¹¹ or a double bond when D is C;
E and G are independently C₁₋₃-alkylene, each optionally substituted with a substituent independently selected from the group consisting of C₁₋₃-alkyl, C₁₋₄-alkoxy, carboxy, fluoro-C₁₋₃-alkyl, hydroxy, hydroxymethyl, and fluoro, provided that the ring formed by D, E, N and G has not more than 7 ring atoms, and further provided that the said ring has 6 or 7 ring atoms when D is N, and yet further provided that the total number of substituents on E and G are not more than 2;
R¹ is C(O)OR², C(O)R², S(O)₂R², C(O)NR²R³ or —CH₂—C(O)NR²R³; or a 5- or 6-membered heteroaryl group linked via a ring carbon atom, wherein the said heteroaryl group is optionally substituted with C₁₋₄-alkyl;
Ar¹ is phenyl which is optionally substituted in one or more positions with a substituent independently selected from:
(a) CF₃SO₃,
(b) halogen selected from chlorine, bromine and fluorine,
(c) C₁₋₄-alkylsulfinyl,
(d) —S(O)₂R⁴,
(e) —S(O)₂NR⁵R⁵,
(f) —NR⁶S(O)₂R⁴,
(g) —CH₂—NR⁶C(O)R⁴,
(h) —NR⁶C(O)R⁴,
(i) —C(O)NR⁵R⁵,
(j) —CH₂—C(O)NR⁵R⁵,
(k) —C(O)R⁴,
(l) H₂N—C(O)O—,
(m) CH₃—NH—C(O)O—,
(n) (CH₃)₂NC(O)O—,
(o) CH₃OC(O)NH—,
(p) C-heterocyclyl, optionally substituted with C₁₋₄-alkyl,
(q) —CN,
(r) —OR⁸,
(s) —SCF₃,
(t) —NO₂,
(u) phosphonooxy,
(v) C-heterocyclylsulfonyl, optionally substituted with C₁₋₄-alkyl,
(w) —NR⁵R⁵,
(x) —C(OH)CH₃CF₃,
(y) [C(OH)CH₃CF₃]—C₁₋₆-alkyl,
(z) cyano-C₁₋₆-alkyl,
(aa) guanidino,
(bb) amidino,
(cc) C₁₋₆-alkyl,
(dd) C₁₋₄-alkoxy-C₁₋₄-alkyl,
(ee) fluoro-C₁₋₄-alkyl,
(ff) C₂₋₆-alkenyl,
(gg) fluoro-C₂₋₄-alkenyl,
(hh) hydroxy-C₁₋₆-alkyl,
(ii) C₁₋₄-alkylsulfonyl-C₁₋₄-alkyl,
(jj) hydroxy-C₂₋₄-alkoxy-C₁₋₄-alkyl,
(kk) C₂₋₃-acyl-C₁₋₃-alkyl,
(ll) C₂₋₆-alkynyl,
(mm) hydroxy-C₃₋₆-cycloalkyl,
(nn) fluoro-C₃₋₆-cycloalkyl,
(oo) methyl-C₃₋₆-cycloalkyl,
(pp) C-heterocyclylcarbonyl, optionally substituted with C₁₋₄-alkyl,
(qq) C₃₋₆-cycloalkyl,
(rr) C₃₋₆-cycloalkyl-C₁₋₄-alkyl,
(ss) R⁵R⁵N—C₁₋₂-alkyl,
(tt) —C(O)OR⁷,
(uu) —CH₂C(O)OR⁷,
(vv) aryl,
(ww) aryl-C₁₋₄-alkyl,
(xx) aryl-C₂₋₄-alkenyl,
(yy) aryl-C₂₋₄-alkynyl,
(zz) heteroaryl,
(aaa) heteroaryl-C₁₋₄-alkyl,
(bbb) heteroaryl-C₂₋₄-alkenyl, and
(ccc) heteroaryl-C₂₋₄-alkynyl,
wherein any aryl or heteroaryl residue, alone or as part of another group, as substituent on Ar¹ is optionally substituted in one or more positions with a substituent independently selected from the group Z¹ consisting of:
(a) halogen selected from chlorine and fluorine,
(b) C₁₋₄-alkyl,
(c) hydroxy,
(d) C₁₋₄-alkoxy,
(e) —OCF₃,
(f) —SCF₃,
(g) —CN,
(h) —C(OH)CH₃CF₃,
(i) hydroxy-C₁₋₄-alkyl,
(j) —CF₃,
(k) —S(O)₂CH₃,
(l) —S(O)₂NH₂,
(m) —S(O)₂NHCH₃,
(n) —S(O)₂N(CH₃)₂,
(o) —N(CH₃)S(O)₂CH₃,
(p) —N(CH₃)C(O)CH₃,
(q) —C(O)NH₂,
(r) —C(O)NHCH₃,
(s) —C(O)N(CH₃)₂,
(t) —C(O)CH₃,
(u) —NH₂,
(v) —NHCH₃,
(w) —N(CH₃)₂,
(x) —NO₂, and
(y) methoxycarbonyl;
R² is selected from:
(a) C₁₋₆-alkyl,
(b) C₁₋₆-alkoxy-C₂₋₆-alkyl,
(c) hydroxy-C₂₋₆-alkyl,
(d) fluoro-C₂₋₆-alkyl,
(e) C₃₋₆-alkynyl,
(f) C₃₋₆-alkenyl,
(g) C₃₋₇-cycloalkyl,
(h) C₅₋₈-cycloalkenyl,
(i) NR⁹R⁹, provided that R¹ is not selected from C(O)OR², C(O)NR²R³ and —CH₂—C(O)NR²R³,
(j) C-heterocyclyl, optionally substituted with C₁₋₄-alkyl,
(k) C₇₋₈-bicyclyl, optionally substituted with hydroxy,
(l) C₇₋₈-bicyclylmethyl,
(m) azabicyclyl, optionally substituted with hydroxy,
(n) C₃₋₇-cycloalkyl-C₁₋₄-alkyl, wherein cycloalkyl is optionally substituted with methyl,
(o) C₁₋₆-alkylsulfonyl-C₂₋₆-alkyl,
(p) C₂₋₃-acyl-C₁₋₄-alkyl,
(q) arylcarbonyl-C₁₋₄-alkyl,
(r) heteroarylcarbonyl-C₁₋₄-alkyl,
(s) [C(OH)CH₃CF₃]—C₁₋₆-alkyl,
(t) N-heterocyclylcarbonyl-C₂₋₄-alkyl, wherein heterocyclyl is optionally substituted with methyl,
(u) C-heterocyclylcarbonyl-C₂₋₄-alkyl, wherein heterocyclyl is optionally substituted with methyl,
(v) aminocarbonyl-C₂₋₆-alkyl,
(w) C₁₋₃-alkylaminocarbonyl-C₂₋₆-alkyl,
(x) di(C₁₋₃-alkyl)aminocarbonyl-C₂₋₆-alkyl,
(y) hydroxy-C₂₋₄-alkoxy-C₂₋₄-alkyl,
(z) hydroxy-C₄₋₆-cycloalkyl,
(aa) oxo-C₄₋₆-cycloalkyl,
(bb) fluoro-C₄₋₆-cycloalkyl,
(cc) C₁₋₃-alkoxy-C₄₋₆-cycloalkyl,
(dd) methyl-C₃₋₆-cycloalkyl,
(ee) oxo-N-heterocyclyl-C₂₋₄-alkyl,
(ff) fluoro-N-heterocyclyl-C₂₋₄-alkyl,
(gg) amino-N-heterocyclyl-C₂₋₄-alkyl,
(hh) hydroxy-N-heterocyclyl-C₂₋₄-alkyl,
(ii) N-heterocyclyl-C₂₋₄-alkyl, wherein heterocyclyl is optionally substituted with methyl,
(jj) C-heterocyclyl-C₁₋₄-alkyl, wherein heterocyclyl is optionally substituted with methyl,
(kk) aryl,
(ll) aryl-C₁₋₄-alkyl,
(mm) aryl-C₃₋₆-alkenyl,
(nn) aryl-C₃₋₆-alkynyl,
(oo) heteroaryl,
(pp) heteroaryl-C₁₋₄-alkyl,
(qq) heteroaryl-C₃₋₆-alkenyl, and
(rr) heteroaryl-C₃₋₆-alkynyl,
wherein any aryl or heteroaryl residue, alone or as part of another group, is optionally independently substituted in one or more position with a substituent selected from the group Z¹;
R³ is selected from:
(a) hydrogen,
(b) C₁₋₆-alkyl,
(c) fluoro-C₂₋₆-alkyl,
(d) hydroxy-C₂₋₆-alkyl,
(e) C₁₋₆-alkoxy-C₂₋₆-alkyl,
(f) amino-C₂₋₆-alkyl,
(g) C₁₋₃-alkylamino-C₂₋₆-alkyl,
(h) di(C₁₋₃-alkyl)amino-C₂₋₆-alkyl,
(i) cyano-C₁₋₆-alkyl, and
(j) C₁₋₆-alkylsulfonyl-C₂₋₆-alkyl;
R⁴ is independently selected from:
(a) C₁₋₆-alkyl,
(b) fluoro-C₁₋₆-alkyl,
(c) hydroxy-C₂₋₆-alkyl,
(d) C₁₋₄-alkoxy-C₂₋₄-alkyl,
(e) C₂₋₄-acyl-C₁₋₄-alkyl,
(f) carboxy-C₁₋₃-alkyl,
(g) C₃₋₆-cycloalkyl,
(h) oxo-C₄₋₆-cycloalkyl,
(i) hydroxy-C₄₋₆-cycloalkyl,
(j) fluoro-C₄₋₆-cycloalkyl,
(k) methyl-C₃₋₆-cycloalkyl,
(l) N-heterocyclylcarbonyl-C₂₋₄-alkyl, wherein heterocyclyl is optionally substituted with methyl,
(m) oxo-N-heterocyclyl-C₂₋₄-alkyl,
(n) fluoro-N-heterocyclyl-C₂₋₄-alkyl,
(o) hydroxy-N-heterocyclyl-C₂₋₄-alkyl,
(p) amino-N-heterocyclyl-C₂₋₄-alkyl,
(q) aminocarbonyl-C₂₋₄-alkyl,
(r) C₁₋₃-alkylaminocarbonyl-C₂₋₄-alkyl,
(s) di(C₁₋₃-alkyl)aminocarbonyl-C₂₋₄-alkyl,
(t) C₂₋₃-acylamino-C₂₋₄-alkyl,
(u) hydroxy-C₂₋₄-alkoxy-C₂₋₄-alkyl,
(v) C-heterocyclylcarbonyl-C₂₋₄-alkyl, wherein heterocyclyl is optionally substituted with methyl,
(w) C₃₋₆-cycloalkyl-C₁₋₂-alkyl,
(x) aryl,
(y) aryl-C₁₋₂-alkyl,
(z) heteroaryl, and
(aa) heteroaryl-C₁₋₂-alkyl,
wherein any aryl or heteroaryl residue, alone or as part of another group, is optionally substituted in one or more positions with a substituent independently selected from the group Z² consisting of:
(a) halogen selected from chlorine and fluorine,
(b) C₁₋₄-alkoxy,
(c) hydroxymethyl,
(d) —CN,
(e) —CF₃,
(f) C₁₋₄-alkyl,
(g) —OCF₃, and
(h) —C(O)CH₃;
R⁵ is each independently selected from:
(a) hydrogen,
(b) C₁₋₆-alkyl,
(c) C₃₋₄-cycloalkyl,
(d) fluoro-C₂₋₄-alkyl,
(e) amino-C₂₋₆-alkyl,
(f) cyano-C₁₋₆-alkyl,
(g) hydroxy-C₂₋₆-alkyl,
(h) dihydroxy-C₂₋₆-alkyl,
(i) C₁₋₄-alkoxy-C₂₋₄-alkyl,
(j) C₁₋₄-alkylamino-C₂₋₄-alkyl,
(k) di(C₁₋₄-alkyl)amino-C₂₋₄-alkyl,
(l) aminocarbonyl-C₁₋₄-alkyl,
(m) C₂₋₃-acylamino-C₂₋₄-alkyl,
(n) C₁₋₄-alkylthio-C₂₋₄-alkyl,
(o) C₂₋₄-acyl-C₁₋₄-alkyl, and
(p) C₁₋₄-alkylsulfonyl-C₁₋₄-alkyl,
or two R⁵ groups together with the nitrogen to which they are attached form a heterocyclic ring, wherein said heterocyclic ring may be optionally substituted with:
i) a substituent selected from:
(aa) hydroxy,
(bb) amino,
(cc) methylamino,
(dd) dimethylamino,
(ee) hydroxymethyl, and
(ff) aminomethyl;
ii) one or two oxo groups; or
iii) one or two fluorine atoms, provided that when the substituent is selected from fluorine, hydroxy, amino, methylamino and dimethylamino, said substituent is attached to the heterocyclic ring at a position other than alpha to a heteroatom; and
when the two R⁵ groups form a piperazine ring, the nitrogen of the piperazine ring that allows the substitution is optionally substituted with C₁₋₄-alkyl;
R⁶ is independently selected from:
(a) hydrogen,
(b) C₁₋₄-alkyl, and
(c) hydroxy-C₂₋₄-alkyl;
R⁷ is independently selected from:
(a) hydrogen, and
(b) C₁₋₄-alkyl;
R⁸ is independently selected from:
(a) hydrogen,
(b) C₁₋₆-alkyl,
(c) fluoro-C₁₋₆-alkyl,
(d) hydroxy-C₂₋₆-alkyl,
(e) amino-C₂₋₆-alkyl,
(f) C₁₋₃-alkylamino-C₂₋₄-alkyl,
(g) di(C₁₋₃-dialkyl)amino-C₂₋₄-alkyl,
(h) C₁₋₄-alkylsulfonyl-C₂₋₄-alkyl,
(i) N-heterocyclyl-C₂₋₄-alkyl, wherein heterocyclyl is optionally substituted with methyl,
(j) C-heterocyclyl, optionally substituted with methyl,
(k) C₂₋₃-acylamino-C₂₋₄-alkyl,
(l) [C(OH)CH₃CF₃]—C₁₋₆-alkyl,
(m) C₃₋₆-cycloalkyl,
(n) methyl-C₃₋₆-cycloalkyl,
(o) C₃₋₆-cycloalkyl-C₁₋₂-alkyl,
(p) aryl, and
(q) heteroaryl,
wherein any aryl or heteroaryl residue is optionally independently substituted in one or two positions with a substituent selected from the group Z²;
R⁹ is each independently selected from:
(a) C₁₋₄-alkoxy-C₂₋₄-alkyl,
(b) amino-C₂₋₄-alkyl,
(c) C₁₋₄-alkylamino-C₂₋₄-alkyl,
(d) di(C₁₋₄-alkyl)amino-C₂₋₄-alkyl,
(e) C₂₋₃-acylamino-C₂₋₄-alkyl,
(f) C₁₋₄-alkylthio-C₂₋₄-alkyl, and
(g) C₂₋₄-acyl-C₁₋₄-alkyl,
or two R⁹ groups together with the nitrogen to which they are attached form a heterocyclic ring, wherein said heterocyclic ring may be optionally substituted with:
i) a substituent selected from:
(aa) hydroxy,
(bb) amino,
(cc) methylamino,
(dd) dimethylamino,
(ee) hydroxymethyl, and
(ff) aminomethyl;
ii) one or two oxo groups; or
iii) one or two fluorine atoms, provided that when the substituent is selected from fluorine, hydroxy, amino, methylamino and dimethylamino, said substituent is attached to the heterocyclic ring at a position other than alpha to a heteroatom; and
when the two R⁹ groups form a piperazine ring, the nitrogen of the piperazine ring that allows the substitution is optionally substituted with C₁₋₄-alkyl;
R¹⁰ is independently selected from:
(a) hydrogen,
(b) C₁₋₆-alkyl,
(c) cyclopropyl,
(d) cyclobutyl,
(e) cyclopropylmethyl,
(f) fluoro-C₂₋₆-alkyl,
(g) hydroxy-C₂₋₆-alkyl,
(h) C₁₋₂-alkoxy-C₂₋₆-alkyl,
(i) amino-C₂₋₆-alkyl,
(j) di(C₁₋₃-alkyl)amino-C₂₋₆-alkyl,
(k) C₁₋₃-alkylamino-C₂₋₆-alkyl,
(l) cyano-C₁₋₄-alkyl,
(m) C₂₋₆-acyl,
(n) C₂₋₆-acyl-C₁₋₆-alkyl,
(o) C₁₋₆-alkylsulfonyl-C₁₋₆-alkyl, and
(p) tetrahydrofuran-2-ylmethyl;
R¹¹ is selected from:
(a) hydrogen,
(b) hydroxy,
(c) fluorine,
(d) C₁₋₄-alkoxy, and
(e) methyl;
R¹² is each independently selected from:
(a) hydrogen,
(b) halogen selected from chlorine and fluorine,
(c) —S(O)₂CH₃,
(d) —S(O)₂CF₃,
(e) —OS(O)₂CF₃,
(f) —S(O)NH₂,
(g) —S(O)₂NHCH₃,
(h) —S(O)₂N(CH₃)₂,
(i) —NHS(O)₂CH₃,
(j) —N(CH₃)S(O)₂CH₃,
(k) —NHC(O)CH₃,
(l) —N(CH₃)C(O)CH₃,
(m) —C(O)NH₂,
(n) —C(O)NHCH₃,
(o) —C(O)N(CH₃)₂,
(p) —CN,
(q) —CF₃,
(r) guanidino,
(s) amidino,
(t) —OH,
(u) C₁₋₄-alkoxy,
(v) —OCF₃,
(w) C₃₋₅-cycloalkyloxy,
(x) —SCF₃,
(y) —NO₂,
(z) —NR⁵R⁵, wherein each R⁵ is independently selected from the group consisting of hydrogen and C₁₋₄-alkyl; or two R⁵ groups together with the nitrogen to which they are attached form a pyrrolidine or an azetidine ring,
(aa) —C(OH)CH₃CF₃,
(bb) C₁₋₃-alkyl,
(cc) C₁₋₃-alkoxy-C₁₋₂-alkyl,
(dd) C₂₋₃-acyl,
(ee) C₂₋₃-alkenyl,
(ff) hydroxy-C₁₋₄-alkyl,
(gg) fluoro-C₂₋₃-alkyl,
(hh) C₂₋₃-alkynyl, and
(ii) C₃₋₅-cycloalkyl.

2. A compound according to claim 1 having Formula (Ib) 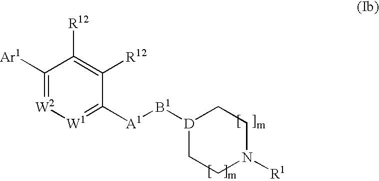
wherein one of W¹ and W² is N and the other is CR¹²;
A¹ is CH₂, O, NR¹⁰, S, S(O) or S(O)₂;
B¹ is CH₂, O, NR O, S, S(O), S(O)₂, C(O) or CONR¹⁰, provided that when B¹ is O, NR¹⁰, S, S(O), S(O)₂, C(O) or CONR¹⁰, then A¹ is CH₂;
m is each independently 0 or 1;
D is N or CR¹¹, provided that D must be CR¹¹ and said R¹¹ must be hydrogen or methyl when B¹ is selected from O, NR¹⁰, S, S(O), S(O)₂, and CONR¹⁰, and further provided that each m is 1 when D is N;
Ar¹, Z¹, Z², R¹ to R⁹ and R¹² are as defined in claim 1;
R¹⁰ is independently selected from:
(a) hydrogen,
(b) C₁₋₄-alkyl,
(c) cyclopropyl,
(d) cyclobutyl,
(e) cyclopropylmethyl,
(f) fluoro-C₂₋₄-alkyl,
(g) C₁₋₂-alkoxy-C₂₋₃-alkyl,
(h) hydroxy-C₂₋₄-alkyl,
(i) C₂₋₃-acyl,
(j) amino-C₂₋₄-alkyl,
(k) methylamino-C₂₋₄-alkyl,
(l) dimethylamino-C₂₋₄-alkyl,
(m) cyano-C₁₋₄-alkyl, and
(n) tetrahydrofuran-2-ylmethyl;
R¹¹ is selected from:
(a) hydrogen,
(b) hydroxy,
(c) fluorine, and
(d) methyl.

3. A compound according to claim 1 having Formula (Ic) 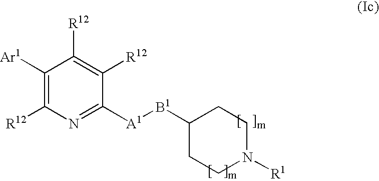
wherein A¹ is CH₂, O or NR¹⁰;
B¹ is CH₂, O or NR¹⁰, provided that when B¹ is O or NR¹⁰, then A¹ is CH₂;
in is each independently 0 or 1;
Z¹, Z², R¹ to R⁷, R⁹ and R¹² are as defined in claim 1, provided that at least two of R¹² are hydrogen;
R¹⁰ is as defined in claim 2;
Ar¹ is phenyl, which is optionally substituted in one or two positions with a substituent independently selected from the group Z³ consisting of:
(a) CF₃SO₃,
(b) halogen selected from bromine, chlorine and fluorine,
(c) C₁₋₄-alkylsulfinyl,
(d) —S(O)₂R⁴,
(e) —S(O)₂NR⁵R⁵,
(f) —NR⁶S(O)₂R⁴,
(g) —NR⁶C(O)R⁴,
(h) —CH₂—NR⁶C(O)R⁴,
(i) —C(O)NR⁵R⁵,
(j) —CH₂—C(O)NR⁵R⁵,
(k) —C(O)R⁴,
(l) H₂N—C(O)O—,
(m) CH₃—NH—C(O)O—,
(n) (CH₃)₂NC(O)O—,
(o) —NHC(O)OCH₃,
(p) C-heterocyclyl, optionally substituted with methyl,
(q) —CN,
(r) —OR⁸,
(s) —SCF₃,
(t) —NO₂,
(u) phosphonooxy,
(v) C-heterocyclylsulfonyl, optionally substituted with methyl,
(w) —NR⁵R⁵,
(x) —C(OH)CH₃CF₃,
(y) cyano-C₁₋₆-alkyl,
(z) guanidino,
(aa) amidino,
(bb) C₁₋₆-alkyl,
(cc) C₁₋₄-alkoxy-C₁₋₄-alkyl,
(dd) fluoro-C₁₋₄-alkyl,
(ee) C₂₋₆-alkenyl,
(ff) fluoro-C₂₋₄-alkenyl,
(gg) hydroxy-C₁₋₆-alkyl,
(hh) C₁₋₄-alkyl sulfonyl-C₁₋₄-alkyl,
(ii) hydroxy-C₂₋₄-alkoxy-C₁₋₄-alkyl,
(jj) C₂₋₃-acyl-C₁₋₃-alkyl,
(kk) C₂₋₆-alkynyl,
(ll) C₃₋₆-cycloalkyl,
(mm) hydroxy-C₃₋₆-cycloalkyl,
(nn) fluoro-C₃₋₆-cycloalkyl,
(oo) methyl-C₃₋₆-cycloalkyl,
(pp) C-heterocyclylcarbonyl, optionally substituted with methyl,
(qq) C₃₋₆-cycloalkyl-C₁₋₄-alkyl,
(rr) R⁵R⁵N—C₁₋₂-alkyl,
(ss) —C(O)OR⁷,
(tt) —CH₂C(O)OR⁷,
(uu) aryl, and
(vv) heteroaryl,
wherein any aryl or heteroaryl residue as substituent on Ar¹ is optionally substituted in one or more positions with a substituent independently selected from the group Z¹ as defined in claim 1;
R⁸ is independently selected from:
(a) hydrogen,
(b) C₁₋₄-alkyl,
(c) CF₃,
(d) C₃₋₅-cycloalkyl,
(e) methyl-C₃₋₅-cycloalkyl, and
(f) C-heterocyclyl, optionally substituted with methyl.

4. A compound according to claim 3, wherein
A¹ is CH₂ and B¹ is O or NR¹⁰, or
A¹ is O or NR¹⁰ and B¹ is CH₂;
and m is each l.

5. A compound according to claim 4, wherein
Ar¹ is phenyl, which is optionally substituted in one or two positions with a substituent independently selected from the group Z⁴ consisting of:
(a) halogen selected from chlorine and fluorine,
(b) C₁₋₄-alkylsulfonyl,
(c) C₁₋₄-alkylsulfinyl,
(d) hydroxy-C₂₋₄-alkylsulfonyl,
(e) C₃₋₅-cycloalkylsulfonyl,
(f) methyl-C₃₋₅-cycloalkylsulfonyl,
(g) trifluoromethylsulfonyl,
(h) —S(O)₂NR^{5A}R^{5A},
(i) C₁₋₄-alkylsulfonamido,
(j) C₂₋₄-acylamino,
(k) C₂₋₄-acylaminomethyl,
(l) carboxy-C₁₋₃-alkylcarbonylamino,
(m) —C(O)NR^{5A}R^{5A},
(n) —CH₂—C(O)NR^{5A}R^{5A}
(o) —NHC(O)OCH₃,
(p) C₂₋₄-acyl,
(q) C₃₋₅-cycloalkylcarbonyl,
(r) C₁₋₄-alkoxy,
(s) C₃₋₅-cycloalkyloxy,
(t) C-heterocyclyl,
(u) —CN,
(v) —OH,
(w) —OCF₃,
(x) —CF₃,
(y) —NO₂,
(z) —NR^{5A}R^{5A},
(aa) —C(OH)CH₃CF₃,
(bb) cyano-C₁₋₂-alkyl,
(cc) C₁₋₄-alkyl,
(dd) C₃₋₅-cycloalkyl,
(ee) C₁₋₂-alkoxy-C₁₋₂-alkyl,
(ff) vinyl,
(gg) ethynyl,
(hh) hydroxy-C₁₋₂-alkyl,
(ii) C-heterocyclyloxy, optionally substituted with methyl,
(jj) R^{5A}R^{5A}N—C₁₋₂-alkyl, and
(kk) —C(O)OR^{7A};
(ll) —CH₂C(O)OR^{7A},
R¹ is a group R^{1A} selected from C(O)OR^{2A}, C(O)R^{2A}, S(O)₂R^{2A}, C(O)NR^{2A}R^{3A}, —CH₂—C(O)NR^{2A}R^{2A}, or a 5- or 6-membered heteroaryl group linked via a ring carbon atom, wherein the said heteroaryl group is optionally substituted with C₁₋₄-alkyl;
R^{2A} is selected from:
(a) C₁₋₆-alkyl,
(b) C₁₋₆-alkoxy-C₂₋₆-alkyl,
(c) hydroxy-C₂₋₆-alkyl,
(d) hydroxy-C₂₋₄-alkoxy-C₂₋₄-alkyl,
(e) fluoro-C₂₋₆-alkyl,
(f) C₃₋₆-alkynyl,
(g) C₃₋₇-cycloalkyl,
(h) C₅₋₈-cycloalkenyl,
(i) NR^{9A}R^{9A} provided that R^{1A} is not selected from C(O)OR^{2A}, C(O)NR^{2A}R^{3A} and —CH₂—C(O)NR^{2A}R^{3A},
(j) C-heterocyclyl, optionally substituted with methyl,
(k) C₇₋₈-bicyclyl,
(l) 2-norbornylmethyl,
(m) azabicyclyl,
(n) C₃₋₆-cycloalkyl-C₁₋₄-alkyl, wherein cycloalkyl is optionally substituted with methyl
(o) C₂₋₃-acyl-C₁₄-alkyl,
(p) arylcarbonyl-C₁₋₄-alkyl,
(q) heteroarylcarbonyl-C₁₋₄-alkyl,
(r) [C(OH)CH₃CF₃]—C₁₋₆-alkyl,
(s) N-heterocyclylcarbonyl-C₂₋₄-alkyl, wherein heterocyclyl is optionally substituted with methyl,
(t) hydroxy-C₄₋₆-cycloalkyl,
(u) oxo-C₄₋₆-cycloalkyl,
(v) fluoro-C₄₋₆-cycloalkyl,
(w) methoxy-C₄₋₆-cycloalkyl,
(x) methyl-C₃₋₆-cycloalkyl,
(y) oxo-N-heterocyclyl-C₂₋₄-alkyl,
(z) hydroxy-N-heterocyclyl-C₂₋₄-alkyl,
(aa) fluoro-N-heterocyclyl-C₂₋₄-alkyl,
(bb) amino-N-heterocyclyl-C₂₋₄-alkyl,
(cc) N-heterocyclyl-C₂₋₄-alkyl, wherein heterocyclyl is optionally substituted with methyl,
(dd) C-heterocyclyl-C₁₋₄-alkyl, wherein heterocyclyl is optionally substituted with methyl,
(ee) aryl,
(ff) aryl-C₁₋₄-alkyl,
(gg) heteroaryl, and
(hh) heteroaryl-C₁₋₄-alkyl,
wherein any aryl or heteroaryl residue, alone or as a part of another group, is optionally substituted in one or more positions with a substituent independently selected from the group Z⁵ consisting of:
(a) halogen selected from chlorine and fluorine,
(b) methyl,
(c) ethyl,
(d) methoxy,
(e) ethoxy,
(f) isopropoxy,
(g) hydroxy,
(h) —OCF₃,
(i) —CF₃,
(j) —CN,
(k) —C(OH)CH₃CF₃,
(l) dimethylamino,
(m) hydroxymethyl,
(n) —S(O)₂CH₃,
(o) —C(O)CH₃, and
(p) —C(O)NH₂;
R^{3A} is selected from:
(a) hydrogen,
(b) C₁₋₄-alkyl,
(c) hydroxy-C₂₋₄-alkyl, and
(d) methoxy-C₂₋₄-alkyl;
R^{5A} is each independently selected from:
(a) hydrogen,
(b) C₁₋₃-alkyl,
(c) C₁₋₂-alkoxy-C₂₋₄-alkyl,
(d) C₃₋₄-cycloalkyl,
(e) hydroxy-C₂₋₄-alkyl,
(f) cyano-C₁₋₃-alkyl,
(g) dihydroxy-C₂₋₄-alkyl,
(h) aminocarbonyl-C₁₋₂-alkyl, and
(i) di(C₁₋₂-alkyl)amino-C₂₋₃-alkyl;
or two R^{5A} groups together with the nitrogen to which they are attached form a heterocyclic ring, wherein said heterocyclic ring may be optionally substituted with:
i) a substituent selected from:
(aa) hydroxy,
(bb) amino,
(cc) methylamino,
(dd) dimethylamino,
(ee) hydroxymethyl, and
(ff) aminomethyl;
ii) one or two oxo groups; or
iii) one or two fluorine atoms, provided that when the substituent is selected from fluorine, hydroxy, amino, methylamino and dimethylamino, said substituent is attached to the heterocyclic ring at a position other than alpha to a heteroatom; and
when the two R^{5A} groups form a piperazine ring, the nitrogen of the piperazine ring that allows the substitution is optionally substituted with methyl;
R^{7A} is independently from:
(a) hydrogen, and
(b) C₁₋₄-alkyl;
Two groups R^{9A} together with the nitrogen to which they are attached form a heterocyclic ring, wherein said heterocyclic ring may be optionally substituted with: i) one hydroxy or amino group, ii) one or two fluorine atoms, or iii) one or two oxo groups, provided that when the substituent is selected from fluorine, hydroxy and amino, said substituent is attached to the heterocyclic ring at a position other than alpha to a heteroatom; and when the two R^{9A} groups form a piperazine ring, the nitrogen of the piperazine ring that allows the substitution is optionally substituted with methyl;
R¹⁰ is independently selected from:
(a) hydrogen, and
(b) C₁₋₃-alkyl;
R¹² is independently selected from:
(a) hydrogen, and
(b) —NO₂.

6. A compound according to claim 5, wherein R^{1A} is selected from C(O)OR^{2A} and C(O)R^{2A}.

7. A compound according to claim 5, wherein R^{1A} is C(O)OR^{2A}, wherein R^{2A} is selected from tert-butyl, benzyl, iso-butyl, ethyl, 4-methoxyphenyl, 2-propynyl, isopropyl, cyclobutyl, 1-cyclopropylethyl, (1S,2R,4R)-bicyclo[2.2.1]hept-2-yl, (3-methyloxetan-3-yl)methyl, (1-methylcyclopropyl)methyl and 3-hydroxy-3-methylbutyl.

8. A compound according to claim 5, wherein R^{1A} is C(O)R^{2A}, wherein R^{2A} is selected from 2-(3-chloro-4-methoxyphenyl)ethyl, bicyclo[2.2.1]hept-2-yl, cyclohexylmethyl, 5-isopropoxy-pyridin-2-yl, cyclohexyl, 4-methoxycyclohexyl, 3-cyanophenyl, 2-hydroxy-2-methyl-propyl, 3,3,3-trifluoro-2-hydroxy-2-methylpropyl, 3-acetylphenyl, phenyl, 3-dimethylaminophenyl, 3-oxo-3-phenylpropyl, 2-pyridinyl, 3-hydroxy-2-pyridinyl, 4-isopropoxyphenyl, 2-cyclopentylethyl, (2,3,6-trifluorophenyl)methyl and n-butyl.

9. A compound according to any one of claims 5 to 8, wherein Ar¹ is selected from methylsulfonylphenyl, (morpholin-4-ylsulfonyl)phenyl and cyanophenyl.

10. A compound according to any one of claims 5 to 8, wherein R¹⁰ is independently selected from hydrogen and methyl.

11. A compound according to claim 1 having formula (Id) 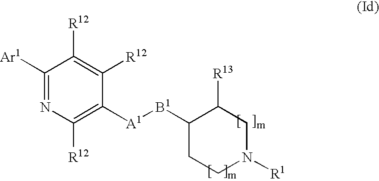
wherein A¹ is CH₂, O or NR¹⁰;
B¹ is CH₂, O or NR¹⁰, provided that when B¹ is O or NR¹⁰, then A¹ is CH₂;
m is each independently 0 or 1;
Z¹, Z², R¹ to R⁷, R⁹ and R¹² are as defined in claim 1, provided that at least two of R¹² are hydrogen;
R⁸ is as defined in claim 3;
R¹⁰ is as defined in claim 2;
R¹³ is hydrogen or methyl;
Ar¹ is phenyl, which is optionally substituted in one or two positions with a substituent independently selected from the group Z³ as defined in claim 3.

12. A compound according to claim 11, wherein
A¹ is CH₂ and B¹ is O or NR¹⁰, or
A¹ is O or NR¹⁰ and B¹ is CH₂; and
m is each 1.

13. A compound according to claim 12, wherein
Ar¹ is phenyl, which is optionally substituted in one or two positions with a substituent independently selected from the group Z⁴ as defined in claim 5;
Z⁵ is as defined in claim 5;
R¹ is a group R^{1A}, wherein R^{1A} is as defined in claim 5;
R^{2A}, R^{3A}, R^{5A}, R^{7A} and R^{9A} are as defined in claim 5;
R¹⁰ is independently selected from:
(a) hydrogen,
(b) C₁₋₃-alkyl;
(c) cyclopropyl,
(d) cyclobutyl,
(e) cyclopropylmethyl,
(f) fluoro-C₂₋₄-alkyl,
(g) hydroxy-C₂₋₄-alkyl,
(h) cyano-C₁₋₄-alkyl, and
(i) tetrahydrofuran-2-ylmethyl;
R¹² is each hydrogen.

14. A compound according to claim 13, wherein Ar¹ is selected from methylsulfonylphenyl, cyanophenyl, [(dimethylamino)carbonyl]phenyl, (morpholin-4-ylcarbonyl)phenyl, (aminocarbonyl)phenyl, [(2-hydroxyethyl)aminocarbonyl]-phenyl, [(methoxycarbonyl)amino]phenyl, [(2-hydroxyethyl)sulfonyl]phenyl, carboxyphenyl, fluoro[(propylamino)carbonyl]phenyl, [(cyclopropylamino)-carbonyl]phenyl, [(ethylamino)carbonyl]phenyl, [(methylamino)carbonyl]phenyl, [(2-cyanoethyl)aminocarbonyl]phenyl, (5,6-dihydro-4H-1,3-oxazin-2-yl)phenyl, (acetylamino)phenyl, [(2-methoxyethyl)aminocarbonyl]phenyl, [(2-hydroxyethyl)-aminocarbonyl]phenyl, [(2-hydroxybutyl)aminocarbonyl]phenyl, [(acetylamino)-methyl]phenyl, [(4-methylpiperazin-1-yl)carbonyl]phenyl, [2-(hydroxymethyl)-morpholin-4-ylcarbonyl]phenyl, [(2-amino-2-oxoethyl)aminocarbonyl]phenyl, [(2-carboxyethyl)carbonylamino]phenyl, (cyanomethyl)phenyl, (methylsulfinyl)phenyl, fluoro(methylsulfonyl)phenyl, (aminocarbonyl)fluorophenyl, (azetidin-1-ylsulfonyl)-phenyl, (carboxymethyl)phenyl, [2-(4-hydroxypiperidin-1-yl)-2-oxoethyl]phenyl, {2-[2-(hydroxymethyl)morpholin-4-yl]-2-oxoethyl}phenyl, and [2-(3-hydroxyazetidin-1-yl)-2-oxoethyl]phenyl.

15. A compound according to claim 13, wherein R^{1A} is selected from C(O)OR^{2A} and C(O)R^{2A}.

16. A compound according to any one of claims 13 or 14, wherein R^{1A} is C(O)OR^{2A}, and wherein R^{2A} is selected from tert-butyl, 2-methoxyethyl, isobutyl, ethyl, isopropyl, benzyl, 2,2-dimethylpropyl, prop-2-yn-1-yl, phenyl, 4-fluorophenyl, 4-methoxyphenyl, 2-fluoro-1-(fluoromethyl)ethyl, (1R)-1-phenylethyl, (1S)-1-phenylethyl, (1S,2R,4R)-bicyclo[2.2.1]hept-2-yl, (1-methylcyclopropyl)methyl, cyclobutyl and 1,3-benzodioxol-5-ylmethyl.

17. A compound according to any one of claims 13 or 14, wherein R^{1A} is C(O)R^{2A}, and wherein R^{2A} is selected from tert-butyl, 2-(4-fluorophenyl)ethyl, 4-isopropoxyphenyl, 3,4-dichlorophenyl, 3-(4-fluorophenyl)propyl, [3-(trifluoromethyl)phenyl]-methyl, cyclohexylmethyl, phenyl, 2-methylpropyl, cyclohexyl, 2,2-dimethylpropyl, 2,4-dichlorophenyl, 2,4-difluorophenyl, 2,5-difluorophenyl, 2-fluorophenyl, 3-fluorophenyl, 4-fluorophenyl, 3-methoxyphenyl and 3-chloro-4-methoxyphenyl.

18. A compound according to claim 1 having Formula (Ie) 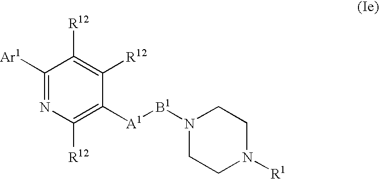
wherein A¹ is CH₂, O or NR¹⁰;
B¹ is CH₂ or C(O);
Z¹, Z², R¹ to R⁷, R⁹ and R¹² are as defined in claim 1, provided that at least two of R¹² are hydrogen;
R⁸ is as defined in claim 3;
R¹⁰ is as defined in claim 2;
Ar¹ is phenyl, which is optionally substituted in one or two positions with a substituent independently selected from the group Z³ as defined in claim 3.

19. A compound according to claim 18, wherein
A¹ is CH₂; and
B¹ is CH₂ or C(O).

20. A compound according to claim 19, wherein
Ar¹ is phenyl, which is optionally substituted in one or two positions with a substituent independently selected from the group Z⁴ as defined in claim 5;
Z⁵ is as defined in claim 5;
R¹ is a group R^{1A}, wherein R^{1A} is as defined in claim 5;
R^{2A}, R^{3A}, R^{5A}, R^{7A} and R^{9A} are as defined in claim 5;
R¹² is each hydrogen;

21. A compound according to claim 20, wherein Ar¹ is C₁₋₄-alkylsulfonylphenyl.

22. A compound according to claim 20 or 21, wherein R^{1A} is selected from C(O)OR^{2A} and C(O)R^{2A}.

23. A compound according to claim 20 or 21, wherein R^{1A} is C(O)OR^{2A} and wherein R^{2A} is C₁₋₆-alkyl.

24. A compound according to claim 20 or 21, wherein R^{1A} is C(O)R^{2A} and wherein R^{2A} is phenyl, which is monosubstituted with a substituent selected from methoxy, ethoxy and isopropoxy.

25. A compound according to claim 1, which is selected from:
tert-Butyl 4-[({5-[4-(methylsulfonyl)phenyl]pyridin-2-yl}oxy)methyl]-piperidine-1-carboxylate;
Benzyl 4-[({5-[4-(methylsulfonyl)phenyl]pyridin-2-yl}oxy)methyl]piperidine-1-carboxylate;
2-({1-[3-(3-Chloro-4-methoxyphenyl)propanoyl]piperidin-4-yl}methoxy)-5-[4-(methylsulfonyl)phenyl]pyridine;
2-{[1-(Bicyclo[2.2.1]hept-2-ylcarbonyl)piperidin-4-yl]methoxy}-5-[4-(methylsulfonyl)phenyl]pyridine;
2-{[1-(Cyclohexylacetyl)piperidin-4-yl]methoxy}-5-[4-(methylsulfonyl)-phenyl]pyridine;
5-Isopropoxy-2-({4-[({5-[4-(methylsulfonyl)phenyl]pyridin-2-yl}oxy)methyl]-piperidin-1-yl}carbonyl)pyridine;
2-{[1-(Cyclohexylcarbonyl)piperidin-4-yl]methoxy}-5-[4-(methylsulfonyl)-phenyl]pyridine;
2-({1-[(4-Methoxycyclohexyl)carbonyl]piperidin-4-yl}methoxy)-5-[4-(methylsulfonyl)phenyl]pyridine;
3-({4-[({5-[4-(Methylsulfonyl)phenyl]pyridin-2-yl}oxy)methyl]piperidin-1-yl}carbonyl)benzonitrile;
2-Methyl-4-{4-[({5-[4-(methylsulfonyl)phenyl]pyridin-2-yl}oxy)methyl]-piperidin-1-yl}-4-oxobutan-2-ol;
1,1,1-Trifluoro-2-methyl-4-{4-[({5-[4-(methylsulfonyl)phenyl]pyridin-2-yl}-oxy)methyl]piperidin-1-yl}-4-oxobutan-2-ol;
1-[3-({4-[({5-[4-(Methylsulfonyl)phenyl]pyridin-2-yl}oxy)methyl]piperidin-1-yl}carbonyl)phenyl]ethanone;
tert-Butyl 4-({[5-(4-cyanophenyl)pyridin-2-yl]oxy}methyl)piperidine-1-carboxylate;
tert-Butyl 4-[({5-[4-(morpholin-4-ylsulfonyl)phenyl]pyridin-2-yl}oxy)methyl]-piperidine-1-carboxylate;
2-[(1-Benzoylpiperidin-4-yl)methoxy]-5-[4-(methylsulfonyl)phenyl]pyridine;
N,N-Dimethyl-3-({4-[({5-[4-(methylsulfonyl)phenyl]pyridin-2-yl}oxy)-methyl]piperidin-1-yl}carbonyl)aniline trifluoroacetate;
4-{4-[({5-[4-(Methylsulfonyl)phenyl]pyridin-2-yl}oxy)methyl]piperidin-1-yl}-4-oxo-1-phenylbutan-1-one;
5-[4-(Methylsulfonyl)phenyl]-2-{[1-(pyridin-2-ylcarbonyl)piperidin-4-yl]-methoxy}pyridine;
2-({4-[({5-[4-(Methylsulfonyl)phenyl]pyridin-2-yl}oxy)methyl]piperidin-1-yl}carbonyl)pyridin-3-ol;
2-{[1-(4-Isopropoxybenzoyl)piperidin-4-yl]methoxy}-5-[4-(methylsulfonyl)-phenyl]pyridine;
tert-Butyl 4-[({5-[4-(methylsulfonyl)phenyl]-3-nitropyridin-2-yl}oxy)methyl]-piperidine-1-carboxylate;
2-{[1-(Cyclohexylacetyl)piperidin-4-yl]methoxy}-5-[4-(methylsulfonyl)-phenyl]-3-nitropyridine;
2-{[1-(Bicyclo[2.2.1]hept-2-ylcarbonyl)piperidin-4-yl]methoxy}-5-[4-(methylsulfonyl)phenyl]-3-nitropyridine;
tert-Butyl 4-[({5-[4-(methylsulfonyl)phenyl]pyridin-2-yl}amino)methyl]-piperidine-1-carboxylate;
Isobutyl 4-[({5-[4-(methylsulfonyl)phenyl]pyridin-2-yl}amino)methyl]-piperidine-1-carboxylate;
Benzyl 4-[({5-[4-(methylsulfonyl)phenyl]pyridin-2-yl}amino)methyl]piperidine-1-carboxylate;
Ethyl 4-[({5-[4-(methylsulfonyl)phenyl]pyridin-2-yl}amino)methyl]piperidine-1-carboxylate;
N-{[1-(Cyclohexylcarbonyl)piperidin-4-yl]methyl}-5-[4-(methylsulfonyl)-phenyl]pyridin-2-amine;
N-{[1-(Cyclohexylacetyl)piperidin-4-yl]methyl}-5-[4-(methylsulfonyl)-phenyl]pyridin-2-amine;
N-{[1-(3-Cyclopentylpropanoyl)piperidin-4-yl]methyl}-5-[4-(methylsulfonyl)-phenyl]pyridin-2-amine;
5-[4-(Methylsulfonyl)phenyl]-N-({1-[(2,3,6-trifluorophenyl)acetyl]piperidin-4-yl}methyl)pyridin-2-amine;
5-[4-(Methylsulfonyl)phenyl]-N-[(1-pentanoylpiperidin-4-yl)methyl]pyridin-2-amine;
tert-Butyl 4-[(methyl {5-[4-(methylsulfonyl)phenyl]pyridin-2-yl}amino)-methyl]piperidine-1-carboxylate;
tert-Butyl 4-({5-[4-(methylsulfonyl)phenyl]pyridin-2-yl}methoxy)piperidine-1-carboxylate;
4-Methoxyphenyl 4-({5-[4-(methylsulfonyl)phenyl]pyridin-2-yl}methoxy)-piperidine-1-carboxylate;
Prop-2-yn-1-yl 4-({5-[4-(methylsulfonyl)phenyl]pyridin-2-yl}methoxy)-piperidine-1-carboxylate;
2-({[1-(Bicyclo[2.2.1]hept-2-ylcarbonyl)piperidin-4-yl]oxy}methyl)-5-[4-(methylsulfonyl)phenyl]pyridine;
Isopropyl 4-({5-[4-(methylsulfonyl)phenyl]pyridin-2-yl}methoxy)piperidine-1-carboxylate;
tert-Butyl 4-[methyl({5-[4-(methylsulfonyl)phenyl]pyridin-2-yl}methyl)-amino]piperidine-1-carboxylate;
(1S,2R,4R)-Bicyclo[2.2.1]hept-2-yl 4-[methyl({5-[4-(methylsulfonyl)phenyl]-pyridin-2-yl}methyl)amino]piperidine-1-carboxylate;
(3-Methyloxetan-3-yl)methyl 4-[methyl({5-[4-(methylsulfonyl)phenyl]pyridin-2-yl}methyl)amino]piperidine-1-carboxylate;
(1-Methylcyclopropyl)methyl 4-[methyl({5-[4-(methylsulfonyl)phenyl]-pyridin-2-yl}methyl)amino]piperidine-1-carboxylate;
tert-Butyl 4-[{[5-(4-cyanophenyl)pyridin-2-yl]methyl}(methyl)amino]-piperidine-1-carboxylate;
Isobutyl 4-[methyl({5-[4-(methylsulfonyl)phenyl]pyridin-2-yl}methyl)amino]-piperidine-1-carboxylate;
Cyclobutyl 4-[methyl({5-[4-(methylsulfonyl)phenyl]pyridin-2-yl}methyl)-amino]piperidine-1-carboxylate;
1-Cyclopropylethyl 4-[methyl({5-[4-(methylsulfonyl)phenyl]pyridin-2-yl}-methyl)amino]piperidine-1-carboxylate;
Isopropyl 4-[methyl({5-[4-(methylsulfonyl)phenyl]pyridin-2-yl}methyl)-amino]piperidine-1-carboxylate;
3-Hydroxy-3-methylbutyl 4-[methyl({5-[4-(methylsulfonyl)phenyl]pyridin-2-yl}methyl)amino]piperidine-1-carboxylate;
tert-Butyl 4-[({6-[4-(methylsulfonyl)phenyl]pyridin-3-yl}methyl)amino]-piperidine-1-carboxylate;
tert-Butyl 4-[methyl({6-[4-(methylsulfonyl)phenyl]pyridin-3-yl}methyl)-amino]piperidine-1-carboxylate;
2-Methoxyethyl 4-[methyl({6-[4-(methylsulfonyl)phenyl]pyridin-3-yl}-methyl)amino]piperidine-1-carboxylate;
Isobutyl 4-[methyl({6-[4-(methylsulfonyl)phenyl]pyridin-3-yl}methyl)amino]-piperidine-1-carboxylate;
Ethyl 4-[methyl({6-[4-(methylsulfonyl)phenyl]pyridin-3-yl}methyl)amino]-piperidine-1-carboxylate;
Isopropyl 4-[methyl({6-[4-(methylsulfonyl)phenyl]pyridin-3-yl}methyl)-amino]piperidine-1-carboxylate;
Benzyl 4-[methyl({6-[4-(methylsulfonyl)phenyl]pyridin-3-yl}methyl)amino]-piperidine-1-carboxylate;
2,2-Dimethylpropyl 4-[methyl({6-[4-(methylsulfonyl)phenyl]pyridin-3-yl}-methyl)amino]piperidine-1-carboxylate;
Prop-2-yn-1-yl 4-[methyl({6-[4-(methylsulfonyl)phenyl]pyridin-3-yl}methyl)-amino]piperidine-1-carboxylate;
Phenyl 4-[methyl({6-[4-(methylsulfonyl)phenyl]pyridin-3-yl}methyl)amino]-0 piperidine-1-carboxylate;
4-Fluorophenyl 4-[methyl({6-[4-(methylsulfonyl)phenyl]pyridin-3-yl}methyl)-amino]piperidine-1-carboxylate;
4-Methoxyphenyl 4-[methyl({6-[4-(methylsulfonyl)phenyl]pyridin-3-yl}-methyl)amino]piperidine-1-carboxylate;
2-Fluoro-1-(fluoromethyl)ethyl 4-[methyl({6-[4-(methylsulfonyl)phenyl]-pyridin-3-yl}methyl)amino]piperidine-1-carboxylate;
(1R)-1-Phenylethyl 4-[methyl({6-[4-(methylsulfonyl)phenyl]pyridin-3-yl}-methyl)amino]piperidine-1-carboxylate;
(1S)-1-Phenylethyl 4-[methyl({6-[4-(methylsulfonyl)phenyl]pyridin-3-yl}-methyl)amino]piperidine-1-carboxylate;
(1S,2R,4R)-Bicyclo[2.2.1]hept-2-yl 4-[methyl({6-[4-(methylsulfonyl)phenyl]-pyridin-3-yl}methyl)amino]piperidine-1-carboxylate;
(1-Methylcyclopropyl)methyl 4-[methyl({6-[4-(methylsulfonyl)phenyl]-pyridin-3-yl}methyl)amino]piperidine-1-carboxylate;
Cyclobutyl 4-[methyl({6-[4-(methylsulfonyl)phenyl]pyridin-3-yl}methyl)-amino]piperidine-1-carboxylate;
1,3-Benzodioxol-5-ylmethyl 4-[methyl({6-[4-(methylsulfonyl)phenyl]pyridin-3-yl}methyl)amino]piperidine-1-carboxylate;
tert-Butyl 4-[(2-fluoroethyl)({6-[4-(methylsulfonyl)phenyl]pyridin-3-yl}-methyl)amino]piperidine-1-carboxylate;
tert-Butyl 4-[(cyclopropylmethyl)({6-[4-(methylsulfonyl)phenyl]pyridin-3-yl}-methyl)amino]piperidine-1-carboxylate;
tert-Butyl 4-[(2-hydroxyethyl)({6-[4-(methylsulfonyl)phenyl]pyridin-3-yl}-methyl)amino]piperidine-1-carboxylate;
tert-Butyl 4-[(cyanomethyl)({6-[4-(methylsulfonyl)phenyl]pyridin-3-yl}-methyl)amino]piperidine-1-carboxylate;
tert-Butyl 4-[ethyl({6-[4-(methylsulfonyl)phenyl]pyridin-3-yl}methyl)amino]-piperidine-1-carboxylate;
tert-Butyl 4-[cyclobutyl({6-[4-(methylsulfonyl)phenyl]pyridin-3-yl}methyl)-amino]piperidine-1-carboxylate;
tert-Butyl 4-[({6-[4-(methylsulfonyl)phenyl]pyridin-3-yl}methyl)(3,3,3-trifluoropropyl)amino]piperidine-1-carboxylate;
tert-Butyl 4-[({6-[4-(methylsulfonyl)phenyl]pyridin-3-yl}methyl)(2,2,2-trifluoroethyl)amino]piperidine-1-carboxylate;
tert-Butyl 4-[isobutyl({6-[4-(methylsulfonyl)phenyl]pyridin-3-yl}methyl)-amino]piperidine-1-carboxylate;
tert-Butyl 4-[({6-[4-(methylsulfonyl)phenyl]pyridin-3-yl}methyl)(tetrahydrofuran-2-ylmethyl)amino]piperidine-1-carboxylate;
tert-Butyl 4-[isopropyl({6-[4-(methylsulfonyl)phenyl]pyridin-3-yl}methyl)-amino]piperidine-1-carboxylate;
Isopropyl 4-[isopropyl({6-[4-(methylsulfonyl)phenyl]pyridin-3-yl}methyl)-amino]piperidine-1-carboxylate;
tert-Butyl 4-[({6-[4-(methyl sulfonyl)phenyl]pyridin-3-yl}methyl)(propyl)-amino]piperidine-1-carboxylate;
Isopropyl 4-[({6-[4-(methylsulfonyl)phenyl]pyridin-3-yl}methyl)(propyl)-amino]piperidine-1-carboxylate;
Isobutyl 4-[({6-[4-(methyl sulfonyl)phenyl]pyridin-3-yl}methyl)(propyl)-amino]piperidine-1-carboxylate;
tert-Butyl 4-[cyclopropyl({6-[4-(methylsulfonyl)phenyl]pyridin-3-yl}methyl)-amino]piperidine-1-carboxylate;
Isopropyl 4-[cyclopropyl({6-[4-(methylsulfonyl)phenyl]pyridin-3-yl}methyl)-amino]piperidine-1-carboxylate;
Isobutyl 4-[cyclopropyl({6-[4-(methyl sulfonyl)phenyl]pyridin-3-yl}methyl)-amino]piperidine-1-carboxylate;
tert-butyl 4-[cyclopropyl({6-[4-(methylsulfinyl)phenyl]pyridin-3-yl}methyl)-amino]piperidine-1-carboxylate;
tert-butyl 4-{cyclopropyl[(6-{4-[(dimethylamino)carbonyl]phenyl}pyridin-3-yl)methyl]amino}piperidine-1-carboxylate;
Isopropyl 4-[cyclopropyl({6-[4-(methylsulfinyl)phenyl]pyridin-3-yl}methyl)-amino]piperidine-1-carboxylate;
Isopropyl 4-{cyclopropyl[(6-{4-[(dimethylamino)carbonyl]phenyl}pyridin-3-yl)methyl]amino}piperidine-1-carboxylate;
tert-Butyl 4-[{[6-(4-cyanophenyl)pyridin-3-yl]methyl}(methyl)amino]-piperidine-1-carboxylate;
tert-Butyl 4-[[(6-{4-[(dimethylamino)carbonyl]phenyl}pyridin-3-yl)methyl]-(methyl)amino]piperidine-1-carboxylate;
tert-Butyl 4-[methyl({6-[4-(morpholin-4-ylcarbonyl)phenyl]pyridin-3-yl}-methyl)amino]piperidine-1-carboxylate;
tert-Butyl 4-[({6-[4-(aminocarbonyl)phenyl]pyridin-3-yl}methyl)(3,3,3-trifluoropropyl)amino]piperidine-1-carboxylate;
tert-Butyl 4-[[(6-{4-[(dimethylamino)carbonyl]phenyl}pyridin-3-yl)methyl]-(3,3,3-trifluoropropyl)amino]piperidine-1-carboxylate;
tert-Butyl 4-[[(6-{4-[(acetylamino)methyl]phenyl}pyridin-3-yl)methyl](3,3,3-trifluoropropyl)amino]piperidine-1-carboxylate;
tert-Butyl 4-[[(6-{3-[(acetylamino)methyl]phenyl}pyridin-3-yl)methyl](3,3,3-trifluoropropyl)amino]piperidine-1-carboxylate;
tert-Butyl 4-[{[6-(3-{[(2-hydroxyethyl)amino]carbonyl}phenyl)pyridin-3-yl]-methyl}(3,3,3-trifluoropropyl)amino]piperidine-1-carboxylate;
tert-Butyl 4-[({6-[3-(aminocarbonyl)phenyl]pyridin-3-yl}methyl)(3,3,3-trifluoropropyl)amino]piperidine-1-carboxylate;
1-(2,2-Dimethylpropanoyl)-N-methyl-N-({6-[4-(methylsulfonyl)phenyl]-pyridin-3-yl}methyl)piperidin-4-amine;
tert-Butyl (3R*,4S*)-3-methyl-4-[methyl({6-[4-(methylsulfonyl)phenyl]-pyridin-3-yl}methyl)amino]piperidine-1-carboxylate;
tert-Butyl (3S*,4S*)-3-methyl-4-[methyl({6-[4-(methylsulfonyl)phenyl]-pyridin-3-yl}methyl)amino]piperidine-1-carboxylate;
1-[3-(4-Fluorophenyl)propanoyl]-N-methyl-N-({6-[4-(methylsulfonyl)phenyl]-pyridin-3-yl}methyl)piperidin-4-amine;
1-(4-Isopropoxybenzoyl)-N-methyl-N-({6-[4-(methylsulfonyl)phenyl]pyridin-3-yl}methyl)piperidin-4-amine;
1-(3,4-Dichlorobenzoyl)-N-methyl-N-({6-[4-(methylsulfonyl)phenyl]pyridin-3-yl}methyl)piperidin-4-amine;
1-[4-(4-Fluorophenyl)butanoyl]-N-methyl-N-({6-[4-(methylsulfonyl)phenyl]-pyridin-3-yl}methyl)piperidin-4-amine;
N-Methyl-N-({6-[4-(methylsulfonyl)phenyl]pyridin-3-yl}methyl)-1-{[3-(trifluoromethyl)phenyl]acetyl}piperidin-4-amine;
1-(Cyclohexylacetyl)-N-methyl-N-({6-[4-(methylsulfonyl)phenyl]pyridin-3-yl}methyl)piperidin-4-amine;
1-Benzoyl-N-methyl-N-({6-[4-(methylsulfonyl)phenyl]pyridin-3-yl}methyl)-piperidin-4-amine;
N-Methyl-1-(3-methylbutanoyl)-N-({6-[4-(methylsulfonyl)phenyl]pyridin-3-yl}methyl)piperidin-4-amine;
1-(Cyclohexylcarbonyl)-N-methyl-N-({6-[4-(methylsulfonyl)phenyl]pyridin-3-yl}methyl)piperidin-4-amine;
1-(3,3-Dimethylbutanoyl)-N-methyl-N-({6-[4-(methylsulfonyl)phenyl]pyridin-3-yl}methyl)piperidin-4-amine;
1-(2,4-Dichlorobenzoyl)-N-methyl-N-({6-[4-(methylsulfonyl)phenyl]pyridin-3-yl}methyl)piperidin-4-amine;
1-(2,4-Difluorobenzoyl)-N-methyl-N-({6-[4-(methylsulfonyl)phenyl]pyridin-3-yl}methyl)piperidin-4-amine;
1-(2,5-Difluorobenzoyl)-N-methyl-N-({6-[4-(methylsulfonyl)phenyl]pyridin-3-yl}methyl)piperidin-4-amine;
1-(2-Fluorobenzoyl)-N-methyl-N-({6-[4-(methylsulfonyl)phenyl]pyridin-3-yl}-methyl)piperidin-4-amine;
1-(3-Fluorobenzoyl)-N-methyl-N-({6-[4-(methylsulfonyl)phenyl]pyridin-3-yl}-methyl)piperidin-4-amine;
1-(4-Fluorobenzoyl)-N-methyl-N-({6-[4-(methylsulfonyl)phenyl]pyridin-3-yl}-methyl)piperidin-4-amine;
1-(3-methoxybenzoyl)-N-methyl-N-({6-[4-(methylsulfonyl)phenyl]pyridin-3-yl}methyl)piperidin-4-amine;
1-(3-Chloro-4-methoxybenzoyl)-N-methyl-N-({6-[4-(methylsulfonyl)phenyl]-pyridin-3-yl}methyl)piperidin-4-amine;
tert-Butyl 4-[({6-[4-(methylsulfonyl)phenyl]pyridin-3-yl}amino)methyl]-piperidine-1-carboxylate;
tert-Butyl 4-({6-[4-(methyl sulfonyl)phenyl]pyridin-3-yl}methoxy)piperidine-1-carboxylate;
tert-Butyl 4-[(6-{4-[(methoxycarbonyl)amino]phenyl}pyridin-3-yl)methoxy]-piperidine-1-carboxylate;
5-[({1-[4-(4-Fluorophenyl)butanoyl]piperidin-4-yl}oxy)methyl]-2-[4-(methylsulfonyl)phenyl]pyridine;
5-({[1-(Cyclohexylacetyl)piperidin-4-yl]oxy}methyl)-2-[4-(methylsulfonyl)-phenyl]pyridine;
tert-Butyl 4-[({6-[4-(methylsulfonyl)phenyl]pyridin-3-yl}oxy)methyl]-piperidine-1-carboxylate;
Isobutyl 4-[({6-[4-(methylsulfonyl)phenyl]pyridin-3-yl}oxy)methyl]piperidine-1-carboxylate;
Ethyl 4-[({6-[4-(methylsulfonyl)phenyl]pyridin-3-yl}oxy)methyl]piperidine-1-carboxylate;
tert-Butyl 4-{[(6-{4-[(2-hydroxyethyl)sulfonyl]phenyl}pyridin-3-yl)oxy]-methyl}piperidine-1-carboxylate;
{4-(5-[1-(tert-Butoxycarbonyl)piperidin-4-yl]methoxy}pyridin-2-yl)benzoic acid;
tert-Butyl 4-{[(6-{3-fluoro-4-[(propylamino)carbonyl]phenyl}pyridin-3-yl)oxy]methyl}piperidine-1-carboxylate;
tert-Butyl 4-{[(6-{4-[(cyclopropylamino)carbonyl]phenyl}pyridin-3-yl)oxy]-methyl}piperidine-1-carboxylate;
tert-Butyl 4-{[(6-{4-[(ethylamino)carbonyl]phenyl}pyridin-3-yl)oxy]methyl}-piperidine-1-carboxylate;
tert-Butyl 4-{[(6-{4-[(methylamino)carbonyl]phenyl}pyridin-3-yl)oxy]-methyl}piperidine-1-carboxylate;
tert-Butyl 4-({[6-(4-{[(2-cyanoethyl)amino]carbonyl}phenyl)pyridin-3-yl]-oxy}methyl)piperidine-1-carboxylate;
tert-Butyl 4-[({6-[4-(5,6-dihydro-4H-1,3-oxazin-2-yl)phenyl]pyridin-3-yl}-oxy)methyl]piperidine-1-carboxylate;
tert-Butyl 4-[({6-[4-(acetylamino)phenyl]pyridin-3-yl}oxy)methyl]piperidine-1-carboxylate;
tert-Butyl 4-({[6-(4-{[(2-methoxyethyl)amino]carbonyl}phenyl)pyridin-3-yl]-oxy}methyl)piperidine-1-carboxylate;
tert-Butyl 4-({[6-(4-{[(2-hydroxyethyl)amino]carbonyl}phenyl)pyridin-3-yl]-oxy}methyl)piperidine-1-carboxylate;
tert-Butyl 4-({[6-(4-{[(2-hydroxybutyl)amino]carbonyl}phenyl)pyridin-3-yl]-oxy}methyl)piperidine-1-carboxylate;
tert-Butyl 4-{[(6-{4-[(acetylamino)methyl]phenyl}pyridin-3-yl)oxy]methyl}-piperidine-1-carboxylate;
tert-Butyl 4-{[(6-{4-[(4-methylpiperazin-1-yl)carbonyl]phenyl}pyridin-3-yl)-oxy]methyl}piperidine-1-carboxylate;
tert-Butyl 4-({[6-(4-{[2-(hydroxymethyl)morpholin-4-yl]carbonyl}phenyl)-pyridin-3-yl]oxy}methyl)piperidine-1-carboxylate;
tert-Butyl 4-({[6-(4-{[(2-amino-2-oxoethyl)amino]carbonyl}phenyl)pyridin-3-yl]oxy}methyl)piperidine-1-carboxylate;
4-{[4-(5-{[1-(tert-Butoxycarbonyl)piperidin-4-yl]methoxy}pyridin-2-yl)-phenyl]amino}-4-oxobutanoic acid;
tert-Butyl 4-[({6-[4-(cyanomethyl)phenyl]pyridin-3-yl}oxy)methyl]piperidine-1-carboxylate;
tert-Butyl 4-[({6-[4-(methylsulfinyl)phenyl]pyridin-3-yl}oxy)methyl]-piperidine-1-carboxylate;
tert-Butyl 4-{[(6-{3-[(acetylamino)methyl]phenyl}pyridin-3-yl)oxy]methyl}-piperidine-1-carboxylate;
tert-Butyl 4-[({6-[3-(cyanomethyl)phenyl]pyridin-3-yl}oxy)methyl]piperidine-1-carboxylate;
tert-Butyl 4-[({6-[2-fluoro-4-(methylsulfonyl)phenyl]pyridin-3-yl}oxy)-methyl]piperidine-1-carboxylate;
tert-Butyl 4-[({6-[4-(aminocarbonyl)-3-fluorophenyl]pyridin-3-yl}oxy)-methyl]piperidine-1-carboxylate;
tert-Butyl 4-[({6-[4-(azetidin-1-ylsulfonyl)phenyl]pyridin-3-yl}oxy)methyl]-piperidine-1-carboxylate;
[4-(5-{[1-(tert-Butoxycarbonyl)piperidin-4-yl]methoxy}pyridin-2-yl)phenyl]-acetic acid;
tert-Butyl 4-{[(6-{4-[2-(4-hydroxypiperidin-1-yl)-2-oxoethyl]phenyl}pyridin-3-yl)oxy]methyl}piperidine-1-carboxylate;
tert-Butyl 4-({[6-(4-{2-[2-(hydroxymethyl)morpholin-4-yl]-2-oxoethyl}-phenyl)pyridin-3-yl]oxy}methyl)piperidine-1-carboxylate;
tert-Butyl 4-{[(6-{4-[2-(3-hydroxyazetidin-1-yl)-2-oxoethyl]phenyl}pyridin-3-yl)oxy]methyl}piperidine-1-carboxylate;
2-{4-[({6-[4-(Methylsulfonyl)phenyl]pyridin-3-yl}oxy)methyl]piperidin-1-yl}-pyrimidine;
tert-Butyl 4-({6-[4-(methylsulfonyl)phenyl]pyridin-3-yl}acetyl)piperazine-1-carboxylate;
tert-Butyl 4-(2-{6-[4-(methylsulfonyl)phenyl]pyridin-3-yl}ethyl)piperazine-1-carboxylate;
1-(4-Isopropoxybenzoyl)-4-(2-{6-[4-(methylsulfonyl)phenyl]pyridin-3-yl}-ethyl)piperazine; and
Isobutyl 4-(2-{6-[4-(methylsulfonyl)phenyl]pyridin-3-yl}ethyl)piperazine-1-carboxylate.

26. A method for the treatment or prophylaxis of disorders relating to GPR119 activity which comprises administering to a mammal, including man, in need of such treatment an effective amount of a compound according to claim 1, wherein said disorders relating to GPR119 activity are selected from the group consisting of Type 1 diabetes, Type 2 diabetes, inadequate glucose tolerance, insulin resistance, hyperglycemia, hyperlipidemia, hypercholesterolemia, dyslipidemia, syndrome X, metabolic syndrome, obesity, hypertension, chronic systemic inflammation, retinopathy, neuropathy, nephropathy, atherosclerosis, reduced fibrinolysis, and endothelial dysfunction.

27. A pharmaceutical formulation containing a compound according to claim 1 as active ingredient in combination with a pharmaceutically acceptable diluent or carrier.

28. A method for the treatment or prophylaxis of disorders relating to GPR119 activity which comprises administering to a mammal, including man, in need of such treatment an effective amount of a compound according to claim 1 in combination with a DPP-IV inhibitor.

29. The pharmaceutical formulation according to claim 27 which in addition comprises a DPP-IV inhibitor.

30. The method according to claim 28, wherein said disorders relating to GPR119 activity are selected from the group consisting of Type 1 diabetes, Type 2 diabetes, inadequate glucose tolerance, insulin resistance, hyperglycemia, hyperlipidemia, hypercholesterolemia, dyslipidemia, syndrome X, metabolic syndrome, obesity, hypertension, chronic systemic inflammation, retinopathy, neuropathy, nephropathy, atherosclerosis, reduced fibrinolysis, and endothelial dysfunction.

31. The compound according to claim 9, wherein R¹⁰ is independently selected from hydrogen and methyl.
